(19) United States
(12) Patent Application Publication
(10) Pub. No.: US 2026/0308157 A1
(43) Pub. Date: Oct. 8, 2026

(54) Pyrrolopyrazine Compounds, Preparation Thereof and Therapeutic Uses Thereof

(71) Applicant: Sanofi, Paris (FR)

(72) Inventors: Franck Slowinski, Paris (FR); Jidong Zhang, Paris (FR); Amélie Dommergue, Paris (FR)

(21) Appl. No.: 19/475,923

(22) PCT Filed: Apr. 18, 2024
(86) PCT No.: PCT/EP2024/060592

§ 371 Date: Oct. 16, 2025

(30) Foreign Application Priority Data
Apr. 19, 2023 (EP) 23315084.6

(51) Int. Cl. C07D 487/04; A61K 31/4985; A61K 31/5377

(52) U.S. Cl. CPC C07D 487/04; A61K 31/4985; A61K 31/5377

(57) ABSTRACT

Compounds are provided which can inhibit ERK5. Also provided are pharmaceutical compositions and medical uses of the same, including the use in treating or preventing conditions such as cancers.

Compounds are provided which can inhibit ERK5. Also provided are pharmaceutical compositions and medical uses of the same, including the use in treating or preventing conditions such as cancers.

### SUMMARY

The mitogen-activated protein kinase (MAPK) cascade is a highly-conserved cellular pathway which transmits signals from the cell surface to the nucleus. The pathway plays an important role in cell proliferation, differentiation, and migration and it is well known to be involved in the development of cancer. Proteins in the pathway include the extracellular signal-regulated kinase (ERK) proteins; among those, ERK5 (expressed from the MAPK7 gene) plays an important role in cell proliferation, as well as epithelial development and neural differentiation (see, e.g., Nishimoto et al., EMBO Reports (2006) 7(8):782-786). ERK5 is unique among the ERK proteins, having a large C-terminal domain which contains a transcriptional activation domain (TAD) as well as a nuclear localization signal and two proline-rich regions (see, e.g., Guo et al., Exp Ther Med. (2020) 19:1997-2007).

Autophosphorylation of the TAD is required for transcriptional activation (see, e.g., Morimoto et al., J Biol Chem. (2007) 282(49):35449-35456).

ERK5 plays an important role in controlling cell proliferation and cell cycle progression, for example via direct or indirect phosphorylation of MEF2C, cMYC, SGK1, RSK, FOS, and FRA1 among others (see, e.g., Paudel et al., Int J Mol Sci. (2021) 22:7594-7614; Terasawa et al., Genes to Cells (2003) 8(3):263-273). The involvement of ERK5 in numerous biological pathways means that its activity is associated with many aspects of cancer progression, including tumour angiogenesis, metastasis, inflammation, sustained proliferation, and evasion of growth suppression. It therefore presents an attractive target for modulating disease pathology and treatment in a wide range of conditions. In previous studies, ERK5 inhibition or down-regulation has been shown to block tumorigenesis in murine leukaemia cells, reduce growth of chronic myeloid leukaemia cells, inhibit growth of breast cancer and multiple myeloma cells, suppress colon cancer cell proliferation, and have to have an impact on renal cell carcinoma, mesothelioma, adenocarcinoma, neuroblastoma and hepatocellular carcinoma cell growth or survival, among others (see, e.g., Stecca et al., Int J Mol Sci. (2019) 20:1426-1446).

ERK5 inhibition thus represents a promising approach to tackle a broad range of cancers. Several ERK5 inhibitors have been developed and some are under clinical review. For example, WO 2019/170543 (Bayer AG and Bayer Pharma AG) discloses compounds which are said to be active as ERK5 inhibitors in the μM to nM concentration range.

Despite recent progress in cancer treatment with the development of targeted therapies and immunotherapies, not all cancer patients can be offered an efficient therapeutic solution. There is therefore a need to identify and develop new drugs. The present disclosure seeks to address this need by providing novel compounds for use as ERK5 inhibitors and for the treatment of ERK5 related diseases and conditions.

Accordingly, a first aspect provides a compound, being of Formula (I)

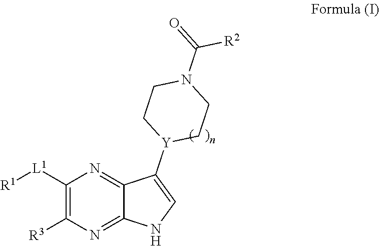

or a pharmaceutically acceptable salt thereof, wherein:
R¹ is selected from —(C₁-C₆)alkyl, —(C₃-C₇)cycloalkyl, and 4- to 10-membered heterocycloalkyl, wherein R¹ is optionally substituted with one or more occurrences of R^{A}
   wherein each R^{A} is independently selected from halo, —OH, oxo, —NH₂, ═N—OH, —(C₁-C₃)alkyl, —O(C₁-C₃)alkyl, and —(C₃-C₆)cycloalkyl, wherein each occurrence of —(C₁-C₃)alkyl is optionally substituted by one or more groups independently selected from halo and —OH;
L¹ is selected from a direct bond, —O—, and —NH—;
R² is selected from —(C₃-C₆)cycloalkyl, and —(C₆-C₁₀)aryl, wherein R² is optionally substituted with one, two, or three occurrences of R^{B},
   wherein each R^{B} is independently selected from halo, —NH₂, —SF₅, —(C₁-C₃)alkyl, —O(C₁-C₃)alkyl, and —O(C₃-C₆)cycloalkyl, wherein each occurrence of —(C₁-C₃)alkyl and —O(C₁-C₃)alkyl is optionally substituted by one or more groups independently selected from halo and —OH;
R³ is selected from —H, (C₁-C₃)alkyl (e.g., —CH₃), and —OH;
Y is CH, or N; and
n is 0, or 1.

In embodiments, R¹ is selected from:

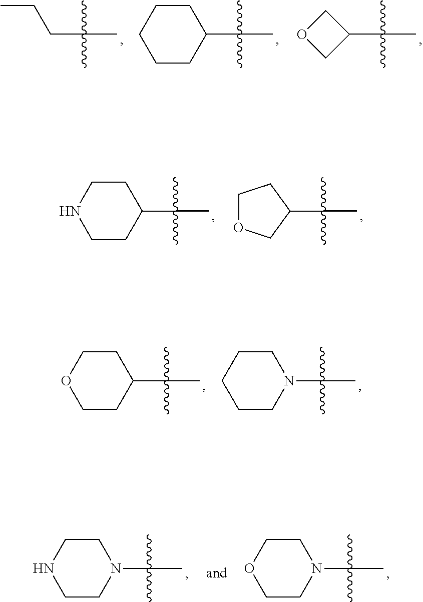

wherein R¹ is optionally substituted by one or two occurrences of R^{A} as defined hereinbefore. In embodiments, each R^{A} is independently selected from the group consisting of —F, —OH, methyl, —OCH₃, and cyclopropyl.

In embodiments, the compound is a compound of Formula (I-G)

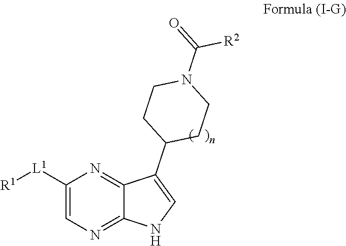

or a pharmaceutically acceptable salt thereof, wherein R¹, R², L¹, and n are as defined hereinbefore.

Another aspect provides a compound, being of Formula (II)

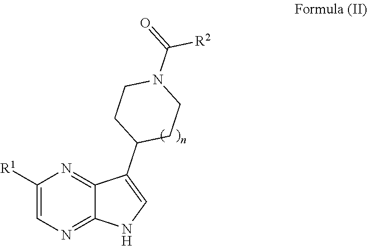

or a pharmaceutically acceptable salt thereof, wherein:
R¹ is selected from —(C₁-C₆)alkyl, —(C₃-C₇)cycloalkyl, and 4- to 10-membered heterocycloalkyl, wherein R¹ is optionally substituted with one or more occurrences of R^{A}wherein each R^{A} is independently selected from halo (e.g., —F), —OH, —(C₁-C₃)alkyl, —O(C₁-C₃)alkyl, and —(C₃-C₆)cycloalkyl;
R² is selected from —(C₃-C₆)cycloalkyl, and —(C₆-C₁₀)aryl, wherein R² is optionally substituted with one or two occurrences of R^{B},
   wherein each R^{B} is independently selected from halo, —NH₂, —SF₅, —(C₁-C₃)alkyl, —O(C₁-C₃)alkyl, and —O(C₃-C₆)cycloalkyl, wherein each occurrence of —(C₁-C₃)alkyl and —O(C₁-C₃)alkyl is substituted by one or more groups independently selected from halo and —OH; and
n is 0, or 1.

In embodiments, R² is —(C₆-C₁₀)aryl substituted with one or two occurrences of R^{B}, wherein each R^{B} is independently selected from halo (e.g., —F), —NH₂, —SF₅, —OCF₃, —O-cyclopropyl, —C(OH)(CF₃)₂, and —CF₂CF₃.

A further aspect provides a compound, being of Formula (III-A)

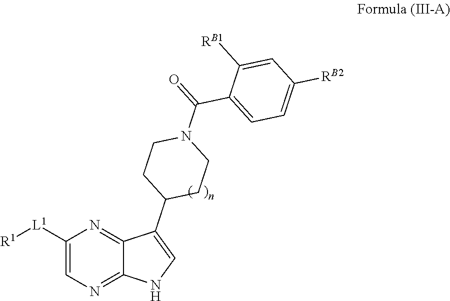

or a pharmaceutically acceptable salt thereof, wherein R¹, L¹, and n are as defined hereinbefore, and wherein:
R^{B1} is either —H, or is selected from the group consisting of —NH₂, and —F; and
R^{B2} is selected from the group consisting of —OCF₃, —SF₅, —CF₂CF₃, —C(OH)(CF₃)₂, and —O-cyclopropyl.

A further aspect provides a compound, being of Formula (IV-A) or Formula (V-A)

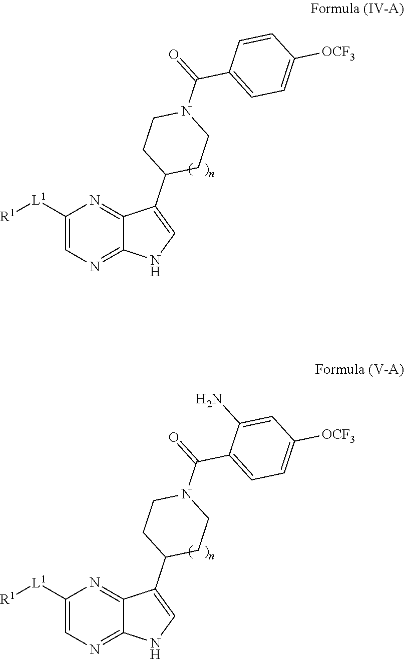

or a pharmaceutically acceptable salt thereof, wherein R¹, L¹, and n are as defined hereinbefore.

A further aspect provides a compound, being of Formula (VI), Formula (VII), or Formula (VIII)

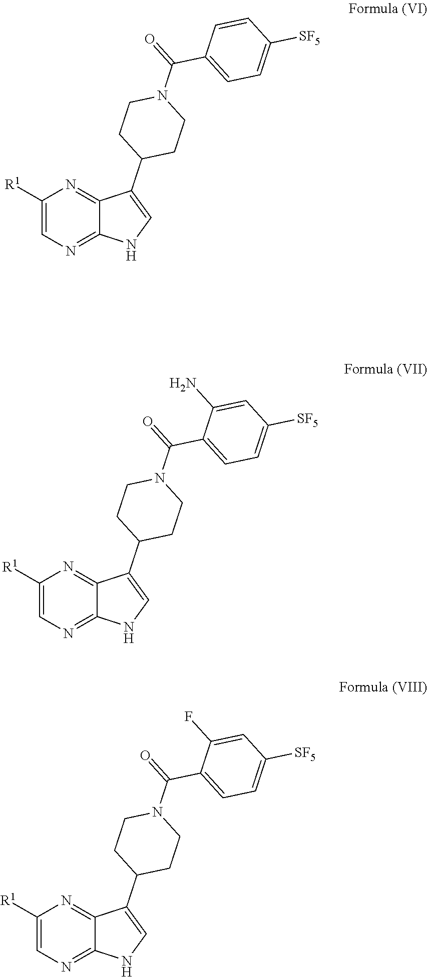

or a pharmaceutically acceptable salt thereof, wherein R¹ is as defined hereinbefore.

A further aspect provides a compound selected from the group consisting of:
[2-amino-4-(trifluoromethoxy)phenyl]-[4-[2-(1-methyl-4-piperidyl)-5H-pyrrolo[2,3-b]pyrazin-7-yl]-1-piperidyl]methanone,
[2-amino-4-(trifluoromethoxy)phenyl]-[4-(2-tetrahydropyran-4-yl-5H-pyrrolo[2,3-b]pyrazin-7-yl)-1-piperidyl]methanone,
[4-(2-tetrahydropyran-4-yl-5H-pyrrolo[2,3-b]pyrazin-7-yl)-1-piperidyl]-[4-(trifluoromethoxy)phenyl]methanone,
[4-[2-(1-methyl-4-piperidyl)-5H-pyrrolo[2,3-b]pyrazin-7-yl]-1-piperidyl]-[4-(trifluoromethoxy)phenyl]methanone,
[4-(2-tetrahydrofuran-3-yl-5H-pyrrolo[2,3-b]pyrazin-7-yl)-1-piperidyl]-[4-(trifluoromethoxy)phenyl]methanone,
[4-[2-(3-methoxypropyl)-5H-pyrrolo[2,3-b]pyrazin-7-yl]-1-piperidyl]-[4-(trifluoromethoxy)phenyl]methanone,
[2-amino-4-(trifluoromethoxy)phenyl]-[4-[2-(3-methoxypropyl)-5H-pyrrolo[2,3-b]pyrazin-7-yl]-1-piperidyl]methanone,
[4-(2-cyclohexyl-5H-pyrrolo[2,3-b]pyrazin-7-yl)-1-piperidyl]-[4-(trifluoromethoxy)phenyl]methanone,
[2-amino-4-(trifluoromethoxy)phenyl]-[4-(2-cyclohexyl-5H-pyrrolo[2,3-b]pyrazin-7-yl)-1-piperidyl]methanone,
[2-amino-4-(trifluoromethoxy)phenyl]-[4-[2-(1-cyclopropyl-4-piperidyl)-5H-pyrrolo[2,3-b]pyrazin-7-yl]-1-piperidyl]methanone,
[4-[2-(1-cyclopropyl-4-piperidyl)-5H-pyrrolo[2,3-b]pyrazin-7-yl]-1-piperidyl]-[4-(trifluoromethoxy)phenyl]methanone,
[2-amino-4-(trifluoromethoxy)phenyl]-[4-(2-tetrahydrofuran-3-yl-5H-pyrrolo[2,3-b]pyrazin-7-yl)-1-piperidyl]methanone,
[4-[2-(1-methyl-4-piperidyl)-5H-pyrrolo[2,3-b]pyrazin-7-yl]-1-piperidyl]-[4-(pentafluoro-λ⁶-sulfanyl)phenyl]methanone,
[2-amino-4-(pentafluoro-λ⁶-sulfanyl)phenyl]-[4-[2-(1-methyl-4-piperidyl)-5H-pyrrolo[2,3-b]pyrazin-7-yl]-1-piperidyl]methanone,
[4-(pentafluoro-λ⁶-sulfanyl)phenyl]-[4-(2-tetrahydropyran-4-yl-5H-pyrrolo[2,3-b]pyrazin-7-yl)-1-piperidyl]methanone,
[2-amino-4-(pentafluoro-λ⁶-sulfanyl)phenyl]-[4-(2-tetrahydropyran-4-yl-5H-pyrrolo[2,3-b]pyrazin-7-yl)-1-piperidyl]methanone,
[2-amino-4-(pentafluoro-λ⁶-sulfanyl)phenyl]-[4-[2-(1-cyclopropyl-4-piperidyl)-5H-pyrrolo[2,3-b]pyrazin-7-yl]-1-piperidyl]methanone,
[4-[2-(1-cyclopropyl-4-piperidyl)-5H-pyrrolo[2,3-b]pyrazin-7-yl]-1-piperidyl]-[4-(pentafluoro-λ⁶-sulfanyl)phenyl]methanone,
[4-(cyclopropoxy)phenyl]-[4-(2-tetrahydropyran-4-yl-5H-pyrrolo[2,3-b]pyrazin-7-yl)-1-piperidyl]methanone,
[4-(1,1,2,2,2-pentafluoroethyl)phenyl]-[4-(2-tetrahydropyran-4-yl-5H-pyrrolo[2,3-b]pyrazin-7-yl)-1-piperidyl]methanone,
cyclohexyl-[4-(2-tetrahydropyran-4-yl-5H-pyrrolo[2,3-b]pyrazin-7-yl)-1-piperidyl]methanone,
[2-amino-4-(trifluoromethoxy)phenyl]-[3-(2-tetrahydropyran-4-yl-5H-pyrrolo[2,3-b]pyrazin-7-yl)pyrrolidine-1-yl]methanone,
[3-(2-tetrahydropyran-4-yl-5H-pyrrolo[2,3-b]pyrazin-7-yl)pyrrolidin-1-yl]-[4-(trifluoromethoxy)phenyl]methanone,
[2-fluoro-4-(pentafluoro-λ⁶-sulfanyl)phenyl]-[4-(2-tetrahydropyran-4-yl-5H-pyrrolo[2,3-b]pyrazin-7-yl)-1-piperidyl]methanone,
[2-amino-4-(trifluoromethoxy)phenyl]-[4-[2-(oxetan-3-ylamino)-5H-pyrrolo[2,3-b]pyrazin-7-yl]-1-piperidyl]methanone,
[2-amino-4-(trifluoromethoxy)phenyl]-[4-[2-[tetrahydrofuran-3-yl]oxy-5H-pyrrolo[2,3-b]pyrazin-7-yl]-1-piperidyl]methanone,
trans-[2-amino-4-(trifluoromethoxy)phenyl]-[4-[2-(4-hydroxycyclohexyl)-5H-pyrrolo[2,3-b]pyrazin-7-yl]-1-piperidyl]methanone,
[4-[2-(4-methylpiperazin-1-yl)-5H-pyrrolo[2,3-b]pyrazin-7-yl]-1-piperidyl]-[4-(trifluoromethoxy)phenyl]methanone,
[4-[2-[[tetrahydrofuran-3-yl]amino]-5H-pyrrolo[2,3-b]pyrazin-7-yl]-1-piperidyl]-[4-(trifluoromethoxy)phenyl]methanone,
[2-amino-4-(trifluoromethoxy)phenyl]-[4-[2-[[tetrahydrofuran-3-yl]amino]-5H-pyrrolo[2,3-b]pyrazin-7-yl]-1-piperidyl]methanone,
[4-[2-(tetrahydropyran-4-ylamino)-5H-pyrrolo[2,3-b]pyrazin-7-yl]-1-piperidyl]-[4-(trifluoromethoxy)phenyl]methanone,
[2-amino-4-(trifluoromethoxy)phenyl]-[4-[2-(tetrahydropyran-4-ylamino)-5H-pyrrolo[2,3-b]pyrazin-7-yl]-1-piperidyl]methanone,
[4-(2-morpholino-5H-pyrrolo[2,3-b]pyrazin-7-yl)-1-piperidyl]-[4-(trifluoromethoxy)phenyl]methanone,
[4-[2-(4,4-difluoro-1-piperidyl)-5H-pyrrolo[2,3-b]pyrazin-7-yl]-1-piperidyl]-[4-(trifluoromethoxy)phenyl]methanone,
[4-(2-tetrahydropyran-4-yl-5H-pyrrolo[2,3-b]pyrazin-7-yl)-1-piperidyl]-[4-[2,2,2-trifluoro-1-hydroxy-1-(trifluoromethyl)ethyl]phenyl]methanone,
[2-amino-4-(pentafluoro-λ⁶-sulfanyl)phenyl]-[3-(2-tetrahydropyran-4-yl-5H-pyrrolo[2,3-b]pyrazin-7-yl)pyrrolidin-1-yl]methanone,
[2-amino-4-(trifluoromethoxy)phenyl]-[4-[2-(oxetan-3-yloxy)-5H-pyrrolo[2,3-b]pyrazin-7-yl]-1-piperidyl]methanone,
   and the pharmaceutically acceptable salts thereof.

A further aspect provides a pharmaceutical composition comprising the compound defined hereinbefore and at least one pharmaceutically acceptable excipient or carrier.

A further aspect provides a compound, or a pharmaceutical composition, as defined hereinbefore for use in therapy.

A further aspect provides a compound, or a pharmaceutical composition, as defined hereinbefore for use in the treatment or prevention of cancer.

In embodiments, the cancer is characterized by increased MAPK7 expression and/or increased ERK5 activity.

In embodiments, the cancer is selected from leukaemia, breast cancer, multiple myeloma, colon cancer, colorectal cancer, lung cancer, pancreatic cancer, renal cell carcinoma, mesothelioma, adenocarcinoma, neuroblastoma, melanoma, and hepatocellular carcinoma.

### DETAILED DESCRIPTION

Although specific embodiments of the present disclosure will now be described with reference to the description and examples, it should be understood that such embodiments are by way of example only and merely illustrative of but a small number of the many possible specific embodiments which can represent applications of the principles of the present disclosure. Various changes and modifications will be obvious to those of skill in the art given the benefit of the present disclosure and are deemed to be within the spirit and scope of the present disclosure as further defined in the appended claims.

### Definitions

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, exemplary methods, devices, and materials are now described. All technical and patent publications cited herein are incorporated herein by reference in their entirety.

The practice of the present disclosure will employ, unless otherwise indicated, conventional techniques of chemical synthesis, tissue culture, immunology, molecular biology, microbiology, cell biology, recombinant DNA, etc., which are within the skill of the art. See, e.g., Michael R. Green and Joseph Sambrook, Molecular Cloning (4^{th} ed., Cold Spring Harbor Laboratory Press 2012); the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Sohail (ed.) (2004) Gene Silencing by RNA Interference: Technology and Application (CRC Press).

All numerical designations, e.g., pH, temperature, time, concentration, molecular weight, etc., including ranges, are approximations which are varied (+) or (−) by increments of, e.g., 0.1 or 1.0, where appropriate. It is to be understood, although not always explicitly stated, that all numerical designations are preceded by the term “about”, which is used to denote a conventional level of variability. For example, a numerical designation which is “about” a given value may vary by ±10% of said value; alternatively, the variation may be ±5%, ±2%, or ±1% of the value. It also is to be understood, although not always explicitly stated, that the reagents described herein are merely exemplary and that equivalents of such are known in the art.

As used in the specification and claims, the singular forms “a”, “an”, and “the” include plural references unless the context clearly dictates otherwise. For example, the term “a cell” includes a plurality of cells, including mixtures thereof. Unless specifically stated or obvious from context, as used herein, the term “or” is understood to be inclusive. The term “including” is used herein to mean, and is used interchangeably with, the phrase “including but not limited to”.

As used herein, the term “comprising” or “comprises” is intended to mean that the compositions and methods include the recited elements, without excluding other elements. “Consisting essentially of” when used to define compositions and methods, shall mean excluding other elements of any essential significance for the stated purpose. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure or process steps to produce a composition or achieve an intended result. Embodiments defined by each of these transition terms are within the scope of this disclosure. Use of the term “comprising” herein is intended to encompass, and to disclose, the corresponding statements in which the term “comprising” is replaced by “consisting essentially of” or “consisting of”.

A “subject,” “individual”, or “patient” is used interchangeably herein, and refers to a vertebrate, such as a mammal. Mammals include, but are not limited to, rodents, farm animals, sport animals, pets, and primates; for example murines, rats, rabbit, simians, bovines, ovines, porcines, canines, felines, equines, and humans. In a particular embodiment, the mammal is a human.

“Administering” is defined herein as a means of providing an agent or a composition containing the agent to a subject in a manner that results in the agent being contacted with (e.g., being inside) the subject's body. Such an administration can be by any route including, without limitation, oral, transdermal (e.g., by the vagina, rectum, or oral mucosa), by injection (e.g., subcutaneous, intravenous, parenteral, intraperitoneal, or into the central nervous system), or by inhalation (e.g., oral or nasal). Administration may also involve providing a substance or composition to a part of the surface of the subject's body, for example by topical administration to the skin. Pharmaceutical preparations are, of course, given by forms suitable for each administration route.

“Treating” or “treatment” of a disease includes: (1) preventing the disease, i.e. causing the clinical symptoms of the disease not to develop in a patient that may be predisposed to the disease but does not yet experience or display symptoms of the disease; (2) inhibiting the disease, i.e. arresting or reducing the development of the disease or its clinical symptoms; and/or (3) relieving the disease, i.e. causing regression of the disease or its clinical symptoms.

The term “suffering” as it relates to the term “treatment” refers to a patient or individual who has been diagnosed with or is predisposed to the disease. A patient may also be referred to being “at risk of suffering” from a disease because of a history of disease in their family lineage or because of the presence of genetic mutations associated with the disease. A patient at risk of a disease has not yet developed all or some of the characteristic pathologies of the disease.

An “effective amount” or “therapeutically effective amount” is an amount sufficient to effect beneficial or desired results. An effective amount can be administered in one or more administrations, applications, or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including, for example, the activity of the specific compound employed, the age, body weight, general health, sex, and diet of the subject, the time of administration, the rate of excretion, the drug combination, the severity of the particular disorder being treated and the form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to treat (e.g., improve) one or more symptoms associated with the condition. The total daily dose may be administered in single or divided doses and may, at the physician's discretion, fall outside of the typical range given herein.

As used herein, the terms “increased” and “elevated” are used interchangeably and encompass any measurable increase in a biological function and/or a biological activity and/or a concentration. For example, an increase can be by at least about 10%, e.g. at least about 15%, 20%, 25%, 30%, 35%, 40%, 45%, 50%, 55%, 60%, 65%, 70%, 75%, 80%, 85%, 90%, or 95%, such as at least about 95%, 96%, 97%, 98%, 99%, or 100%. Thus, an increase can be by at least about 2-fold, 3-fold, 4-fold, 5-fold, 6-fold, 7-fold, 8-fold, 9-fold, or 10-fold, such as at least about 20-fold, 25-fold, 50-fold, 100-fold, or higher, relative to a control or baseline amount or function, or activity, or concentration.

As used herein, the terms “increased expression” and/or “increased activity” of a substance, such as ERK5, in a sample or cancer or patient, typically refers to an increase in the amount of the substance (e.g., of the MAPK7 gene product or ERK5 protein), although it may also denote an increase in the biological activity of the substance (e.g., constitutive activation of phosphorylation and/or reduced discrimination of phosphorylation sites of ERK5). For example, an increase can be by an amount of about 5%, e.g., about 10%, 15%, 20%, 25%, 30%, 35%, 40%, 45%, 50%, 55%, 60%, 65%, 70%, 75%, 80%, 85%, 90%, or 95%, such as about 96%, 97%, 98%, 99%, or 100%. Thus, the increase can be about 2-fold, 3-fold, 4-fold, 5-fold, 6-fold, 7-fold, 8-fold, 9-fold, or 10-fold, such as about 20-fold, 25-fold, 50-fold, 100-fold, or higher, relative to the amount (or activity) of the substance, such as ERK5, in a control sample or control samples, such as an individual or group of individuals who are not suffering from the disease or disorder (e.g. cancer) or an internal control, as determined by techniques known in the art. A subject can also be determined to have an “increased expression” or “increased activity” of ERK5 if the expression and/or activity of ERK5 is increased by one standard deviation, two standard deviations, three standard deviations, four standard deviations, five standard deviations, or more, relative to the mean (average) or median amount of ERK5 in a control group of samples or a baseline group of samples or a retrospective analysis of patient samples. As practiced in the art, such control or baseline expression levels can be previously determined, or measured prior to the measurement in the sample or cancer or subject, or can be obtained from a database of such control samples.

As used herein, the term “pharmaceutically acceptable excipient” encompasses any of the standard pharmaceutical excipients, for example as described in Remington's Pharmaceutical Sciences (20th ed., Mack Publishing Co. 2000). Such excipients include carriers such as a phosphate buffered saline solution, water, and emulsions, such as an oil/water or water/oil emulsion, and various types of wetting agents. Pharmaceutical compositions also can include stabilizers, preservatives, adjuvants, fillers, binders, lubricants, and the like.

As used herein, the term “alkyl” means a saturated linear or branched free radical consisting essentially of carbon atoms and a corresponding number of hydrogen atoms. Exemplary alkyl groups include methyl, ethyl, n-propyl, isopropyl, n-butyl, isobutyl, etc. Other alkyl groups will be readily apparent to those of skill in the art given the benefit of the present disclosure. The terms “(C₁-C₃)alkyl”, “(C₁-C₆)alkyl”, etc., have equivalent meanings, i.e., a saturated linear or branched free radical consisting essentially of 1 to 3 (or 1 to 6) carbon atoms and a corresponding number of hydrogen atoms. The definition of “alkyl” also applies in the context of other groups which comprise alkyl groups, such as “—O(C₁-C₃)alkyl”. The term “haloalkyl” means an alkyl group which is substituted by one or more halogens. Exemplary haloalkyl groups include trifluoromethyl, trifluoroethyl, difluoroethyl, pentafluoroethyl, chloromethyl, etc. One or more carbon atoms in the backbone of the alkyl group may be substituted by (or bonded to) a heteroatom by a multiple bond (e.g., a double bond); for example, a carbon atom of the alkyl group may be bonded to oxygen via a double bond (i.e., substituted by oxo to provide a carbonyl function). The presence of such a substituent does not prevent the carbon backbone of the free radical being considered as an alkyl group.

As used herein, the term “cyclic group” means a saturated, partially or fully unsaturated, or aromatic group having at least 3 to 10 atoms (i.e., ring atoms) that form a ring. Where a cyclic group is defined as having a certain number of members, the term “members”, “membered” and the like is used to denote the number of ring atoms in said cyclic group. For example, a 5-membered cyclic group (e.g., a 5-membered heterocyclic group) contains 5 ring atoms. It will be appreciated that a cyclic group may be part of a larger cyclic system; for example, bicyclo[4.3.0]nonane comprises two carbocyclic groups, namely a cyclohexane group and a cyclopentane group, which are fused to form the carbocyclic system which makes up the molecule. The term “cyclic group” is intended to encompass both carbocyclic groups as well as heterocyclic groups. The term “carbocyclic” refers to a group having at least 3 to 9 carbon atoms that form a ring. The term “heterocyclic” refers to a group having at least 3 to 10 atoms that form a ring, wherein at least 1 to 9 of said ring atoms are carbon and the remaining at least 1 to 9 ring atom(s) (i.e., hetero ring atom(s)) are selected independently from the group consisting of nitrogen, sulphur, and oxygen.

The term “spiro” or “spirocyclic” as used herein in relation to cyclic groups denotes that a first cyclic group within a multicyclic system is attached to a second cyclic group within said multicyclic system, wherein the ring atoms of said first cyclic group and the ring atoms of said second cyclic group have only one atom in common, i.e., said first and second cyclic groups share only one common ring atom. For example, the spiro[5.5]undecanyl group comprises two cyclohexane rings which have a single carbon ring atom in common.

The term “fused” as used herein in relation to cyclic groups denotes that a first cyclic group within a multicyclic system is attached to a second cyclic group within said multicyclic system, wherein the ring atoms of said first cyclic group and the ring atoms of said second cyclic group have two adjacent atoms in common, i.e., said first and second cyclic groups share two common ring atoms. For example, the bicyclo[4.4.0]decanyl group comprises two cyclohexane rings which have two adjacent carbon ring atoms in common.

The term “bridged” as used herein in relation to cyclic groups denotes that a first cyclic group within a multicyclic system is attached to a second cyclic group within said multicyclic system, wherein the ring atoms of said first cyclic group and the ring atoms of said second cyclic group have more than two adjacent atoms in common, i.e., said first and second cyclic groups share three or more common ring atoms. For example, the bicyclo[3.3.1]nonanyl group comprises two cyclohexane rings which have three adjacent carbon ring atoms in common.

Within the structural formulae described herein, any ring system (including any spiro, fused, or bridged ring system) may be connected to other parts of a molecule through any atom having suitable valency. For example, a bicyclic ring may be connected to another part of the molecule through a ring atom (e.g., a secondary carbon atom or heteroatom such as N), or a bridgehead (e.g., a tertiary carbon atom). Spiro, fused, and bridged rings may be fully unsaturated, partially unsaturated, or fully saturated, and may have aromatic character in one or more of their constituent rings.

As used herein, the term “cycloalkyl” means a saturated free radical having at least 3 to 9 carbon atoms (i.e., ring atoms) that form a ring. Exemplary cycloalkyl groups include cyclopropyl, cyclobutyl, cyclopentyl, and cyclohexyl. It will be appreciated that the cycloalkyl group may be monocyclic or multicyclic (e.g., fused, bridged, or spirocyclic). In the case of multicyclic cycloalkyl groups, there are further rings, e.g. 1 or more further rings, all of which contain from 3 to 7 carbon atoms (i.e., ring atoms). Exemplary cycloalkyl groups having such further rings include bicyclo[1.1.1]pentanyl. The term “(C₃-C₇)cycloalkyl” denotes that the cycloalkyl group contains from 3 to 7 carbon atoms in the ring portion of the group, which may be monocyclic or multicyclic (e.g., fused, bridged, or spirocyclic), for example cyclopropanyl (having 3 ring carbon atoms) or bicyclo[1.1.1]pentanyl (having 5 ring carbon atoms). One or more ring atoms of the cycloalkyl group may be substituted by (i.e., bonded to) a heteroatom by a double bond (e.g., cycloalkyl substituted by oxo). The presence of such a substituent does not prevent the carbon backbone of the free radical being considered as a cycloalkyl group.

As used herein, the term “aryl” means an aromatic free radical having at least 6 carbon atoms (i.e., ring atoms) that form a ring. It will be appreciated that the aryl group may be monocyclic or multicyclic (e.g., fused). In the case of multicyclic aryl groups, there are further rings, e.g. 1 or more further rings, all of which contain at least 3 carbon atoms (i.e., ring atoms). The further rings may also contain one or more heteroatoms and they may be saturated, unsaturated, or aromatic. A multicyclic aryl group is typically attached to the rest of the molecule via an aromatic ring, and typically not via a ring containing a heteroatom. In embodiments, the multicyclic aryl group does not contain any ring heteroatoms. Examples of aryl groups include phenyl and naphthalenyl, as well as indenyl and indanyl groups. Other aryl groups include, for example, tetrahydroisoquinolinyl bonded to the rest of the molecule via its phenyl ring. The term “(C₆-C₁₀)aryl” denotes that the aryl group contains from 6 to 10 carbon atoms in the ring portion of the group, which may be monocyclic or multicyclic (e.g., fused), for example phenyl (having 6 ring carbon atoms) or indanyl (having 9 ring carbon atoms). In embodiments, (C₆-C₁₀)aryl is phenyl.

As used herein, the term “heterocycloalkyl” means a saturated free radical having at least 3 to 10 atoms (i.e., ring atoms) that form a ring, wherein at least 1 to 9 of said ring atoms are carbon and the remaining at least 1 to 9 ring atom(s) (i.e., hetero ring atom(s)) are selected independently from the group consisting of nitrogen, sulphur, and oxygen. In embodiments, the hetero ring atom(s) are selected independently from the group consisting of nitrogen and oxygen. For example, the term “4- to 10-membered heterocycloalkyl” means a saturated free radical containing from 4 to 10 ring atoms, of which one or more is a hetero ring atom. Heterocycloalkyl rings may have oxo substituents, typically adjacent to a heteroatom (e.g., 2-oxopyrrolidinyl), but the oxygen atom does not form part of the ring and is excluded from the number of ring atoms. The presence of such a substituent does not prevent the ring (or rings) of the free radical being considered as a heterocycloalkyl group. Exemplary heterocycloalkyl groups include tetrahydrofuranyl, piperidinyl, morpholinyl and piperazinyl. Any ring sulphur atom may optionally carry one or more pendant (i.e., non-ring) oxygen atoms, as found in, e.g., a sulfolanyl group. In the case of multicyclic heterocyclic groups, there are further rings, e.g. 1 or more further rings, all of which contain from 3 to 7 ring atoms selected from carbon, nitrogen, sulphur, and oxygen. The further rings may be saturated, or partially or fully unsaturated (e.g., having aromatic character). Multicyclic heterocyclic groups include fused, bridged and spirocyclic ring systems. Where a multicyclic heterocycloalkyl group contains an unsaturated fused ring, the group is typically not bonded to the rest of the molecule via that fused ring. Where a heterocycloalkyl group is described as being “X- to Y-membered” (where X and Y are integers), this means that the heterocycloalkyl group contains a total number of ring atoms from X to Y. Thus, for example, a “4- to 7-membered heterocycloalkyl group” contains a total of 4, 5, 6, or 7 ring atoms, for example tetrahydropyranyl (6 ring atoms).

As used herein, the terms “halo” and “halogen” mean fluorine, chlorine, bromine, or iodine. These terms are used interchangeably and may refer to a halogen free radical group or to a halogen atom as such. Those of skill in the art will readily be able to ascertain the identification of which in view of the context in which this term is used in the present disclosure. In embodiments, the halogen is fluorine.

As used herein, the term “oxo” means a free radical wherein an oxygen atom is connected to the atom bearing this radical via a double bond. For example, where a carbon atom carries an oxo radical it forms a carbon-oxygen double bond. It will be appreciated that not all atoms within a given structure can be substituted by oxo, and that this will depend on the free valency of the atom to be substituted.

The compounds of the present disclosure are described, inter alia, by way of structural formulae. It will be appreciated that these formulae typically show only one form (e.g., resonance form, tautomeric form, etc.) of the compound, whereas certain compounds may exist in more than one such form. This will be readily apparent to the skilled reader. The present disclosure includes all possible tautomers of the compounds characterised by the structural formulae herein, including as single tautomers, or as any mixture of tautomers in any ratio. For example, a pyrrolopyrazine moiety (as shown, e.g., in Formula (I), and which may be referred to as 4H-pyrrolo[2,3-b]pyrazine or 4,7-diazaindole) may be illustrated by any of the below tautomeric forms, which are used interchangeably throughout this description:

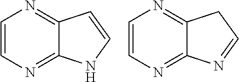

It will also be appreciated that certain of the present compounds may exist in one or more isomeric (e.g., stereoisomeric) forms. The present disclosure includes all possible stereoisomers, enantiomers, diastereomers, etc. of the compounds described hereinbefore and below, as well as cis- and trans-forms and conformers of the same. The purification and the separation of isomers may be accomplished by methods described hereinafter, as well as by techniques known in the art. For example, optical isomers of the compounds can be obtained by resolution of the racemic mixture of diastereoisomeric salts thereof (e.g., using an optically active acid or base, or by the formation of covalent diastereomers). A different process for separation of optical isomers involves the use of chiral chromatography (e.g., HPLC columns using a chiral phase), with or without conventional derivatization. Enzymatic separation, with or without derivatisation, may also be useful, and optically active compounds of the present disclosure can likewise be obtained by chiral syntheses utilizing optically active starting materials. The present disclosure includes all possible stereoisomers of the compounds described herein as single stereoisomers, or as any mixture of said stereoisomers, e.g. (R)- or (S)-isomers, in any ratio.

The compounds of the disclosure may exist in the form of free acids or bases, or may exist as addition salts with suitable acids or bases. For example, basic compounds of Formula (I) may be provided as pharmaceutically acceptable acid addition salts with an acid such as HCl, TFA, or formic acid (e.g., formic acid). Methods for forming salts are described below and are also known in the art (see, e.g., Berge et al., J Pharm Sci. (1977) 66:1-19).

As used herein, the term “pharmaceutically acceptable” when used in connection with salts means a salt of a currently disclosed compound that may be administered without any resultant substantial undesirable biological effect(s) or any resultant deleterious interaction(s) with any other component of a pharmaceutical composition in which it may be contained.

A group defined as “optionally substituted” may be either unsubstituted, or substituted with one or more substituents, e.g. 1, 2, 3, 4, 5, 6, or more substituents. In embodiments, a substituted group has 1 to 4 substituents, e.g. 1, 2, or 3 substituents. In embodiments, a substituted group has 1 or 2 substituents. In embodiments, a substituted group has 3 substituents.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Compositions and methods provided herein may be combined with one or more of any of the other compositions and methods provided herein.

The following abbreviations and empirical formulae are used herein:

| | |
|---|---|
| ABC | ammonium bicarbonate |
| Ac | acetyl |
| ACN/MeCN | acetonitrile |
| ATP | adenosine triphosphate |
| Boc | tert-butyloxy carbonyl |
| Cbz | benzyloxycarbonyl |
| DAD | diode-array detection |
| DCM | dichloromethane |
| DIPEA | diisopropylethylamine |
| DMF | N,N-dimethylformamide |
| DMSO | dimethyl sulfoxide |
| 4EBP1 | eukaryotic translation initiation factor 4E-binding |
| | protein 1 |
| EDC | 1-ethyl-3-(3-dimethylaminopropyl)carbodiimide |
| ERK | extracellular signal-regulated kinase |
| EtOAc/ | ethyl acetate |
| AcOEt | |
| FRET | Forster resonance energy transfer |
| HATU | hexafluorophosphate azabenzotriazole tetramethyl uronium |
| HOBt | hydroxybenzotriazole |
| HPLC | high performance liquid chromatography |
| LC/MS | liquid chromatography/mass spectrometry |
| LiHMDS | lithium bis(trimethylsilyl)amide |
| MS | mass spectrometry |
| NBS | N-bromosuccinimide |
| NCS | N-chlorosuccinimide |
| NIS | N-iodosuccinimide |
| NMR | nuclear magnetic resonance |
| Pd/C | palladium on carbon |
| PdCl₂(dppf) | [1,1′-bis(diphenylphosphino)ferrocene]dichloro- |
| | palladium(II) |
| Pd₂(dba)₃ | tris(dibenzylideneacetone)dipalladium(0) |
| PE | petroleum ether |
| rac | racemic mixture |
| RPMI | Roswell Park Memorial Institute medium |
| SEM | 2-trimethylsilylethoxymethyl |
| SGC | silica gel chromatography |
| TAD | transcriptional activation domain |
| TATU | 1-[bis(dimethylamino)methylene]-1H-1,2,3-triazolo[4,5- |
| | b]pyridinium 3-oxide tetrafluoroborate |
| TBTU | 2-(1H-benzotriazole-1-yl)-1,1,3,3-tetramethylaminium |
| | tetrafluoroborate |
| TEA | triethylamine |
| TFA | trifluoroacetic acid |
| THF | tetrahydrofuran |
| UPLC | ultra-high performance liquid chromatography |
| UV | ultraviolet |
| XPhosPdG4 | dicyclohexyl-[2-[2,4,6-tri(propan-2- |
| | yl)phenyl]phenyl]phosphanium; methanesulfonic acid; |
| | N-methyl-2-phenylaniline; palladium |

### Compounds

In a first aspect the present disclosure provides a compound being of Formula (I)

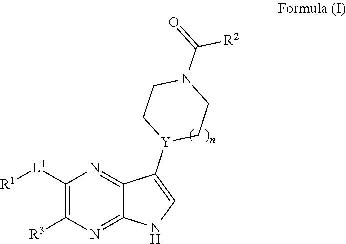

or a pharmaceutically acceptable salt thereof, wherein:
R¹ is selected from —(C₁-C₆)alkyl, —(C₃-C₇)cycloalkyl, and 4- to 10-membered heterocycloalkyl, wherein R¹ is optionally substituted with one or more occurrences of R^{A},
   wherein each R^{A} is independently selected from halo, —OH, oxo, —NH₂, ═N—OH, —(C₁-C₃)alkyl, —O(C₁-C₃)alkyl, and —(C₃-C₆)cycloalkyl, wherein each occurrence of —(C₁-C₃)alkyl is optionally substituted by one or more groups independently selected from halo and —OH;
L¹ is selected from a direct bond, —O—, and —NH—;
R² is selected from —(C₃-C₆)cycloalkyl, and —(C₆-C₁₀)aryl, wherein R² is optionally substituted with one, two, or three occurrences of R^{B},
   wherein each R^{B} is independently selected from halo, —NH₂, —SF₅, —(C₁-C₃)alkyl, —O(C₁-C₃)alkyl, and —O(C₃-C₆)cycloalkyl, wherein each occurrence of —(C₁-C₃)alkyl and —O(C₁-C₃)alkyl is optionally substituted by one or more groups independently selected from halo and —OH;
R³ is selected from —H, —(C₁-C₃)alkyl (e.g., —CH₃), and —OH;
Y is CH, or N; and
n is 0, or 1.

In embodiments, the compound is a compound of Formula (I) or a pharmaceutically acceptable salt thereof, wherein:
R¹ is selected from —(C₁-C₆)alkyl, —(C₃-C₇)cycloalkyl, and 4- to 10-membered heterocycloalkyl, wherein R¹ is optionally substituted with one or more occurrences of R^{A},
   wherein each R^{A} is independently selected from halo, —OH, —NH₂, —(C₁-C₃)alkyl, —O(C₁-C₃)alkyl, and —(C₃-C₆)cycloalkyl, wherein each occurrence of —(C₁-C₃)alkyl is optionally substituted by one or more groups independently selected from halo and —OH;
L¹ is selected from a direct bond, —O—, and —NH—;
R² is selected from —(C₃-C₆)cycloalkyl, and —(C₆-C₁₀)aryl, wherein R² is optionally substituted with one, two, or three occurrences of R^{B},
   wherein each R^{B} is independently selected from halo, —NH₂, —SF₅, —(C₁-C₃)alkyl, —O(C₁-C₃)alkyl, and —O(C₃-C₆)cycloalkyl, wherein each occurrence of —(C₁-C₃)alkyl and —O(C₁-C₃)alkyl is optionally substituted by one or more halo;
R³ is selected from —H, —CH₃, and —OH;
Y is CH, or N; and
n is 0, or 1.

In embodiments, the compound is a compound of Formula (I) or a pharmaceutically acceptable salt thereof, wherein:
R¹ is selected from —(C₁-C₆)alkyl, —(C₃-C₇)cycloalkyl, and 4- to 10-membered heterocycloalkyl, wherein R¹ is optionally substituted with one or more occurrences of R^{A},
   wherein each R^{A} is independently selected from halo, —OH, —(C₁-C₃)alkyl, —O(C₁-C₃)alkyl, and —(C₃-C₆)cycloalkyl;
L¹ is selected from a direct bond, —O—, and —NH—;
R² is selected from —(C₃-C₆)cycloalkyl, and —(C₆-C₁₀)aryl, wherein R² is optionally substituted with one or two occurrences of R^{B},
   wherein each R^{B} is independently selected from halo, —NH₂, —SF₅, —(C₁-C₃)alkyl, —O(C₁-C₃)alkyl, and —O(C₃-C₆)cycloalkyl, wherein each occurrence of —(C₁-C₃)alkyl and —O(C₁-C₃)alkyl is optionally substituted by one or more groups independently selected from halo and —OH;
R³ is —H;
Y is CH; and
n is 0, or 1.

In embodiments, the compound is a compound of Formula (I) or a pharmaceutically acceptable salt thereof, wherein:
R¹ is selected from —(C₁-C₆)alkyl, —(C₃-C₇)cycloalkyl, and 4- to 10-membered heterocycloalkyl, wherein R¹ is optionally substituted with one or more occurrences of R^{A},
   wherein each R^{A} is independently selected from halo, —OH, —(C₁-C₃)alkyl, —O(C₁-C₃)alkyl, and —(C₃-C₆)cycloalkyl;
L¹ is selected from a direct bond, —O—, and —NH—;
R² is selected from —(C₃-C₆)cycloalkyl, and —(C₆-C₁₀)aryl, wherein R² is optionally substituted with one or two occurrences of R^{B},
   wherein each R^{B} is independently selected from halo, —NH₂, —SF₅, —(C₁-C₃)alkyl, —O(C₁-C₃)alkyl, and —O(C₃-C₆)cycloalkyl, wherein each occurrence of —(C₁-C₃)alkyl and —O(C₁-C₃)alkyl is optionally substituted by one or more halo;
R³ is —H;
Y is CH; and
n is 0, or 1.

In embodiments, R¹ is selected from —(C₁-C₃)alkyl (e.g., propyl), —(C₅-C₇)cycloalkyl, and 4- to 7-membered heterocycloalkyl, wherein R¹ is optionally substituted with one or more occurrences of R^{A} as defined herein. In embodiments, R¹ is selected from —(C₁-C₃)alkyl (e.g., propyl), —(C₅-C₆)cycloalkyl, and 4- to 6-membered heterocycloalkyl, wherein R¹ is optionally substituted with one or more occurrences of R^{A} as defined herein.

In embodiments, R¹ is selected from —(C₁-C₃)alkyl, —(C₅-C₆)cycloalkyl, and 5- to 6-membered heterocycloalkyl, wherein R¹ is optionally substituted with one or more occurrences of R^{A} as defined herein.

In embodiments, R¹ is unsubstituted.

In other embodiments, R¹ is substituted with one, two, or three occurrences of R^{A} as defined herein. In embodiments, R¹ is substituted with one, or two occurrences of R^{A}. In embodiments, R¹ is substituted with one occurrence of R^{A}. In embodiments, R¹ is substituted with two occurrences of R^{A}. In embodiments, R¹ is substituted with three occurrences of R^{A}.

In embodiments, each R^{A} is independently selected from halo (e.g., —F), —OH, —NH₂, —(C₁-C₃)alkyl, —OCH₃, and cyclopropyl, wherein each occurrence of —(C₁-C₃)alkyl is optionally substituted by one or more groups independently selected from halo (e.g., —F) and —OH. In embodiments, each R^{A} is independently selected from —F, —OH, methyl, —OCH₃, and cyclopropyl. In embodiments, R¹ is substituted by one or two occurrences of R^{A}, wherein each R^{A} is independently selected from —F, —OH, methyl, —OCH₃, and cyclopropyl. In embodiments, R¹ is selected from:

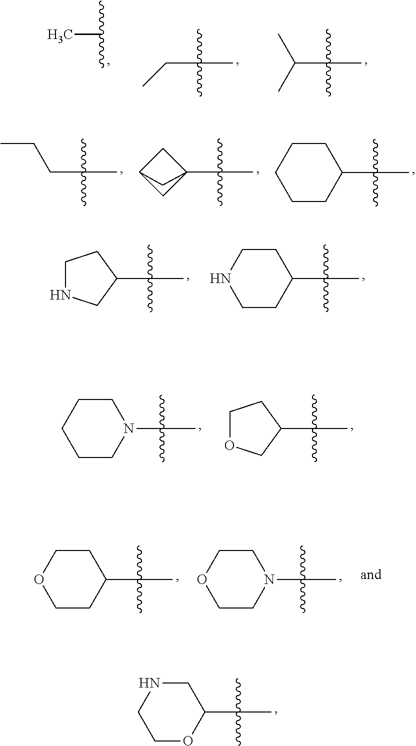

wherein R¹ is optionally substituted by one or more occurrences of R^{A} as defined herein. In embodiments, each R^{A} is independently selected from the group consisting of halo (e.g., —F), —OH, —NH₂, —(C₁-C₃)alkyl (e.g., methyl, or ethyl), —O(C₁-C₃)alkyl (e.g., —OCH₃), —(C₃-C₆)cycloalkyl (e.g. cyclopropyl), wherein each occurrence of (C₁-C₃)alkyl may be optionally substituted by one or more substituents selected from halo and —OH.

In embodiments, R¹ is selected from:

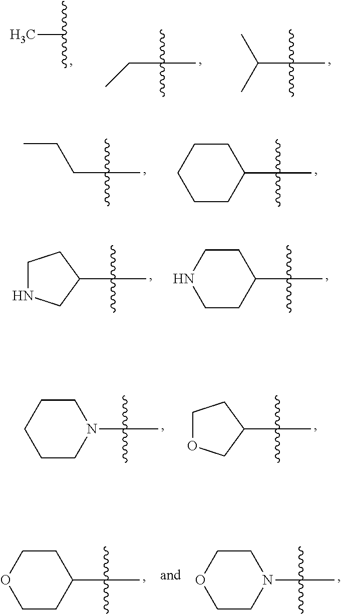

wherein R¹ is optionally substituted by one or more occurrences of R^{A} as defined herein. In embodiments, each R^{A} is independently selected from the group consisting of halo (e.g., —F), —OH, —NH₂, —(C₁-C₃)alkyl (e.g., methyl, or ethyl), —O(C₁-C₃)alkyl (e.g., —OCH₃), and —(C₃-C₆)cycloalkyl (e.g. cyclopropyl), wherein each occurrence of (C₁-C₃)alkyl may be optionally substituted by one or more substituents selected from halo (e.g., —F) and —OH.

In embodiments, R¹ is selected from:

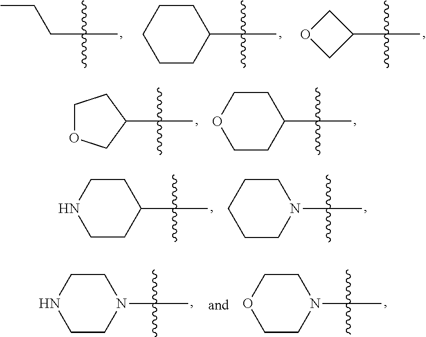

wherein R¹ is optionally substituted by one or two occurrences of R^{A} as defined herein. In embodiments, each R^{A} is independently selected from the group consisting of —F, —OH, methyl, —OCH₃, and cyclopropyl.

In embodiments, R¹ is selected from:

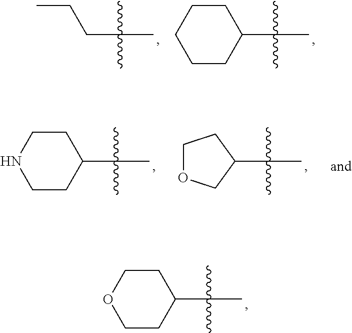

wherein R¹ is optionally substituted by one occurrence of R^{A} as defined herein. In embodiments, R^{A} is selected from the group consisting of methyl, —OCH₃, and cyclopropyl.

In embodiments, R¹ is selected from:

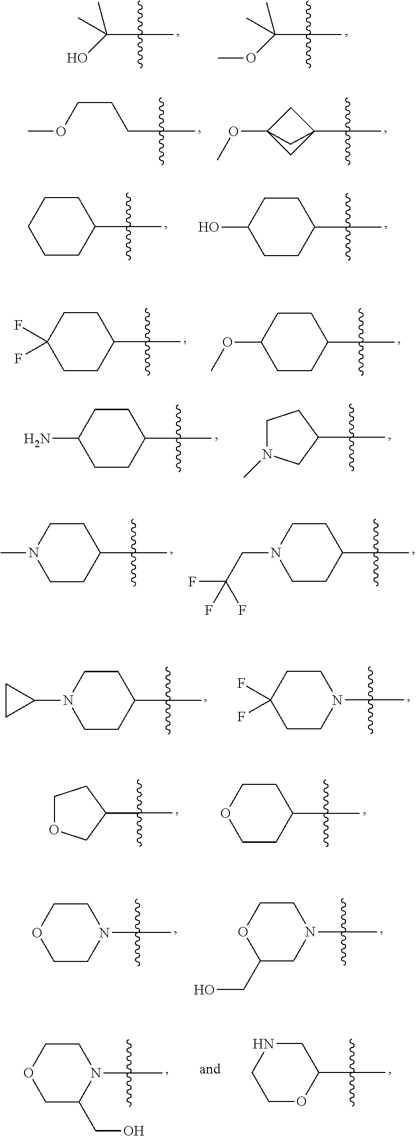

In embodiments, R¹ is selected from:

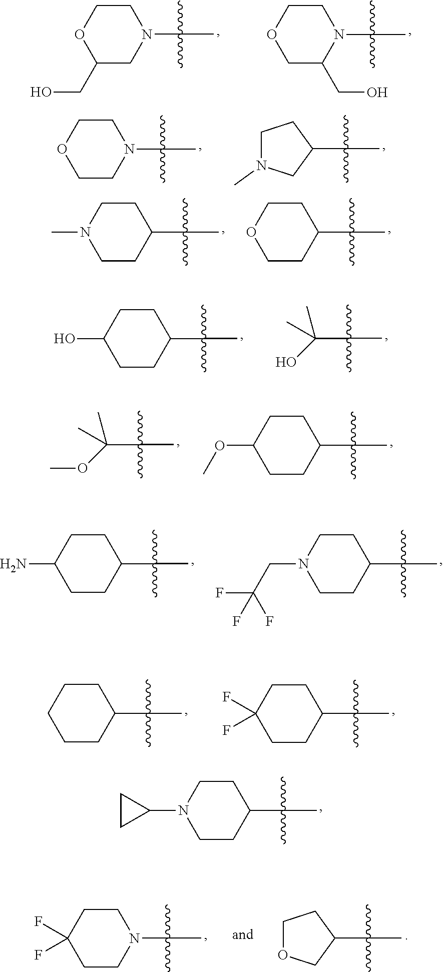

In embodiments, R¹ is cyclohexyl optionally substituted by one or more occurrences of R^{A} as defined herein. In embodiments, R¹ is unsubstituted cyclohexyl.

In embodiments, R¹ is 5- to 6-membered heterocycloalkyl, wherein the heterocycloalkyl group comprises one or two atoms independently selected from O and N, and wherein the heterocycloalkyl group is optionally and independently substituted by one or more occurrences of R^{A} as defined herein. In embodiments, each R^{A} is independently selected from —CH₃, —OCH₃, and cyclopropyl.

In embodiments, R¹ is selected from:

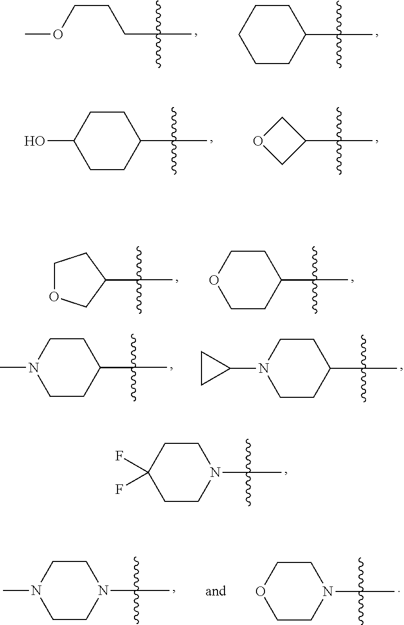

In embodiments, R¹ is selected from:

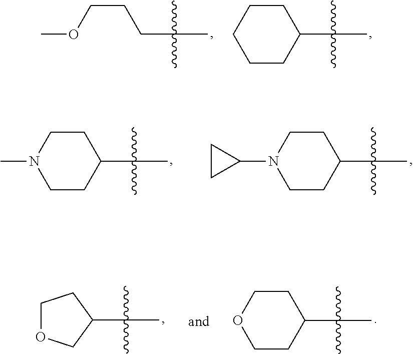

In embodiments, R² is —(C₃-C₆)cycloalkyl optionally substituted with one, two, or three occurrences of R^{B} as defined herein. In embodiments, R² is unsubstituted —(C₃-C₆)cycloalkyl.

In embodiments, R² is unsubstituted cyclohexyl.

In embodiments, R² is —(C₆-C₁₀)aryl substituted with one, two, or three occurrences of R^{B} as defined herein. In embodiments, R² is phenyl substituted with one, two, or three occurrences of R^{B}. In embodiments, R² is —(C₆-C₁₀)aryl substituted with one, or two occurrences of R^{B}. In embodiments, R² is phenyl substituted with one, or two occurrences of R^{B}. In embodiments, R² is —(C₆-C₁₀)aryl substituted with one occurrence of R^{B}. In embodiments, R² is phenyl substituted with one occurrence of R^{B}. In embodiments, R² is —(C₆-C₁₀)aryl substituted with two occurrences of R^{B}. In embodiments, R² is phenyl substituted with two occurrences of R^{B}.

In embodiments, R² is cyclohexyl or phenyl optionally substituted with one, two, or three occurrences of R^{B} as defined herein. In embodiments, R² is unsubstituted cyclohexyl, or R² is phenyl substituted with one, two, or three occurrences of R^{B}. In embodiments, R² is phenyl substituted with one or two occurrences of R^{B}.

In embodiments, R² is unsubstituted. In other embodiments, R² is substituted with one or two occurrences of R^{B} as defined herein. In embodiments, R² is substituted with one occurrence of R^{B}.

In embodiments, each R^{B} is independently selected from halo (e.g., —F), —NH₂, —SF₅, —CF₂CF₃, —C(OH)(CF₃)₂, —OCF₃, and —O-cyclopropyl. In embodiments, each R^{B} is independently selected from halo (e.g., —F), —NH₂, —SF₅, —OCF₃, —O-cyclopropyl, and —CF₂CF₃. In embodiments, R² is phenyl and each R^{B} is independently selected from halo (e.g., —F), —NH₂, —SF₅, —OCF₃, —O-cyclopropyl, and —CF₂CF₃.

In embodiments, each R^{B} is independently selected from halo (e.g., —F), —NH₂, —SF₅, —OCF₃, —O-cyclopropyl, and —CF₂CF₃. In embodiments, R² is substituted with one, or two occurrences of R^{B}, and each R^{B} is independently selected from —F, —NH₂, —SF₅, —OCF₃, —O-cyclopropyl, and —CF₂CF₃. In embodiments, R² is substituted with two, or three occurrences of R^{B}, and each R^{B} is independently selected from halo (e.g., —F), —NH₂, —SF₅, and —OCF₃. In embodiments, R² is substituted with three occurrences of R^{B}, and each R^{B} is independently selected from —F, —NH₂, and —OCF₃.

In embodiments, at least one occurrence of R^{B} is —SF₅. In embodiments, one occurrence of R^{B} is —SF₅. In embodiments, R² is substituted with one occurrence of R^{B}, and R^{B} is —SF₅. In embodiments, R² is substituted with two, or three occurrences of R^{B}, wherein one occurrence of R^{B} is —SF₅, and the other occurrence(s) of R^{B} are independently selected from halo (e.g., —F), and —NH₂. In embodiments, R² is substituted with two occurrences of R^{B}, wherein one occurrence of R^{B} is —SF₅, and the other occurrence of R^{B} is selected from —F and —NH₂.

In embodiments, at least one occurrence of R^{B} is halo. In embodiments, each halo is —F. In embodiments, at least one occurrence of R^{B} is —F. In embodiments, R² is phenyl substituted with one, two, or three occurrences of R^{B}, and each R^{B} is independently selected from halo (e.g., —F), —NH₂, —SF₅, —OCF₃, —O-cyclopropyl, and —CF₂CF₃. In embodiments, R² is phenyl substituted with two, or three occurrences of R^{B}, and each R^{B} is independently selected from halo (e.g., —F), —NH₂, —SF₅, —OCF₃, —O-cyclopropyl, and —CF₂CF₃. In embodiments, R² is phenyl substituted with one occurrence of R^{B}, and R^{B} is selected from —SF₅, —OCF₃, —O-cyclopropyl, and —CF₂CF₃. In embodiments, R² is phenyl substituted with two occurrences of R^{B}, and each R^{B} is independently selected from halo (e.g., —F), —NH₂, —SF₅, —OCF₃. In embodiments, R² is phenyl substituted with three occurrences of R^{B}, and each R^{B} is independently selected from —F, —NH₂, and —OCF₃.

In embodiments, R² is phenyl substituted with one, two, or three occurrences of R^{B}, and at least one occurrence of R^{B} is —SF₅. In embodiments, R² is phenyl substituted with one, two, or three occurrences of R^{B}, and one occurrence of R^{B} is —SF₅. In embodiments, R² is phenyl substituted with one, or two occurrences of R^{B}, wherein one occurrence of R^{B} is —SF₅, and the other occurrence of R^{B} (where present) is selected from —F, and —NH₂. In embodiments, R² is phenyl substituted with two occurrences of R^{B}, wherein one occurrence of R^{B} is —SF₅, and the other occurrence of R^{B} is selected from —F, and —NH₂. In embodiments, R² is phenyl substituted with one occurrence of R^{B}, and R^{B} is —SF₅.

In embodiments, R² is selected from

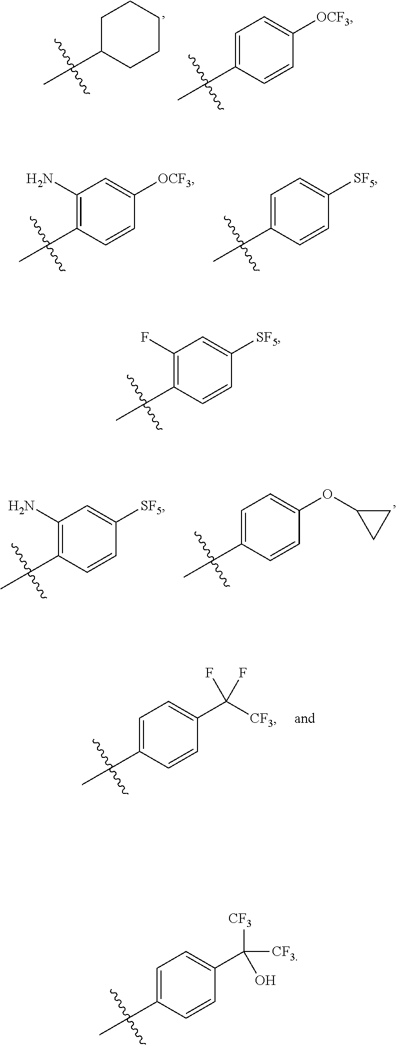

In embodiments, R² is selected from:

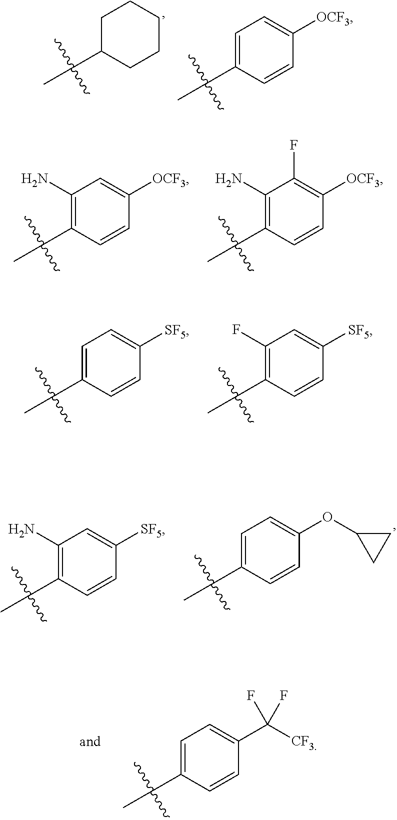

In embodiments, R² is selected from

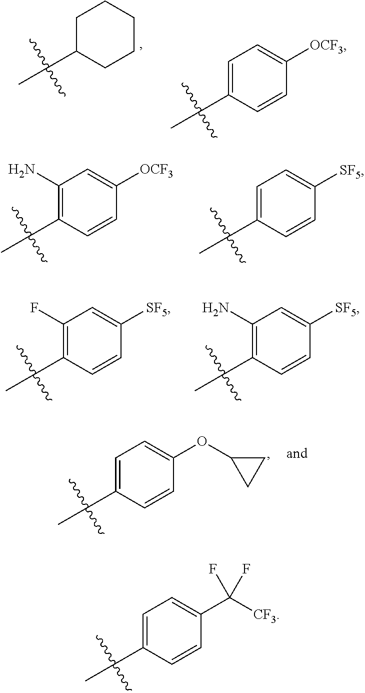

In embodiments, R³ is selected from —H and —(C₁-C₃)alkyl. In embodiments, R³ is selected from —H and —CH₃. In embodiments, R³ is —CH₃. In embodiments, R³ is —H.

In embodiments, Y is CH. In embodiments, Y is N.

In embodiments, n is 0. In embodiments, n is 1.

In embodiments, Y is N, and n is 1. Viewed from this aspect, the disclosure provides a compound of Formula (I-A)

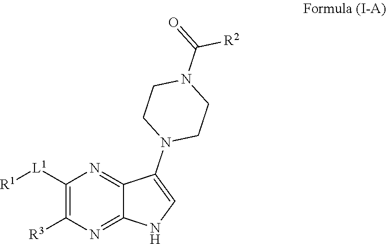

or a pharmaceutically acceptable salt thereof, wherein R¹, R², R³, and L¹ are as defined herein.

In embodiments, Y is CH, and n is 0. Viewed from this aspect, the disclosure provides a compound of Formula (I-B)

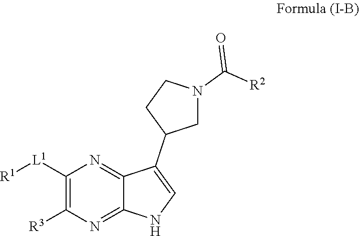

or a pharmaceutically acceptable salt thereof, wherein R¹, R², R³, and L¹ are as defined herein.

In embodiments, R² is phenyl substituted with one or two occurrences of R^{B}, wherein each R^{B} is independently selected from —NH₂, —OCF₃, and —SF₅.

In embodiments, Y is CH, and n is 1.

In embodiments, L¹ is a direct bond.

In embodiments, R³ is —H.

In embodiments, L¹ is selected from —O— and —NH—. Viewed from this aspect, the disclosure provides a compound of Formula (I-C) or Formula (I-D)

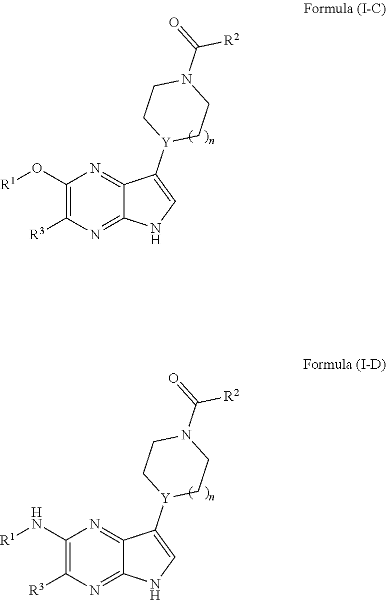

or a pharmaceutically acceptable salt thereof, wherein R¹, R², R³, Y, and n are as defined herein. In embodiments, L¹ is —O—, i.e., the compound is a compound of Formula (I-C), or a pharmaceutically acceptable salt thereof. In other embodiments, L¹ is —NH—, i.e., the compound is a compound of Formula (I-D), or a pharmaceutically acceptable salt thereof.

In embodiments, n is 1. In embodiments, R³ is —H.

In embodiments, Y is CH and L¹ is selected from —O— and —NH—. Viewed from this aspect, the disclosure provides a compound of Formula (I-E) or Formula (I-F)

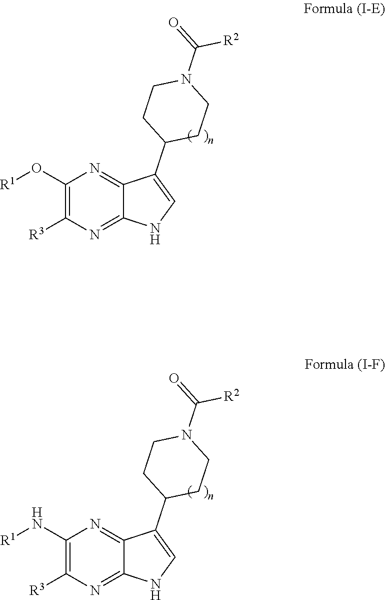

or a pharmaceutically acceptable salt thereof, wherein R¹, R², R³, and n are as defined herein.

In embodiments, L¹ is —O—, i.e., the compound is a compound of Formula (I-E), or a pharmaceutically acceptable salt thereof. In other embodiments, L¹ is —NH—, i.e., the compound is a compound of Formula (I-F), or a pharmaceutically acceptable salt thereof.

In embodiments, n is 1. In embodiments, R³ is —H. In embodiments, n is 1 and R³ is —H.

In embodiments, Y is CH and R³ is —H. Viewed from this aspect, the disclosure provides a compound of Formula (I-G)

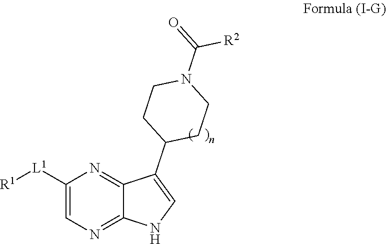

or a pharmaceutically acceptable salt thereof, wherein R¹, R², L¹, and n are as defined herein.

In embodiments, L¹ is —O—. In other embodiments, L¹ is —NH—. In embodiments, n is 1.

In embodiments, the compound is a compound of Formula (II)

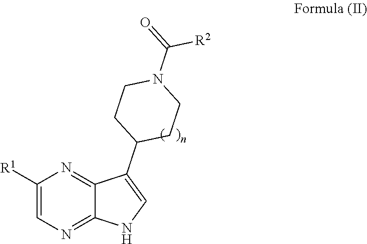

or a pharmaceutically acceptable salt thereof, wherein:
R¹ is selected from —(C₁-C₆)alkyl, —(C₃-C₇)cycloalkyl, and 4- to 10-membered heterocycloalkyl, wherein R¹ is optionally substituted with one or more occurrences of R^{A},
   wherein each R^{A} is independently selected from halo (e.g., —F), —OH, —(C₁-C₃)alkyl, —O(C₁-C₃)alkyl, and —(C₃-C₆)cycloalkyl;
R² is selected from —(C₃-C₆)cycloalkyl, and —(C₆-C₁₀)aryl, wherein R² is optionally substituted with one or two occurrences of R^{B},
   wherein each R^{B} is independently selected from halo, —NH₂, —SF₅, —(C₁-C₃)alkyl, —O(C₁-C₃)alkyl, and —O(C₃-C₆)cycloalkyl, wherein each occurrence of —(C₁-C₃)alkyl and —O(C₁-C₃)alkyl is substituted by one or more groups independently selected from halo and —OH; and
n is 0, or 1.

In embodiments, the compound is a compound of Formula (II) or a pharmaceutically acceptable salt thereof, wherein:
R¹ is selected from —(C₁-C₆)alkyl, —(C₃-C₇)cycloalkyl, and 4- to 10-membered heterocycloalkyl, wherein R¹ is optionally substituted with one or more occurrences of R^{A},
   wherein each R^{A} is independently selected from —(C₁-C₃)alkyl, —O(C₁-C₃)alkyl, and —(C₃-C₆)cycloalkyl;
R² is selected from —(C₃-C₆)cycloalkyl, and —(C₆-C₁₀)aryl, wherein R² is optionally substituted with one or two occurrences of R^{B},
   wherein each R^{B} is independently selected from halo, —NH₂, —SF₅, —(C₁-C₃)alkyl, —O(C₁-C₃)alkyl, and —O(C₃-C₆)cycloalkyl, wherein each occurrence of —(C₁-C₃)alkyl and —O(C₁-C₃)alkyl is substituted by one or more halo; and
n is 0, or 1.

In embodiments, R¹ is 4- to 6-membered heterocycloalkyl, wherein the heterocycloalkyl group comprises one or two ring heteroatom(s) independently selected from O and N, and wherein the heterocycloalkyl group is optionally and independently substituted by one or more occurrences of R^{A} as defined herein. In embodiments, R¹ is 5- to 6-membered heterocycloalkyl, wherein the heterocycloalkyl group comprises one ring heteroatom selected from O and N, and wherein the heterocycloalkyl group is optionally and independently substituted by one or more occurrences of R^{A} as defined herein. In embodiments, each R^{A} is independently selected from —F, —OH, methyl, —OCH₃, and cyclopropyl. In embodiments, each R^{A} is independently selected from —CH₃, —OCH₃, and cyclopropyl.

In embodiments, R is selected from:

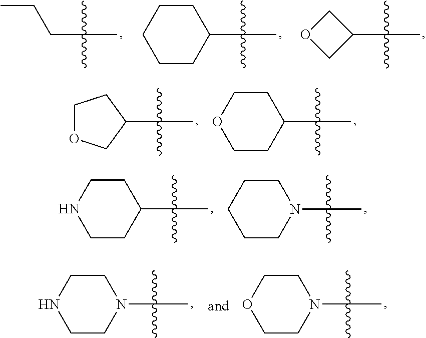

wherein R¹ is optionally substituted by one or two occurrences of R^{A}, wherein each occurrence of R^{A} is independently selected from —F, —OH, methyl, —OCH₃, and cyclopropyl.

In embodiments, R¹ is selected from:

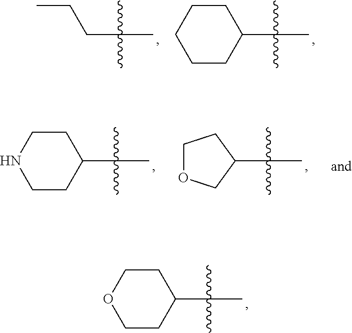

wherein R¹ is optionally substituted by one occurrence of R^{A}, wherein R^{A} is methyl, —OCH₃, or cyclopropyl.

In embodiments, R¹ is selected from:

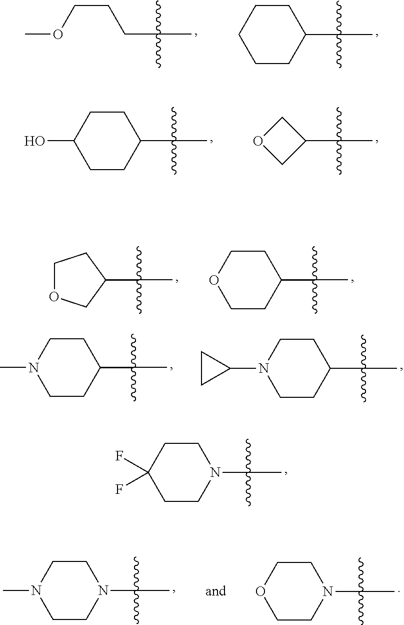

In embodiments, R¹ is selected from:

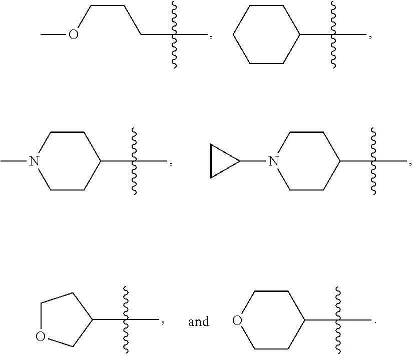

In embodiments, n is 0. In embodiments, n is 1.

In embodiments, R² is —(C₃-C₆)cycloalkyl (e.g., cyclohexyl) optionally substituted with one or two occurrences of R^{B} as defined herein. In embodiments, R² is unsubstituted —(C₃-C₆)cycloalkyl. In embodiments, R² is unsubstituted cyclohexyl.

In embodiments, R² is —(C₆-C₁₀)aryl substituted with one, or two occurrences of R^{B}. In embodiments, R² is phenyl substituted with one, or two occurrences of R^{B}. In embodiments, R² is —(C₆-C₁₀)aryl substituted with one occurrence of R^{B}. In embodiments, R² is phenyl substituted with one occurrence of R^{B}.

In embodiments, R² is cyclohexyl or phenyl optionally substituted with one or two occurrences of R^{B} as defined herein. In embodiments, R² is unsubstituted cyclohexyl, or R² is phenyl substituted with one or two occurrences of R^{B}.

In embodiments, R² is substituted with one or two occurrences of R^{B} as defined herein. In embodiments, R² is substituted with one occurrence of R^{B}. In embodiments, R² is unsubstituted.

In embodiments, each R^{B} is independently selected from halo (e.g., —F), —NH₂, —SF₅, —OCF₃, —O-cyclopropyl, —CF₂CF₃, and —C(OH)(CF₃)₂. In embodiments, each R^{B} is independently selected from halo (e.g., —F), —NH₂, —SF₅, —OCF₃, —O-cyclopropyl, and —CF₂CF₃. In embodiments, R² is substituted with one or two occurrences of R^{B}, and each R^{B} is independently selected from halo (e.g., —F), —NH₂, —SF₅, and —OCF₃. In embodiments, R² is substituted with one occurrence of R^{B}, and R^{B} is selected from —OCF₃, —SF₅, —O-cyclopropyl, and —CF₂CF₃. In embodiments, R² is substituted with two occurrences of R^{B}, and each R^{B} is independently selected from halo (e.g., —F), —NH₂, —SF₅, and —OCF₃.

In embodiments, at least one occurrence of R^{B} is halo. In embodiments, each halo is —F. In embodiments, at least one occurrence of R^{B} is —F.

In embodiments, at least one occurrence of R^{B} is —SF₅. In embodiments, one occurrence of R^{B} is —SF₅. In embodiments, R² is substituted with one or two occurrences of R^{B}, wherein one occurrence of R^{B} is —SF₅, and the other occurrence of R^{B} (where present) is selected from —F, and —NH₂. In embodiments, R² is substituted with two occurrences of R^{B}, one occurrence of R^{B} is —SF₅, and the other occurrence of R^{B} is selected from —F, and —NH₂. In embodiments, R² is substituted with one occurrence of R^{B}, and R^{B} is —SF₅.

In embodiments, n is 1. In other embodiments, n is 0. Viewed from this aspect, the disclosure provides a compound of Formula (II-A) or Formula (II-B)

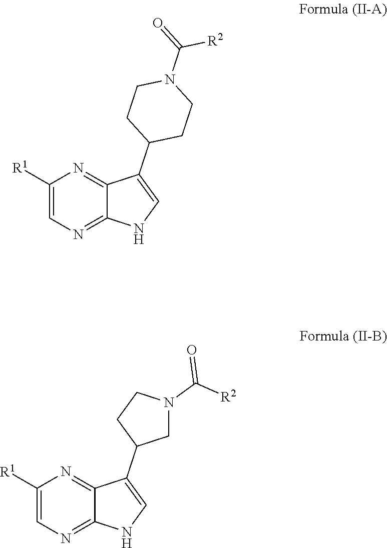

or a pharmaceutically acceptable salt thereof, wherein R¹ and R² are as defined herein. In embodiments, the compound is a compound of Formula (II-A), or a pharmaceutically acceptable salt thereof. In other embodiments, the compound is a compound of Formula (II-B), or a pharmaceutically acceptable salt thereof.

In embodiments, the compound is a compound of Formula (III-A)

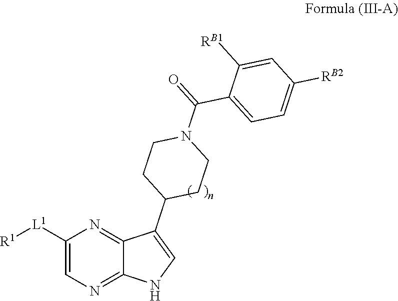

or a pharmaceutically acceptable salt thereof, wherein R¹, L¹, and n are as defined herein, and wherein:
R^{B1} is either —H, or is selected from the group consisting of —NH₂, and —F; and
R^{B2} is selected from the group consisting of —OCF₃, —SF₅, —CF₂CF₃, —C(OH)(CF₃)₂, and —O-cyclopropyl.

In embodiments, the compound is a compound of Formula (III-A) or a pharmaceutically acceptable salt thereof, wherein R¹, L¹ and n are as defined herein, and wherein:
R^{B1} is either —H, or is selected from the group consisting of —NH₂, and —F; and
R^{B2} is selected from the group consisting of —OCF₃, —SF₅, —CF₂CF₃, and —O-cyclopropyl.

In embodiments, L¹ is —NH—. In other embodiments, L¹ is —O—.

In embodiments, the compound is a compound of Formula (III)

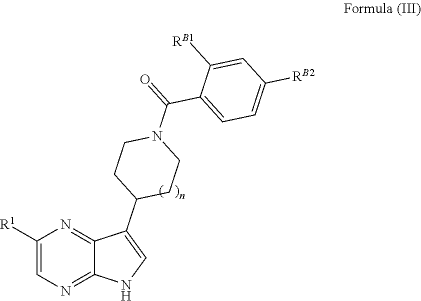

or a pharmaceutically acceptable salt thereof, wherein R¹ and n are as defined herein, and wherein:
R^{B1} is either —H, or is selected from the group consisting of —NH₂, and —F; and
R^{B2} is selected from the group consisting of —OCF₃, —SF₅, —CF₂CF₃, —C(OH)(CF₃)₂, and —O-cyclopropyl.

In embodiments, the compound is a compound of Formula (III) or a pharmaceutically acceptable salt thereof, wherein R¹ and n are as defined herein, and wherein:
R^{B1} is either —H, or is selected from the group consisting of —NH₂, and —F; and
R^{B2} is selected from the group consisting of —OCF₃, —SF₅, —CF₂CF₃, and —O— cyclopropyl.

In embodiments, R^{B1} is —H. In embodiments, R^{B1} is —NH₂. In embodiments, R^{B1} is —F.

In embodiments, R^{B2} is —OCF₃. In embodiments, R^{B2} is —SF₅. In embodiments, R^{B2} is —CF₂CF₃. In embodiments, R^{B2} is —O-cyclopropyl.

In embodiments, R^{B1} is —H and R^{B2} is —OCF₃. In embodiments, R^{B1} is —NH₂ and R^{B2} is —OCF₃.

In embodiments, R^{B1} is —H and R^{B2} is —SF₅. In embodiments, R^{B1} is —NH₂ and R^{B2} is —SF₅. In embodiments, R^{B1} is —F and R^{B2} is —SF₅. In embodiments, R^{B1} is —H and R^{B2} is —O— cyclopropyl. In embodiments, R^{B1} is —H and R^{B2} is —CF₂CF₃.

In embodiments, the compound is a compound of Formula (IV-A) or Formula (V-A)

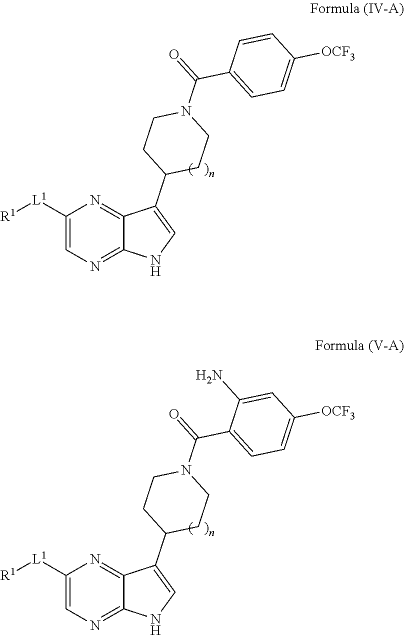

or a pharmaceutically acceptable salt thereof, wherein R¹, L¹ and n are as defined herein. In embodiments, the compound is a compound of Formula (IV-A), or a pharmaceutically acceptable salt thereof. In other embodiments, the compound is a compound of Formula (V-A), or a pharmaceutically acceptable salt thereof.

In embodiments, L¹ is —NH—. In other embodiments, L¹ is —O—.

In embodiments, the compound is a compound of Formula (IV) or Formula (V)

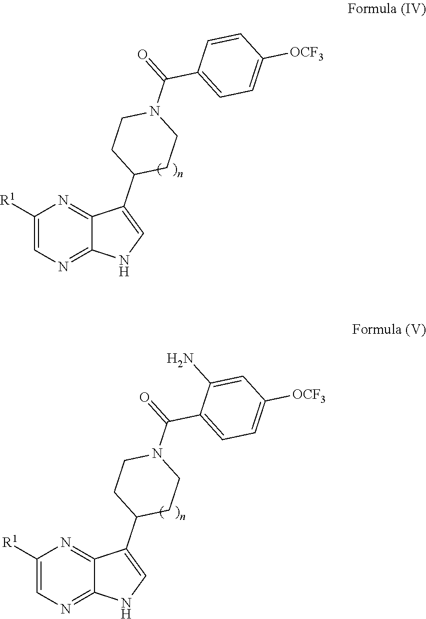

or a pharmaceutically acceptable salt thereof, wherein R¹ and n are as defined herein. In embodiments, the compound is a compound of Formula (IV), or a pharmaceutically acceptable salt thereof. In other embodiments, the compound is a compound of Formula (V), or a pharmaceutically acceptable salt thereof.

In embodiments, the compound is a compound of Formula (VI), Formula (VII), or Formula (VIII)

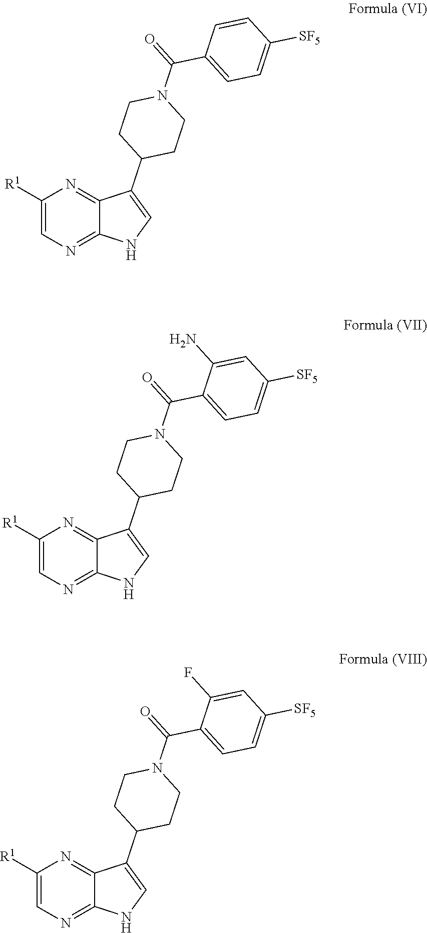

or a pharmaceutically acceptable salt thereof, wherein R¹ is as defined herein. In embodiments, the compound is a compound of Formula (VI), or a pharmaceutically acceptable salt thereof. In other embodiments, the compound is a compound of Formula (VII), or a pharmaceutically acceptable salt thereof. In other embodiments, the compound is a compound of Formula (VIII), or a pharmaceutically acceptable salt thereof.

In embodiments, the compound is selected from the group consisting of:
[2-amino-4-(trifluoromethoxy)phenyl]-[4-[2-(1-methyl-4-piperidyl)-5H-pyrrolo[2,3-b]pyrazin-7-yl]-1-piperidyl]methanone,
[2-amino-4-(trifluoromethoxy)phenyl]-[4-(2-tetrahydropyran-4-yl-5H-pyrrolo[2,3-b]pyrazin-7-yl)-1-piperidyl]methanone,
[4-(2-tetrahydropyran-4-yl-5H-pyrrolo[2,3-b]pyrazin-7-yl)-1-piperidyl]-[4-(trifluoromethoxy)phenyl]methanone,
[4-[2-(1-methyl-4-piperidyl)-5H-pyrrolo[2,3-b]pyrazin-7-yl]-1-piperidyl]-[4-(trifluoromethoxy)phenyl]methanone,
[4-(2-tetrahydrofuran-3-yl-5H-pyrrolo[2,3-b]pyrazin-7-yl)-1-piperidyl]-[4-(trifluoromethoxy)phenyl]methanone,
[4-[2-(3-methoxypropyl)-5H-pyrrolo[2,3-b]pyrazin-7-yl]-1-piperidyl]-[4-(trifluoromethoxy)phenyl]methanone,
[2-amino-4-(trifluoromethoxy)phenyl]-[4-[2-(3-methoxypropyl)-5H-pyrrolo[2,3-b]pyrazin-7-yl]-1-piperidyl]methanone,
[4-(2-cyclohexyl-5H-pyrrolo[2,3-b]pyrazin-7-yl)-1-piperidyl]-[4-(trifluoromethoxy)phenyl]methanone,
[2-amino-4-(trifluoromethoxy)phenyl]-[4-(2-cyclohexyl-5H-pyrrolo[2,3-b]pyrazin-7-yl)-1-piperidyl]methanone,
[2-amino-4-(trifluoromethoxy)phenyl]-[4-[2-(1-cyclopropyl-4-piperidyl)-5H-pyrrolo[2,3-b]pyrazin-7-yl]-1-piperidyl]methanone,
[4-[2-(1-cyclopropyl-4-piperidyl)-5H-pyrrolo[2,3-b]pyrazin-7-yl]-1-piperidyl]-[4-(trifluoromethoxy)phenyl]methanone,
[2-amino-4-(trifluoromethoxy)phenyl]-[4-(2-tetrahydrofuran-3-yl-5H-pyrrolo[2,3-b]pyrazin-7-yl)-1-piperidyl]methanone,
[4-[2-(1-methyl-4-piperidyl)-5H-pyrrolo[2,3-b]pyrazin-7-yl]-1-piperidyl]-[4-(pentafluoro-λ⁶-sulfanyl)phenyl]methanone,
[2-amino-4-(pentafluoro-λ⁶-sulfanyl)phenyl]-[4-[2-(1-methyl-4-piperidyl)-5H-pyrrolo[2,3-b]pyrazin-7-yl]-1-piperidyl]methanone,
[4-(pentafluoro-λ⁶-sulfanyl)phenyl]-[4-(2-tetrahydropyran-4-yl-5H-pyrrolo[2,3-b]pyrazin-7-yl)-1-piperidyl]methanone,
[2-amino-4-(pentafluoro-λ⁶-sulfanyl)phenyl]-[4-(2-tetrahydropyran-4-yl-5H-pyrrolo[2,3-b]pyrazin-7-yl)-1-piperidyl]methanone,
[2-amino-4-(pentafluoro-λ⁶-sulfanyl)phenyl]-[4-[2-(1-cyclopropyl-4-piperidyl)-5H-pyrrolo[2,3-b]pyrazin-7-yl]-1-piperidyl]methanone,
[4-[2-(1-cyclopropyl-4-piperidyl)-5H-pyrrolo[2,3-b]pyrazin-7-yl]-1-piperidyl]-[4-(pentafluoro-λ⁶-sulfanyl)phenyl]methanone,
[4-(cyclopropoxy)phenyl]-[4-(2-tetrahydropyran-4-yl-5H-pyrrolo[2,3-b]pyrazin-7-yl)-1-piperidyl]methanone,
[4-(1,1,2,2,2-pentafluoroethyl)phenyl]-[4-(2-tetrahydropyran-4-yl-5H-pyrrolo[2,3-b]pyrazin-7-yl)-1-piperidyl]methanone,
cyclohexyl-[4-(2-tetrahydropyran-4-yl-5H-pyrrolo[2,3-b]pyrazin-7-yl)-1-piperidyl]methanone,
[2-amino-4-(trifluoromethoxy)phenyl]-[3-(2-tetrahydropyran-4-yl-5H-pyrrolo[2,3-b]pyrazin-7-yl)pyrrolidine-1-yl]methanone,
[3-(2-tetrahydropyran-4-yl-5H-pyrrolo[2,3-b]pyrazin-7-yl)pyrrolidin-1-yl]-[4-(trifluoromethoxy)phenyl]methanone,
[2-fluoro-4-(pentafluoro-λ⁶-sulfanyl)phenyl]-[4-(2-tetrahydropyran-4-yl-5H-pyrrolo[2,3-b]pyrazin-7-yl)-1-piperidyl]methanone,
[2-amino-4-(trifluoromethoxy)phenyl]-[4-[2-(oxetan-3-ylamino)-5H-pyrrolo[2,3-b]pyrazin-7-yl]-1-piperidyl]methanone,
[2-amino-4-(trifluoromethoxy)phenyl]-[4-[2-[tetrahydrofuran-3-yl]oxy-5H-pyrrolo[2,3-b]pyrazin-7-yl]-1-piperidyl]methanone,
trans-[2-amino-4-(trifluoromethoxy)phenyl]-[4-[2-(4-hydroxycyclohexyl)-5H-pyrrolo[2,3-b]pyrazin-7-yl]-1-piperidyl]methanone,
[4-[2-(4-methylpiperazin-1-yl)-5H-pyrrolo[2,3-b]pyrazin-7-yl]-1-piperidyl]-[4-(trifluoromethoxy)phenyl]methanone,
[4-[2-[[tetrahydrofuran-3-yl]amino]-5H-pyrrolo[2,3-b]pyrazin-7-yl]-1-piperidyl]-[4-(trifluoromethoxy)phenyl]methanone,
[2-amino-4-(trifluoromethoxy)phenyl]-[4-[2-[[tetrahydrofuran-3-yl]amino]-5H-pyrrolo[2,3-b]pyrazin-7-yl]-1-piperidyl]methanone,
[4-[2-(tetrahydropyran-4-ylamino)-5H-pyrrolo[2,3-b]pyrazin-7-yl]-1-piperidyl]-[4-(trifluoromethoxy)phenyl]methanone,
[2-amino-4-(trifluoromethoxy)phenyl]-[4-[2-(tetrahydropyran-4-ylamino)-5H-pyrrolo[2,3-b]pyrazin-7-yl]-1-piperidyl]methanone,
[4-(2-morpholino-5H-pyrrolo[2,3-b]pyrazin-7-yl)-1-piperidyl]-[4-(trifluoromethoxy)phenyl]methanone,
[4-[2-(4,4-difluoro-1-piperidyl)-5H-pyrrolo[2,3-b]pyrazin-7-yl]-1-piperidyl]-[4-(trifluoromethoxy)phenyl]methanone,
[4-(2-tetrahydropyran-4-yl-5H-pyrrolo[2,3-b]pyrazin-7-yl)-1-piperidyl]-[4-[2,2,2-trifluoro-1-hydroxy-1-(trifluoromethyl)ethyl]phenyl]methanone,
[2-amino-4-(pentafluoro-λ⁶-sulfanyl)phenyl]-[3-(2-tetrahydropyran-4-yl-5H-pyrrolo[2,3-b]pyrazin-7-yl)pyrrolidin-1-yl]methanone,
[2-amino-4-(trifluoromethoxy)phenyl]-[4-[2-(oxetan-3-yloxy)-5H-pyrrolo[2,3-b]pyrazin-7-yl]-1-piperidyl]methanone,
   and the pharmaceutically acceptable salts thereof.

In embodiments, the compound is selected from the group consisting of:
[2-amino-4-(trifluoromethoxy)phenyl]-[4-[2-(1-methyl-4-piperidyl)-5H-pyrrolo[2,3-b]pyrazin-7-yl]-1-piperidyl]methanone,
[2-amino-4-(trifluoromethoxy)phenyl]-[4-(2-tetrahydropyran-4-yl-5H-pyrrolo[2,3-b]pyrazin-7-yl)-1-piperidyl]methanone,
[4-(2-tetrahydropyran-4-yl-5H-pyrrolo[2,3-b]pyrazin-7-yl)-1-piperidyl]-[4-(trifluoromethoxy)phenyl]methanone,
[4-[2-(1-methyl-4-piperidyl)-5H-pyrrolo[2,3-b]pyrazin-7-yl]-1-piperidyl]-[4-(trifluoromethoxy)phenyl]methanone,
[4-(2-tetrahydrofuran-3-yl-5H-pyrrolo[2,3-b]pyrazin-7-yl)-1-piperidyl]-[4-(trifluoromethoxy)phenyl]methanone,
[4-[2-(3-methoxypropyl)-5H-pyrrolo[2,3-b]pyrazin-7-yl]-1-piperidyl]-[4-(trifluoromethoxy)phenyl]methanone,
[2-amino-4-(trifluoromethoxy)phenyl]-[4-[2-(3-methoxypropyl)-5H-pyrrolo[2,3-b]pyrazin-7-yl]-1-piperidyl]methanone,
[4-(2-cyclohexyl-5H-pyrrolo[2,3-b]pyrazin-7-yl)-1-piperidyl]-[4-(trifluoromethoxy)phenyl]methanone,
[2-amino-4-(trifluoromethoxy)phenyl]-[4-(2-cyclohexyl-5H-pyrrolo[2,3-b]pyrazin-7-yl)-1-piperidyl]methanone,
[2-amino-4-(trifluoromethoxy)phenyl]-[4-[2-(1-cyclopropyl-4-piperidyl)-5H-pyrrolo[2,3-b]pyrazin-7-yl]-1-piperidyl]methanone,
[4-[2-(1-cyclopropyl-4-piperidyl)-5H-pyrrolo[2,3-b]pyrazin-7-yl]-1-piperidyl]-[4-(trifluoromethoxy)phenyl]methanone,
[2-amino-4-(trifluoromethoxy)phenyl]-[4-(2-tetrahydrofuran-3-yl-5H-pyrrolo[2,3-b]pyrazin-7-yl)-1-piperidyl]methanone,
[4-[2-(1-methyl-4-piperidyl)-5H-pyrrolo[2,3-b]pyrazin-7-yl]-1-piperidyl]-[4-(pentafluoro-λ⁶-sulfanyl)phenyl]methanone,
[2-amino-4-(pentafluoro-λ⁶-sulfanyl)phenyl]-[4-[2-(1-methyl-4-piperidyl)-5H-pyrrolo[2,3-b]pyrazin-7-yl]-1-piperidyl]methanone,
[4-(pentafluoro-λ⁶-sulfanyl)phenyl]-[4-(2-tetrahydropyran-4-yl-5H-pyrrolo[2,3-b]pyrazin-7-yl)-1-piperidyl]methanone,
[2-amino-4-(pentafluoro-λ⁶-sulfanyl)phenyl]-[4-(2-tetrahydropyran-4-yl-5H-pyrrolo[2,3-b]pyrazin-7-yl)-1-piperidyl]methanone,
[2-amino-4-(pentafluoro-λ⁶-sulfanyl)phenyl]-[4-[2-(1-cyclopropyl-4-piperidyl)-5H-pyrrolo[2,3-b]pyrazin-7-yl]-1-piperidyl]methanone,
[4-[2-(1-cyclopropyl-4-piperidyl)-5H-pyrrolo[2,3-b]pyrazin-7-yl]-1-piperidyl]-[4-(pentafluoro-λ⁶-sulfanyl)phenyl]methanone,
[4-(cyclopropoxy)phenyl]-[4-(2-tetrahydropyran-4-yl-5H-pyrrolo[2,3-b]pyrazin-7-yl)-1-piperidyl]methanone,
[4-(1,1,2,2,2-pentafluoroethyl)phenyl]-[4-(2-tetrahydropyran-4-yl-5H-pyrrolo[2,3-b]pyrazin-7-yl)-1-piperidyl]methanone,
cyclohexyl-[4-(2-tetrahydropyran-4-yl-5H-pyrrolo[2,3-b]pyrazin-7-yl)-1-piperidyl]methanone,
[2-amino-4-(trifluoromethoxy)phenyl]-[3-(2-tetrahydropyran-4-yl-5H-pyrrolo[2,3-b]pyrazin-7-yl)pyrrolidine-1-yl]methanone,
[3-(2-tetrahydropyran-4-yl-5H-pyrrolo[2,3-b]pyrazin-7-yl)pyrrolidin-1-yl]-[4-(trifluoromethoxy)phenyl]methanone,
[2-fluoro-4-(pentafluoro-λ⁶-sulfanyl)phenyl]-[4-(2-tetrahydropyran-4-yl-5H-pyrrolo[2,3-b]pyrazin-7-yl)-1-piperidyl]methanone,
   and the pharmaceutically acceptable salts thereof.

In embodiments where the compound has enantiomeric forms (e.g., where the compound has a chiral centre, such as a chiral carbon atom), the compound is present as a racemic mixture of enantiomers. In embodiments where the compound has a chiral centre (e.g., a chiral carbon atom), the compound is present as the (R) isomer. In other embodiments where the compound has a chiral centre (e.g., a chiral carbon atom), the compound is present as the (S) isomer.

Thus, in embodiments the compound is selected from the group consisting of:
(rac)-[4-(2-tetrahydrofuran-3-yl-5H-pyrrolo[2,3-b]pyrazin-7-yl)-1-piperidyl]-[4-(trifluoromethoxy)phenyl]methanone,
(rac)-[2-amino-4-(trifluoromethoxy)phenyl]-[4-(2-tetrahydrofuran-3-yl-5H-pyrrolo[2,3-b]pyrazin-7-yl)-1-piperidyl]methanone,
[2-amino-4-(trifluoromethoxy)phenyl]-[4-[2-[(3S)-tetrahydrofuran-3-yl]-5H-pyrrolo[2,3-b]pyrazin-7-yl]-1-piperidyl]methanone,
[2-amino-4-(trifluoromethoxy)phenyl]-[4-[2-[(3R)-tetrahydrofuran-3-yl]-5H-pyrrolo[2,3-b]pyrazin-7-yl]-1-piperidyl]methanone,
(rac)-[2-amino-4-(trifluoromethoxy)phenyl]-[3-(2-tetrahydropyran-4-yl-5H-pyrrolo[2,3-b]pyrazin-7-yl)pyrrolidine-1-yl]methanone,
(rac)-[3-(2-tetrahydropyran-4-yl-5H-pyrrolo[2,3-b]pyrazin-7-yl)pyrrolidin-1-yl]-[4-(trifluoromethoxy)phenyl]methanone,
   and the pharmaceutically acceptable salts thereof.

In embodiments, the compound is selected from the group consisting of:
(rac)-[4-(2-tetrahydrofuran-3-yl-5H-pyrrolo[2,3-b]pyrazin-7-yl)-1-piperidyl]-[4-(trifluoromethoxy)phenyl]methanone,
(rac)-[2-amino-4-(trifluoromethoxy)phenyl]-[4-(2-tetrahydrofuran-3-yl-5H-pyrrolo[2,3-b]pyrazin-7-yl)-1-piperidyl]methanone,
[2-amino-4-(trifluoromethoxy)phenyl]-[4-[2-[(3S)-tetrahydrofuran-3-yl]-5H-pyrrolo[2,3-b]pyrazin-7-yl]-1-piperidyl]methanone,
[2-amino-4-(trifluoromethoxy)phenyl]-[4-[2-[(3R)-tetrahydrofuran-3-yl]-5H-pyrrolo[2,3-b]pyrazin-7-yl]-1-piperidyl]methanone,
(rac)-[2-amino-4-(trifluoromethoxy)phenyl]-[3-(2-tetrahydropyran-4-yl-5H-pyrrolo[2,3-b]pyrazin-7-yl)pyrrolidine-1-yl]methanone,
(rac)-[3-(2-tetrahydropyran-4-yl-5H-pyrrolo[2,3-b]pyrazin-7-yl)pyrrolidin-1-yl]-[4-(trifluoromethoxy)phenyl]methanone,
[2-amino-4-(trifluoromethoxy)phenyl]-[4-[2-[(3R)-tetrahydrofuran-3-yl]oxy-5H-pyrrolo[2,3-b]pyrazin-7-yl]-1-piperidyl]methanone,
[2-amino-4-(trifluoromethoxy)phenyl]-[4-[2-[(3S)-tetrahydrofuran-3-yl]oxy-5H-pyrrolo[2,3-b]pyrazin-7-yl]-1-piperidyl]methanone,
(rac)-trans-[2-amino-4-(trifluoromethoxy)phenyl]-[4-[2-(4-hydroxycyclohexyl)-5H-pyrrolo[2,3-b]pyrazin-7-yl]-1-piperidyl]methanone,
[4-[2-[[(3R)-tetrahydrofuran-3-yl]amino]-5H-pyrrolo[2,3-b]pyrazin-7-yl]-1-piperidyl]-[4-(trifluoromethoxy)phenyl]methanone,
[4-[2-[[(3S)-tetrahydrofuran-3-yl]amino]-5H-pyrrolo[2,3-b]pyrazin-7-yl]-1-piperidyl]-[4-(trifluoromethoxy)phenyl]methanone,
[2-amino-4-(trifluoromethoxy)phenyl]-[4-[2-[[(3R)-tetrahydrofuran-3-yl]amino]-5H-pyrrolo[2,3-b]pyrazin-7-yl]-1-piperidyl]methanone,
[2-amino-4-(trifluoromethoxy)phenyl]-[4-[2-[[(3S)-tetrahydrofuran-3-yl]amino]-5H-pyrrolo[2,3-b]pyrazin-7-yl]-1-piperidyl]methanone,
(rac)-[4-[2-(tetrahydrofuran-3-ylamino)-5H-pyrrolo[2,3-b]pyrazin-7-yl]-1-piperidyl]-[4-(trifluoromethoxy)phenyl]methanone,
(rac)-[2-amino-4-(trifluoromethoxy)phenyl]-[4-[2-(tetrahydrofuran-3-ylamino)-5H-pyrrolo[2,3-b]pyrazin-7-yl]-1-piperidyl]methanone,
(rac)-[2-amino-4-(pentafluoro-λ⁶-sulfanyl)phenyl]-[3-(2-tetrahydropyran-4-yl-5H-pyrrolo[2,3-b]pyrazin-7-yl)pyrrolidin-1-yl]methanone,
   and the pharmaceutically acceptable salts thereof.

In embodiments, the compound is selected from the compounds produced in Examples 1 to 44 (i.e., from the group consisting of Compounds 1-44), and the pharmaceutically acceptable salts thereof. In other embodiments, the compound is selected from the compounds obtainable by the synthetic methods described in any one of Examples 1 to 44 (i.e., the methods for synthesising Compounds 1-44), and the pharmaceutically acceptable salts thereof.

In embodiments, the compound is selected from the compounds produced in Examples 1 to 26 (i.e., from the group consisting of Compounds 1-26), and the pharmaceutically acceptable salts thereof. In other embodiments, the compound is selected from the compounds obtainable by the synthetic methods described in any one of Examples 1 to 26 (i.e., the methods for synthesising Compounds 1-26), and the pharmaceutically acceptable salts thereof. In embodiments, the compound is selected from the group consisting of:
[2-amino-4-(trifluoromethoxy)phenyl]-[4-[2-(1-methyl-4-piperidyl)-5H-pyrrolo[2,3-b]pyrazin-7-yl]-1-piperidyl]methanone,
[2-amino-4-(trifluoromethoxy)phenyl]-[4-(2-tetrahydropyran-4-yl-5H-pyrrolo[2,3-b]pyrazin-7-yl)-1-piperidyl]methanone,
[4-(2-tetrahydropyran-4-yl-5H-pyrrolo[2,3-b]pyrazin-7-yl)-1-piperidyl]-[4-(trifluoromethoxy)phenyl]methanone,
[4-[2-(1-methyl-4-piperidyl)-5H-pyrrolo[2,3-b]pyrazin-7-yl]-1-piperidyl]-[4-(trifluoromethoxy)phenyl]methanone,
(rac)-[4-(2-tetrahydrofuran-3-yl-5H-pyrrolo[2,3-b]pyrazin-7-yl)-1-piperidyl]-[4-(trifluoromethoxy)phenyl]methanone,
[4-[2-(3-methoxypropyl)-5H-pyrrolo[2,3-b]pyrazin-7-yl]-1-piperidyl]-[4-(trifluoromethoxy)phenyl]methanone,
[2-amino-4-(trifluoromethoxy)phenyl]-[4-[2-(3-methoxypropyl)-5H-pyrrolo[2,3-b]pyrazin-7-yl]-1-piperidyl]methanone,
[4-(2-cyclohexyl-5H-pyrrolo[2,3-b]pyrazin-7-yl)-1-piperidyl]-[4-(trifluoromethoxy)phenyl]methanone,
[2-amino-4-(trifluoromethoxy)phenyl]-[4-(2-cyclohexyl-5H-pyrrolo[2,3-b]pyrazin-7-yl)-1-piperidyl]methanone,
[2-amino-4-(trifluoromethoxy)phenyl]-[4-[2-(1-cyclopropyl-4-piperidyl)-5H-pyrrolo[2,3-b]pyrazin-7-yl]-1-piperidyl]methanone,
[4-[2-(1-cyclopropyl-4-piperidyl)-5H-pyrrolo[2,3-b]pyrazin-7-yl]-1-piperidyl]-[4-(trifluoromethoxy)phenyl]methanone,
(rac)-[2-amino-4-(trifluoromethoxy)phenyl]-[4-(2-tetrahydrofuran-3-yl-5H-pyrrolo[2,3-b]pyrazin-7-yl)-1-piperidyl]methanone,
[2-amino-4-(trifluoromethoxy)phenyl]-[4-[2-[(3S)-tetrahydrofuran-3-yl]-5H-pyrrolo[2,3-b]pyrazin-7-yl]-1-piperidyl]methanone,
[2-amino-4-(trifluoromethoxy)phenyl]-[4-[2-[(3R)-tetrahydrofuran-3-yl]-5H-pyrrolo[2,3-b]pyrazin-7-yl]-1-piperidyl]methanone,
[4-[2-(1-methyl-4-piperidyl)-5H-pyrrolo[2,3-b]pyrazin-7-yl]-1-piperidyl]-[4-(pentafluoro-λ⁶-sulfanyl)phenyl]methanone,
[2-amino-4-(pentafluoro-λ⁶-sulfanyl)phenyl]-[4-[2-(1-methyl-4-piperidyl)-5H-pyrrolo[2,3-b]pyrazin-7-yl]-1-piperidyl]methanone,
[4-(pentafluoro-λ⁶-sulfanyl)phenyl]-[4-(2-tetrahydropyran-4-yl-5H-pyrrolo[2,3-b]pyrazin-7-yl)-1-piperidyl]methanone,
[2-amino-4-(pentafluoro-λ⁶-sulfanyl)phenyl]-[4-(2-tetrahydropyran-4-yl-5H-pyrrolo[2,3-b]pyrazin-7-yl)-1-piperidyl]methanone,
[2-amino-4-(pentafluoro-λ⁶-sulfanyl)phenyl]-[4-[2-(1-cyclopropyl-4-piperidyl)-5H-pyrrolo[2,3-b]pyrazin-7-yl]-1-piperidyl]methanone,
[4-[2-(1-cyclopropyl-4-piperidyl)-5H-pyrrolo[2,3-b]pyrazin-7-yl]-1-piperidyl]-[4-(pentafluoro-λ⁶-sulfanyl)phenyl]methanone,
[4-(cyclopropoxy)phenyl]-[4-(2-tetrahydropyran-4-yl-5H-pyrrolo[2,3-b]pyrazin-7-yl)-1-piperidyl]methanone,
[4-(1,1,2,2,2-pentafluoroethyl)phenyl]-[4-(2-tetrahydropyran-4-yl-5H-pyrrolo[2,3-b]pyrazin-7-yl)-1-piperidyl]methanone,
cyclohexyl-[4-(2-tetrahydropyran-4-yl-5H-pyrrolo[2,3-b]pyrazin-7-yl)-1-piperidyl]methanone,
(rac)-[2-amino-4-(trifluoromethoxy)phenyl]-[3-(2-tetrahydropyran-4-yl-5H-pyrrolo[2,3-b]pyrazin-7-yl)pyrrolidine-1-yl]methanone,
(rac)-[3-(2-tetrahydropyran-4-yl-5H-pyrrolo[2,3-b]pyrazin-7-yl)pyrrolidin-1-yl]-[4-(trifluoromethoxy)phenyl]methanone,
[2-fluoro-4-(pentafluoro-λ⁶-sulfanyl)phenyl]-[4-(2-tetrahydropyran-4-yl-5H-pyrrolo[2,3-b]pyrazin-7-yl)-1-piperidyl]methanone, and the pharmaceutically acceptable salts thereof.

In embodiments, the compound of the disclosure is characterised according to its inhibitory activity against ERK5, e.g., as measured according to the cell-based assay or cell-free assay described in the examples below. In embodiments, the compound has an IC₅₀ value of less than about 10 μM against ERK5 (e.g., when measured according to the cell-free assay described below). In embodiments, the compound has an IC₅₀ value of less than about 5 μM against ERK5 (e.g., when measured according to the cell-free assay described below). In embodiments, the compound has an IC₅₀ value of less than about 2 μM, e.g., less than about 1 μM, 0.5 μM, 0.2 μM, 100 nM, or 50 nM against ERK5 (e.g., when measured according to the cell-free assay described below). In embodiments, the compound has an IC₅₀ value of less than about 10 μM against ERK5 when measured according to the cell-free assay described below. In embodiments, the compound has an IC₅₀ value of less than about 5 μM, e.g., less than about 2 μM, 1 μM, 0.5 μM, 0.2 μM, 100 nM, or 50 nM against ERK5 when measured according to the cell-free assay described below. In embodiments, the compound has an IC₅₀ value of less than about 10 μM against ERK5 when measured according to the cell-based assay described below. In embodiments, the compound has an IC₅₀ value of less than about 5 μM, e.g., less than about 2 μM, 1 μM, 0.5 μM, 0.2 μM, 100 nM, or 50 nM against ERK5 when measured according to the cell-based assay described below.

In embodiments, the compound is selected from the compound of Examples 1, 2, 3, 4, 5, 6, 7, 8, 9, 10, 11, 12, 13, 14, 15, 16, 17, 18, 19, 20, 21, 22, 24, 25, and 26. In other embodiments, the compound is selected from the compound of Examples 1, 2, 3, 4, 5, 6, 7, 9, 10, 11, 12, 13, 14, 15, 16, 17, 18, 19, and 26. In other embodiments, the compound is selected from the compound of Examples 1, 2, 3, 4, 5, 7, 10, 12, 13, 14, 15, 16, 18, and 19. In other embodiments, the compound is selected from the compound of Examples 1, 2, 3, 10, 12, 13, 14, 16, and 18.

In embodiments, the compound is selected from the compound of Examples 1, 2, 3, 4, 5, 6, 7, 8, 9, 10, 11, 12, 13, 14, 15, 16, 17, 18, 19, 20, 21, 22, 23, and 26. In other embodiments, the compound is selected from the compound of Examples 1, 2, 3, 4, 5, 6, 7, 10, 11, 12, 13, 14, 15, 16, 17, 18, 19, 20, 21, and 26. In other embodiments, the compound is selected from the compound of Examples 1, 2, 3, 4, 5, 6, 7, 10, 11, 12, 13, 14, 15, 16, 18, 19, 20, and 21. In other embodiments, the compound is selected from the compound of Examples 1, 2, 3, 4, 7, 10, 11, 12, 13, 14, 15, 16, and 19. In other embodiments, the compound is selected from the compound of Examples 1, 2, 4, 10, 11, 13, 14, 15, and 16. In other embodiments, the compound is selected from the compound of Examples 1, 2, 10, and 16.

In embodiments, the compound is selected from the compound of Examples 1, 2, 3, 4, 5, 6, 7, 8, 9, 10, 11, 12, 13, 14, 15, 16, 17, 18, 19, 20, 21, 22, 24, 25, 26, 28, 29, 30, 31, 32, 33, 35, 36, 37, 38, 39, 40, 41, 42, 43, and 44. In other embodiments, the compound is selected from the compound of Examples 1, 2, 3, 4, 5, 6, 7, 9, 10, 11, 12, 13, 14, 15, 16, 17, 18, 19, 26, 28, 30, 31, 32, 33, 36, 37, 38, 39, and 40. In other embodiments, the compound is selected from the compound of Examples 1, 2, 3, 4, 5, 7, 10, 12, 13, 14, 15, 16, 18, 19, 30, 31, 32, 37, and 39. In other embodiments, the compound is selected from the compound of Examples 1, 2, 3, 10, 12, 13, 14, 16, 18, 30, and 39.

In embodiments, the compound is selected from the compound of Examples 1, 2, 3, 4, 5, 6, 7, 8, 9, 10, 11, 12, 13, 14, 15, 16, 17, 18, 19, 20, 21, 22, 23, 26, 27, 28, 29, 30, 31, 32, 33, 34, 35, 36, 37, 38, 39, 40, 43, and 44. In other embodiments, the compound is selected from the compound of Examples 1, 2, 3, 4, 5, 6, 7, 10, 11, 12, 13, 14, 15, 16, 17, 18, 19, 20, 21, 26, 28, 29, 30, 31, 32, 33, 34, 35, 36, 37, 38, and 39. In other embodiments, the compound is selected from the compound of Examples 1, 2, 3, 4, 5, 6, 7, 10, 11, 12, 13, 14, 15, 16, 18, 19, 20, 21, 28, 29, 30, 31, 32, 33, 34, 35, 36, 37, and 39. In other embodiments, the compound is selected from the compound of Examples 1, 2, 3, 4, 7, 10, 11, 12, 13, 14, 15, 16, 19, 30, 31, 34, 35, and 37. In other embodiments, the compound is selected from the compound of Examples 1, 2, 4, 10, 11, 13, 14, 15, 16, 30, 34, and 37. In other embodiments, the compound is selected from the compound of Examples 1, 2, 4, 10, 11, 13, 14, 15, 16, and 30.

### Pharmaceutical Compositions

The present disclosure provides a pharmaceutical composition comprising a compound described herein (e.g., a compound of Formula (I) or a pharmaceutically acceptable salt thereof), and at least one pharmaceutically acceptable excipient or carrier.

In embodiments, the pharmaceutical composition comprises a compound of Formula (I) or a pharmaceutically acceptable salt thereof. In embodiments, the pharmaceutical composition comprises a compound of Formula (I-A) or a pharmaceutically acceptable salt thereof. In embodiments, the pharmaceutical composition comprises a compound of Formula (I-B) or a pharmaceutically acceptable salt thereof. In embodiments, the pharmaceutical composition comprises a compound of Formula (I-C) or a pharmaceutically acceptable salt thereof. In embodiments, the pharmaceutical composition comprises a compound of Formula (I-D) or a pharmaceutically acceptable salt thereof. In embodiments, the pharmaceutical composition comprises a compound of Formula (I-E) or a pharmaceutically acceptable salt thereof. In embodiments, the pharmaceutical composition comprises a compound of Formula (I-F) or a pharmaceutically acceptable salt thereof. In embodiments, the pharmaceutical composition comprises a compound of Formula (I-G) or a pharmaceutically acceptable salt thereof. In embodiments, the pharmaceutical composition comprises a compound of Formula (II) or a pharmaceutically acceptable salt thereof. In embodiments, the pharmaceutical composition comprises a compound of Formula (II-A) or a pharmaceutically acceptable salt thereof. In embodiments, the pharmaceutical composition comprises a compound of Formula (II-B) or a pharmaceutically acceptable salt thereof. In embodiments, the pharmaceutical composition comprises a compound of Formula (III) or a pharmaceutically acceptable salt thereof. In embodiments, the pharmaceutical composition comprises a compound of Formula (III-A) or a pharmaceutically acceptable salt thereof. In embodiments, the pharmaceutical composition comprises a compound of Formula (IV) or a pharmaceutically acceptable salt thereof. In embodiments, the pharmaceutical composition comprises a compound of Formula (IV-A) or a pharmaceutically acceptable salt thereof. In embodiments, the pharmaceutical composition comprises a compound of Formula (V) or a pharmaceutically acceptable salt thereof. In embodiments, the pharmaceutical composition comprises a compound of Formula (V-A) or a pharmaceutically acceptable salt thereof. In embodiments, the pharmaceutical composition comprises a compound of Formula (VI) or a pharmaceutically acceptable salt thereof. In embodiments, the pharmaceutical composition comprises a compound of Formula (VII) or a pharmaceutically acceptable salt thereof. In embodiments, the pharmaceutical composition comprises a compound of Formula (VIII) or a pharmaceutically acceptable salt thereof.

The pharmaceutical compositions of the disclosure may be formulated for administration in solid or liquid form, e.g., using conventional carriers or excipients. Compositions may be adapted for, e.g., oral administration (e.g., as a solution, suspension, tablet, or capsule), parenteral administration (e.g., as a solution, dispersion, suspension, or emulsion, or as a dry powder for reconstitution), or topical application (e.g., as a cream, ointment, patch, or spray to be applied to the skin) using techniques known in the art.

### Medical Uses

Compounds of the present disclosure act as inhibitors of ERK5, which gives them utility in the treatment of ERK5-associated disorders and conditions. In particular, compounds of the disclosure are useful in the treatment of cancers.

Viewed from this aspect, the disclosure provides a method of treatment comprising administering to a subject in need thereof a therapeutically effective amount of a compound of the disclosure (e.g., a compound of Formula (I) or a pharmaceutically acceptable salt thereof). In a related aspect, the disclosure provides the use of a compound of the disclosure (e.g., a compound of Formula (I) or a pharmaceutically acceptable salt thereof) in the manufacture of a medicament. In a further related aspect, the disclosure provides a compound of the disclosure (e.g., a compound of Formula (I) or a pharmaceutically acceptable salt thereof) for use in therapy.

Compounds of the present disclosure are useful for treating or preventing: diseases or deleterious conditions in which ERK5, or a variant or mutant thereof, is known to play a role; diseases or disorders associated with increased MAPK7 (i.e., ERK5 gene) expression and/or increased ERK5 activity; and diseases or disorders in which inhibition or antagonism of ERK5 activity is beneficial.

In one aspect, the present disclosure provides a method of treating or preventing a disease or disorder mediated by ERK5, or a disease or disorder in which ERK5 is implicated, in a subject in need thereof, the method comprising administering to the subject an effective amount of a compound of the disclosure (e.g., a compound of Formula (I) or a pharmaceutically acceptable salt thereof). In a related aspect, the disclosure provides the use of a compound of the disclosure (e.g., a compound of Formula (I) or a pharmaceutically acceptable salt thereof) in the manufacture of a medicament for the treatment or prevention of a disease or disorder mediated by ERK5, or a disease or disorder in which ERK5 is implicated. In a further related aspect, the disclosure provides a compound of the disclosure (e.g., a compound of Formula (I) or a pharmaceutically acceptable salt thereof) for use in the treatment or prevention of a disease or disorder mediated by ERK5, or a disease or disorder in which ERK5 is implicated.

In another aspect, the present disclosure provides a method of treating or preventing a disease or disorder associated with ERK5 (e.g., cancer) in a subject in need thereof, the method comprising administering to the subject an effective amount of a compound of the disclosure (e.g., a compound of Formula (I) or a pharmaceutically acceptable salt thereof). In a related aspect, the disclosure provides the use of a compound of the disclosure (e.g., a compound of Formula (I) or a pharmaceutically acceptable salt thereof) in the manufacture of a medicament for the treatment or prevention of a disease or disorder associated with ERK5 (e.g., cancer). In a further related aspect, the disclosure provides a compound of the disclosure (e.g., a compound of Formula (I) or a pharmaceutically acceptable salt thereof) for use in the treatment or prevention of a disease or disorder associated with ERK5 (e.g., cancer).

In another aspect, the present disclosure provides a method of treating or preventing cancer in a subject in need thereof, the method comprising administering to the subject an effective amount of a compound of the disclosure (e.g., a compound of Formula (I) or a pharmaceutically acceptable salt thereof). In a related aspect, the disclosure provides the use of a compound of the disclosure (e.g., a compound of Formula (I) or a pharmaceutically acceptable salt thereof) in the manufacture of a medicament for the treatment or prevention of cancer. In a further related aspect, the disclosure provides a compound of the disclosure (e.g., a compound of Formula (I) or a pharmaceutically acceptable salt thereof) for use in the treatment or prevention of cancer.

In embodiments, the compound reduces angiogenesis, reduces or prevents metastasis, reduces inflammation, blocks tumorigenesis (e.g., in part or completely), reduces evasion of growth suppression, reduces or inhibits growth of cancerous or pre-cancerous cells, suppresses proliferation of cancerous or pre-cancerous cells, and/or reduces the survival of cancerous or pre-cancerous cells.

In embodiments, the cancer is characterized by increased MAPK7 (i.e., ERK5 gene) expression and/or increased ERK5 activity. In embodiments, the cancer has elevated ERK5 activity. In embodiments, the cancer overexpresses ERK5. In embodiments, the cancer is characterised by MAPK7 genomic amplification and/or constitutively active ERK5 signalling. In embodiments, the cancer has genomically amplified ERK5. In embodiments, the cancer has constitutively active ERK5 signalling.

In embodiments, the cancer is a solid tumour (e.g., a melanoma, carcinoma, or blastoma). In other embodiments, the cancer is leukaemia (e.g., chronic lymphocytic leukaemia, CLL; acute myelogenous leukaemia, AML; or chronic myelogenous leukaemia, CVL).

In embodiments, the cancer is a primary tumour. In other embodiments, the cancer is a secondary tumour (e.g., a metastatic tumour).

In embodiments, the cancer is selected from breast cancer (e.g., ductal breast carcinoma, or breast adenocarcinoma), liver cancer, kidney cancer (e.g., hepatocellular carcinoma), prostate cancer, colorectal cancer (CRC), lung cancer (e.g., non-small cell lung cancer, NSCLC; lung adenocarcinoma; or lung squamous cell carcinoma), pancreatic cancer (e.g., adenocarcinoma), ovarian cancer, brain cancer (e.g., glioblastoma), cervical cancer (e.g., adenocarcinoma), gastric cancer, skin cancer (e.g., melanoma), bile duct cancer (e.g., cholangiocarcinoma), nervous system cancer (e.g., neuroblastoma), and melanoma.

In embodiments, the cancer is selected from leukaemia (e.g., acute leukaemia, acute lymphocytic leukaemia, acute myelocytic leukaemia, acute myeloblastic leukaemia, acute promyelocytic leukaemia, acute myelomonocytic leukaemia, acute monocytic leukaemia, acute erythroleukemia, chronic leukaemia, chronic myelocytic leukaemia, or chronic lymphocytic leukaemia), polycythaemia vera, lymphoma (e.g., Hodgkin's disease or non-Hodgkin's disease), Waldenstrom macroglobulinemia, and multiple myeloma.

In embodiments, the cancer is selected from leukaemia (e.g., chronic myeloid leukaemia), breast cancer, multiple myeloma, colon cancer, colorectal cancer, lung cancer, pancreatic cancer, renal cell carcinoma, mesothelioma, adenocarcinoma, neuroblastoma, melanoma, and hepatocellular carcinoma.

In embodiments, the cancer is selected from leukaemia (e.g., chronic myeloid leukaemia), breast cancer, multiple myeloma, colon cancer, renal cell carcinoma, mesothelioma, adenocarcinoma, neuroblastoma, and hepatocellular carcinoma.

In another aspect, the disclosure provides a method of inhibiting ERK5 activity, the method comprising contacting ERK5 (e.g., a cell comprising ERK5) with a compound of the present disclosure (e.g., a compound of Formula (I) or a pharmaceutically acceptable salt thereof). In embodiments, the method is an in vitro or ex vivo method. In other embodiments the method is an in vivo method. In a related aspect, the disclosure provides an in vitro method of inhibiting ERK5 activity in a cell, the method comprising contacting the cell with a compound of the present disclosure (e.g., a compound of Formula (I) or a pharmaceutically acceptable salt thereof).

Compounds of the present disclosure (e.g., compounds of Formula (I)) and the pharmaceutically acceptable salts thereof may be administered as pharmaceutical compositions, which may optionally comprise one or more pharmaceutically acceptable excipients.

It will be appreciated that the methods and treatments of the various aspects of this disclosure may be effected by administering to a subject an effective amount of a compound of the disclosure (e.g., a compound of Formula (I) or a pharmaceutically acceptable salt thereof), in the form of a pharmaceutical composition, which may optionally comprise one or more pharmaceutically acceptable excipients, as described herein.

The compounds of the disclosure may be used alone (e.g., as a monotherapy) or in combination with one or more cancer therapies.

Having been generally described herein, the follow non-limiting examples are provided to further illustrate this disclosure.

### EXAMPLES

### General Synthetic Schemes

The following scheme, Scheme 1A, illustrates an exemplary way of preparing compounds in accordance with the present disclosure and examples:

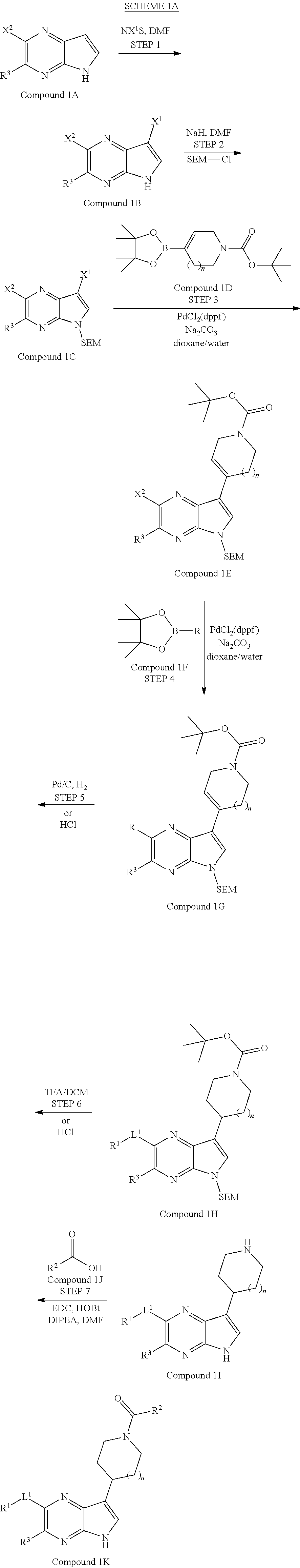

According to Scheme 1A (in which R¹, R², and n may be, e.g., defined as described above; R³ is —H or —CH₃; and L¹ is a direct bond), Compound 1B (in which X¹ may be, e.g., I, or Br; and X² may be, e.g., Br, or Cl) can be obtained in STEP 1 by halogenation of Compound 1A with either NIS or NBS (i.e. X¹═I, or Br), for example; that reagent may be chosen such that X¹ and X² are different halogens. Compound 1C can be protected by N-alkylation of Compound 1B in STEP 2 using, for example, a base such as sodium hydride and an alkylating agent such as SEM-Cl. Compound 1E can be obtained by Suzuki coupling in STEP 3 between Compound 1C and Compound 1D using, for example, a catalyst such as [1,1′-bis(diphenylphosphino)ferrocene]dichloropalladium(II) in a mixture of dioxane and water and in the presence of a base, such as sodium carbonate, by heating to reflux. Compound 1G can be prepared by Suzuki coupling in STEP 4 between Compound 1E and Compound 1F (in which R is a precursor to R¹, e.g. an oxidised or protected analog of R) using, for example, a catalyst such as [1,1′-bis(diphenylphosphino)ferrocene]dichloropalladium(II) in a mixture of dioxane and water and in the presence of a base, such as sodium carbonate, by heating up to reflux of solvent. Compound 1G can be reduced in STEP 5 to Compound 1H by hydrogenation with a catalyst such as Pd/C under hydrogen pressure (H₂) around 5 bars at 40° C., for example. STEP 5 may optionally further comprise modification of R to R¹, for example by hydrogenation with Pd/C and H₂ or by reduction with LiAlH₄ and etherification with an alkyl halide (e.g. as in Example 6). Compound 1H can then be converted to Compound 11 in STEP 6 by deprotection using TFA or HCl in DCM. Compound 1K can then be prepared from Compound 11 in STEP 7 with carboxylic acid Compound 1J, using conditions known by the person skilled in the art such as EDC in a solvent like DMF in presence of a base such as DIPEA. STEP 7 may optionally further comprise modification of R¹. For example, compounds in which R¹ is substituted with oxo may be reduced, e.g. using a hydride reagent, to afford the corresponding compound in which R¹ is substituted with —OH.

In the preparation of compounds of the disclosure in which L¹ is —O—, the conversion of Compound 1C to Compound 1H′ may be effected according to the following scheme, Scheme 1B:

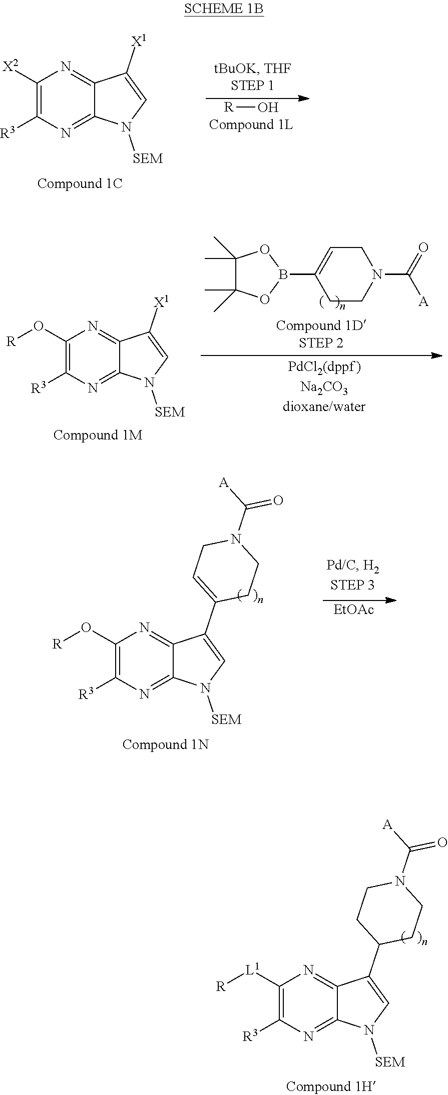

According to Scheme 1B (in which R¹, R³, X¹, X², and n may be, e.g., defined as described above; and L¹ is —O—), Compound 1M can be prepared in STEP 1 by nucleophilic substitution of Compound 1C by Compound 1L (in which R may be R¹, or a precursor to R¹, e.g. a protected analog of R¹) using a base such as tBuOK. Compound 1N can then be prepared in STEP 2 by Suzuki coupling between Compound 1M and Compound 1D′ (in which A is selected from R² as defined herein, a precursor to R² (e.g., having an —NO₂ group in place of an —NH₂ group), or taken together with the carbonyl to which it is attached forms a protecting group (e.g., Boc where A is —O-tBu, or Cbz where A is -OBn)) using, for example, a catalyst such as [1,1′-bis(diphenylphosphino)ferrocene]dichloropalladium(II) in a mixture of dioxane and water and in the presence of a base, such as sodium carbonate, by heating to reflux. Compound 1N can be reduced in STEP 3 to Compound 1H by hydrogenation with a catalyst such as Pd/C under hydrogen pressure (H₂) around 5 bars at 40° C., for example. STEP 3 may optionally further comprise modification of R to R¹, for example by deprotection (such as removal of a Boc group, e.g. using TFA in DCM, or removal of a Cbz group by the hydrogenation of STEP 3), and optionally further functionalisation, e.g., reductive amination with an aldehyde such as formaldehyde in the presence of a hydride reagent such as sodium cyanoborohydride. Compound 1H′ may then be converted to a compound of the disclosure (e.g., a compound of Formula (I) in which L¹ is —O—) by following STEPS 6 and 7 of Scheme 1A above.

In the preparation of compounds in which L¹ is —NH—, the conversion of Compound 1C to Compound 1H′ may be effected according to the following scheme, Scheme 1C:

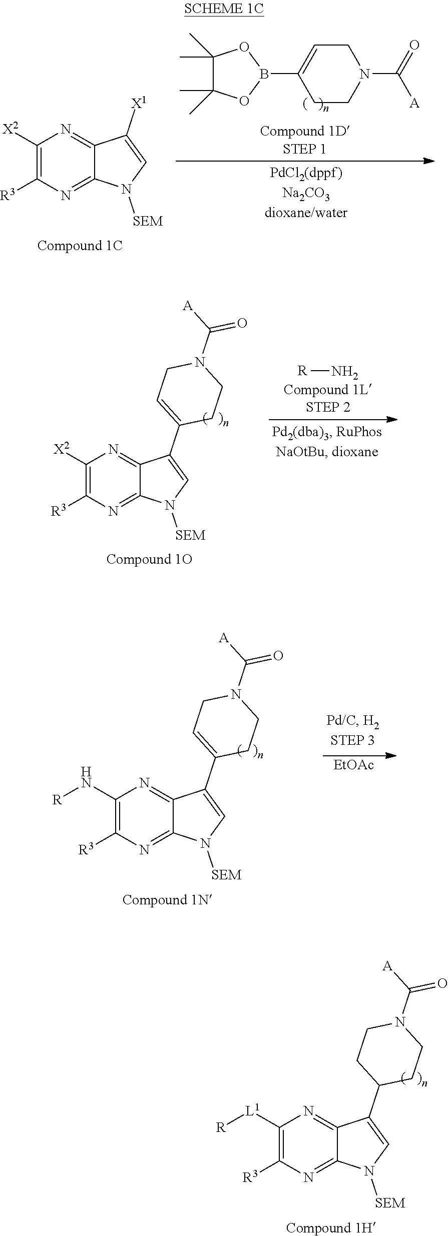

According to Scheme 1C (in which R¹, R³, X², and n may be, e.g., defined as described above; and L¹ is —NH—), Compound 10 can be prepared in STEP 1, by Suzuki coupling between Compound 1C and Compound 1D′ (in which A may be, e.g., defined as described above) using, for example, a catalyst such as [1,1′-bis(diphenylphosphino)ferrocene]dichloropalladium(II) in a mixture of dioxane and water and in the presence of a base, such as sodium carbonate, by heating to reflux. Compound 1N′ can then be prepared in STEP 2 by Buchwald coupling between Compound 1O and Compound 1L′ (in which R may be R¹, or a precursor to R¹, e.g. a protected analog of R¹) using, for example, a catalyst such as tris(dibenzylideneacetone)dipalladium and RuPhos in dioxane and in the presence of a base, such as tBuONa, by heating to reflux. Compound 1N′ can be reduced in STEP 3 to Compound 1H′ by hydrogenation with a catalyst such as Pd/C under hydrogen pressure (H₂) around 5 bars at 40° C., for example. STEP 3 may optionally further comprise modification of R to R¹, for example by deprotection (such as removal of a Boc group, e.g. using TFA in DCM), and optionally further functionalisation, e.g., reductive amination with an aldehyde such as formaldehyde in the presence of a hydride reagent such as sodium cyanoborohydride. Compound 1H′ may then be converted to a compound of the disclosure (e.g., a compound of Formula (I) in which L¹ is —NH—) by following STEPS 6 and 7 of Scheme 1A above.

In Schemes 1B and 1C, the group A in Compound 1D′ may be selected such that the optional modification of R to R¹ in STEP 3 of Scheme 1B or 1C may be effected without modifying A. For example, when A is —O-tBu, R may contain an amine having a Cbz protecting group which can be modified, e.g. as described above, without affecting the protecting group comprising A.

Alternatively, the group A in Compound 1D′ may be selected such that the transformation(s) in STEP 3 of Scheme 1B or 1C also modify that group, e.g. by converting A to R² (where A is a precursor to R²), or by removing a protecting group to furnish Compound 1I as described in Scheme 1A above. For example, compounds of the disclosure in which R² contains an —NH₂ group may be obtained by reduction of —NO₂, e.g. by hydrogenation with H₂ and Pd/C such as in STEP 3 of Scheme 1B or 1C as described above; in this scenario, STEP 3 of Scheme 1B or 1C furnishes Compound 1H, i.e. Compound 1H′ in which A is a group R² as defined herein. In another example, where A is -OBn (i.e., forming a Cbz protecting group on the nitrogen atom of the heterocyclic group), the hydrogenation in STEP 3 of Scheme 1B or 1C can also remove the Cbz group and furnish Compound 1I as described in Scheme 1A above. Compound 1I may then be converted to a compound of the disclosure (e.g., a compound of Formula (I) in which L¹ is —O— or —NH—) by following STEP 7 of Scheme 1A above. These and other suitable protection/deprotection strategies will be readily apparent to a person of skill in the art in view of the present disclosure.

If not commercially available, Compound 1D′ may be prepared, for example, by the following scheme, Scheme 1D.

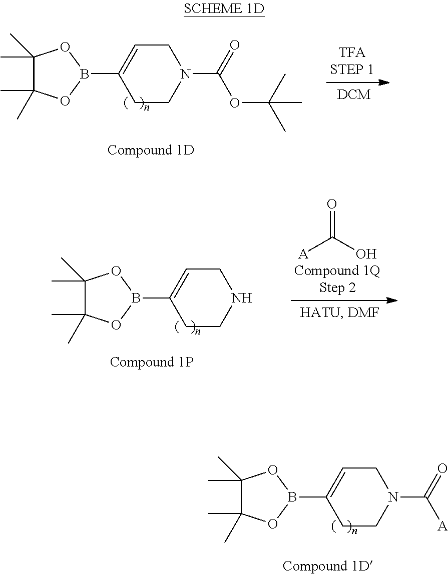

According to Scheme 1D (in which n may be, e.g., defined as described above), Compound 1D can be deprotected, e.g. using TFA in DCM to give Compound 1P. Compound 1D′ (in which A may be, e.g., defined as described above) can then be prepared from Compound 1P and Compound 1Q, using amide coupling conditions such as HATU in DMF. If not commercially available, Compound 1Q may be obtained, for example, from the corresponding nitrile compound using, e.g., H₂SO₄ and AcOH in water. Other methods for preparing carboxylic acids such as Compound 1Q will be apparent to a person of skill in the art in view of the present disclosure.

In the preparation of compounds in which L¹ is —O—, the conversion of Compound 1E to Compound 1H may be effected according to the following scheme, Scheme 1E:

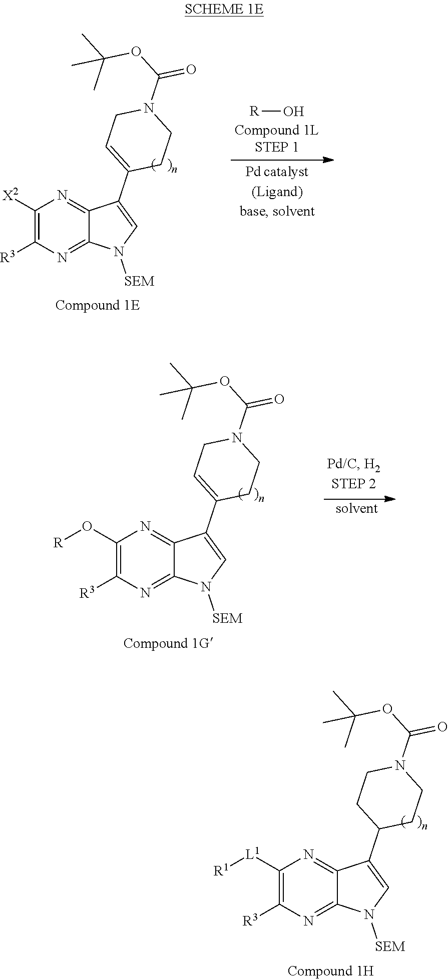

According to Scheme 1E (in which R¹, X², and n may be, e.g., defined as described above; L¹ is —O—; and R³ is —H or —(C₁-C₃)alkyl) Compound 1G′ can be obtained by palladium coupling reaction in STEP 1 between Compound 1E and Compound 1L (in which R may be R¹, or a precursor to R¹, e.g. a protected analog of R) using, for example, a catalyst such as XPhosPdG4 in dioxane and in the presence of a base, such as tBuONa, by heating to reflux. Compound 1G′ can be reduced in STEP 2 to Compound 1H by hydrogenation with a catalyst such as Pd/C under hydrogen pressure (H₂) at 1 to 5 bars at room temperature to 40° C., for example. STEP 2 may optionally further comprise modification of R to R¹, for example by deprotection (such as removal of a Cbz group, e.g. by the hydrogenation of STEP 2), and optionally further functionalisation, e.g., reductive amination with an aldehyde such as formaldehyde in the presence of a hydride reagent such as sodium cyanoborohydride.

In the preparation of compounds in which R¹ is a heterocycloalkyl group which is connected to the rest of the molecule via a heteroatom (e.g., a nitrogen atom), and L¹ is a direct bond, the conversion of Compound 1E to Compound 1H may be effected according to the following scheme, Scheme 1F:

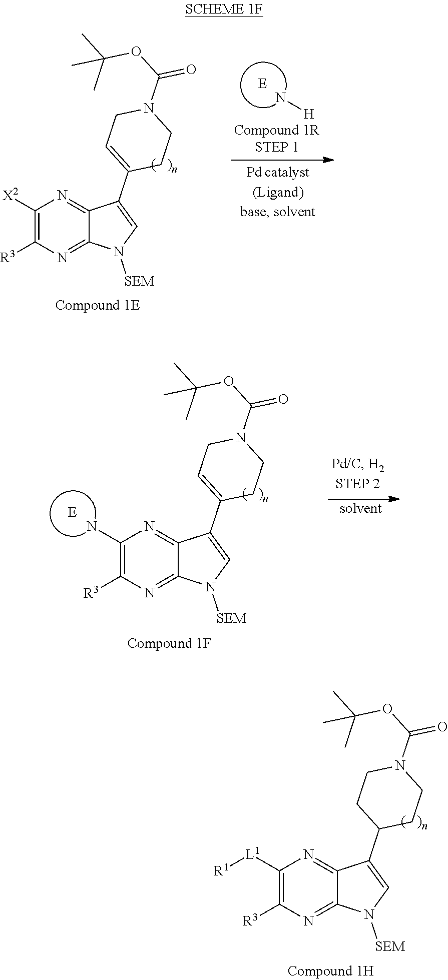

According to Scheme 1F (in which R¹, X², and n may be, e.g., defined as described above; Lt is a direct bond; and R³ is —H or —(C₁-C₃)alkyl), Compound 1S can be obtained by palladium coupling reaction in STEP 1 between Compound 1E and Compound 1R (in which ring E is R¹, or a precursor to R¹, e.g. a protected analog of R¹) using, for example, a catalyst such as XPhosPdG4 in dioxane and in the presence of a base, such as tBuONa, by heating to reflux. Compound 1S can be reduced in STEP 2 to Compound 1H by hydrogenation with a catalyst such as Pd/C under hydrogen pressure (H₂) at 1 to 5 bars at room temperature to 40° C., for example.

Compounds of the disclosure in which Y is N may be synthesized by analogy to the methods shown above, e.g., using a modified version of Scheme 1A, 1B or 1C. Scheme 2A below depicts how such compounds may be prepared by analogy to Scheme 1A.

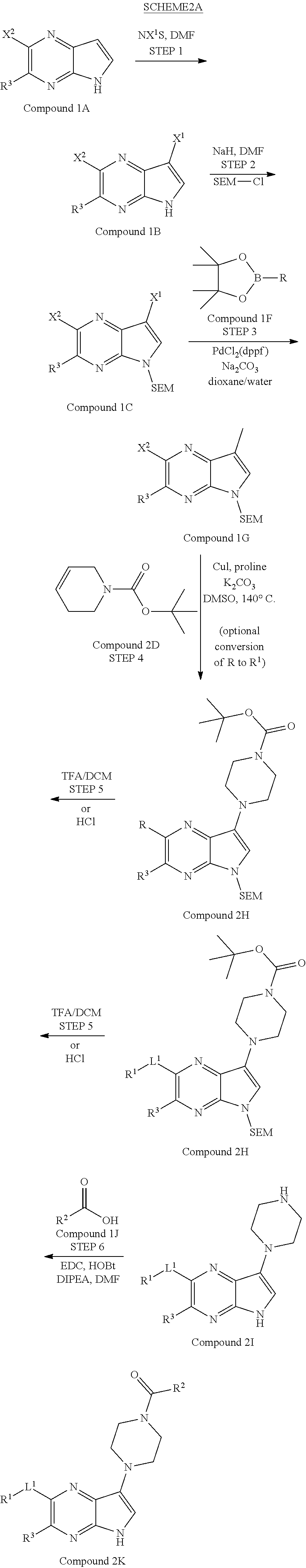

According to Scheme 2A (in which R¹, and R² may be, e.g., defined as described above; R³ is —H or —CH₃; and L¹ is a direct bond), Compound 1B (in which X¹ may be, e.g., Br, or Cl; and X² may be, e.g., I, or Br) can be obtained in STEP 1 by halogenation of Compound 1A with either NBS or NCS (i.e. X¹═Br, or Cl), for example; that reagent may be chosen such that X¹ and X² are different halogens. Compound 1C can be protected by N-alkylation of Compound 1B in STEP 2 using, for example, a base such as sodium hydride and an alkylating agent such as SEM-Cl. Compound 2A can be obtained in STEP 3 by Suzuki coupling between Compound 1C (in which X² may be, e.g., I, or Br) and Compound 1F (in which R is either R¹ or a precursor to R¹, e.g. an oxidised or protected analog of R) using, for example, a catalyst such as [1,1′-bis(diphenylphosphino)ferrocene]dichloropalladium(II) in a mixture of dioxane and water and in the presence of a base, such as sodium carbonate, by heating up to reflux of solvent. Compound 2H can be obtained in STEP 4 by a coupling reaction between Compound 2A and Compound 2D using, for example, a catalyst such as CuI in DMSO and in the presence of proline and a base, such as potassium carbonate, by heating at 140° C. STEP 4 may optionally further comprise a step of converting R to R¹; for example, if R is a precursor to R¹ which possesses a double bond, STEP 4 may further comprise reduction by hydrogenation, e.g. with a catalyst such as Pd/C under hydrogen pressure (H₂) around 5 bars at 40° C. Deprotection of Compound 2H in STEP 5 using TFA or HCl in DCM, for example, can lead to Compound 21. Compound 2K can then be prepared from Compound 21 in STEP 6 with carboxylic acid Compound 1J, using conditions known by the person skilled in the art such as EDC in a solvent like DMF in presence of a base such as DIPEA. Corresponding compounds in which L¹ is —O— or —NH— may be obtained, e.g., by making analogous changes to Schemes 1B and 1C above. For example, Compound 1M may be used in place of Compound 2A in the scheme above (to yield compounds in which L¹ is —O—), or Compound 1C may be reacted with Compound 2D (rather than Compound 1D) in STEP 3 of Scheme 1A to yield a product which can then be taken through a process analogous to Scheme 1C (to yield compounds in which L¹ is —NH—).

Compounds in which R³ is —OH may be accessible, for example, following the synthetic procedures described in, e.g., US 2005/0288299 A1. Intermediates in which R³ is —OH may be protected, for example by etherification, for some of the synthetic steps described above. Compounds in which R¹ is directly bonded to the pyrrolopyrazine core via a tertiary carbon atom (e.g., where R¹ is a bicyclo[1.1.1]pentanyl group) may be obtained, e.g., by a modified version of Scheme 1A in which a heteroaryl halide such as, e.g., Compound 1E is reacted directly with R¹ in the form of a redox active ester, e.g., as described in Polites et al., Org. Lett. (2021) 23(12):4828-4833. Compounds in which R¹ is directly bonded to the pyrrolopyrazine core via a tertiary carbon atom which has one oxygen substituent (e.g., where R^{A} is —OH, or —O(C₁-C₃)alkyl) may be obtained, e.g., by alkylation of a carbonyl group directly bonded to the pyrrolopyrazine core at that position using, for example, an alkyl lithium or alkyl Grignard reagent (such as by the procedure described in WO 2021/195781), optionally with further alkylation of the resulting —OH group with an alkylating agent such as Mel in the presence of a base such as NaH.

### Experimental Techniques

¹H NMR Spectra at 400 and 500 MHz were performed on a Bruker Avance DRX-400 and Bruker Avance DPX-500 spectrometer, respectively, with the chemical shifts (6 in ppm) in the solvent dimethyl sulfoxide-d₆ (DMSO-d₆) referenced at 2.5 ppm at the quoted temperatures. Coupling constants (J) are given in Hertz.

The liquid chromatography/mass spectra (LC/MS) were obtained on a UPLC Acquity Waters instrument, light scattering detector Sedere and SQD Waters mass spectrometer using UV detection DAD 210<1<400 nm and column Acquity UPLC CSH C18 1.7 in, dimension 2.1×30 mm, mobile phase H₂O+0.10 HCN₂H/CH₃CN+0.10 HCO₂H.

All synthetic reactions were performed under an inert atmosphere, unless otherwise stated. In the following examples, when the source of the starting products is not specified, it should be understood that said products are known compounds (e.g., commercially available compounds from suppliers such as Sigma-Aldrich).

### Examples 1 to 44—Compounds

Table 1 below lists the compounds synthesized in the following synthetic examples.

TABLE 1

| Example | | |
|---|---|---|
| No. | Structure | Name |
| 1 | 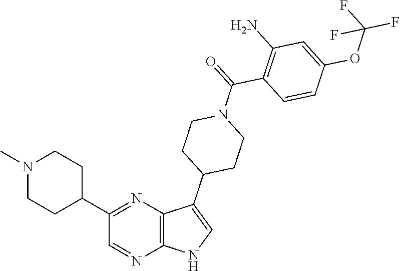 [SMILES: CN1CCC(c2cnc3[nH]cc(C4CCN(C(=O)c5ccc(OC(F)(F)F)cc5N)CC4)c3n2)CC1] | [2-amino-4-(trifluoromethoxy)phenyl]-[4-[2- (1-methyl-4-piperidyl)-5H-pyrrolo[2,3- b]pyrazin-7-yl]-1-piperidyl]methanone |
| 2 | 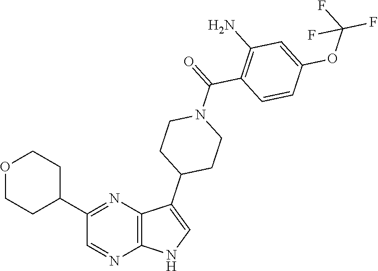 [SMILES: Nc1cc(OC(F)(F)F)ccc1C(=O)N1CCC(c2c[nH]c3ncc(C4CCOCC4)nc23)CC1] | [2-amino-4-(trifluoromethoxy)phenyl]-[4-(2- tetrahydropyran-4-yl-5H-pyrrolo[2,3- b]pyrazin-7-yl)-1-piperidyl]methanone |
| 3 | 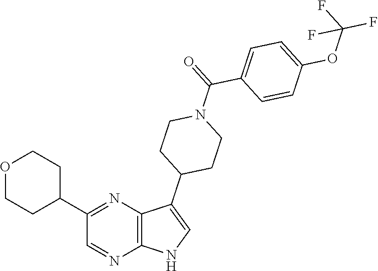 [SMILES: O=C(c1ccc(OC(F)(F)F)cc1)N1CCC(c2c[nH]c3ncc(C4CCOCC4)nc23)CC1] | [4-(2-tetrahydropyran-4-yl-5H-pyrrolo[2,3- b]pyrazin-7-yl)-1-piperidyl]-[4- (trifluoromethoxy)phenyl]methanone |
| 4 | 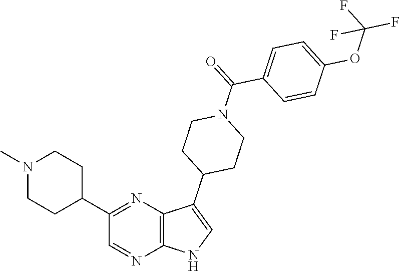 [SMILES: CN1CCC(c2cnc3[nH]cc(C4CCN(C(=O)c5ccc(OC(F)(F)F)cc5)CC4)c3n2)CC1] | [4-[2-(1-methyl-4-piperidyl)-5H- pyrrolo[2,3-b]pyrazin-7-yl]-1-piperidyl]-[4- (trifluoromethoxy)phenyl]methanone |
| 5 | 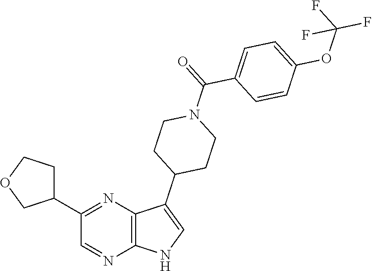 [SMILES: O=C(c1ccc(OC(F)(F)F)cc1)N1CCC(c2c[nH]c3ncc(C4CCOC4)nc23)CC1] | (rac)-[4-(2-tetrahydrofuran-3-yl-5H- pyrrolo[2,3-b]pyrazin-7-yl)-1-piperidyl]-[4- (trifluoromethoxy)phenyl]methanone |
| 6 | 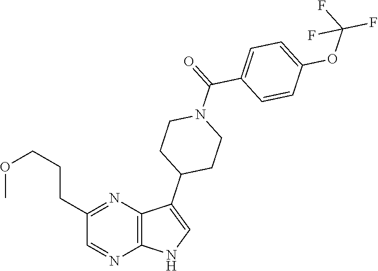 [SMILES: COCCCc1cnc2[nH]cc(C3CCN(C(=O)c4ccc(OC(F)(F)F)cc4)CC3)c2n1] | [4-[2-(3-methoxypropyl)-5H-pyrrolo[2,3- b]pyrazin-7-yl]-1-piperidyl]-[4- (trifluoromethoxy)phenyl]methanone |
| 7 | 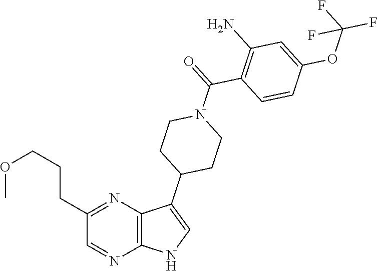 [SMILES: COCCCc1cnc2[nH]cc(C3CCN(C(=O)c4ccc(OC(F)(F)F)cc4N)CC3)c2n1] | [2-amino-4-(trifluoromethoxy)phenyl]-[4-[2- (3-methoxypropyl)-5H-pyrrolo[2,3- b]pyrazin-7-yl]-1-piperidyl]methanone |
| 8 | 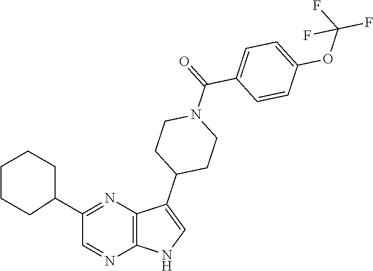 [SMILES: O=C(c1ccc(OC(F)(F)F)cc1)N1CCC(c2c[nH]c3ncc(C4CCCCC4)nc23)CC1] | [4-(2-cyclohexyl-5H-pyrrolo[2,3-b]pyrazin- 7-yl)-1-piperidyl]-[4- (trifluoromethoxy)phenyl]methanone |
| 9 | 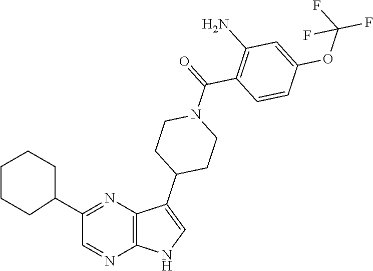 [SMILES: Nc1cc(OC(F)(F)F)ccc1C(=O)N1CCC(c2c[nH]c3ncc(C4CCCCC4)nc23)CC1] | [2-amino-4-(trifluoromethoxy)phenyl]-[4-(2- cyclohexyl-5H-pyrrolo[2,3-b]pyrazin-7-yl)- 1-piperidyl]methanone |
| 10 | 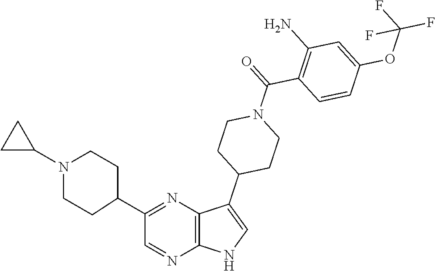 [SMILES: Nc1cc(OC(F)(F)F)ccc1C(=O)N1CCC(c2c[nH]c3ncc(C4CCN(C5CC5)CC4)nc23)CC1] | [2-amino-4-(trifluoromethoxy)phenyl]-[4-[2- (1-cyclopropyl-4-piperidyl)-5H-pyrrolo[2,3- b]pyrazin-7-yl]-1-piperidyl]methanone |
| 11 | 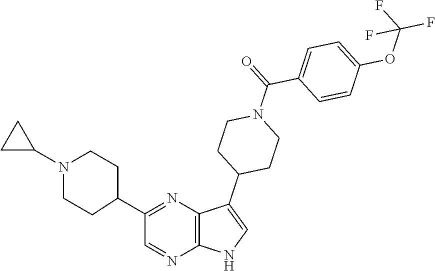 [SMILES: O=C(c1ccc(OC(F)(F)F)cc1)N1CCC(c2c[nH]c3ncc(C4CCN(C5CC5)CC4)nc23)CC1] | [4-[2-(1-cyclopropyl-4-piperidyl)-5H- pyrrolo[2,3-b]pyrazin-7-yl]-1-piperidyl]-[4- (trifluoromethoxy)phenyl]methanone |
| 12 | 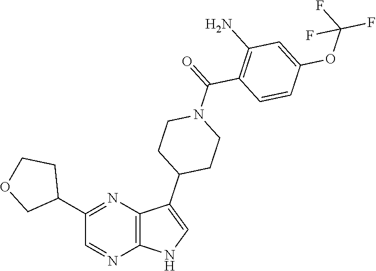 [SMILES: Nc1cc(OC(F)(F)F)ccc1C(=O)N1CCC(c2c[nH]c3ncc(C4CCOC4)nc23)CC1] | (rac)-[2-amino-4-(trifluoromethoxy)phenyl]- [4-(2-tetrahydrofuran-3-yl-5H-pyrrolo[2,3- b]pyrazin-7-yl)-1-piperidyl]methanone |
| 13 | 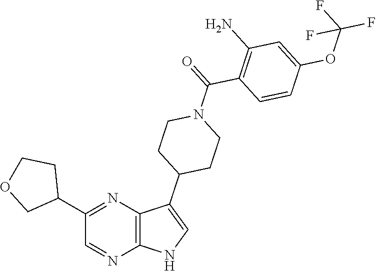 [SMILES: Nc1cc(OC(F)(F)F)ccc1C(=O)N1CCC(c2c[nH]c3ncc(C4CCOC4)nc23)CC1] | [2-amino-4-(trifluoromethoxy)phenyl]-[4-[2- [(tetrahydrofuran-3-yl]-5H-pyrrolo[2,3- b]pyrazin-7-yl]-1-piperidyl]methanone, Isomer 1 |
| 14 | 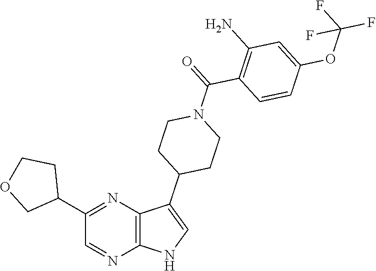 [SMILES: Nc1cc(OC(F)(F)F)ccc1C(=O)N1CCC(c2c[nH]c3ncc(C4CCOC4)nc23)CC1] | [2-amino-4-(trifluoromethoxy)phenyl]-[4-[2- [(tetrahydrofuran-3-yl]-5H-pyrrolo[2,3- b]pyrazin-7-yl]-1-piperidyl]methanone, Isomer 2 |
| 15 | 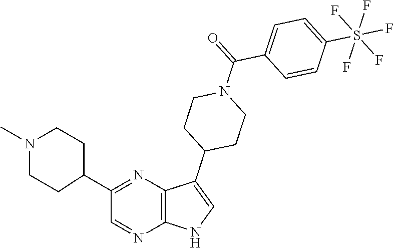 [SMILES: CN1CCC(c2cnc3[nH]cc(C4CCN(C(=O)c5ccc(S(F)(F)(F)(F)F)cc5)CC4)c3n2)CC1] | [4-[2-(1-methyl-4-piperidyl)-5H- pyrrolo[2,3-b]pyrazin-7-yl]-1-piperidyl]-[4- (pentafluoro-λ⁶-sulfanyl)phenyl]methanone |
| 16 | 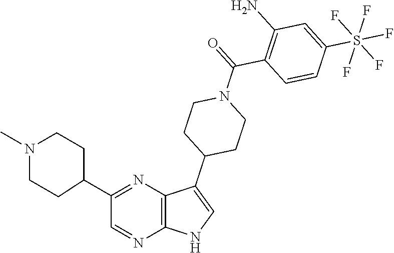 [SMILES: CN1CCC(c2cnc3[nH]cc(C4CCN(C(=O)c5ccc(S(F)(F)(F)(F)F)cc5N)CC4)c3n2)CC1] | [2-amino-4-(pentafluoro-λ⁶- sulfanyl)phenyl]-[4-[2-(1-methyl-4- piperidyl)-5H-pyrrolo[2,3-b]pyrazin-7-yl]-1- piperidyl]methanone |
| 17 | 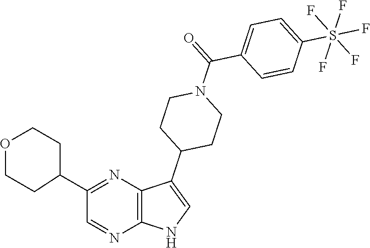 [SMILES: O=C(c1ccc(S(F)(F)(F)(F)F)cc1)N1CCC(c2c[nH]c3ncc(C4CCOCC4)nc23)CC1] | [4-(pentafluoro-λ⁶-sulfanyl)phenyl]-[4-(2- tetrahydropyran-4-yl-5H-pyrrolo[2,3- b]pyrazin-7-yl)-1-piperidyl]methanone |
| 18 | 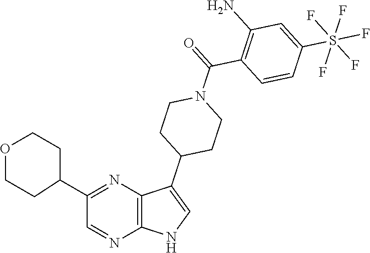 [SMILES: Nc1cc(S(F)(F)(F)(F)F)ccc1C(=O)N1CCC(c2c[nH]c3ncc(C4CCOCC4)nc23)CC1] | [2-amino-4-(pentafluoro-λ⁶- sulfanyl)phenyl]-[4-(2-tetrahydropyran-4-yl- 5H-pyrrolo[2,3-b]pyrazin-7-yl)-1- piperidyl]methanone |
| 19 | 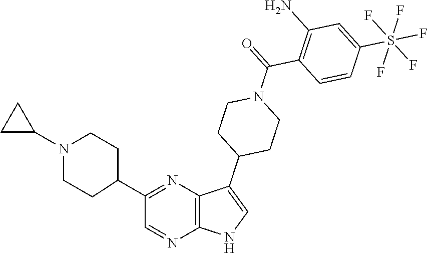 [SMILES: Nc1cc(S(F)(F)(F)(F)F)ccc1C(=O)N1CCC(c2c[nH]c3ncc(C4CCN(C5CC5)CC4)nc23)CC1] | [2-amino-4-(pentafluoro-λ⁶- sulfanyl)phenyl]-[4-[2-(1-cyclopropyl-4- piperidyl)-5H-pyrrolo[2,3-b]pyrazin-7-yl]-1- piperidyl]methanone |
| 20 | 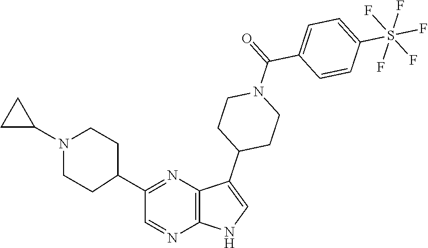 [SMILES: O=C(c1ccc(S(F)(F)(F)(F)F)cc1)N1CCC(c2c[nH]c3ncc(C4CCN(C5CC5)CC4)nc23)CC1] | [4-[2-(1-cyclopropyl-4-piperidyl)-5H- pyrrolo[2,3-b]pyrazin-7-yl]-1-piperidyl]-[4- (pentafluoro-λ⁶-sulfanyl)phenyl]methanone |
| 21 | 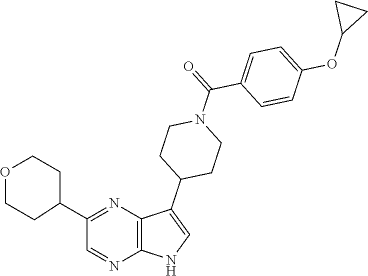 [SMILES: O=C(c1ccc(OC2CC2)cc1)N1CCC(c2c[nH]c3ncc(C4CCOCC4)nc23)CC1] | [4-(cyclopropoxy)phenyl]-[4-(2- tetrahydropyran-4-yl-5H-pyrrolo[2,3- b]pyrazin-7-yl)-1-piperidyl]methanone |
| 22 | 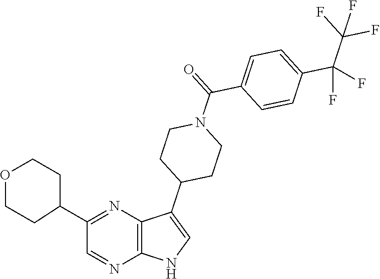 [SMILES: O=C(c1ccc(C(F)(F)C(F)(F)F)cc1)N1CCC(c2c[nH]c3ncc(C4CCOCC4)nc23)CC1] | [4-(1,1,2,2,2-pentafluoroethyl)phenyl]-[4-(2- tetrahydropyran-4-yl-5H-pyrrolo[2,3- b]pyrazin-7-yl)-1-piperidyl]methanone |
| 23 | 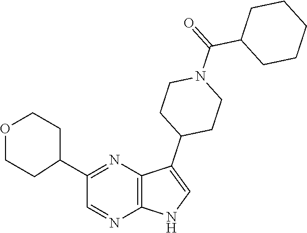 [SMILES: O=C(C1CCCCC1)N1CCC(c2c[nH]c3ncc(C4CCOCC4)nc23)CC1] | cyclohexyl-[4-(2-tetrahydropyran-4-yl-5H- pyrrolo[2,3-b]pyrazin-7-yl)-1- piperidyl]methanone |
| 24 | 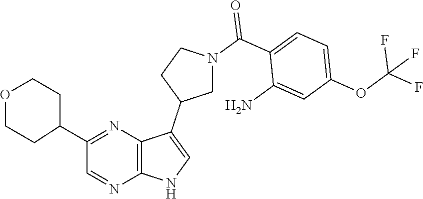 [SMILES: Nc1cc(OC(F)(F)F)ccc1C(=O)N1CCC(c2c[nH]c3ncc(C4CCOCC4)nc23)C1] | (rac)-[2-amino-4-(trifluoromethoxy)phenyl]- [3-(2-tetrahydropyran-4-yl-5H-pyrrolo[2,3- b]pyrazin-7-yl)pyrrolidine-1-yl]methanone |
| 25 | 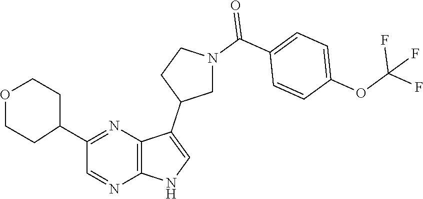 [SMILES: O=C(c1ccc(OC(F)(F)F)cc1)N1CCC(c2c[nH]c3ncc(C4CCOCC4)nc23)C1] | (rac)-[3-(2-tetrahydropyran-4-yl-5H- pyrrolo[2,3-b]pyrazin-7-yl)pyrrolidin-1-yl]- [4-(trifluoromethoxy)phenyl]methanone |
| 26 | 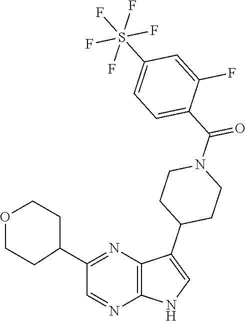 [SMILES: O=C(c1ccc(S(F)(F)(F)(F)F)cc1F)N1CCC(c2c[nH]c3ncc(C4CCOCC4)nc23)CC1] | [2-fluoro-4-(pentafluoro-λ⁶- sulfanyl)phenyl]-[4-(2-tetrahydropyran-4-yl- 5H-pyrrolo[2,3-b]pyrazin-7-yl)-1- piperidyl]methanone |
| 27 | 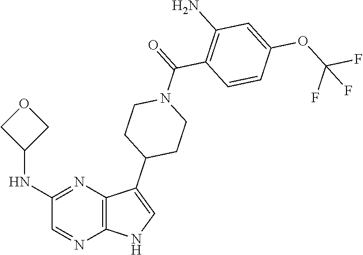 [SMILES: Nc1cc(OC(F)(F)F)ccc1C(=O)N1CCC(c2c[nH]c3ncc(NC4COC4)nc23)CC1] | [2-amino-4-(trifluoromethoxy)phenyl]-[4-[2- (oxetan-3-ylamino)-5H-pyrrolo[2,3- b]pyrazin-7-yl]-1-piperidyl]methanone |
| 28 | 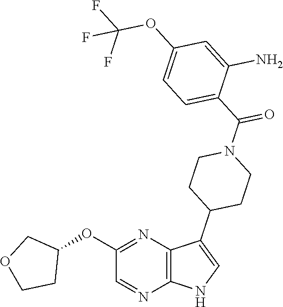 [SMILES: Nc1cc(OC(F)(F)F)ccc1C(=O)N1CCC(c2c[nH]c3ncc(O[C@@H]4CCOC4)nc23)CC1] | [2-amino-4-(trifluoromethoxy)phenyl]-[4-[2- [(3R)-tetrahydrofuran-3-yl]oxy-5H- pyrrolo[2,3-b]pyrazin-7-yl]-1- piperidyl]methanone |
| 29 | 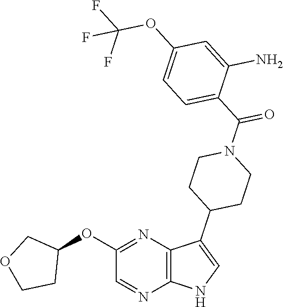 [SMILES: Nc1cc(OC(F)(F)F)ccc1C(=O)N1CCC(c2c[nH]c3ncc(O[C@H]4CCOC4)nc23)CC1] | [2-amino-4-(trifluoromethoxy)phenyl]-[4-[2- [(3S)-tetrahydrofuran-3-yl]oxy-5H- pyrrolo[2,3-b]pyrazin-7-yl]-1- piperidyl]methanone |
| 30 | 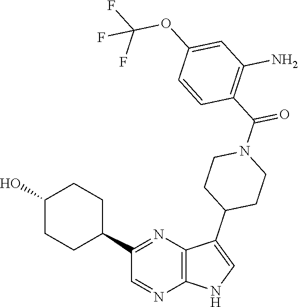 [SMILES: Nc1cc(OC(F)(F)F)ccc1C(=O)N1CCC(c2c[nH]c3ncc([C@H]4CC[C@H](O)CC4)nc23)CC1] | (rac)-trans-[2-amino-4- (trifluoromethoxy)phenyl]-[4-[2-(4- hydroxycyclohexyl)-5H-pyrrolo[2,3- b]pyrazin-7-yl]-1-piperidyl]methanone |
| 31 | 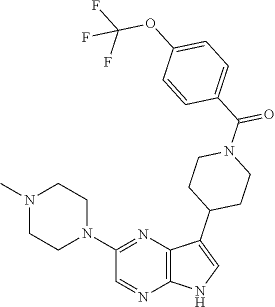 [SMILES: CN1CCN(c2cnc3[nH]cc(C4CCN(C(=O)c5ccc(OC(F)(F)F)cc5)CC4)c3n2)CC1] | [4-[2-(4-methylpiperazin-1-yl)-5H- pyrrolo[2,3-b]pyrazin-7-yl]-1-piperidyl]-[4- (trifluoromethoxy)phenyl]methanone, formic acid |
| | 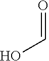 | |
| 32 | 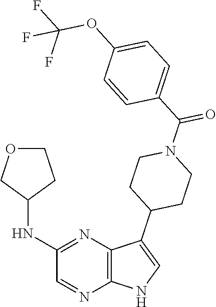 [SMILES: O=C(c1ccc(OC(F)(F)F)cc1)N1CCC(c2c[nH]c3ncc(NC4CCOC4)nc23)CC1] | [4-[2-[[tetrahydrofuran-3-yl]amino]-5H- pyrrolo[2,3-b]pyrazin-7-yl]-1-piperidyl]-[4- (trifluoromethoxy)phenyl]methanone, Isomer 1 |
| | Isomer 1 | |
| 33 | 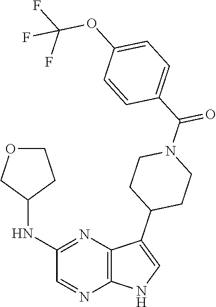 [SMILES: O=C(c1ccc(OC(F)(F)F)cc1)N1CCC(c2c[nH]c3ncc(NC4CCOC4)nc23)CC1] | [4-[2-[[tetrahydrofuran-3-yl]amino]-5H- pyrrolo[2,3-b]pyrazin-7-yl]-1-piperidyl]-[4- (trifluoromethoxy)phenyl]methanone, Isomer 2 |
| | Isomer 2 | |
| 34 | 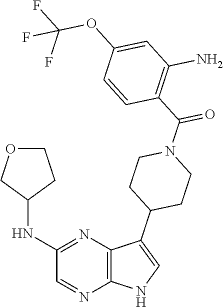 [SMILES: Nc1cc(OC(F)(F)F)ccc1C(=O)N1CCC(c2c[nH]c3ncc(NC4CCOC4)nc23)CC1] | [2-amino-4-(trifluoromethoxy)phenyl]-[4-[2- [[tetrahydrofuran-3-yl]amino]-5H- pyrrolo[2,3-b]pyrazin-7-yl]-1- piperidyl]methanone, Isomer 1 |
| | Isomer 1 | |
| 35 | 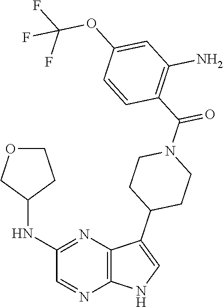 [SMILES: Nc1cc(OC(F)(F)F)ccc1C(=O)N1CCC(c2c[nH]c3ncc(NC4CCOC4)nc23)CC1] | [2-amino-4-(trifluoromethoxy)phenyl]-[4-[2- [[tetrahydrofuran-3-yl]amino]-5H- pyrrolo[2,3-b]pyrazin-7-yl]-1- piperidyl]methanone, Isomer 2 |
| | Isomer 2 | |
| 36 | 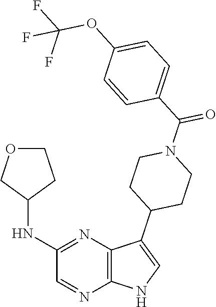 [SMILES: O=C(c1ccc(OC(F)(F)F)cc1)N1CCC(c2c[nH]c3ncc(NC4CCOC4)nc23)CC1] | (rac)-[4-[2-[[tetrahydrofuran-3-yl]amino]- 5H-pyrrolo[2,3-b]pyrazin-7-yl]-1-piperidyl]- [4-(trifluoromethoxy)phenyl]methanone |
| 37 | 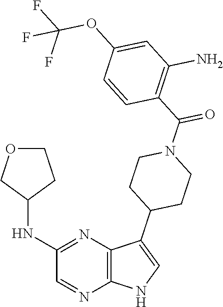 [SMILES: Nc1cc(OC(F)(F)F)ccc1C(=O)N1CCC(c2c[nH]c3ncc(NC4CCOC4)nc23)CC1] | (rac)-[2-amino-4-(trifluoromethoxy)phenyl]- [4-[2-[[tetrahydrofuran-3-yl]amino]-5H- pyrrolo[2,3-b]pyrazin-7-yl]-1- piperidyl]methanone |
| 38 | 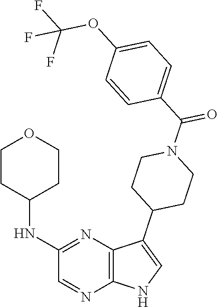 [SMILES: O=C(c1ccc(OC(F)(F)F)cc1)N1CCC(c2c[nH]c3ncc(NC4CCOCC4)nc23)CC1] | [4-[2-(tetrahydropyran-4-ylamino)-5H- pyrrolo[2,3-b]pyrazin-7-yl]-1-piperidyl]-[4- (trifluoromethoxy)phenyl]methanone |
| 39 | 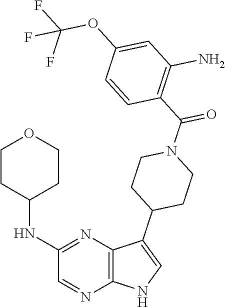 [SMILES: Nc1cc(OC(F)(F)F)ccc1C(=O)N1CCC(c2c[nH]c3ncc(NC4CCOCC4)nc23)CC1] | [2-amino-4-(trifluoromethoxy)phenyl]-[4-[2- (tetrahydropyran-4-ylamino)-5H- pyrrolo[2,3-b]pyrazin-7-yl]-1- piperidyl]methanone |
| 40 | 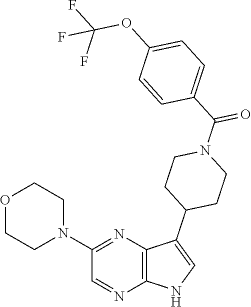 [SMILES: O=C(c1ccc(OC(F)(F)F)cc1)N1CCC(c2c[nH]c3ncc(N4CCOCC4)nc23)CC1] | [4-(2-morpholino-5H-pyrrolo[2,3-b]pyrazin- 7-yl)-1-piperidyl]-[4- (trifluoromethoxy)phenyl]methanone |
| 41 | 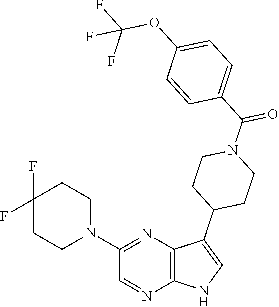 [SMILES: O=C(c1ccc(OC(F)(F)F)cc1)N1CCC(c2c[nH]c3ncc(N4CCC(F)(F)CC4)nc23)CC1] | [4-[2-(4,4-difluoro-1-piperidyl)-5H- pyrrolo[2,3-b]pyrazin-7-yl]-1-piperidyl]-[4- (trifluoromethoxy)phenyl]methanone |
| 42 | 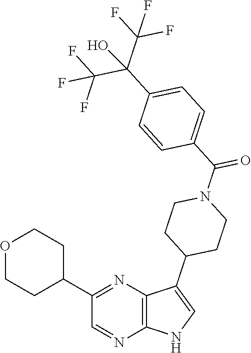 [SMILES: O=C(c1ccc(C(O)(C(F)(F)F)C(F)(F)F)cc1)N1CCC(c2c[nH]c3ncc(C4CCOCC4)nc23)CC1] | [4-(2-tetrahydropyran-4-yl-5H-pyrrolo[2,3- b]pyrazin-7-yl)-1-piperidyl]-[4-[2,2,2- trifluoro-1-hydroxy-1- (trifluoromethyl)ethyl]phenyl]methanone |
| 43 | 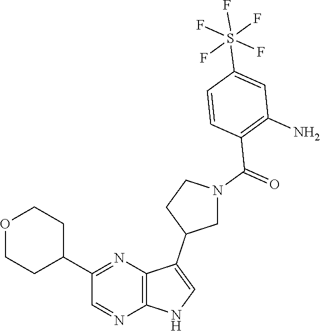 [SMILES: Nc1cc(S(F)(F)(F)(F)F)ccc1C(=O)N1CCC(c2c[nH]c3ncc(C4CCOCC4)nc23)C1] | (rac)-[2-amino-4-(pentafluoro-λ⁶- sulfanyl)phenyl]-[3-(2-tetrahydropyran-4-yl- 5H-pyrrolo[2,3-b]pyrazin-7-yl)pyrrolidin-1- yl]methanone |
| 44 | 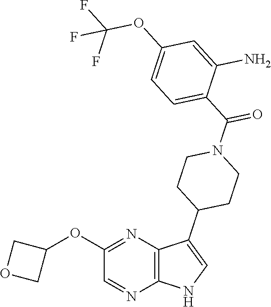 [SMILES: Nc1cc(OC(F)(F)F)ccc1C(=O)N1CCC(c2c[nH]c3ncc(OC4COC4)nc23)CC1] | [2-amino-4-(trifluoromethoxy)phenyl]-[4-[2- (oxetan-3-yloxy)-5H-pyrrolo[2,3-b]pyrazin- 7-yl]-1-piperidyl]methanone |

### Example 1: [2-Amino-4-(trifluoromethoxy)phenyl]-[4-[2-(1-methyl-4-piperidyl)-5H-pyrrolo[2,3-b]pyrazin-7-yl]-1-piperidyl]methanone

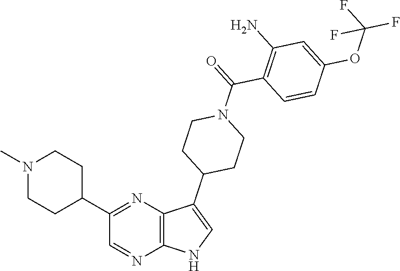 [SMILES: CN1CCC(c2cnc3[nH]cc(C4CCN(C(=O)c5ccc(OC(F)(F)F)cc5N)CC4)c3n2)CC1]

### Step 1: 2-Bromo-7-iodo-5H-pyrrolo[2,3-b]pyrazine

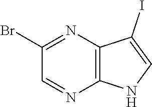 [SMILES: Brc1cnc2[nH]cc(I)c2n1]

A mixture of 2-bromo-5H-pyrrolo[2,3-b]pyrazine (5.00 g, 025.2 mmol), N-iodosuccinimide (6.82 g, 30.3 mmol) in N,N-dimethylformamide (40 mL) was stirred at room temperature for 16 hours. The mixture was then diluted with brine (200 mL) and extracted with ethyl acetate (100 mL×3). The organic layers were combined, washed with a saturated aqueous solution of sodium chloride (100 mL×2), dried over Na₂SO₄, filtered and concentrated in vacuo to give 2-bromo-7-iodo-5H-pyrrolo[2,3-b]pyrazine (8.17 g, 24.2 mmol, yield: 96%) as a yellow solid. LC/MS (m/z, M+H): calc. 324.9, found 324.9

### Step 2: 2-[(2-Bromo-7-iodo-pyrrolo[2,3-b]pyrazin-5-yl)methoxy]ethyl-trimethyl-silane

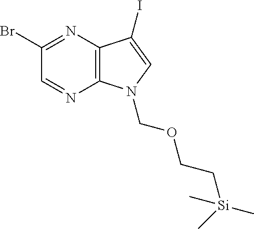 [SMILES: C[Si](C)(C)CCOCn1cc(I)c2nc(Br)cnc21]

To a mixture of 2-bromo-7-iodo-5H-pyrrolo[2,3-b]pyrazine (8.17 g, 24.1 mmol) in N,N-dimethylformamide (60 mL) was added sodium hydride 60% dispersion in oil (1.45 g, 15.1 mmol) at 0° C. After addition, the mixture was stirred for 1 hour at room temperature. Then, 2-(chloromethoxy)ethyl-trimethyl-silane (4.82 g, 28.9 mmol) was slowly added. The resulting mixture was stirred at room temperature for 2 hours. The mixture was then diluted with brine (200 mL) and extracted with ethyl acetate (100 mL×3). The organic layers were combined and washed with brine (100 mL×2), dried over Na₂SO₄, concentrated in vacuo and the resulting residue was purified by silica gel flash chromatography (0-5% ethyl acetate in petroleum ether) to give 2-[(2-bromo-7-iodo-pyrrolo[2,3-b]pyrazin-5-yl)methoxy]ethyl-trimethyl-silane (9.05 g, 19.9 mmol, yield: 83%) as a yellow solid. LC/MS (m/z, M+H): calc. 455.2, found 455.9

### Step 3: tert-Butyl 4-[2-bromo-5-(2-trimethylsilylethoxymethyl)pyrrolo[2,3-b]pyrazin-7-yl]-3,6-dihydro-2H-pyridine-1-carboxylate

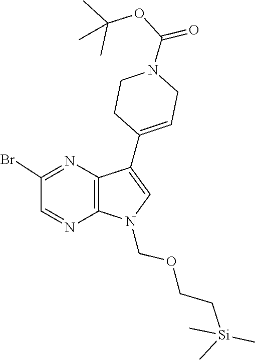 [SMILES: CC(C)(C)OC(=O)N1CC=C(c2cn(COCC[Si](C)(C)C)c3ncc(Br)nc23)CC1]

A mixture of 2-[(2-bromo-7-iodo-pyrrolo[2,3-b]pyrazin-5-yl)methoxy]ethyl-trimethyl-silane (9.05 g, 0.0199 mol), tert-butyl 4-(4,4,5,5-tetramethyl-1,3,2-dioxaborolan-2-yl)-3,6-dihydro-2H-pyridine-1-carboxylate (6.16 g, 19.9 mmol), [1,1′-bis(diphenylphosphino)ferrocene]dichloropalladium(II) (0.729 g, 0.996 mmol), sodium bicarbonate (6.34 g, 59.8 mmol) in 1,4-dioxane (200 mL) and water (40 mL) was stirred at 80° C. for 3 hours under N₂ atmosphere. The mixture was then diluted with water (200 mL) and extracted with ethyl acetate (100 mL×3). The organic layers were combined, washed with brine (100 mL×2), dried over Na₂SO₄, filtered and concentrated. The resulting residue was purified by silica gel flash chromatography (0-6% ethyl acetate in petroleum ether) to give tert-butyl 4-[2-bromo-5-(2-trimethylsilylethoxymethyl)pyrrolo[2,3-b]pyrazin-7-yl]-3,6-dihydro-2H-pyridine-1-carboxylate (4.62 g, 9.07 mmol, yield: 45%) as a yellow solid. LC/MS (m/z, M-tBu+H): calc. 453.1, found 453.0

### Step 4: tert-Butyl 4-[2-(1-methyl-3,6-dihydro-2H-pyridin-4-yl)-5-(2-trimethylsilylethoxymethyl)pyrrolo[2,3-b]pyrazin-7-yl]-3,6-dihydro-2H-pyridine-1-carboxylate

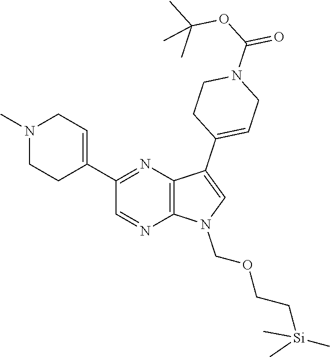

A mixture of tert-butyl 4-[2-bromo-5-(2-trimethylsilylethoxymethyl)pyrrolo[2,3-b]pyrazin-7-yl]-3,6-dihydro-2H-pyridine-1-carboxylate (1.5 g, 2.94 mmol), 1-methyl-4-(4,4,5,5-tetramethyl-1,3,2-dioxaborolan-2-yl)-3,6-dihydro-2H-pyridine (0.657 g, 2.94 mmol), [1,1′-Bis(diphenylphosphino)ferrocene]dichloropalladium(II) (0.108 g, 0.147 mmol), sodium carbonate (0.936 g, 8.83 mmol) in 1,4-dioxane (15 mL) and water (3 mL) was stirred at 80° C. for 3 hours under N₂ atmosphere. The mixture was then diluted with water (40 mL) and extracted with ethyl acetate (40 mL×3). The organic layers were combined, washed with brine (40 mL×2), dried over Na₂SO₄, filtered and concentrated in vacuo. The resulting residue was purified by silica gel flash chromatography (0-15% ethyl acetate in petroleum ether) to give tert-butyl 4-[2-(1-methyl-3,6-dihydro-2H-pyridin-4-yl)-5-(2-trimethylsilylethoxymethyl)pyrrolo[2,3-b]pyrazin-7-yl]-3,6-dihydro-2H-pyridine-1-carboxylate (1.20 g, 2.28 mmol, yield: 77%). LC/MS (m/z, M+H): 526

### Step 5: tert-Butyl 4-[2-(1-methyl-4-piperidyl)-5-(2-trimethylsilylethoxymethyl)pyrrolo[2,3-b]pyrazin-7-yl]piperidine-1-carboxylate

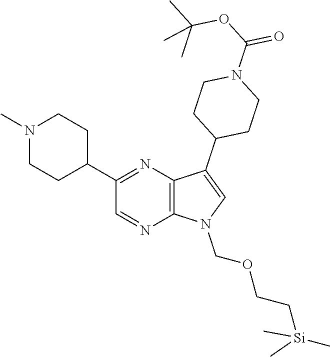

A mixture of tert-butyl 4-[2-(1-methyl-3,6-dihydro-2H-pyridin-4-yl)-5-(2-trimethylsilylethoxymethyl)pyrrolo[2,3-b]pyrazin-7-yl]-3,6-dihydro-2H-pyridine-1-carboxylate (1.20 g, 2.28 mmol), 5% Pd/C (1 g) in ethyl acetate (40 mL) was stirred at room temperature for 3 hours under 1 atmosphere of H₂. The mixture was then filtered and concentrated in vacuo to give tert-butyl 4-[2-(1-methyl-4-piperidyl)-5-(2-trimethylsilylethoxymethyl)pyrrolo[2,3-b]pyrazin-7-yl]piperidine-1-carboxylate (1.10 g, crude). LC/MS (m/z, M+H): calc. 530.3, found 530.3

### Step 6: 2-(1-Methyl-4-piperidyl)-7-(4-piperidyl)-5H-pyrrolo[2,3-b]pyrazine

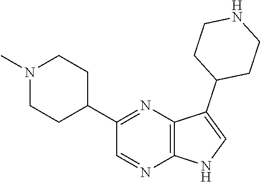 [SMILES: CN1CCC(c2cnc3[nH]cc(C4CCNCC4)c3n2)CC1]

A mixture of tert-butyl 4-[2-(1-methyl-4-piperidyl)-5-(2-trimethylsilylethoxymethyl)pyrrolo[2,3-b]pyrazin-7-yl]piperidine-1-carboxylate (1.10 g, 2.08 mmol) in trifluoroacetic acid (4 mL) and dichloromethane (4 mL) was stirred at room temperature for 2 hours. The mixture was then concentrated. The resulting residue was dissolved in methanol (30 mL) and 30% aqueous ammonia solution (5 mL) was added. The resulting mixture was stirred at room temperature for 4 hours. The mixture was then concentrated and the resulting residue was purified by reversed phase chromatography (0-40% acetonitrile in aqueous trifluoroacetic acid (0.5%)) to give 2-(1-methyl-4-piperidyl)-7-(4-piperidyl)-5H-pyrrolo[2,3-b]pyrazine (0.400 g, 1.34 mol, yield: 64%). LC/MS (m/z, M+H): calc. 300.2, found 300.2

### Step 7: tert-Butyl N-[2-[4-[2-(1-methyl-4-piperidyl)-5H-pyrrolo[2,3-b]pyrazin-7-yl]piperidine-1-carbonyl]-5-(trifluoromethoxy)phenyl]carbamate

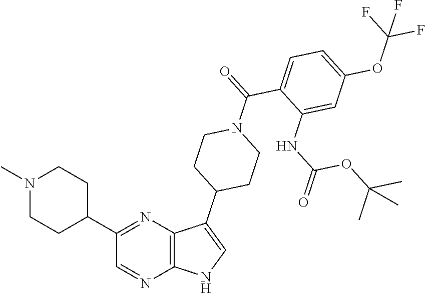 [SMILES: CN1CCC(c2cnc3[nH]cc(C4CCN(C(=O)c5ccc(OC(F)(F)F)cc5NC(=O)OC(C)(C)C)CC4)c3n2)CC1]

A mixture of 2-(1-methyl-4-piperidyl)-7-(4-piperidyl)-5H-pyrrolo[2,3-b]pyrazine (0.200 g, 0.651 mmol), 2-(tert-butoxycarbonylamino)-4-(trifluoromethoxy)benzoic acid (0.209 g, 0.651 mmol), 2-(1H-benzotriazole-1-yl)-1,1,3,3-tetramethyluronium tetrafluoroborate (0.313 g, 0.976 mmol), N-ethyl-N-isopropyl-propan-2-amine (0.252 g, 0.195 mmol) in N,N-dimethylformamide (8 mL) was stirred at room temperature for 3 hours. The mixture was then diluted with brine (40 mL), extracted with ethyl acetate (30 mL×3). The organic layers were combined, washed with brine (20 mL×2), dried over Na₂SO₄, filtered and concentrated. The residue was purified by preparative HPLC to give tert-butyl N-[2-[4-[2-(1-methyl-4-piperidyl)-5H-pyrrolo[2,3-b]pyrazin-7-yl]piperidine-1-carbonyl]-5-(trifluoromethoxy)phenyl]carbamate (0.120 g, 0.199 mmol, yield: 31%). LC/MS (m/z, M+H): calc. 603.3, found 603.3

### Step 8: [2-Amino-4-(trifluoromethoxy)phenyl]-[4-[2-(1-methyl-4-piperidyl)-5H-pyrrolo[2,3-b]pyrazin-7-yl]-1-piperidyl]methanone

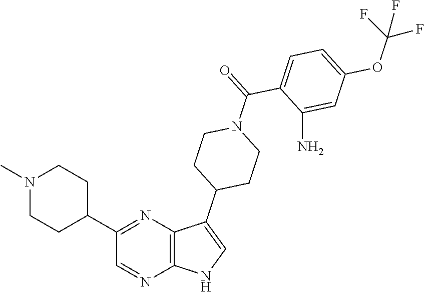 [SMILES: CN1CCC(c2cnc3[nH]cc(C4CCN(C(=O)c5ccc(OC(F)(F)F)cc5N)CC4)c3n2)CC1]

To a solution of tert-butyl N-[2-[4-[2-(1-methyl-4-piperidyl)-5H-pyrrolo[2,3-b]pyrazin-7-yl]piperidine-1-carbonyl]-5-(trifluoromethoxy)phenyl]carbamate (0.120 g, 0.199 mmol) in dichloromethane (2 mL) was added trifluoroacetic acid (1 mL). The reaction mixture was stirred at room temperature for 1 hour. Then, the reaction was concentrated and the residue was purified by preparative HPLC to afford [2-amino-4-(trifluoromethoxy)phenyl]-[4-[2-(1-methyl-4-piperidyl)-5H-pyrrolo[2,3-b]pyrazin-7-yl]-1-piperidyl]methanone (0.031 g, 0.062 mmol, yield: 31%) as a white solid. LC/MS (m/z, M+H): calc. 503.2, found 503.4; ¹H NMR (400 MHz, DMSO-d6, 100° C.) δ ppm 1.76-1.96 (m, 6H), 2.00-2.12 (m, 4H), 2.22 (s, 3H), 2.70-2.81 (m, 1H), 2.88 (br d, J=12 Hz, 2H), 3.05-3.25 (m, 3H), 4.09 (br d, J=13 Hz, 2H), 5.31 (s, 2H), 6.47 (br dd, J=8, 1 Hz, 1H), 6.67 (br d, J=1 Hz, 1H), 7.13 (d, J=8 Hz, 1H), 7.48 (s, 1H), 8.09 (s, 1H), 11.06-11.48 (m, 1H)

### Example 2: [2-Amino-4-(trifluoromethoxy)phenyl]-[4-(2-tetrahydropyran-4-yl-5H-pyrrolo[2,3-b]pyrazin-7-yl)-1-piperidyl]methanone

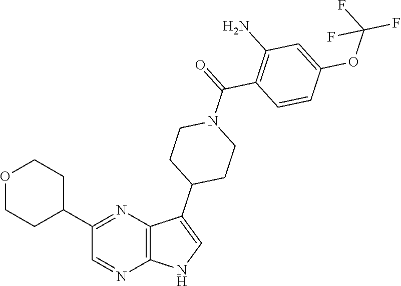 [SMILES: Nc1cc(OC(F)(F)F)ccc1C(=O)N1CCC(c2c[nH]c3ncc(C4CCOCC4)nc23)CC1]

### Step 1: tert-Butyl 4-[2-(3,6-dihydro-2H-pyran-4-yl)-5-(2-trimethylsilylethoxymethyl)pyrrolo[2,3-b]pyrazin-7-yl]-3,6-dihydro-2H-pyridine-1-carboxylate

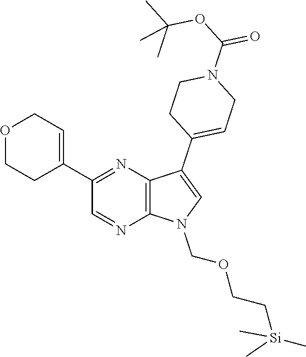 [SMILES: CC(C)(C)OC(=O)N1CC=C(c2cn(COCC[Si](C)(C)C)c3ncc(C4=CCOCC4)nc23)CC1]

A mixture of tert-butyl 4-[2-bromo-5-(2-trimethylsilylethoxymethyl)pyrrolo[2,3-b]pyrazin-7-yl]-3,6-dihydro-2H-pyridine-1-carboxylate (1.5 g, 2.94 mmol), 2-(3,6-dihydro-2H-pyran-4-yl)-4,4,5,5-tetramethyl-1,3,2-dioxaborolane (0.618 g, 2.94 mmol), [1,1′-Bis(diphenylphosphino)ferrocene]dichloropalladium(II) (0.108 g, 0.147 mmol), sodium carbonate (0.936 g, 8.83 mmol) in 1,4-dioxane (15 mL) and water (3 mL) was stirred at 80° C. for 3 hours under N₂ atmosphere. The mixture was then diluted with water (40 mL) and extracted with ethyl acetate (40 mL×3). The organic layers were combined, washed with brine (40 mL×2), dried over Na₂SO₄, filtered and concentrated in vacuo. The resulting residue was purified by silica gel flash chromatography (0-15% ethyl acetate in petroleum ether) to give tert-butyl 4-[2-(3,6-dihydro-2H-pyran-4-yl)-5-(2-trimethylsilylethoxymethyl)pyrrolo[2,3-b]pyrazin-7-yl]-3,6-dihydro-2H-pyridine-1-carboxylate (1.10 g, 2.15 mmol, yield: 73%). LC/MS (m/z, M+H): calc. 513.3, found 513.3

### Step 2: tert-Butyl 4-[2-tetrahydropyran-4-yl-5-(2-trimethylsilylethoxymethyl)pyrrolo[2,3-b]pyrazin-7-yl]piperidine-1-carboxylate

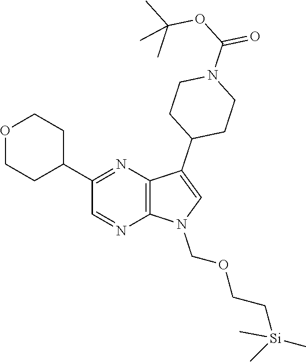 [SMILES: CC(C)(C)OC(=O)N1CCC(c2cn(COCC[Si](C)(C)C)c3ncc(C4CCOCC4)nc23)CC1]

A mixture of tert-butyl 4-[2-(3,6-dihydro-2H-pyran-4-yl)-5-(2-trimethylsilylethoxymethyl)pyrrolo[2,3-b]pyrazin-7-yl]-3,6-dihydro-2H-pyridine-1-carboxylate (1.10 g, 2.15 mmol), 5% Pd/C (1 g) in ethyl acetate (40 mL) was stirred at room temperature for 3 hours under 1 atmosphere of H₂. The mixture was filtered and concentrated to give tert-butyl 4-[2-tetrahydropyran-4-yl-5-(2-trimethylsilylethoxymethyl)pyrrolo[2,3-b]pyrazin-7-yl]piperidine-1-carboxylate (1.10 g, crude). LC/MS (m/z, M+H): calc. 517.3, found 517.3

### Step 3: 7-(4-Piperidyl)-2-tetrahydropyran-4-yl-5H-pyrrolo[2,3-b]pyrazine

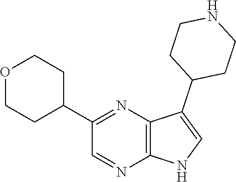 [SMILES: c1nc2[nH]cc(C3CCNCC3)c2nc1C1CCOCC1]

A mixture of tert-butyl 4-[2-tetrahydropyran-4-yl-5-(2-trimethylsilylethoxymethyl)pyrrolo[2,3-b]pyrazin-7-yl]piperidine-1-carboxylate (1.00 g, 1.94 mmol) in trifluoroacetic acid (4 mL) and dichloromethane (1 mL) was stirred at room temperature for 2 hours. The mixture was then concentrated. The resulting residue was dissolved in methanol (30 mL), 30% aqueous ammonia solution (5 mL) was added, and the resulting mixture was stirred at room temperature for 4 hours. The mixture was concentrated and the resulting residue was purified by reverse phase chromatography (0-40% acetonitrile in aq. trifluoroacetic acid (0.5%)) to give 7-(4-piperidyl)-2-tetrahydropyran-4-yl-5H-pyrrolo[2,3-b]pyrazine (0.400 g, 1.40 mmol, yield: 72%). LC/MS (m/z, M+H): calc. 287.2, found 287.2

### Step 4: tert-Butyl N-[2-[4-(2-tetrahydropyran-4-yl-5H-pyrrolo[2,3-b]pyrazin-7-yl)piperidine-1-carbonyl]-5-(trifluoromethoxy)phenyl]carbamate

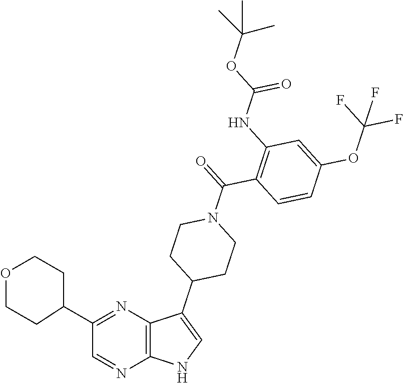 [SMILES: CC(C)(C)OC(=O)Nc1cc(OC(F)(F)F)ccc1C(=O)N1CCC(c2c[nH]c3ncc(C4CCOCC4)nc23)CC1]

A mixture of 7-(4-piperidyl)-2-tetrahydropyran-4-yl-5H-pyrrolo[2,3-b]pyrazine (0.200 g, 0.70 mmol), 2-(tert-butoxycarbonylamino)-4-(trifluoromethoxy)benzoic acid (0.224 g, 0.70 mmol), 2-(1H-benzotriazole-1-yl)-1,1,3,3-tetramethyluronium tetrafluoroborate (0.336 g, 1.05 mmol), N,N-diisopropylethylamine (0.271 g, 2.10 mmol) in N,N-dimethylformamide (8 mL) was stirred at room temperature for 3 hours. The mixture was diluted with brine (40 mL), extracted with ethyl acetate (30 mL×3). The organic layers were combined, washed with brine (20 mL×2), dried over Na₂SO₄, filtered and concentrated in vacuo. The resulting residue was purified by preparative HPLC to give tert-butyl N-[2-[4-(2-tetrahydropyran-4-yl-5H-pyrrolo[2,3-b]pyrazin-7-yl)piperidine-1-carbonyl]-5-(trifluoromethoxy)phenyl]carbamate (0.247 g, 4.19 mmol, yield: 60%). LC/MS (m/z, M+H): calc. 590.2, found 590.2

### Step 5: [2-Amino-4-(trifluoromethoxy)phenyl]-[4-(2-tetrahydropyran-4-yl-5H-pyrrolo[2,3-b]pyrazin-7-yl)-1-piperidyl]methanone

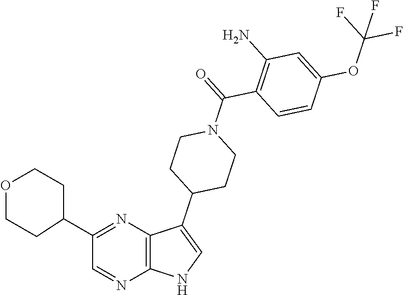 [SMILES: Nc1cc(OC(F)(F)F)ccc1C(=O)N1CCC(c2c[nH]c3ncc(C4CCOCC4)nc23)CC1]

To a solution of tert-butyl N-[2-[4-(2-tetrahydropyran-4-yl-5H-pyrrolo[2,3-b]pyrazin-7-yl)piperidine-1-carbonyl]-5-(trifluoromethoxy)phenyl]carbamate (0.411 g, 0.70 mmol) in dichloromethane (2 mL) was added trifluoroacetic acid (1 mL). The reaction mixture was stirred at room temperature for 1 hour. Then, the reaction was concentrated and the crude residue was then purified by preparative HPLC to afford [2-amino-4-(trifluoromethoxy)phenyl]-[4-(2-tetrahydropyran-4-yl-5H-pyrrolo[2,3-b]pyrazin-7-yl)-1-piperidyl]methanone (0.0627 g, 0.128 mmol, yield: 18%) as a white solid. LC/MS (m/z, M+H): calc. 490.2, found 490.2; ¹H NMR (400 MHz, DMSO-d6, 100° C.) δ ppm 1.66-1.99 (m, 6H), 2.03-2.20 (m, 2H), 2.99-3.31 (m, 4H), 3.50 (ddd, J=11.4, 11.4, 2.9 Hz, 2H), 3.90-4.03 (m, 2H), 4.09 (br d, J=13.3 Hz, 2H), 5.31 (s, 2H), 6.47 (br d, J=8.2 Hz, 1H), 6.66-6.70 (m, 1H), 7.12 (d, J=8.4 Hz, 1H), 7.50 (s, 1H), 8.11 (s, 1H), 11.18-11.38 (m, 1H)

### Example 3: [4-(2-Tetrahydropyran-4-yl-5H-pyrrolo[2,3-b]pyrazin-7-yl)-1-piperidyl]-[4-(trifluoromethoxy)phenyl]methanone

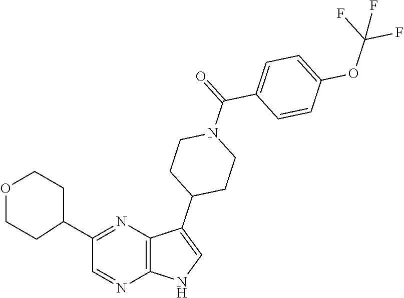 [SMILES: O=C(c1ccc(OC(F)(F)F)cc1)N1CCC(c2c[nH]c3ncc(C4CCOCC4)nc23)CC1]

A mixture of 7-(4-piperidyl)-2-tetrahydropyran-4-yl-5H-pyrrolo[2,3-b]pyrazine (0.200 g, 0.70 mmol), 4-(trifluoromethoxy)benzoic acid (0.144 g, 0.70 mmol), 2-(1H-benzotriazole-1-yl)-1,1,3,3-tetramethyluronium tetrafluoroborate (0.336 g, 1.05 mmol), N-ethyl-N-isopropyl-propan-2-amine (0.271 g, 2.10 mmol) in N,N-dimethylformamide (4 mL) was stirred at room temperature for 3 hours. The mixture was then diluted with brine (40 mL) and extracted with ethyl acetate (30 mL×3). The organic layers were combined, washed with brine (20 mL×2), dried over Na₂SO₄, filtered and concentrated in vacuo. The resulting residue was purified by preparative HPLC to give [4-(2-tetrahydropyran-4-yl-5H-pyrrolo[2,3-b]pyrazin-7-yl)-1-piperidyl]-[4-(trifluoromethoxy)phenyl]methanone (0.0501 g, 0.106 mmol, yield: 15%). LC/MS (m/z, M+H): calc. 475.2, found 475.3; ¹H NMR (400 MHz, DMSO-d6, 120° C.) δ ppm 1.78-1.98 (m, 6H), 2.10 (br dd, J=13, 3 Hz, 2H), 3.02-3.29 (m, 4H), 3.51 (td, J=11, 3 Hz, 2H), 3.93-4.01 (m, 2H), 4.08 (br d, J=12 Hz, 2H), 7.36 (br d, J=9 Hz, 2H), 7.49 (d, J=2 Hz, 1H), 7.53 (d, J=9 Hz, 2H), 8.11 (s, 1H), 11.16 (br s, 1H)

### Example 4: [4-[2-(1-Methyl-4-piperidyl)-5H-pyrrolo[2,3-b]pyrazin-7-yl]-1-piperidyl]-[4-(trifluoromethoxy)phenyl]methanone

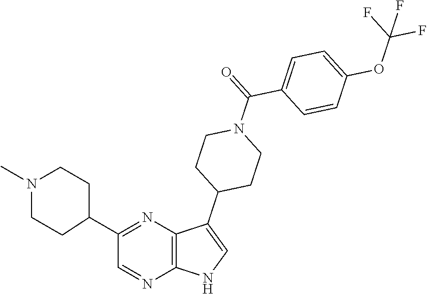 [SMILES: CN1CCC(c2cnc3[nH]cc(C4CCN(C(=O)c5ccc(OC(F)(F)F)cc5)CC4)c3n2)CC1]

A mixture of 2-(1-methyl-4-piperidyl)-7-(4-piperidyl)-5H-pyrrolo[2,3-b]pyrazine (0.200 g, 0.67 mmol), 4-(trifluoromethoxy)benzoic acid (0.138 g, 0.67 mmol), 2-(1H-benzotriazole-1-yl)-1,1,3,3-tetramethyluronium tetrafluoroborate (0.322 g, 1.00 mmol), N-ethyl-N-isopropyl-propan-2-amine (0.259 g, 2.00 mmol) in N,N-dimethylformamide (4 mL) was stirred at room temperature for 3 hours. The mixture was diluted with brine (40 mL) and extracted with ethyl acetate (30 mL×3). The organic layers were combined, washed with brine (20 mL×2), dried over Na₂SO₄, concentrated in vacuo and the resulting residue was purified by preparative HPLC to give [4-[2-(1-methyl-4-piperidyl)-5H-pyrrolo[2,3-b]pyrazin-7-yl]-1-piperidyl]-[4-(trifluoromethoxy)phenyl]methanone (0.0210 g, 0.043 mmol, yield: 6.5%). LC/MS (m/z, M+H): calc. 488.2, found 488.2; ¹H NMR (400 MHz, DMSO-d6, 120° C.) δ ppm 1.79-1.97 (m, 6H), 2.02-2.15 (m, 4H), 2.22 (s, 3H), 2.69-3.00 (m partially hidden, 3H), 3.09-3.31 (m, 3H), 4.04-4.14 (m, 2H), 7.36 (br d, J=8 Hz, 2H), 7.47 (s, 1H), 7.53 (d, J=8 Hz, 2H), 8.08 (s, 1H), 10.47-11.73 (m, 1H)

### Example 5: (rac)-[4-(2-Tetrahydrofuran-3-yl-5H-pyrrolo[2,3-b]pyrazin-7-yl)-1-piperidyl]-[4-(trifluoromethoxy)phenyl]methanone

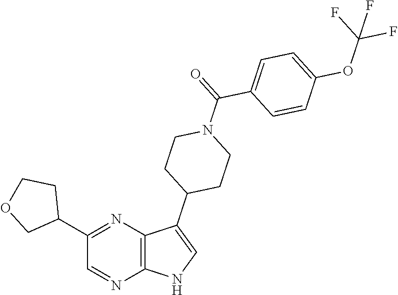 [SMILES: O=C(c1ccc(OC(F)(F)F)cc1)N1CCC(c2c[nH]c3ncc(C4CCOC4)nc23)CC1]

### Step 1: tert-Butyl 4-[2-(2,5-dihydrofuran-3-yl)-5-(2-trimethylsilylethoxymethyl)pyrrolo[2,3-b]pyrazin-7-yl]-3,6-dihydro-2H-pyridine-1-carboxylate

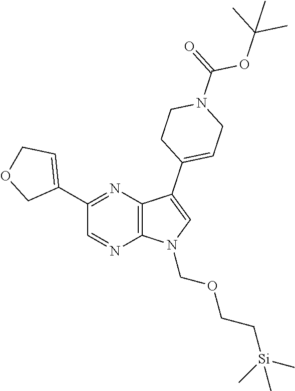 [SMILES: CC(C)(C)OC(=O)N1CC=C(c2cn(COCC[Si](C)(C)C)c3ncc(C4=CCOC4)nc23)CC1]

A mixture of tert-butyl 4-[2-bromo-5-(2-trimethylsilylethoxymethyl)pyrrolo[2,3-b]pyrazin-7-yl]-3,6-dihydro-2H-pyridine-1-carboxylate (100 mg, 0.20 mmol), 2-(2,5-dihydrofuran-3-yl)-4,4,5,5-tetramethyl-1,3,2-dioxaborolane (0.0404 g, 0.21 mmol), [1,1′-bis(diphenylphosphino)ferrocene]dichloropalladium(II) (0.0144 g, 0.02 mmol), sodium carbonate (0.0624 g, 0.59 mmol) in 1,4-dioxane (2 mL) and water (0.5 mL) was stirred at 80° C. for 4 hours under N₂ atmosphere. The mixture was then diluted with water (40 mL) and extracted with ethyl acetate (40 mL×3). The organic layers were combined, washed with brine (40 mL×2), dried over Na₂SO₄, filtered and concentrated in vacuo. The resulting residue was purified by SGC (eluting with 0-15% ethyl acetate in petroleum ether) to give tert-butyl 4-[2-(2,5-dihydrofuran-3-yl)-5-(2-trimethylsilylethoxymethyl)pyrrolo[2,3-b]pyrazin-7-yl]-3,6-dihydro-2H-pyridine-1-carboxylate as a white solid (805 mg, 82%). LC/MS (m/z, M+H): calc. 499.3, found 499.2

### Step 2: (rac)-tert-Butyl 4-[2-tetrahydrofuran-3-yl-5-(2-trimethylsilylethoxymethyl)pyrrolo[2,3-b]pyrazin-7-yl]piperidine-1-carboxylate

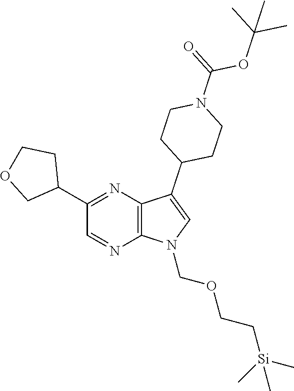 [SMILES: CC(C)(C)OC(=O)N1CCC(c2cn(COCC[Si](C)(C)C)c3ncc(C4CCOC4)nc23)CC1]

To a solution of tert-butyl 4-[2-(2,5-dihydrofuran-3-yl)-5-(2-trimethylsilylethoxymethyl)pyrrolo[2,3-b]pyrazin-7-yl]-3,6-dihydro-2H-pyridine-1-carboxylate (800 mg, 1.50 mmol) in ethyl acetate (10 mL) was added Pd/C (20 wt %, 160 mg). The mixture was stirred for 24 hours at room temperature under 1 atmosphere of hydrogen. The reaction mixture was then filtered and the filtrate was concentrated under reduced pressure. The resulting residue was then purified by silica gel chromatography (15% ethyl acetate in petroleum ether) to give (rac)-tert-butyl 4-[2-tetrahydrofuran-3-yl-5-(2-trimethylsilylethoxymethyl)pyrrolo[2,3-b]pyrazin-7-yl]piperidine-1-carboxylate (0.670 mg, 0.88 mmol, yield: 55%). LC/MS (m/z, M+H): calc. 503.3, found 503.0

### Step 3: (rac)-7-(4-Piperidyl)-2-tetrahydrofuran-3-yl-pyrrolo[2,3-b]pyrazin-5-yl]methanol

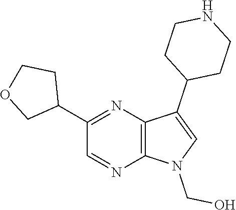 [SMILES: OCn1cc(C2CCNCC2)c2nc(C3CCOC3)cnc21]

To a solution of (rac)-tert-butyl 4-[2-tetrahydrofuran-3-yl-5-(2-trimethylsilylethoxymethyl)pyrrolo[2,3-b]pyrazin-7-yl]piperidine-1-carboxylate (620 mg, 0.82 mmol) in dichloromethane (6 mL) was added trifluoroacetic acid (6 mL) at 0° C. The resulting mixture was stirred at room temperature for 1 hour. The reaction mixture was then concentrated to get a yellow oil (510 mg, 0.82 mmol, crude) which was used in the next step without further purification. LC/MS (m/z, M+30): calc. 303.2, found 303.3

### Step 4: (rac)-[4-(2-Tetrahydrofuran-3-yl-5H-pyrrolo[2,3-b]pyrazin-7-yl)-1-piperidyl]-[4-(trifluoromethoxy)phenyl]methanone

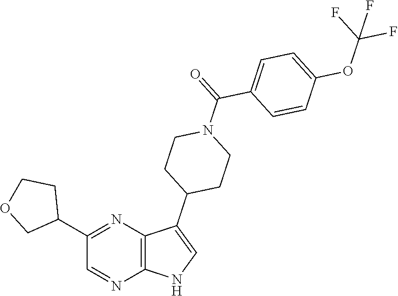 [SMILES: O=C(c1ccc(OC(F)(F)F)cc1)N1CCC(c2c[nH]c3ncc(C4CCOC4)nc23)CC1]

To a solution of (rac)-7-(4-piperidyl)-2-tetrahydrofuran-3-yl-pyrrolo[2,3-b]pyrazin-5-yl]methanol (510 mg, 0.82 mmol) and 4-(trifluoromethoxy)benzoic acid (343 mg, 1.66 mmol) in N,N-dimethylformamide (5 mL) was added 1-hydroxybenzotriazole hydrate (255 mg, 1.66 mmol), N′-(ethylcarbonimidoyl)-N,N-dimethylpropane-1,3-diamine hydrochloride (319 mg, 1.66 mmol) and N-ethyl-N-isopropyl-propan-2-amine (537 mg, 4.16 mmol). The resulting mixture was stirred for 16 hours at room temperature. The reaction mixture was diluted with water (50 mL) and extracted with ethyl acetate (3×50 mL). The organic layers were combined and concentrated to give a crude oil. The crude product was dissolved in a mixed solution of methanol (10 mL) and 30% aqueous ammonia solution (15 mL). After stirred for 1 hour, the resulting solution was diluted with water and extracted with ethyl acetate. The organic layer were washed with water, brine, dried over anhydrous Na₂SO₄, filtered and concentrated to give a crude oil which was purified by silica gel chromatography (3% methanol in dichloromethane) to give (rac)-[4-(2-tetrahydrofuran-3-yl-5H-pyrrolo[2,3-b]pyrazin-7-yl)-1-piperidyl]-[4-(trifluoromethoxy)phenyl]methanone (288 mg, yield: 75%) as a colorless solid. LC/MS (m/z, M+H): calc. 461.2, found 461.0; ¹H NMR (400 MHz, DMSO-d6, 100° C.) δ ppm 1.78-1.93 (m, 2H), 2.03-2.13 (m, 2H), 2.16-2.37 (m, 2H), 3.06-3.27 (m, 3H), 3.68 (quin, J=8 Hz, 1H), 3.76-3.90 (m, 2H), 3.97 (td, J=8, 5 Hz, 1H), 4.01-4.20 (m, 1H), 4.11 (t, J=8 Hz, 2H), 7.38 (d, J=8 Hz, 2H), 7.50-7.59 (m, 3H), 8.13 (s, 1H), 11.00-11.68 (m, 1H)

### Example 6. [4-[2-(3-Methoxypropyl)-5H-pyrrolo[2,3-b]pyrazin-7-yl]-1-piperidyl]-[4-(trifluoromethoxy)phenyl]methanone

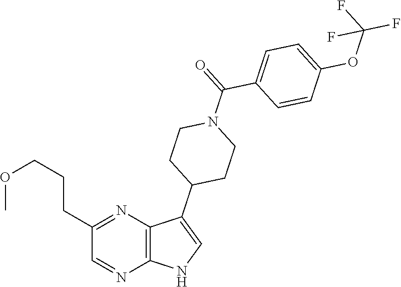 [SMILES: COCCCc1cnc2[nH]cc(C3CCN(C(=O)c4ccc(OC(F)(F)F)cc4)CC3)c2n1]

### Step 1: tert-Butyl 4-[2-[(E)-3-ethoxy-3-oxo-prop-1-enyl]-5-(2-trimethyl silylethoxymethyl)pyrrolo[2,3-b]pyrazin-7-yl]-3,6-dihydro-2H-pyri dine-1l-carboxylate

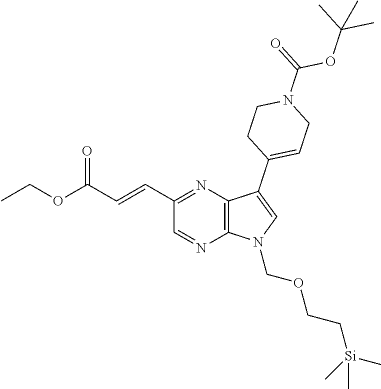 [SMILES: CCOC(=O)/C=C/c1cnc2c(n1)c(C1=CCN(C(=O)OC(C)(C)C)CC1)cn2COCC[Si](C)(C)C]

A mixture of ethyl (E)-3-(4,4,5,5-tmeraethyl-1,3,2-dioxaborolan-2-yl)prop-2-enoate (1.33 g, 5.89 mmol), tert-butyl 4-[2-bromo-5-(2-trimethylsilylethoxymethyl)pyrrolo[2,3-b]pyrazin-7-yl]-3,6-dihydro-2H-pyridine-1-carboxylate (3.00 g, 5.89 mmol), [1,1′-bis(diphenylphosphino)ferrocene]dichloropalladium(II) (0.129 g, 0.18 mmol), sodium carbonate (1.87 g, 17.7 mmol) in 1,4-dioxane (30 mL) and water (6 mL) was stirred at 80° C. for 3 hours under argon atmosphere. The reaction mixture was diluted with brine (30 mL) and extracted with ethyl acetate (20 mL×3). The organic layers were combined, washed with brine (20 mL×2), dried over Na₂SO₄, concentrated and the resulting residue was purified by silica gel flash chromatography (0-20% ethyl acetate in petroleum ether) to give tert-butyl 4-[2-[(E)-3-ethoxy-3-oxo-prop-1-enyl]-5-(2-trimethylsilylethoxymethyl)pyrrolo[2,3-b]pyrazin-7-yl]-3,6-dihydro-2H-pyridine-1-carboxylate as a yellow solid (2.6 g, 83%). LC/MS (m/z, M-tBu): calc. 473.3, found 473.3

### Step 2: tert-Butyl 4-[2-(3-ethoxy-3-oxo-propyl)-5-(2-trimethylsilylethoxymethyl)pyrrolo[2,3-b]pyrazin-7-yl]piperidine-1-carboxylate

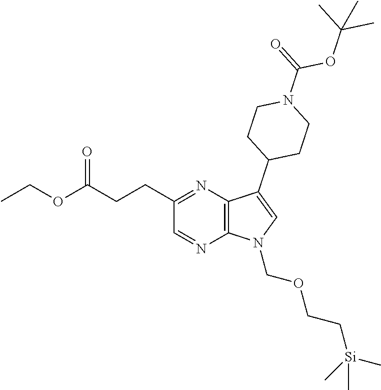 [SMILES: CCOC(=O)CCc1cnc2c(n1)c(C1CCN(C(=O)OC(C)(C)C)CC1)cn2COCC[Si](C)(C)C]

A mixture of tert-butyl 4-[2-[(E)-3-ethoxy-3-oxo-prop-1-enyl]-5-(2-trimethylsilylethoxymethyl)pyrrolo[2,3-b]pyrazin-7-yl]-3,6-dihydro-2H-pyridine-1-carboxylate (2.60 g, 4.92 mmol), 10% Pd/C (10.0%, 1.30 g) in ethyl acetate (50 mL) was stirred at room temperature for 48 hours under 1 atmosphere of H₂. The reaction mixture was then filtered, concentrated and the resulting residue was purified by silica gel flash chromatography (0-15% ethyl acetate in petroleum ether) to give tert-butyl 4-[2-(3-ethoxy-3-oxo-propyl)-5-(2-trimethylsilylethoxymethyl)pyrrolo[2,3-b]pyrazin-7-yl]piperidine-1-carboxylate as a yellow solid (2.3 g, 88%). LC/MS (m/z, M+H): calc. clcd 533.3, found 533.0

### Step 3: tert-Butyl 4-[2-(3-hydroxypropyl)-5-(2-trimethylsilylethoxymethyl)pyrrolo[2,3-b]pyrazin-7-yl]piperidine-1-carboxylate

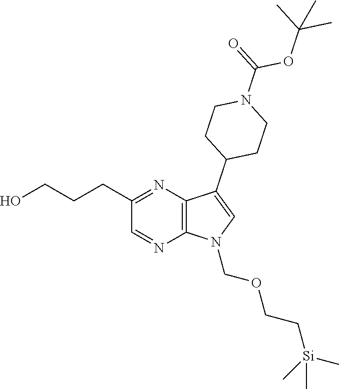 [SMILES: CC(C)(C)OC(=O)N1CCC(c2cn(COCC[Si](C)(C)C)c3ncc(CCCO)nc23)CC1]

To a mixture of tert-butyl 4-[2-(3-ethoxy-3-oxo-propyl)-5-(2-trimethylsilylethoxymethyl)pyrrolo[2,3-b]pyrazin-7-yl]piperidine-1-carboxylate (2.30 g, 0.00432 mol) in tetrahydrofuran (30 mL) was slowly added 1N LiAlH₄ in tetrahydrofuran (1.00 mol/L, 5.18 mL, 0.00518 mol) at 0° C. After addition, the mixture was stirred at 0° C. for 1 hour. The reaction mixture was then quenched with Na₂SO₄.10H₂O (5 g) and filtered. The solvents were combined and concentrated. The resulting residue was purified by silica gel flash chromatography (0-25% ethyl acetate in petroleum ether) to give tert-butyl 4-[2-(3-hydroxypropyl)-5-(2-trimethylsilylethoxymethyl)pyrrolo[2,3-b]pyrazin-7-yl]piperidine-1-carboxylate as a yellow solid (1.8 g, 85%) LC/MS (m/z, M+H): calc. 491.3, found 491.0

### Step 4: tert-Butyl 4-[2-(3-methoxypropyl)-5-(2-trimethylsilylethoxymethyl)pyrrolo[2,3-b]pyrazin-7-yl]piperidine-1-carboxylate

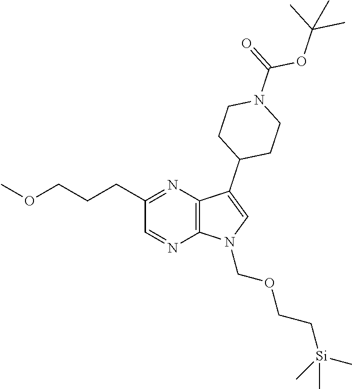

To a mixture of tert-butyl 4-[2-(3-hydroxypropyl)-5-(2-trimethylsilylethoxymethyl)pyrrolo[2,3-b]pyrazin-7-yl]piperidine-1-carboxylate (1.80 g, 3.67 mmol) in tetrahydrofuran (30 mL) was added sodium hydride (60%, 0.220 g, 5.50 mmol) at 0° C. After addition, the mixture was stirred for 1 hour. Then, iodomethane (0.625 g, 4.40 mmol) was added dropwise. The resulting mixture was stirred at room temperature for 72 hours. The reaction mixture was then diluted with brine (40 mL) and extracted with ethyl acetate (30 mL×3). The organic layers were combined, washed with brine (30 mL×2), dried over Na₂SO₄, filtered and concentrated. The resulting residue was purified by silica gel flash chromatography (0-10% ethyl acetate in petroleum ether) to give the tert-butyl 4-[2-(3-methoxypropyl)-5-(2-trimethylsilylethoxymethyl)pyrrolo[2,3-b]pyrazin-7-yl]piperidine-1-carboxylate as a yellow solid (700 mg, 38%). LC/MS (m/z, M+H): calc. 505.3, found 505.2

### Step 5: 2-(3-Methoxypropyl)-7-(4-piperidyl)pyrrolo[2,3-b]pyrazin-5-yl]methanol

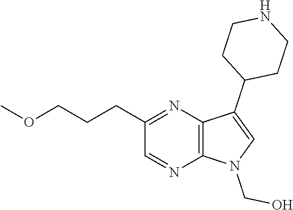 [SMILES: COCCCc1cnc2c(n1)c(C1CCNCC1)cn2CO]

A mixture of tert-butyl 4-[2-(3-methoxypropyl)-5-(2-trimethylsilylethoxymethyl)pyrrolo[2,3-b]pyrazin-7-yl]piperidine-1-carboxylate (0.200 g, 0.4 mmol) in trifluoroacetic acid (2 mL) and dichloromethane (4 mL) was stirred at room temperature for 1 hour. The reaction mixture was then concentrated. The resulting residue was dissolved in methanol (30 mL) and aqueous ammonium hydroxide 30% in solution (5 mL) was added. The resulting mixture was stirred at room temperature for 4 hours. The mixture was concentrated to give [2-(3-methoxypropyl)-7-(4-piperidyl)pyrrolo[2,3-b]pyrazin-5-yl]methanol (0.100 g, crude). LC/MS (m/z, M+H): calc. 305.2, found 305.3

### Step 6: 2-(3-Methoxypropyl)-7-(4-piperidyl)-5H-pyrrolo[2,3-b]pyrazine

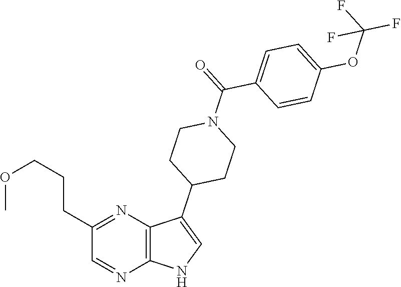 [SMILES: COCCCc1cnc2[nH]cc(C3CCN(C(=O)c4ccc(OC(F)(F)F)cc4)CC3)c2n1]

A mixture of [2-(3-methoxypropyl)-7-(4-piperidyl)pyrrolo[2,3-b]pyrazin-5-yl]methanol (0.140 g, 0.51 mmol), 4-(trifluoromethoxy)benzoic acid (0.105 g, 0.51 mmol), 3-(((ethylimino)methylene)amino)-N,N-dimethylpropan-1-amine hydrochloride (0.146 g, 0.765 mmol), 1-hydroxybenzotriazole (0.207 g, 1.53 mmol), N-ethyl-N-isopropyl-propan-2-amine (0.330 g, 2.55 mmol) in N,N-dimethylformamide (4 mL) was stirred at room temperature for 3 hours. The reaction mixture was then diluted with brine (40 mL) and extracted with ethyl acetate (30 mL×3). The organic layers were combined, washed with brine (20 mL×2), dried over Na₂SO₄, concentrated to give a crude oil. This crude product was dissolved in a mixed solution of methanol (10 mL) and 30% solution of aqueous ammonia (15 mL). After stirring for 1 hour, the resulting solution was diluted with water (50 mL) and extracted with ethyl acetate (3×50 mL). The organic layers were diluted with water, brine, dried over anhydrous Na₂SO₄, filtered, concentrated to give a crude oil which was purified by preparative HPLC to give [4-[2-(3-methoxypropyl)-5H-pyrrolo[2,3-b]pyrazin-7-yl]-1-piperidyl]-[4-(trifluoromethoxy)phenyl]methanone (0.118 g, 0.255 mmol, yield: 50%) as a white solid. LC/MS (m/z, M+H): calc. 463.2, found 463.3; ¹H NMR (400 MHz, DMSO-d6, 120° C.) δ ppm 1.78-1.90 (m, 2H), 1.92-2.03 (m, 2H), 2.09 (br dd, J=13, 3 Hz, 2H), 2.85-2.93 (m partially hidden, 2H), 3.08-3.23 (m, 3H), 3.24 (s, 3H), 3.41 (t, J=7 Hz, 2H), 4.08 (br d, J=13 Hz, 2H), 7.36 (d, J=9 Hz, 2H), 7.47 (s, 1H), 7.53 (d, J=9 Hz, 2H), 8.05 (s, 1H), 10.93-11.42 (m, 1H)

### Example 7: [2-Amino-4-(trifluoromethoxy)phenyl]-[4-[2-(3-methoxypropyl)-5H-pyrrolo[2,3-b]pyrazin-7-yl]-1-piperidyl]methanone

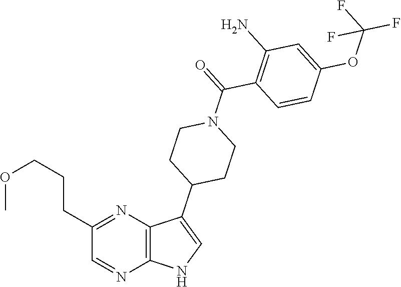 [SMILES: COCCCc1cnc2[nH]cc(C3CCN(C(=O)c4ccc(OC(F)(F)F)cc4N)CC3)c2n1]

A mixture of [2-(3-methoxypropyl)-7-(4-piperidyl)pyrrolo[2,3-b]pyrazin-5-yl]methanol (0.080 g, 0.292 mmol), 2-amino-4-(trifluoromethoxy)benzoic acid (0.0645 g, 0.292 mmol), 3-(((ethylimino)methylene)amino)-N,N-dimethylpropan-1-amine hydrochloride (0.0835 g, 0.44 mmol), 1-hydroxybenzotriazole (0.197 g, 1.46 mmol), N-ethyl-N-isopropyl-propan-2-amine (0.188 g, 1.46 mmol) in N,N-dimethylformamide (4 mL) was stirred at room temperature for 3 hours. The reaction mixture was diluted with brine (40 mL) and extracted with ethyl acetate (30 mL×3). The organic layers were combined, washed with brine (20 mL×2), dried over Na₂SO₄, concentrated to get a crude oil. This crude product was dissolved in a mixed solution of methanol (10 mL) and 30% solution aqueous ammonia (15 mL). After stirring for 1 hour, the resulting solution was diluted with water (50 mL) and extracted with ethyl acetate (3×50 mL). The organic layers were washed with water, brine, dried over anhydrous Na₂SO₄, filtered and concentrated to give a crude oil which was purified by preparative HPLC to give [2-amino-4-(trifluoromethoxy)phenyl]-[4-[2-(3-methoxypropyl)-5H-pyrrolo[2,3-b]pyrazin-7-yl]-1-piperidyl]methanone (36 mg, yield: 26%) as a white solid. LC/MS (m/z, M+H): calc. 478.2, found 478.2; ¹H NMR (400 MHz, DMSO-d6, 100° C.) δ ppm 1.75-1.90 (m, 2H), 1.92-2.03 (m, 2H), 2.09 (br dd, J=13, 3 Hz, 2H), 2.82-2.89 (m, 2H), 3.06-3.24 (m, 3H), 3.24 (s, 3H), 3.40 (t, J=7 Hz, 2H), 4.08 (br d, J=13 Hz, 2H), 5.31 (s, 2H), 6.35-6.52 (m, 1H), 6.67 (br d, J=1 Hz, 1H), 7.12 (d, J=8 Hz, 1H), 7.48 (s, 1H), 8.05 (s, 1H), 10.84-11.77 (m, 1H)

### Example 8: [4-(2-Cyclohexyl-5H-pyrrolo[2,3-b]pyrazin-7-yl)-1-piperidyl]-[4-(trifluoromethoxy)phenyl]methanone

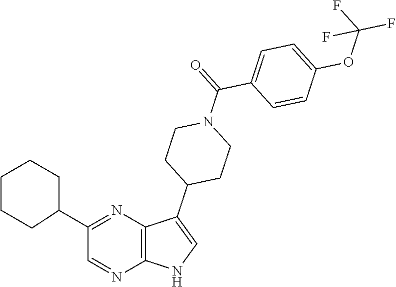 [SMILES: O=C(c1ccc(OC(F)(F)F)cc1)N1CCC(c2c[nH]c3ncc(C4CCCCC4)nc23)CC1]

### Step 1: tert-Butyl 4-[2-(cyclohexen-1-yl)-5-(2-trimethylsilylethoxymethyl)pyrrolo[2,3-b]pyrazin-7-yl]-3,6-dihydro-2H-pyridine-1-carboxylate

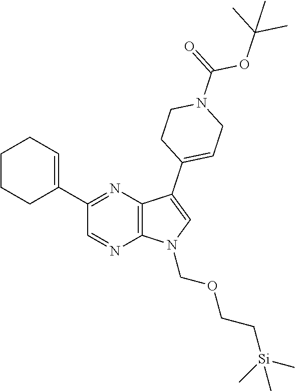 [SMILES: CC(C)(C)OC(=O)N1CC=C(c2cn(COCC[Si](C)(C)C)c3ncc(C4=CCCCC4)nc23)CC1]

A mixture of tert-butyl 4-[2-bromo-5-(2-trimethylsilylethoxymethyl)pyrrolo[2,3-b]pyrazin-7-yl]-3,6-dihydro-2H-pyridine-1-carboxylate (1.70 g, 0.00334 mol), 2-(cyclohexen-1-yl)-4,4,5,5-tetramethyl-1,3,2-dioxaborolane (0.694 g, 3.34 mmol), [1,1′-bis(diphenylphosphino)ferrocene]dichloropalladium(II) (0.244 g, 0.334 mmol), sodium bicarbonate (1.06 g, 10.0 mmol) in 1,4-dioxane (20 mL) and water (5 mL) was stirred at 80° C. for 3 hours under N₂ atmosphere. The mixture was diluted with water (50 mL) and extracted with ethyl acetate (50 mL×3). The organic layers were combined and washed with brine (100 mL), dried over Na₂SO₄, filtered and concentrated in vacuo. The resulting residue was purified by silica gel flash chromatography (0-10% ethyl acetate in petroleum ether) to give tert-butyl 4-[2-(cyclohexen-1-yl)-5-(2-trimethylsilylethoxymethyl)pyrrolo[2,3-b]pyrazin-7-yl]-3,6-dihydro-2H-pyridine-1-carboxylate (1.22 g, 2.4 mmol, yield: 71%) as an oil. LC/MS (m/z, M+H): calc. 511.3, found 511.1

### Step 2: tert-Butyl 4-[2-cyclohexyl-5-(2-trimethylsilylethoxymethyl)pyrrolo[2,3-b]pyrazin-7-yl]piperidine-1-carboxylate

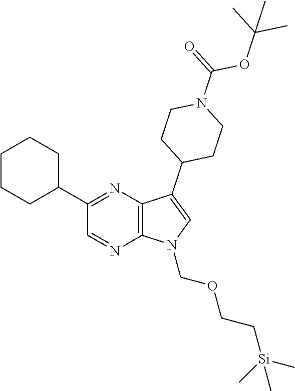 [SMILES: CC(C)(C)OC(=O)N1CCC(c2cn(COCC[Si](C)(C)C)c3ncc(C4CCCCC4)nc23)CC1]

To a solution of tert-butyl 4-[2-(cyclohexen-1-yl)-5-(2-trimethylsilylethoxymethyl)pyrrolo[2,3-b]pyrazin-7-yl]-3,6-dihydro-2H-pyridine-1-carboxylate (1.22 g, 2.4 mmol) in ethyl acetate (20 mL) was added Pd/C (20 wt %, 244 mg). The mixture was stirred for 24 hours at room temperature under 1 atmosphere of hydrogen. The reaction mixture was filtered and the filtrate was concentrated under reduced pressure and purified by silica gel chromatography (10% ethyl acetate in petroleum ether) to give tert-butyl 4-[2-cyclohexyl-5-(2-trimethylsilylethoxymethyl)pyrrolo[2,3-b]pyrazin-7-yl]piperidine-1-carboxylate (1.15 g, 2.23 mmol, yield: 94%). LC/MS (m/z, M+H): calc. 515.3, found 515.2

### Step 3: 2-Cyclohexyl-7-(4-piperidyl)pyrrolo[2,3-b]pyrazin-5-yl]methanol

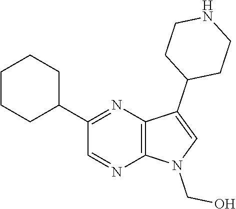 [SMILES: OCn1cc(C2CCNCC2)c2nc(C3CCCCC3)cnc21]

To a solution of tert-butyl 4-[2-cyclohexyl-5-(2-trimethylsilylethoxymethyl)pyrrolo[2,3-b]pyrazin-7-yl]piperidine-1-carboxylate (600 mg, 1.17 mmol) in dichloromethane (5 mL) was added trifluoroacetic acid (5 mL) at 0° C. The resulting mixture was stirred at room temperature for 1 hour. The reaction mixture was then concentrated to afford a yellow oil (430 mg, crude) which was used in the next step without further purification. LC/MS (m/z, M+H): calc. 315.2, found 315.1

### Step 4: [4-(2-Cyclohexyl-5H-pyrrolo[2,3-b]pyrazin-7-yl)-1-piperidyl]-[4-(trifluoromethoxy)phenyl]methanone

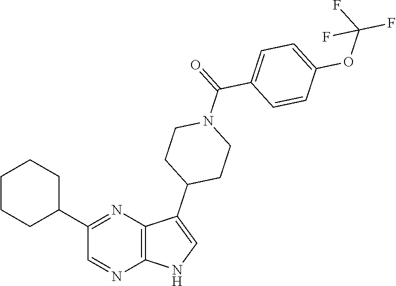 [SMILES: O=C(c1ccc(OC(F)(F)F)cc1)N1CCC(c2c[nH]c3ncc(C4CCCCC4)nc23)CC1]

To a solution of [2-cyclohexyl-7-(4-piperidyl)pyrrolo[2,3-b]pyrazin-5-yl]methanol (430 mg, 1.32 mmol) and 4-(trifluoromethoxy)benzoic acid (343 mg, 1.66 mmol) in N,N-dimethylformamide (5 mL) was added 1-hydroxybenzotriazole hydrate (255 mg, 1.66 mmol), N′-(ethylcarbonimidoyl)-N,N-dimethylpropane-1,3-diamine hydrochloride (319 mg, 1.66 mmol) and N-ethyl-N-isopropyl-propan-2-amine (537 mg, 04.16 mmol). The resulting mixture was stirred for 16 hours at room temperature. The reaction mixture was diluted with water (50 mL) and extracted with ethyl acetate (3×50 mL). The organic layers were combined and concentrated to give a crude oil. The crude product was dissolved in a mixed solution of methanol (10 mL) and aqueous ammonia purified (15 mL). After stirred for 1 hour, the resulting solution was treated with water and extracted with ethyl acetate. The combined organic layers were washed with water, brine, dried over anhydrous Na₂SO₄, filtered and concentrated to give a crude oil which was purified by silica gel chromatography (3% methanol in dichloromethane) to give [4-(2-cyclohexyl-5H-pyrrolo[2,3-b]pyrazin-7-yl)-1-piperidyl]-[4-(trifluoromethoxy)phenyl]methanone (750 mg, 2.77 mmol, 53% yield) as a white solid. LC/MS (m/z, M+H): calc. 473.2, found 473.3; ¹H NMR (400 MHz, DMSO-d6, 120° C.) δ ppm 1.31 (tt, J=12, 3 Hz, 1H), 1.44 (qt, J=12, 3 Hz, 2H), 1.62 (qd, J=12, 3 Hz, 2H), 1.69-1.77 (m, 1H), 1.79-1.89 (m, 4H), 1.89-1.99 (m, 2H), 2.09 (br dd, J=13, 3 Hz, 2H), 2.75-2.91 (m partially hidden, 1H), 3.09-3.31 (m, 3H), 4.02-4.15 (m, 2H), 7.36 (d, J=9 Hz, 2H), 7.46 (s, 1H), 7.53 (d, J=9 Hz, 2H), 8.07 (s, 1H), 11.13 (br s, 1H)

### Example 9: [2-Amino-4-(trifluoromethoxy)phenyl]-[4-(2-cyclohexyl-5H-pyrrolo[2,3-b]pyrazin-7-yl)-1-piperidyl]methanone

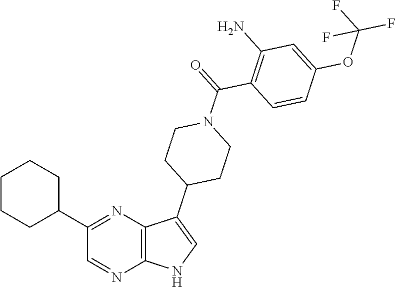 [SMILES: Nc1cc(OC(F)(F)F)ccc1C(=O)N1CCC(c2c[nH]c3ncc(C4CCCCC4)nc23)CC1]

### Step 1: [2-Cyclohexyl-7-(4-piperidyl)pyrrolo[2,3-b]pyrazin-5-yl]methanol

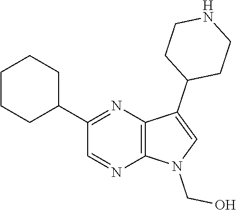 [SMILES: OCn1cc(C2CCNCC2)c2nc(C3CCCCC3)cnc21]

To a solution of tert-butyl 4-[2-cyclohexyl-5-(2-trimethylsilylethoxymethyl)pyrrolo[2,3-b]pyrazin-7-yl]piperidine-1-carboxylate (550 mg, 1.07 mmol) in dichloromethane (5 mL) was added trifluoroacetic acid (5 mL) at 0° C. The resulting mixture was stirred at room temperature for 1 hour. The reaction mixture was then concentrated to afford a yellow oil [2-cyclohexyl-7-(4-piperidyl)pyrrolo[2,3-b]pyrazin-5-yl]methanol (0.390 g, 1.07 mmol, yield: 100%) which was used in the next step without further purification. LC/MS (m/z, M+H): calc. 315.2, found 315.1

### Step 2: [2-Amino-4-(trifluoromethoxy)phenyl]-[4-(2-cyclohexyl-5H-pyrrolo[2,3-b]pyrazin-7-yl)-1-piperidyl]methanone

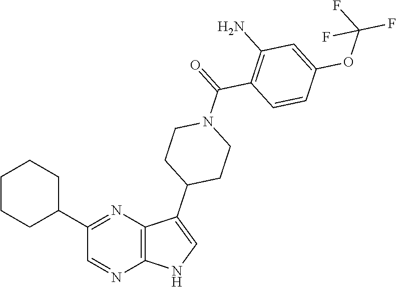 [SMILES: Nc1cc(OC(F)(F)F)ccc1C(=O)N1CCC(c2c[nH]c3ncc(C4CCCCC4)nc23)CC1]

To a solution of [2-cyclohexyl-7-(4-piperidyl)pyrrolo[2,3-b]pyrazin-5-yl]methanol (384 mg, 1.07 mmol) and 2-amino-4-(trifluoromethoxy)benzoic acid (150 mg, 0.68 mmol) in N,N-dimethylformamide (5 mL) was added 1-hydroxybenzotriazole; hydrate (0.328 g, 2.14 mmol), N′-(ethylcarbonimidoyl)-N,N-dimethylpropane-1,3-diamine hydrochloride (0.411 g, 2.14 mmol) and N-ethyl-N-isopropyl-propan-2-amine (0.692 g, 5.36 mmol). The resulting mixture was stirred for 16 hours at room temperature. The reaction mixture was then diluted with water (50 mL) and extracted with ethyl acetate (3×50 mL). The organic layers were combined and concentrated to give a crude oil. The crude product was dissolved in a mixed solution of methanol (10 mL) and 30% aqueous ammonia solution (15 mL). After stirred for 1 hour, the resulting solution was treated with water (50 mL) and extracted with ethyl acetate (3×50 mL). The organic layers were combined, washed with water and brine, dried over anhydrous Na₂SO₄, filtered and concentrated to give a crude oil which was purified by silica gel chromatography (3% methanol in dichloromethane) and preparative HPLC to give [2-amino-4-(trifluoromethoxy)phenyl]-[4-(2-cyclohexyl-5H-pyrrolo[2,3-b]pyrazin-7-yl)-1-piperidyl]methanone (100.8 mg, 0.205 mmol, yield: 19%) as a white solid. LC/MS (m/z, M+H): calc. 488.2, found 488.1; ¹H NMR (400 MHz, DMSO-d6, 120° C.) δ ppm 1.31 (tt, J=12, 3 Hz, 1H), 1.43 (qt, J=12, 3 Hz, 2H), 1.62 (qd, J=12, 3 Hz, 2H), 1.68-1.76 (m, 1H), 1.77-1.99 (m, 6H), 2.09 (br dd, J=13, 3 Hz, 2H), 2.71-2.85 (m partially hidden, 1H), 3.05-3.32 (m, 3H), 4.08 (br d, J=13 Hz, 2H), 5.24 (br s, 2H), 6.40-6.53 (m, 1H), 6.68 (br d, J=1 Hz, 1H), 7.12 (d, J=8 Hz, 1H), 7.45 (s, 1H), 8.07 (s, 1H), 10.79-11.49 (m, 1H)

### Example 10: [2-Amino-4-(trifluoromethoxy)phenyl]-[4-[2-(1-cyclopropyl-4-piperidyl)-5H-pyrrolo[2,3-b]pyrazin-7-yl]-1-piperidyl]methanone

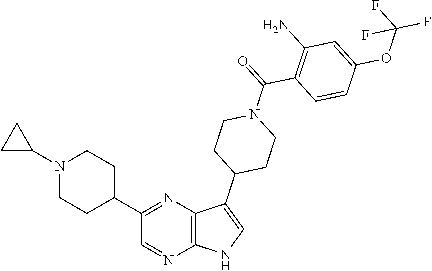 [SMILES: Nc1cc(OC(F)(F)F)ccc1C(=O)N1CCC(c2c[nH]c3ncc(C4CCN(C5CC5)CC4)nc23)CC1]

### Step 1: 4-(4,4,5,5-Tetramethyl-1,3,2-dioxaborolan-2-yl)-1,2,3,6-tetrahydropyridine

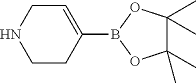

A solution of tert-butyl 4-(4,4,5,5-tetramethyl-1,3,2-dioxaborolan-2-yl)-3,6-dihydro-2H-pyridine-1-carboxylate (5.00 g, 16.2 mmol) in dichloromethane (20 mL) and trifluoroacetic acid (10 mL) was stirred for 2 hours at room temperature. The resulting solution was then concentrated under vacuum to give the crude product 4-(4,4,5,5-tetramethyl-1,3,2-dioxaborolan-2-yl)-1,2,3,6-tetrahydropyridine (4.00 g, 16.2 mmol, yield: 100%) which was used in the next step without further purification. LC/MS (m/z, M+H): calc. 210.2, found 210.1

### Step 2: 1-Cyclopropyl-4-(4,4,5,5-tetramethyl-1,3,2-dioxaborolan-2-yl)-3,6-dihydro-2H-pyridine

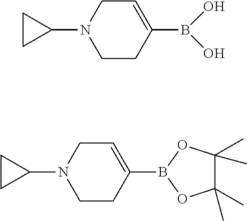 [SMILES: OB(O)C1=CCN(C2CC2)CC1 ; CC1(C)OB(C2=CCN(C3CC3)CC2)OC1(C)C]

A mixture of 4-(4,4,5,5-tetramethyl-1,3,2-dioxaborolan-2-yl)-1,2,3,6-tetrahydropyridine (4.00 g, 16.2 mmol), (1-ethoxycyclopropoxy)-trimethyl-silane (4.25 g, 24.4 mmol) and acetic acid (1.86 mL, 32.5 mmol) in methanol (40 mL) was stirred at room temperature for 0.5 hour. Sodium cyanoborohydride (3.06 g, 48.7 mmol) was then added portion-wise and the resulting mixture was warmed up to 60° C. and stirred for 12 hours. After cooling down to room temperature, the reaction mixture was concentrated and the resulting residue was purified by reverse phase chromatography using a gradient of acetonitrile (5-60%) in water (with NH₄HCO₃) to give (1-cyclopropyl-3,6-dihydro-2H-pyridin-4-yl)boronic acid (P1; 2.00 g, 9.96 mmol, yield: 61%) as a white powder and 1-cyclopropyl-4-(4,4,5,5-tetramethyl-1,3,2-dioxaborolan-2-yl)-3,6-dihydro-2H-pyridine (P2; 0.760 g, 2.66 mmol, yield: 16%) as a light yellow powder. LC/MS, P1 (m/z, M+H): calc. 168.1, found 168.1; P2 (m/z, M+H): calc. 250.2, found 250.2

### Step 3: tert-Butyl 4-[2-(1-cyclopropyl-3,6-dihydro-2H-pyridin-4-yl)-5-(2-trimethylsilylethoxymethyl)pyrrolo[2,3-b]pyrazin-7-yl]-3,6-dihydro-2H-pyridine-1-carboxylate

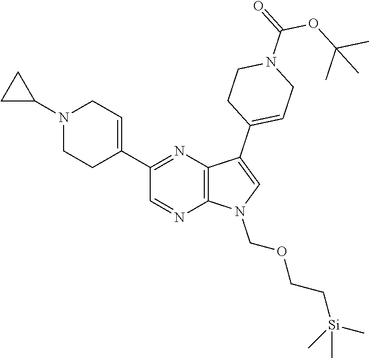 [SMILES: CC(C)(C)OC(=O)N1CC=C(c2cn(COCC[Si](C)(C)C)c3ncc(C4=CCN(C5CC5)CC4)nc23)CC1]

A mixture of tert-butyl 4-[2-bromo-5-(2-trimethylsilylethoxymethyl)pyrrolo[2,3-b]pyrazin-7-yl]-3,6-dihydro-2H-pyridine-1-carboxylate (2.00 g, 0.00297 mol), (1-cyclopropyl-3,6-dihydro-2H-pyridin-4-yl)boronic acid (0.596 g, 2.97 mmol), [1,1′-bis(diphenylphosphino)ferrocene]dichloropalladium(II) (0.217 g, 0.297 mmol), sodium carbonate (0.943 g, 8.90 mmol) in 1,4-dioxane (40 mL) and water (10 mL) was stirred at 80° C. for 16 hours under N₂ atmosphere. The reaction mixture was diluted with water (50 mL) and extracted with ethyl acetate (50 mL×3). The organic layers were combined, washed with brine (50 mL×2), dried over Na₂SO₄, filtered and concentrated in vacuo. The resulting residue was purified by silica gel flash chromatography (0-20% ethyl acetate in petroleum ether) to give tert-butyl 4-[2-(1-cyclopropyl-3,6-dihydro-2H-pyridin-4-yl)-5-(2-trimethylsilylethoxymethyl)pyrrolo[2,3-b]pyrazin-7-yl]-3,6-dihydro-2H-pyridine-1-carboxylate (0.920 g, 1.28 mmol, yield: 43%) as a yellow oil. LC/MS (m/z, M+H): calc. 552.3, found 552.3

### Step 4: tert-Butyl 4-[2-(1-cyclopropyl-4-piperidyl)-5-(2-trimethylsilylethoxymethyl)pyrrolo[2,3-b]pyrazin-7-yl]piperidine-1-carboxylate

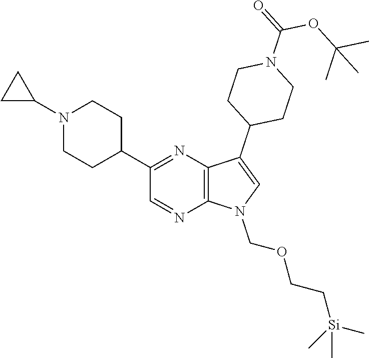 [SMILES: CC(C)(C)OC(=O)N1CCC(c2cn(COCC[Si](C)(C)C)c3ncc(C4CCN(C5CC5)CC4)nc23)CC1]

To a solution of tert-butyl 4-[2-(1-cyclopropyl-3,6-dihydro-2H-pyridin-4-yl)-5-(2-trimethylsilylethoxymethyl)pyrrolo[2,3-b]pyrazin-7-yl]-3,6-dihydro-2H-pyridine-1-carboxylate (300 mg, 0.544 mmol) in ethyl acetate (10 mL) was added Pd/C (20 wt %, 60 mg). The mixture was stirred for 24 hours at room temperature under 1 atmosphere of hydrogen. The reaction mixture was then filtered and the filtrate was concentrated under reduced pressure to give tert-butyl 4-[2-(1-cyclopropyl-4-piperidyl)-5-(2-trimethylsilylethoxymethyl)pyrrolo[2,3-b]pyrazin-7-yl]piperidine-1-carboxylate (0.334 g, 0.542 mmol, yield: 100%) which was used in the next step without further purification. LC/MS (m/z, M+H): calc. 556.4, found 556.3

### Step 5: [2-(1-cyclopropyl-4-piperidyl)-7-(4-piperidyl)pyrrolo[2,3-b]pyrazin-5-yl]methanol

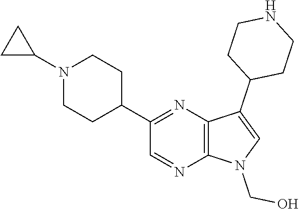 [SMILES: OCn1cc(C2CCNCC2)c2nc(C3CCN(C4CC4)CC3)cnc21]

To a solution of tert-butyl 4-[2-(1-cyclopropyl-4-piperidyl)-5-(2-trimethylsilylethoxymethyl)pyrrolo[2,3-b]pyrazin-7-yl]piperidine-1-carboxylate (167 mg, 0.271 mmol) in dichloromethane (3 mL) was added trifluoroacetic acid (3 mL) at 0° C. The resulting mixture was stirred at room temperature for 1 hour. The reaction mixture was then concentrated to give 2-(1-cyclopropyl-4-piperidyl)-7-(4-piperidyl)-5H-pyrrolo[2,3-b]pyrazine (98 mg, 0.266 mmol, yield: 98%) as a yellow oil, which was used in the next step without further purification. LC/MS (m/z, M+H): calc. 356.2, found 356.2

### Step 6: [2-Amino-4-(trifluoromethoxy)phenyl]-[4-[2-(1-cyclopropyl-4-piperidyl)-5H-pyrrolo[2,3-b]pyrazin-7-yl]-1-piperidyl]methanone

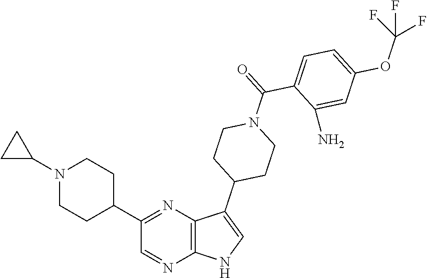 [SMILES: Nc1cc(OC(F)(F)F)ccc1C(=O)N1CCC(c2c[nH]c3ncc(C4CCN(C5CC5)CC4)nc23)CC1]

To a solution of [2-(1-cyclopropyl-4-piperidyl)-7-(4-piperidyl)pyrrolo[2,3-b]pyrazin-5-yl]methanol (96.0 mg, 0.266 mmol) and 2-amino-4-(trifluoromethoxy)benzoic acid (58.8 mg, 0.266 mmol) in N,N-dimethylformamide (5 mL) was added 1-hydroxybenzotriazole hydrate (0.0815 g, 0.532 mmol), N′-(ethylcarbonimidoyl)-N,N-dimethylpropane-1,3-diamine hydrochloride (0.102 g, 0.532 mmol) and N-ethyl-N-isopropyl-propan-2-amine (0.172 g, 1.33 mmol). The resulting mixture was stirred for 16 hours at room temperature. The reaction mixture was then diluted with water (30 mL) and extracted with ethyl acetate (3×30 mL). The organic layers were combined and concentrated to give a crude oil. The crude product was dissolved in a mixed solution of methanol (5 mL) and 30% aqueous ammonia solution (5 mL). After stirring for 1 hour, the resulting solution was treated with water and extracted with ethyl acetate. The organic layers were combined, washed with water, brine, dried over anhydrous Na₂SO₄, filtered and concentrated to give a crude oil which was purified by preparative HPLC to give [2-amino-4-(trifluoromethoxy)phenyl]-[4-[2-(1-cyclopropyl-4-piperidyl)-5H-pyrrolo[2,3-b]pyrazin-7-yl]-1-piperidyl]methanone (0.0118 g, 0.022 mmol, yield: 8.2%). LC/MS (m/z, M+H): calc. 529.2, found 529.3; ¹H NMR (400 MHz, DMSO-d6, 100° C.) δ ppm 0.28-0.51 (m, 4H), 1.59-1.70 (m, 1H), 1.71-1.93 (m, 6H), 2.07 (br dd, J=13, 3 Hz, 2H), 2.32 (td, J=11, 4 Hz, 2H), 2.76-2.87 (m, 1H), 3.00-3.29 (m, 5H), 4.08 (br d, J=13 Hz, 2H), 5.30 (s, 2H), 6.46 (br dd, J=8, 1 Hz, 1H), 6.67 (br d, J=1 Hz, 1H), 7.12 (d, J=8 Hz, 1H), 7.48 (d, J=2 Hz, 1H), 8.08 (s, 1H), 11.23 (br s, 1H)

### Example 11: [4-[2-(1-Cyclopropyl-4-piperidyl)-5H-pyrrolo[2,3-b]pyrazin-7-yl]-1-piperidyl]-[4-(trifluoromethoxy)phenyl]methanone

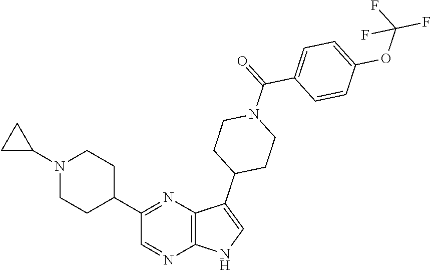 [SMILES: O=C(c1ccc(OC(F)(F)F)cc1)N1CCC(c2c[nH]c3ncc(C4CCN(C5CC5)CC4)nc23)CC1]

To a solution of [2-(1-cyclopropyl-4-piperidyl)-7-(4-piperidyl)pyrrolo[2,3-b]pyrazin-5-yl]methanol (97.0 mg, 0.27 mmol) and 4-(trifluoromethoxy)benzoic acid (55.4 mg, 0.27 mmol) in N,N-dimethylformamide (5 mL) was added 1-hydroxybenzotriazole; hydrate (0.0823 g, 0.54 mmol), N-(3-(dimethylamino)propyl)propionimidamide (0.0846 g, 0.54 mmol) and N-ethyl-N-isopropyl-propan-2-amine (0.174 g, 1.34 mmol). The resulting mixture was stirred for 16 hours at room temperature. The reaction mixture was then diluted with water (30 mL) and extracted with ethyl acetate (3×30 mL). The organic layers were combined and concentrated to give a crude oil. The crude product was dissolved in a mixed solution of methanol (5 mL) 30% aqueous ammonia solution (5 mL). After stirred for 1 hour, the resulting solution was treated with water and extracted with ethyl acetate (30 mL×3). The organic layers were combined, washed with water, brine, dried over anhydrous Na₂SO₄, filtered and concentrated to give a crude oil which was purified by preparative HPLC to give [4-[2-(1-cyclopropyl-4-piperidyl)-5H-pyrrolo[2,3-b]pyrazin-7-yl]-1-piperidyl]-[4-(trifluoromethoxy)phenyl]methanone (0.0136 g, 0.026 mmol, yield: 10%). LC/MS (m/z, M+H): calc. 514.2, found 514.3; ¹H NMR (400 MHz, DMSO-d6, 100° C.) δ ppm 0.28-0.51 (m, 4H), 1.60-1.70 (m, 1H), 1.73-1.93 (m, 6H), 2.08 (br dd, J=13, 2 Hz, 2H), 2.33 (td, J=11, 3 Hz, 2H), 2.76-2.88 (m, 1H), 3.06 (br d, J=12 Hz, 2H), 3.09-3.29 (m, 3H), 3.92-4.29 (m, 2H), 7.37 (br d, J=9 Hz, 2H), 7.49 (br s, 1H), 7.54 (d, J=9 Hz, 2H), 8.09 (s, 1H), 11.24 (br s, 1H)

### Example 12: (rac)-[2-Amino-4-(trifluoromethoxy)phenyl]-[4-(2-tetrahydrofuran-3-yl-5H-pyrrolo[2,3-b]pyrazin-7-yl)-1-piperidyl]methanone

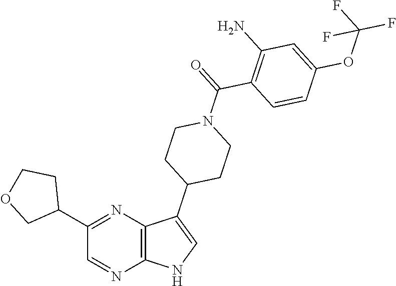 [SMILES: Nc1cc(OC(F)(F)F)ccc1C(=O)N1CCC(c2c[nH]c3ncc(C4CCOC4)nc23)CC1]

To a solution of (rac)-7-(4-piperidyl)-2-tetrahydrofuran-3-yl-pyrrolo[2,3-b]pyrazin-5-yl]methanol (300 mg, 0.83 mmol) and 2-amino-4-(trifluoromethoxy)benzoic acid (150 mg, 0.68 mmol) in N,N-dimethylformamide (5 mL) was added 1-hydroxybenzotriazole hydrate (255 mg, 1.66 mmol), N′-(ethylcarbonimidoyl)-N,N-dimethylpropane-1,3-diamine hydrochloride (319 mg, 1.66 mmol) and N-ethyl-N-isopropyl-propan-2-amine (537 mg, 4.16 mmol). The resulting mixture was stirred for 16 hours at room temperature. The reaction mixture was diluted with water (50 mL) and extracted with ethyl acetate (3×50 mL). The organic layers were combined and concentrated to give a crude oil. The crude product was dissolved in a mixed solution of methanol (10 mL) and 30% aqueous ammonia solution (15 mL). After stirred for 1 hour, the resulting solution was treated with water and extracted with ethyl acetate. The organic layers were combined, washed with water, brine, dried over anhydrous Na₂SO₄, filtered and concentrated to give a crude oil which was purified by silica gel chromatography (3% methanol in dichloromethane) to give (rac)-[2-amino-4-(trifluoromethoxy)phenyl]-[4-(2-tetrahydrofuran-3-yl-5H-pyrrolo[2,3-b]pyrazin-7-yl)-1-piperidyl]methanone (81.6 mg, 0.172 mmol, yield: 21%) as a white solid. LC/MS (m/z, M+H): calc. 476.2, found 476.1; ¹H NMR (400 MHz, DMSO-d6, 120° C.) δ ppm 1.78-1.93 (m, 2H), 2.08 (br dd, J=13, 3 Hz, 2H), 2.16-2.39 (m, 2H), 3.07-3.27 (m, 3H), 3.68 (quin, J=8 Hz, 1H), 3.79-3.88 (m, 2H), 3.96 (td, J=8, 5 Hz, 1H), 4.04-4.14 (m, 3H), 5.24 (br s, 2H), 6.42-6.55 (m, 1H), 6.68 (br d, J=1 Hz, 1H), 7.11 (d, J=8 Hz, 1H), 7.49 (s, 1H), 8.12 (s, 1H), 11.18 (br s, 1H)

### Examples 13 and 14: [2-Amino-4-(trifluoromethoxy)phenyl]-[4-(2-tetrahydrofuran-3-yl-5H-pyrrolo[2,3-b]pyrazin-7-yl)-1-piperidyl]methanone, Isomers 1 and 2

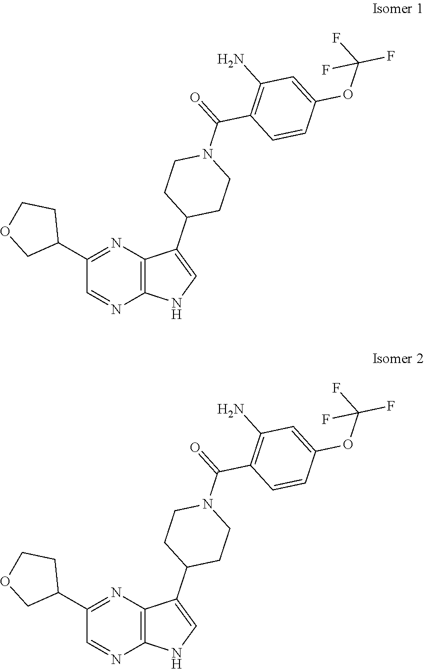 [SMILES: Nc1cc(OC(F)(F)F)ccc1C(=O)N1CCC(c2c[nH]c3ncc(C4CCOC4)nc23)CC1 ; Nc1cc(OC(F)(F)F)ccc1C(=O)N1CCC(c2c[nH]c3ncc(C4CCOC4)nc23)CC1]

Chiral separation of (rac)-[2-amino-4-(trifluoromethoxy)phenyl]-[4-(2-tetrahydrofuran-3-yl-5H-pyrrolo[2,3-b]pyrazin-7-yl)-1-piperidyl]methanone (61 mg, 0.13 mmol), using a chiralpak IF column (5 μm, 250×30 mm), eluting with 30% EtOH in n-Heptane+0.1% TEA (flow rate 40 mL/min, UV detection at 240 nm), gave 29 mg (47%) of the first eluting Isomer (Example 13, Isomer 1) and 33 mg (54%) of the second eluting Isomer (Example 14, Isomer 2) as white solids.

Isomer 1 (Example 13): LC/MS (m/z, M+H): 476; ¹H NMR (400 MHz, DMSO-d6, 100° C.) δ ppm 1.77-1.89 (m, 2H), 2.03-2.12 (m, 2H), 2.17-2.33 (m, 2H), 3.08-3.24 (m, 3H), 3.68 (quin, J=7.8 Hz, 1H), 3.78-3.89 (m, 2H), 3.93-4.00 (m, 1H), 4.04-4.15 (m, 3H), 5.30 (br s, 2H), 6.47 (br d, J=8.3 Hz, 1H), 6.68 (br s, 1H), 7.11 (d, J=8.3 Hz, 1H), 7.51 (s, 1H), 8.12 (s, 1H), 11.21-11.45 (m, 1H)

Isomer 2 (Example 14): LC/MS (m/z, M+H): 476; ¹H NMR (400 MHz, DMSO-d6, 100° C.) δ ppm 1.76-1.91 (m, 2H), 2.03-2.14 (m, 2H), 2.17-2.38 (m, 2H), 3.08-3.27 (m, 3H), 3.68 (quin, J=8 Hz, 1H), 3.78-3.90 (m, 2H), 3.92-4.1 (m, 1H), 4.04-4.16 (m, 3H), 5.30 (s, 2H), 6.47 (br d, J=8.3, 1H), 6.68 (br s, 1H), 7.12 (d, J=8.3 Hz, 1H), 7.51 (br s, 1H), 8.12 (s, 1H), 11.27-11.36 (m, 1H)

### Example 15: [4-[2-(1-Methyl-4-piperidyl)-5H-pyrrolo[2,3-b]pyrazin-7-yl]-1-piperidyl]-[4-(pentafluoro-λ⁶-sulfanyl)phenyl]methanone

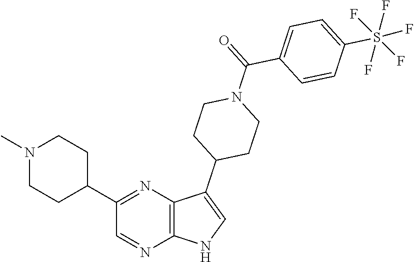 [SMILES: CN1CCC(c2cnc3[nH]cc(C4CCN(C(=O)c5ccc(S(F)(F)(F)(F)F)cc5)CC4)c3n2)CC1]

At room temperature, under argon atmosphere, to a solution of 2-(1-methyl-4-piperidyl)-7-(4-piperidyl)-5H-pyrrolo[2,3-b]pyrazine (60 mg, 0.2 mmol) in DMF (1 mL), were added triethylamine (140 μL, 1 mmol), 4-(pentafluorothio)benzoic acid (51 mg, 0.2 mmol) and TATU (77 mg, 0.24 mmol). The reaction mixture was then stirred for 0.5 hours at room temperature, then diluted with AcOEt and transferred to a separating funnel containing an aqueous saturated solution of NaCl. The whole was extracted three times with AcOEt. The combined organic layers were dried over MgSO₄, filtered and concentrated to dryness. The resulting residue was purified by flash chromatography on silica gel (12 g) eluting with DCM/MeOH (90/10) to give 15 mg (14% yield) of [4-[2-(1-methyl-4-piperidyl)-5H-pyrrolo[2,3-b]pyrazin-7-yl]-1-piperidyl]-[4-(pentafluoro-λ⁶-sulfanyl)phenyl]methanone as a white solid. LC/MS (m/z, M+H): calc. 530.6, found 530.2; ¹H NMR (400 MHz, DMSO-d6, 100° C.) δ ppm 1.78-2.26 (m, 12H), 2.31 (s, 3H), 2.77-2.86 (partially hidden, m, 1H), 3.10-3.29 (m, 3H), 3.89-4.26 (m, 2H), 7.51 (d, J=2.6 Hz, 1H), 7.63 (br d, J=8.5 Hz, 2H), 7.93 (d, J=8.8 Hz, 2H), 8.10 (s, 1H), 11.30 (m, 1H)

### Example 16: [2-Amino-4-(pentafluoro-λ⁶-sulfanyl)phenyl]-[4-[2-(1-methyl-4-piperidyl)-5H-pyrrolo[2,3-b]pyrazin-7-yl]-1-piperidyl]methanone

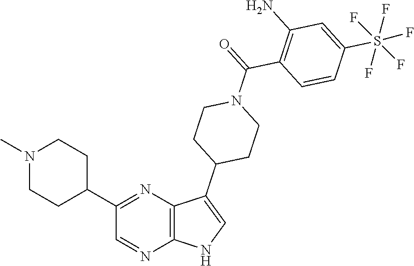 [SMILES: CN1CCC(c2cnc3[nH]cc(C4CCN(C(=O)c5ccc(S(F)(F)(F)(F)F)cc5N)CC4)c3n2)CC1]

At room temperature, under argon atmosphere, to a solution of 2-(1-methyl-4-piperidyl)-7-(4-piperidyl)-5H-pyrrolo[2,3-b]pyrazine (120 mg, 0.4 mmol) in DMF (2 mL), were added triethylamine (280 μL, 2 mmol), 2-amino-4-(pentafluoro-λ⁶-sulfanyl)benzoic acid; hydrochloride (126 mg, 0.42 mmol) and TBTU (154 mg, 0.48 mmol). The reaction mixture was then stirred for 0.5 hours at room temperature, then diluted with AcOEt, transferred in a separating funnel containing an aqueous saturated solution of NaCl. The whole was extracted three times with AcOEt. The combined organic layers were dried over MgSO₄, filtered and concentrated to dryness. The resulting residue was purified by flash chromatography on silical gel (12 g) eluting with DCM/MeOH/NH₄₀H (90/10/1), to give 30 mg (14% yield) of [2-amino-4-(pentafluoro-λ⁶-sulfanyl)phenyl]-[4-[2-(1-methyl-4-piperidyl)-5H-pyrrolo[2,3-b]pyrazin-7-yl]-1-piperidyl]methanone as a white solid. LC/MS (m/z, M+H): calc. 545.6, found 545.2; ¹H NMR (400 MHz, DMSO-d6, 100° C.) δ ppm 1.82-1.98 (m, 6H), 2.04-2.15 (m, 4H), 2.25 (s, 3H), 2.74-2.83 (m, 1H), 2.88-2.93 (partially hidden, m, 3H), 3.12-3.28 (m, 3H), 4.04-4.15 (m, 2H), 5.44 (br s, 2H), 7.03 (dd, J=8.5, 2.3 Hz, 1H), 7.24 (d, J=8.5 Hz, 1H), 7.29 (d, J=2.3 Hz, 1H), 7.51 (s, 1H), 8.12 (s, 1H), 11.23-11.30 (m, 1H)

### Example 17: [4-(Pentafluoro-X⁶-sulfanyl)phenyl]-[4-(2-tetrahydropyran-4-yl-5H-pyrrolo[2,3-b]pyrazin-7-yl)-1-piperidyl]methanone

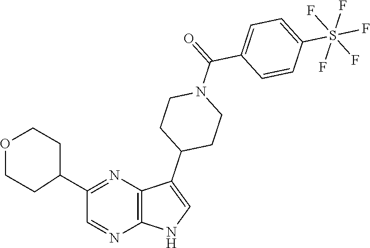 [SMILES: O=C(c1ccc(S(F)(F)(F)(F)F)cc1)N1CCC(c2c[nH]c3ncc(C4CCOCC4)nc23)CC1]

At room temperature, under argon atmosphere, to a solution of 4-(pentafluoro-λ⁶-sulfanyl)benzoic acid (36 mg, 0.14 mmol) in DMF (1 mL), were added TBTU (54 mg, 0.17 mmol), N,N-diisopropylethylamine (40 μL, 0.23 mmol). Then, at 0° C., to the resulting mixture was added a solution of 7-(4-piperidyl)-2-tetrahydropyran-4-yl-5H-pyrrolo[2,3-b]pyrazine (40 mg, 0.14 mmol) in DMF (1.5 mL). The reaction mixture was then stirred for 10 minutes, then diluted with AcOEt and transferred to a separating funnel containing water. The whole was extracted three times with AcOEt. The combined organic layers were dried over MgSO₄, filtered and concentrated to dryness. The resulting residue was purified by flash chromatography on silica gel (12 g) eluting with DCM/MeOH (90/10), to give 55 mg (79% yield) of [4-(pentafluoro-λ⁶-sulfanyl)phenyl]-[4-(2-tetrahydropyran-4-yl-5H-pyrrolo[2,3-b]pyrazin-7-yl)-1-piperidyl]methanone as a white solid. LC/MS (m/z, M+H): calc. 517.2, found 517.1; ¹H NMR (400 MHz, DMSO-d6, 120° C.) δ ppm 1.78-1.98 (m, 6H), 2.10 (br dd, J=13, 3 Hz, 2H), 3.02-3.29 (m, 4H), 3.51 (td, J=11, 3 Hz, 2H), 3.93-4.01 (m, 2H), 4.08 (br d, J=12 Hz, 2H), 7.36 (br d, J=9 Hz, 2H), 7.49 (d, J=2 Hz, 1H), 7.53 (d, J=9 Hz, 2H), 8.11 (s, 1H), 11.16 (br s, 1H)

### Example 18: [2-Amino-4-(pentafluoro-λ⁶-sulfanyl)phenyl]-[4-(2-tetrahydropyran-4-yl-5H-pyrrolo[2,3-b]pyrazin-7-yl)-1-piperidyl]methanone

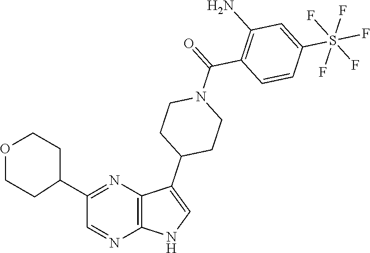 [SMILES: Nc1cc(S(F)(F)(F)(F)F)ccc1C(=O)N1CCC(c2c[nH]c3ncc(C4CCOCC4)nc23)CC1]

Example 18 was prepared following the procedure described in Example 17, using 2-amino-4-(pentafluoro-λ⁶-sulfanyl)benzoic acid (46 mg, 0.17 mmol, 100 mass %), TBTU (67 mg, 0.21 mmol), N,N-diisopropylethylamine (0.11 mL, 0.61 mmol) and 7-(4-piperidyl)-2-tetrahydropyran-4-yl-5H-pyrrolo[2,3-b]pyrazine (50 mg, 0.17 mmol) to give 63 mg (68% yield) of [2-amino-4-(pentafluoro-λ⁶-sulfanyl)phenyl]-[4-(2-tetrahydropyran-4-yl-5H-pyrrolo[2,3-b]pyrazin-7-yl)-1-piperidyl]methanone as a white solid. LC/MS (m/z, M+H): calc. 532.5, found 532.0; ¹H NMR (400 MHz, DMSO-d6, 100° C.) δ ppm 1.77-1.95 (m, 6H), 2.10 (br dd, J=12.9, 2.8 Hz, 2H), 3.03-3.26 (m, 4H), 3.50 (ddd, J=11.3, 11.3, 2.9 Hz, 2H), 3.94-4.14 (m, 4H), 5.42 (br s, 2H), 6.99 (dd, J=8.4, 2.3 Hz, 1H), 7.21 (d, J=8.4 Hz, 1H), 7.27 (d, J=2.3 Hz, 1H), 7.50 (s, 1H), 8.11 (s, 1H), 11.23-11.34 (m, 1H)

### Example 19: [2-Amino-4-(pentafluoro-λ⁶-sulfanyl)phenyl]-[4-[2-(1-cyclopropyl-4-piperidyl)-5H-pyrrolo[2,3-b]pyrazin-7-yl]-1-piperidyl]methanone

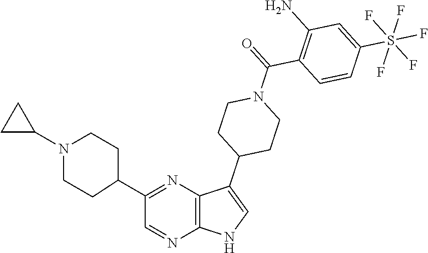 [SMILES: Nc1cc(S(F)(F)(F)(F)F)ccc1C(=O)N1CCC(c2c[nH]c3ncc(C4CCN(C5CC5)CC4)nc23)CC1]

Example 19 was prepared following the procedure described in Example 17, using 2-amino-4-(pentafluoro-λ⁶-sulfanyl)benzoic acid (50 mg, 0.19 mmol), TBTU (70 mg, 0.22 mmol), N,N-diisopropylethylamine (40 L, 0.23 mmol), 2-(1-cyclopropyl-4-piperidyl)-7-(4-piperidyl)-5H-pyrrolo[2,3-b]pyrazine; dihydrochloride (45 mg, 0.11 mmol) to give 34 mg (53% yield) of [2-amino-4-(pentafluoro-λ⁶-sulfanyl)phenyl]-[4-[2-(1-cyclopropyl-4-piperidyl)-5H-pyrrolo[2,3-b]pyrazin-7-yl]-1-piperidyl]methanone as a white solid. LC/MS (m/z, M+H): calc. 571.6, found 571.1; ¹H NMR (400 MHz, DMSO-d6, 100° C.) δ ppm 0.29-0.36 (m, 2H), 0.37-0.45 (m, 2H), 1.62-1.69 (m, 1H), 1.72-1.92 (m, 6H), 2.04-2.14 (m, 2H), 2.28-2.37 (m, 2H), 2.76-2.86 (m, 1H), 3.01-3.09 (m, 2H), 3.09-3.24 (m, 3H), 3.99-4.12 (m, 2H), 5.41 (s, 2H), 6.98 (dd, J=8.5, 2.2 Hz, 1H), 7.20 (br d, J=8.5 Hz, 1H), 7.26 (d, J=2.2 Hz, 1H), 7.48 (s, 1H), 8.08 (s, 1H), 11.23 (m, 1H)

### Example 20: [4-[2-(1-Cyclopropyl-4-piperidyl)-5H-pyrrolo[2,3-b]pyrazin-7-yl]-1-piperidyl]-[4-(pentafluoro-λ⁶-sulfanyl)phenyl]methanone

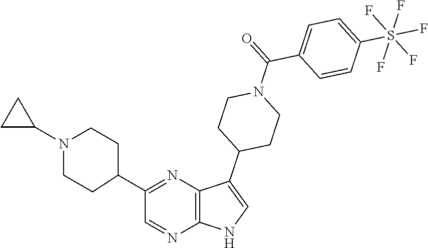 [SMILES: O=C(c1ccc(S(F)(F)(F)(F)F)cc1)N1CCC(c2c[nH]c3ncc(C4CCN(C5CC5)CC4)nc23)CC1]

Example 20 was prepared following the procedure described in Example 17, using 4-(pentafluoro-λ⁶-sulfanyl)benzoic acid (32 mg, 0.13 mmol), TBTU (46 mg, 0.14 mmol), N,N-diisopropylethylamine (60 L, 0.34 mmol), 2-(1-cyclopropyl-4-piperidyl)-7-(4-piperidyl)-5H-pyrrolo[2,3-b]pyrazine; dihydrochloride (35 mg, 0.09 mmol) to give 29 mg (60% yield) of [4-[2-(1-cyclopropyl-4-piperidyl)-5H-pyrrolo[2,3-b]pyrazin-7-yl]-1-piperidyl]-[4-(pentafluoro-λ⁶-sulfanyl)phenyl]methanone as a white solid. LC/MS (m/z, M+H): calc. 556.6, found 556.1; ¹H NMR (400 MHz, DMSO-d6, 100° C.) δ ppm 0.27-0.36 (m, 2H), 0.37-0.48 (m, 2H), 1.61-1.72 (m, 1H), 1.73-1.94 (m, 6H), 2.03-2.16 (m, 2H), 2.26-2.39 (m, 2H), 2.76-2.87 (m, 1H), 3.01-3.10 (br d, m, 2H), 3.10-3.29 (m, 3H), 3.87-4.29 (m, 2H), 7.49 (s, 1H), 7.62 (br d, J=8.5 Hz, 2H), 7.92 (d, J=8.5 Hz, 2H), 8.09 (s, 1H), 11.24 (m, 1H)

### Example 21: [4-(Cyclopropoxy)phenyl]-[4-(2-tetrahydropyran-4-yl-5H-pyrrolo[2,3-b]pyrazin-7-yl)-1-piperidyl]methanone

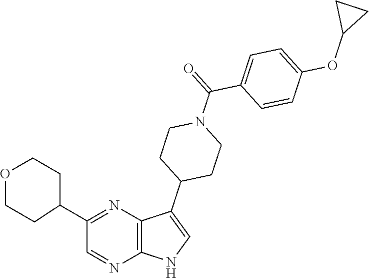 [SMILES: O=C(c1ccc(OC2CC2)cc1)N1CCC(c2c[nH]c3ncc(C4CCOCC4)nc23)CC1]

Example 21 was prepared following the procedure described in Example 17, using 4-(cyclopropoxy)benzoic acid (35 mg, 0.20 mmol), TBTU (54 mg, 0.17 mmol), 7-(4-piperidyl)-2-tetrahydropyran-4-yl-5H-pyrrolo[2,3-b]pyrazine; hydrochloride (40 mg, 0.12 mmol), N,N-diisopropylethylamine (65 μL, 0.37 mmol) to give 36 mg (65% yield) of [4-(cyclopropoxy)phenyl]-[4-(2-tetrahydropyran-4-yl-5H-pyrrolo[2,3-b]pyrazin-7-yl)-1-piperidyl]methanone as a white solid. LC/MS (m/z, M+H): calc. 447.5, found 447.2; ¹H NMR (400 MHz, DMSO-d6, 100° C.) δ ppm 0.62-0.84 (m, 4H), 1.76-1.95 (m, 6H), 2.03-2.13 (m, 2H), 3.02-3.27 (m, 4H), 3.45-3.50 (ddd, J=11.3, 11.3, 2.8 Hz, 2H), 3.83-3.91 (m, 1H), 3.95-4.01 (m, 2H), 4.10-4.20 (m, 2H), 7.07 (d, J=8.6 Hz, 2H), 7.37 (d, J=8.6 Hz, 2H), 7.51 (d, J=2.8 Hz, 1H), 8.11 (s, 1H), 11.26 (m, 1H)

### Example 22: [4-(1,1,2,2,2-Pentafluoroethyl)phenyl]-[4-(2-tetrahydropyran-4-yl-5H-pyrrolo[2,3-b]pyrazin-7-yl)-1-piperidyl]methanone

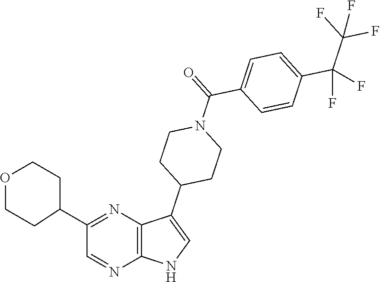 [SMILES: O=C(c1ccc(C(F)(F)C(F)(F)F)cc1)N1CCC(c2c[nH]c3ncc(C4CCOCC4)nc23)CC1]

Example 22 was prepared following the procedure described in Example 17, using 4-(1,1,2,2,2-pentafluoroethyl)benzoic acid (31 mg, 0.13 mmol), TBTU (47 mg, 0.15 mmol), 7-(4-piperidyl)-2-tetrahydropyran-4-yl-5H-pyrrolo[2,3-b]pyrazine; hydrochloride (42 mg, 0.13 mmol), N,N-diisopropylethylamine (87 μL, 0.50 mmol) to give 40 mg (61% yield) of [4-(1,1,2,2,2-pentafluoroethyl)phenyl]-[4-(2-tetrahydropyran-4-yl-5H-pyrrolo[2,3-b]pyrazin-7-yl)-1-piperidyl]methanone as a white solid. LC/MS (m/z, M+H): calc. 509.5, found 509.2; ¹H NMR (400 MHz, DMSO-d6, 100° C.) δ ppm 1.76-1.95 (m, 6H), 2.03-2.17 (m, 2H), 3.02-3.28 (m, 4H), 3.50 (ddd, J=11.4, 11.4, 2.9 Hz, 2H), 3.87-4.27 (m, 4H), 7.51 (d, J=2.8 Hz, 1H), 7.65 (d, J=8.1 Hz, 2H), 7.75 (d, J=8.1 Hz, 2H), 8.12 (s, 1H), 11.28 (br s, 1H)

### Example 23: Cyclohexyl-[4-(2-tetrahydropyran-4-yl-5H-pyrrolo[2,3-b]pyrazin-7-yl)-1-piperidyl]methanone

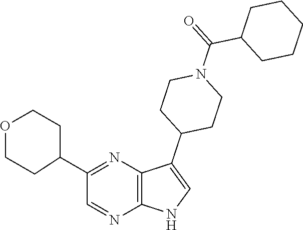 [SMILES: O=C(C1CCCCC1)N1CCC(c2c[nH]c3ncc(C4CCOCC4)nc23)CC1]

Example 23 was prepared following the procedure described for example 17, using cyclohexanecarboxylic acid (19 mg, 0.15 mmol), TBTU (46 mg, 0.14 mmol), DIPEA (68 μL, 0.39 mmol), 7-(4-piperidyl)-2-tetrahydropyran-4-yl-5H-pyrrolo[2,3-b]pyrazine; hydrochloride (42 mg, 0.13 mmol) to give 30 mg (58% yield) of cyclohexyl-[4-(2-tetrahydropyran-4-yl-5H-pyrrolo[2,3-b]pyrazin-7-yl)-1-piperidyl]methanone as a white solid. LC/MS (m/z, M+H): calc. 397.5, found 397.3; ¹H NMR (400 MHz, DMSO-d6, 100° C.) δ ppm 1.13-1.47 (m, 4H), 1.60-1.97 (m, 12H), 2.07 (br d, J=12.7 Hz, 2H), 2.55-2.67 (m, 1H), 2.96-3.21 (partially hidden m, 4H), 3.50 (ddd, J=11.3, 2.9 Hz, 2H), 3.93-4.01 (m, 2H), 4.17-4.30 (m, 2H), 7.48 (s, 1H), 8.11 (s, 1H), 10.75-12.14 (m, 1H)

### Example 24: (rac)-[2-Amino-4-(trifluoromethoxy)phenyl]-[3-(2-tetrahydropyran-4-yl-5H-pyrrolo[2,3-b]pyrazin-7-yl)pyrrolidine-1-yl]methanone

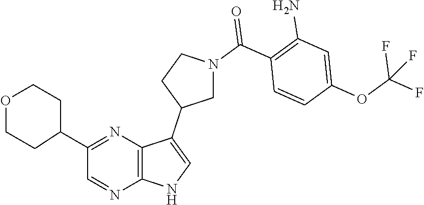 [SMILES: Nc1cc(OC(F)(F)F)ccc1C(=O)N1CCC(c2c[nH]c3ncc(C4CCOCC4)nc23)C1]

### Step 1: tert-Butyl 3-[2-bromo-5-(2-trimethylsilylethoxymethyl)pyrrolo[2,3-b]pyrazin-7-yl]-2,5-dihydropyrrole-1-carboxylate

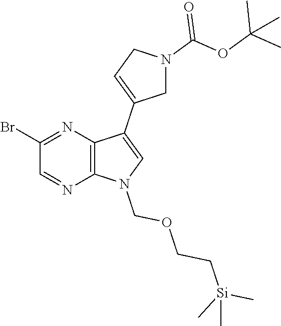 [SMILES: CC(C)(C)OC(=O)N1CC=C(c2cn(COCC[Si](C)(C)C)c3ncc(Br)nc23)C1]

To a solution of 2-[(2-bromo-7-iodo-pyrrolo[2,3-b]pyrazin-5-yl)methoxy]ethyl-trimethyl-silane (13 g, 28.6 mmol) and tert-butyl 3-(4,4,5,5-tetramethyl-1,3,2-dioxaborolan-2-yl)-2,5-dihydropyrrole-1-carboxylate (9.29 mg, 31.5 mmol) in a mixture of dioxane/water (130 mL/26 mL) was added sequentially Pd(dppf)Cl₂ (622 mg), and Na₂CO₃ (9.1 g, 85.8 mmol). The reaction mixture was refluxed for 5 hours under nitrogen atmosphere. After five hours, the reaction mixture was cooled down to room temperature, diluted with brine (200 mL), extracted with EtOAc (3×150 mL). The combined organic layers were washed with brine (2×200 mL), dried over anhydrous Na₂SO₄, filtered and concentrated to give a crude oil. The crude product was purified by silica gel chromatography (EtOAc/PE 25/75) to give 8.3 g (58% yield) of tert-butyl 3-[2-bromo-5-(2-trimethylsilylethoxymethyl)pyrrolo[2,3-b]pyrazin-7-yl]-2,5-dihydropyrrole-1-carboxylate as a yellow solid. LC/MS (m/z, M+H): calc. 496.5, found 496.1

### Step 2: tert-Butyl 3-[2-(3,6-dihydro-2H-pyran-4-yl)-5-(2-trimethylsilylethoxymethyl)pyrrolo[2,3-b]pyrazin-7-yl]-2,5-dihydropyrrole-1-carboxylate

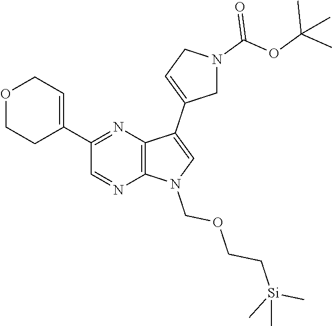 [SMILES: CC(C)(C)OC(=O)N1CC=C(c2cn(COCC[Si](C)(C)C)c3ncc(C4=CCOCC4)nc23)C1]

To a solution of tert-butyl 3-[2-bromo-5-(2-trimethylsilylethoxymethyl)pyrrolo[2,3-b]pyrazin-7-yl]-2,5-dihydropyrrole-1-carboxylate (8.4 g, 17 mmol) and 2-(3,6-dihydro-2H-pyran-4-yl)-4,4,5,5-tetramethyl-1,3,2-dioxaborolane (3.9 g, 18.6 mmol) in a mixture of dioxane/water (100 mL/20 mL), was added sequentially Pd(dppf)Cl₂ (370 mg), and Na₂CO₃ (5.4 g, 51 mmol). The reaction mixture was stirred at 80° C. for 5 hours under nitrogen atmosphere. After 5 hours, the reaction mixture was diluted with brine (150 mL) and extracted with EtOAc (3×150 mL). The combined organic layers were washed with brine (2×150 mL), dried over anhydrous Na₂SO₄, filtered and concentrated to give a crude oil. The crude product was purified by silica gel chromatography (EtOAc/PE 25/75) to give 6 g (82% yield) of tert-butyl 3-[2-(3,6-dihydro-2H-pyran-4-yl)-5-(2-trimethylsilylethoxymethyl)pyrrolo[2,3-b]pyrazin-7-yl]-2,5-dihydropyrrole-1-carboxylate as a yellow solid. LC/MS (m/z, M+H): calc. 499.7, found 499.1

### Step 3: tert-Butyl 3-[2-tetrahydropyran-4-yl-5-(2-trimethylsilylethoxymethyl)pyrrolo[2,3-b]pyrazin-7-yl]pyrrolidine-1-carboxylate

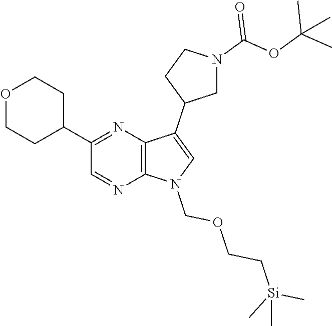 [SMILES: CC(C)(C)OC(=O)N1CCC(c2cn(COCC[Si](C)(C)C)c3ncc(C4CCOCC4)nc23)C1]

To a solution of tert-butyl 3-[2-(3,6-dihydro-2H-pyran-4-yl)-5-(2-trimethylsilylethoxymethyl)pyrrolo[2,3-b]pyrazin-7-yl]-2,5-dihydropyrrole-1-carboxylate (6 g, 12 mmol) in ethyl acetate (120 mL) was added Pd on carbon (20 wt %, 6 g). The mixture was stirred for 48 hours at room temperature under hydrogen atmosphere (1 atmosphere). The reaction mixture was then filtered through diatomaceous earth, and the filtrate was concentrated under reduced pressure to give 5 g (83% yield) of crude tert-butyl 3-[2-tetrahydropyran-4-yl-5-(2-trimethylsilylethoxymethyl)pyrrolo[2,3-b]pyrazin-7-yl]pyrrolidine-1-carboxylate as a white solid. LC/MS (m/z, M+H): calc. 503.7, found 503.2 STEP 4: (7-Pyrrolidin-3-yl-2-tetrahydropyran-4-yl-pyrrolo[2,3-b]pyrazin-5-yl)methanol; 2,2,2-trifluoroacetic acid

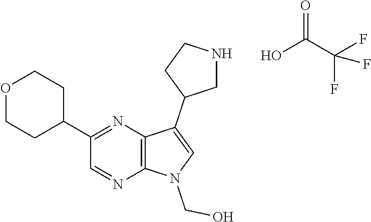 [SMILES: OCn1cc(C2CCNC2)c2nc(C3CCOCC3)cnc21 ; O=C(O)C(F)(F)F]

To a solution of tert-butyl 3-[2-tetrahydropyran-4-yl-5-(2-trimethylsilylethoxymethyl)pyrrolo[2,3-b]pyrazin-7-yl]pyrrolidine-1-carboxylate (1.6 g, 3.18 mol) in DCM (10 mL) was added TFA (5 mL) at 0° C. The resulting mixture was stirred at room temperature for 2 hours. The resulting reaction mixture was then concentrated to give 0.96 g (99% yield) of crude (7-pyrrolidin-3-yl-2-tetrahydropyran-4-yl-pyrrolo[2,3-b]pyrazin-5-yl)methanol; 2,2,2-trifluoroacetic acid as a light yellow oil. LC/MS (m/z, M+H-TFA): calc. 303.4, found 303.1

### Step 5: (rac)-[2-Amino-4-(trifluoromethoxy)phenyl]-[3-(2-tetrahydropyran-4-yl-5H-pyrrolo[2,3-b]pyrazin-7-yl)pyrrolidine-1-yl]methanone

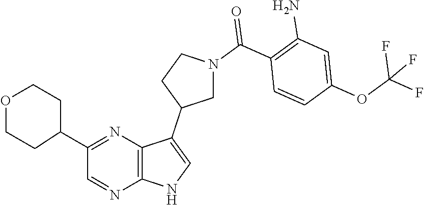 [SMILES: Nc1cc(OC(F)(F)F)ccc1C(=O)N1CCC(c2c[nH]c3ncc(C4CCOCC4)nc23)C1]

To a solution of (7-pyrrolidin-3-yl-2-tetrahydropyran-4-yl-pyrrolo[2,3-b]pyrazin-5-yl)methanol; 2,2,2-trifluoroacetic acid (445 mg, 1.07 mmol) and 2-amino-4-(trifluoromethoxy)benzoic acid (150 mg, 0.68 mmol) in N,N-dimethylformamide (5 mL) was added 1-hydroxybenzotriazole; hydrate (328 mg, 2.14 mmol), N′-(ethylcarbonimidoyl)-N,N-dimethylpropane-1,3-diamine hydrochloride (411 mg, 2.14 mmol) and DIPEA (692 mg, 5.4 mmol). The resulting mixture was stirred for 16 hours at room temperature. Then, the reaction mixture was diluted with water (50 mL) and extracted with EtOAc (3×50 mL). The combined organic layers were concentrated to give a crude oil. The crude product was dissolved in a mixed solution of methanol (10 mL) and 30% aqueous NH₄₀H (15 mL). After stirring for 1 hour, the resulting solution was treated with water (50 mL) and extracted with EtOAc (3×50 mL). The combined organic layers were washed with water and brine, dried over anhydrous Na₂SO₄, filtered and concentrated to give a crude oil which was purified by silica gel chromatography (3% methanol in dichloromethane) and preparative HPLC to give 101 mg (19% yield) of [2-amino-4-(trifluoromethoxy)phenyl]-[4-(2-cyclohexyl-5H-pyrrolo[2,3-b]pyrazin-7-yl)-1-piperidyl] as a colorless solid. LC/MS (m/z, M+H): calc. 476.5, found 476.3; ¹H NMR (400 MHz, DMSO-d6, 100° C.) δ ppm 1.73-1.93 (m, 4H), 2.22-2.32 (m, 1H), 2.33-2.43 (m, 1H), 3.00-3.12 (m, 2H), 3.48 (ddd, J=11.2, 2.9 Hz, 2H), 3.56-3.80 (m, 4H), 3.91-4.04 (m, 3H), 7.35 (br d, J=8.4 Hz, 2H), 7.58 (s, 1H), 7.64 (br d, J=8.6 Hz, 2H), 8.13 (s, 1H), 11.23-11.52 (m, 1H)

### Example 25: (rac)-[3-(2-Tetrahydropyran-4-yl-5H-pyrrolo[2,3-b]pyrazin-7-yl)pyrrolidin-1-yl]-[4-(trifluoromethoxy)phenyl]methanone

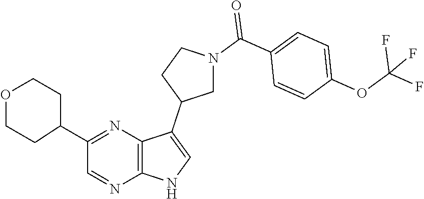 [SMILES: O=C(c1ccc(OC(F)(F)F)cc1)N1CCC(c2c[nH]c3ncc(C4CCOCC4)nc23)C1]

Example 25 was prepared following the procedure used in STEP 5 of Example 24, using (7-pyrrolidin-3-yl-2-tetrahydropyran-4-yl-pyrrolo[2,3-b]pyrazin-5-yl)methanol; 2,2,2-trifluoroacetic acid (0.48 g, 1.15 mmol) and 4-(trifluoromethoxy)benzoic acid (0.36 g, 1.74 mmol) in N,N-dimethylformamide (10 mL) was added 1-hydroxybenzotriazole; hydrate (0.32 g, 2.4 mmol), N′-(ethylcarbonimidoyl)-N,N-dimethylpropane-1,3-diamine hydrochloride (0.46 g, 2.4 mmol) and DIPEA (3.1 g, 24 mmol) to give 290 mg (55% yield) of (rac)-[3-(2-tetrahydropyran-4-yl-5H-pyrrolo[2,3-b]pyrazin-7-yl)pyrrolidin-1-yl]-[4-(trifluoromethoxy)phenyl]methanone as a colorless solid. LC/MS (m/z, M+H): calc. 461.4, found 461.2; ¹H NMR (400 MHz, DMSO-d6, 100° C.) δ ppm 1.73-1.93 (m, 4H), 2.22-2.32 (m, 1H), 2.33-2.43 (m, 1H), 3.00-3.12 (m, 2H), 3.48 (ddd, J=11.2, 2.9 Hz, 2H), 3.56-3.80 (m, 4H), 3.91-4.04 (m, 3H), 7.35 (br d, J=8.4 Hz, 2H), 7.58 (s, 1H), 7.64 (br d, J=8.6 Hz, 2H), 8.13 (s, 1H), 11.23-11.52 (m, 1H)

### Example 26: [2-Fluoro-4-(pentafluoro-λ⁶-sulfanyl)phenyl]-[4-(2-tetrahydropyran-4-yl-5H-pyrrolo[2,3-b]pyrazin-7-yl)-1-piperidyl]methanone

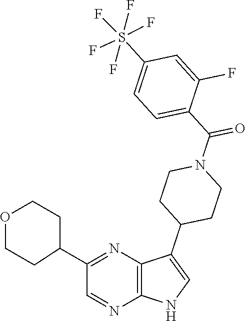 [SMILES: O=C(c1ccc(S(F)(F)(F)(F)F)cc1F)N1CCC(c2c[nH]c3ncc(C4CCOCC4)nc23)CC1]

Example 26 was prepared following the procedure described in Example 17, using 2-fluoro-4-(pentafluoro-λ⁶-sulfanyl)benzoic acid (91 mg, 0.34 mmol), TBTU (121 mg, 0.38 mmol), N,N-diisopropylethylamine (170 μL, 0.97 mmol), 7-(4-piperidyl)-2-tetrahydropyran-4-yl-5H-pyrrolo[2,3-b]pyrazine; hydrochloride (97 mg, 0.30 mmol) to give 75 mg (46% yield) of [2-fluoro-4-(pentafluoro-λ⁶-sulfanyl)phenyl]-[4-(2-tetrahydropyran-4-yl-5H-pyrrolo[2,3-b]pyrazin-7-yl)-1-piperidyl]methanone as a white solid. LC/MS (m/z, M+H): calc. 535.5, found 535.2; ¹H NMR (400 MHz, DMSO-d6, 100° C.) δ ppm 1.77-1.95 (m, 6H), 2.04-2.22 (m, 2H), 2.88-3.59 (m, 2H), 3.03-3.12 (m, 1H), 3.18-3.27 (m, 1H), 3.50 (ddd, J=11.4, 11.4, 2.8 Hz, 2H), 3.92-4.02 (m, 2H), 3.93-4.75 (m, 2H), 7.50 (s, 1H), 7.67 (br t, J=7.8. Hz, 1H), 7.80 (dd, J=8.6, 2.2 Hz, 1H), 7.87 (dd, J=9.6, 2.2 Hz, 1H), 8.12 (s, 1H), 11.25-11.34 (m, 1H)

### Example 27: [2-amino-4-(trifluoromethoxy)phenyl]-[4-[2-(oxetan-3-ylamino)-5H-pyrrolo[2,3-b]pyrazin-7-yl]-1-piperidyl]methanone

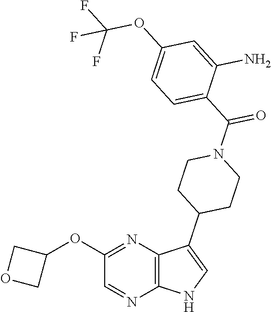 [SMILES: Nc1cc(OC(F)(F)F)ccc1C(=O)N1CCC(c2c[nH]c3ncc(OC4COC4)nc23)CC1]

### Step 1: tert-butyl 4-[2-(oxetan-3-ylamino)-5-(2-trimethylsilylethoxymethyl)pyrrolo[2,3-b]pyrazin-7-yl]-3,6-dihydro-2H-pyridine-1-carboxylate

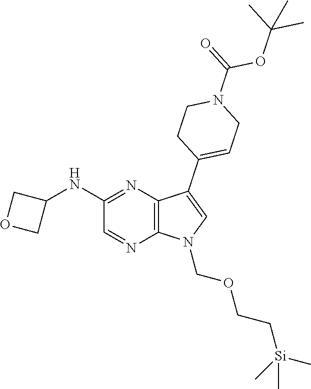 [SMILES: CC(C)(C)OC(=O)N1CC=C(c2cn(COCC[Si](C)(C)C)c3ncc(NC4COC4)nc23)CC1]

To a stirred solution of tert-butyl 4-[2-bromo-5-(2-trimethylsilylethoxymethyl)pyrrolo[2,3-b]pyrazin-7-yl]-3,6-dihydro-2H-pyridine-1-carboxylate (1.00 g, 1.96 mmol, prepared in step 3 of Example 1) and oxetan-3-amine (717 mg, 9.81 mmol) in 1,4-dioxane (10 mL), was added sodium 2-methylpropan-2-olate (566 mg, 5.89 mmol) at room temperature and the resulting reaction mixture was bubbled with argon for 10 minutes. Then, XPhosPdG4 (237 mg, 0.28 mmol) was added under nitrogen atmosphere and the resulting reaction mixture was submitted to microwave irradiation for 1 hour at 100° C. After 1 hour, the reaction mixture was diluted with water (30 mL), extracted with EtOAc (2×100 mL) and the combined organic layers were dried over sodium sulfate and concentrated under vacuum. The resulting residue was purified by flash chromatography eluting with 20% of ethyl acetate in hexane to give 550 mg (52% yield) of tert-butyl 4-[2-(oxetan-3-ylamino)-5-(2-trimethylsilylethoxymethyl)pyrrolo[2,3-b]pyrazin-7-yl]-3,6-dihydro-2H-pyridine-1-carboxylate as a yellow sticky solid. LC/MS (m/z, M+H): calc. 502.3, found 502.1.

### Step 2: tert-butyl 4-[2-(oxetan-3-ylamino)-5-(2-trimethylsilylethoxymethyl)pyrrolo[2,3-b]pyrazin-7-yl]piperidine-1-carboxylate

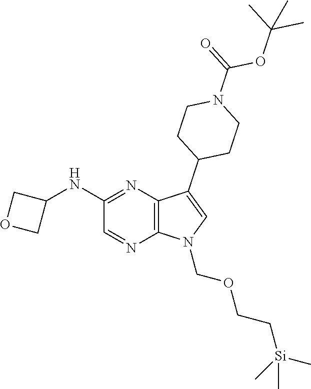 [SMILES: CC(C)(C)OC(=O)N1CCC(c2cn(COCC[Si](C)(C)C)c3ncc(NC4COC4)nc23)CC1]

To a stirred solution of tert-butyl 4-[2-(oxetan-3-ylamino)-5-(2-trimethylsilylethoxymethyl)pyrrolo[2,3-b]pyrazin-7-yl]-3,6-dihydro-2H-pyridine-1-carboxylate (550 mg, 1.1 mmol) in methanol (5 mL) was added 10% Pd/C (583 mg, 5.5 mmol) at room temperature and the resulting mixture was hydrogenated for 12 hours under 1 atmosphere of H₂. Then, after 12 hours, the reaction mixture was filtered through celite, and the resulting filtrate was dried over sodium sulfate and concentrated under vacuum to give 500 mg (88% yield) of tert-butyl 4-[2-(oxetan-3-ylamino)-5-(2-trimethylsilylethoxymethyl)pyrrolo[2,3-b]pyrazin-7-yl]piperidine-1-carboxylate as a yellow liquid. LC/MS (m/z, M+H): calc. 504.3, found 504.1.

### Step 3: [2-(oxetan-3-ylamino)-7-(4-piperidyl)pyrrolo[2,3-b]pyrazin-5-yl]methanol, 2,2,2-trifluoroacetic acid

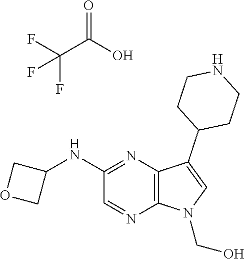 [SMILES: OCn1cc(C2CCNCC2)c2nc(NC3COC3)cnc21 ; O=C(O)C(F)(F)F]

A stirred solution of tert-butyl 4-[2-(oxetan-3-ylamino)-5-(2-trimethylsilylethoxymethyl)pyrrolo[2,3-b]pyrazin-7-yl]piperidine-1-carboxylate (500 mg, 0.99 mmol) in dichloromethane (5 mL) was cooled to 0° C., and then was added trifluoroacetic acid (226 mg, 1.99 mmol) and the resulting reaction mixture was stirred for 3 hours at room temperature. After 3 hours, the reaction mixture was concentrated under vacuum, and the resulting residue was triturated and washed in n-hexane (2×25 mL) to give 417 mg (100% yield) of [2-(oxetan-3-ylamino)-7-(4-piperidyl)pyrrolo[2,3-b]pyrazin-5-yl]methanol, 2,2,2-trifluoroacetic acid as a yellow solid. LC/MS (m/z, M+H−TFA): calc. 304.2, found 304.5.

### Step 4: [2-amino-4-(trifluoromethoxy)phenyl]-[4-[2-(oxetan-3-ylamino)-5H-pyrrolo[2,3-b]pyrazin-7-yl]-1-piperidyl]methanone

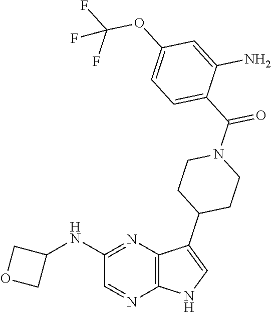 [SMILES: Nc1cc(OC(F)(F)F)ccc1C(=O)N1CCC(c2c[nH]c3ncc(NC4COC4)nc23)CC1]

To a stirred solution of [2-(oxetan-3-ylamino)-7-(4-piperidyl)pyrrolo[2,3-b]pyrazin-5-yl]methanol, 2,2,2-trifluoroacetic acid (280 mg, 0.67 mmol) and 2-amino-4-(trifluoromethoxy)benzoic acid (102 mg, 0.46 mmol) in N,N-dimethylformamide (5 mL), were added benzotriazol-1-yloxy(tripyrrolidin-1-yl)phosphonium; hexafluorophosphate (720 mg, 1.38 mmol) and N,N-diethylethanamine (280 mg, 2.77 mmol) at 0° C. and the resulting reaction mixture was stirred for 2 hours at room temperature. After 2 hours, the reaction mixture was diluted with water (20 mL), extracted with ethyl acetate (2×50 mL) and the combined organic layers were dried over sodium sulfate and concentrated under vacuum. The resulting residue was purified by preparative HPLC (conditions: Mobile Phase: A=10 mM ABC in water, B=ACN; Column: Gemini NX (250 mm×21.2 mm), 5.0 μm; Flow rate: 18 mL/min), and pure fractions were lyophilized to give 15 mg (3.4% yield) of [2-amino-4-(trifluoromethoxy)phenyl]-[4-[2-(oxetan-3-ylamino)-5H-pyrrolo[2,3-b]pyrazin-7-yl]-1-piperidyl]methanone as an off-white solid. LC/MS (m/z, M+H): calc. 477.2, found 477.1. ¹H NMR (400 MHz, DMSO-d⁶) δ ppm 7.39 (s, 1H) 7.13 (d, J=8.36 Hz, 1H) 6.65 (s, 1H) 6.48 (br d, J=8.36 Hz, 1H) 5.58 (s, 2H) 4.89 (br s, 1H) 4.48 (br s, 1H) 4.13-4.29 (m, 2H) 3.39-3.61 (m, 3H) 2.99 (br s, 3H) 1.84 (br s, 2H) 1.57 (br d, J=12.91 Hz, 2H).

### Example 28: [2-amino-4-(trifluoromethoxy)phenyl]-[4-[2-[(3R)-tetrahydrofuran-3-yl]oxy-5H-pyrrolo[2,3-b]pyrazin-7-yl]-1-piperidyl]methanone

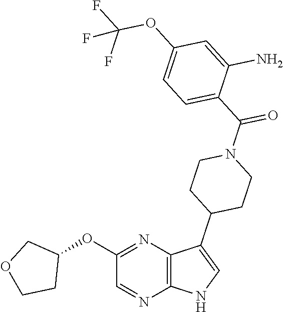 [SMILES: Nc1cc(OC(F)(F)F)ccc1C(=O)N1CCC(c2c[nH]c3ncc(O[C@@H]4CCOC4)nc23)CC1]

### Step 1: tert-butyl 4-[2-[(3R)-tetrahydrofuran-3-yl]oxy-5-(2-trimethylsilylethoxymethyl)pyrrolo[2,3-b]pyrazin-7-yl]-3,6-dihydro-2H-pyridine-1-carboxylate

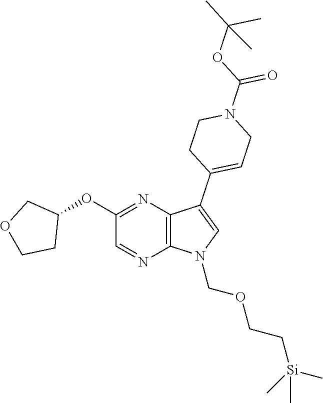 [SMILES: CC(C)(C)OC(=O)N1CC=C(c2cn(COCC[Si](C)(C)C)c3ncc(O[C@@H]4CCOC4)nc23)CC1]

To a stirred solution of tert-butyl 4-[2-bromo-5-(2-trimethylsilylethoxymethyl)pyrrolo[2,3-b]pyrazin-7-yl]-3,6-dihydro-2H-pyridine-1-carboxylate (1.00 g, 1.96 mmol, prepared in step 3 of Example 1) and (3R)-tetrahydrofuran-3-ol (865 mg, 9.81 mmol) in 1,4-dioxane (3 mL), was added sodium 2-methylpropan-2-olate (566 mg, 5.89 mmol) at room temperature and the resulting reaction mixture was bubbled with nitrogen for 10 minutes. Then, XPhosPdG4 (169 mg, 0.2 mmol) was added under nitrogen atmosphere and the resulting mixture was submitted to microwave irradiation for 1 hour at 100° C. After 1 hour, the reaction mixture was quenched with water (30 mL), extracted with EtOAc (2×100 mL), and the combined organic layers were dried over sodium sulfate and concentrated under vacuum. The resulting residue was purified by flash chromatography eluting with 12% ethyl acetate in hexane to give 250 mg (23% yield) of tert-butyl 4-[2-[(3R)-tetrahydrofuran-3-yl]oxy-5-(2-trimethylsilylethoxymethyl)pyrrolo[2,3b]pyrazin-7-yl]-3,6-dihydro-2H-pyridine-1-carboxylate as a yellow solid. LC/MS (m/z, M+H): calc. 517.3, found 517.3.

### Step 2: tert-butyl 4-[2-[(3R)-tetrahydrofuran-3-yl]oxy-5-(2-trimethylsilylethoxymethyl)pyrrolo[2,3-b]pyrazin-7-yl]piperidine-1-carboxylate

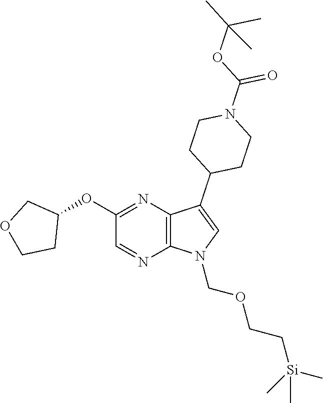 [SMILES: CC(C)(C)OC(=O)N1CCC(c2cn(COCC[Si](C)(C)C)c3ncc(O[C@@H]4CCOC4)nc23)CC1]

To a stirred solution of tert-butyl 4-[2-[(3R)-tetrahydrofuran-3-yl]oxy-5-(2-trimethylsilylethoxymethyl)pyrrolo[2,3-b]pyrazin-7-yl]-3,6-dihydro-2H-pyridine-1-carboxylate (250 mg, 0.48 mmol) in methanol (2 mL), was added 10% Pd/C (302 mg, 2.84 mmol) at room temperature and the resulting mixture was hydrogenated for 12 hours under 1 atmosphere of H₂. After 12 hours, the reaction mixture was filtered through celite, then washed with MeOH (2×5 mL), and the resulting filtrate was dried over sodium sulfate and concentrated under vacuum to give 180 mg (72% yield) of tert-butyl 4-[2-[(3R)-tetrahydrofuran-3-yl]oxy-5-(2-trimethylsilylethoxymethyl)pyrrolo[2,3-b]pyrazin-7-yl]piperidine-1-carboxylate. LC/MS (m/z, M+H): calc. 519.3, found 519.3.

### Step 3: [7-(4-piperidyl)-2-[(3R)-tetrahydrofuran-3-yl]oxy-pyrrolo[2,3-b]pyrazin-5-yl]methanol, 2,2,2-trifluoroacetic acid

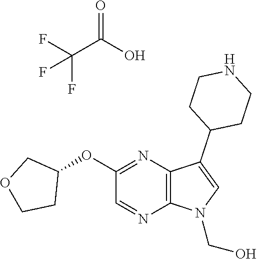 [SMILES: OCn1cc(C2CCNCC2)c2nc(O[C@@H]3CCOC3)cnc21 ; O=C(O)C(F)(F)F]

To a stirred solution of tert-butyl 4-[2-[(3R)-tetrahydrofuran-3-yl]oxy-5-(2-trimethylsilylethoxymethyl)pyrrolo[2,3-b]pyrazin-7-yl]piperidine-1-carboxylate (180 mg, 0.35 mmol) in dichloromethane (5.0 mL), was added trifluoroacetic acid (2 mL) at 0° C. and the resulting reaction mixture was stirred for 3 hours at room temperature. After 3 hours, the reaction mixture was concentrated under vacuum. The crude material was triturated and washed with n-hexane (2×10 mL) to give 150 mg (100% yield) of [7-(4-piperidyl)-2-[(3R)-tetrahydrofuran-3-yl]oxy-pyrrolo[2,3-b]pyrazin-5-yl]methanol, 2,2,2-trifluoroacetic acid as a yellow solid. LC/MS (m/z, M+H−TFA): calc. 319.2, found 319.0.

### Step 4: [2-amino-4-(trifluoromethoxy)phenyl]-[4-[2-[(3R)-tetrahydrofuran-3-yl]oxy-5H-pyrrolo[2,3-b]pyrazin-7-yl]-1-piperidyl]methanone

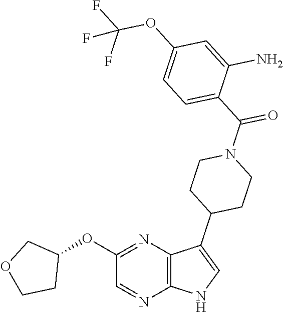 [SMILES: Nc1cc(OC(F)(F)F)ccc1C(=O)N1CCC(c2c[nH]c3ncc(O[C@@H]4CCOC4)nc23)CC1]

To a stirred solution of 2-amino-4-(trifluoromethoxy)benzoic acid (115 mg, 0.52 mmol) in N,N-dimethylformamide (5 mL), was added 3-(ethyliminomethyleneamino)-N,N-dimethyl-propan-1-amine, hydrochloride (150 mg, 0.78 mmol) and 1-hydroxybenzotriazole (84 mg, 0.62 mmol) cooled to 0° C., was added N,N-diethylethanamine (158 mg, 1.56 mmol) at 0° C. followed, after 10 minutes, by [7-(4-piperidyl)-2-[(3R)-tetrahydrofuran-3-yl]oxy-pyrrolo[2,3-b]pyrazin-5-yl]methanol (150 mg, 0.47 mmol). Then, the reaction mixture was stirred for 16 hours at room temperature. After 16 hours, the reaction mixture was concentrated under vacuum. The resulting residue was purified by preparative HPLC (Preparative conditions: Mobile Phase: A=10 mM ABC in water, B=ACN; Column: Gemini NX (250 mm×21.2 mm), 5.0 μm; Flow rate: 18 mL/min; Gradient Programmer (Time (minutes) % B): 0 min 30%, 2 min 40%, 8 min 70%), pure fractions were concentrated to give 6 mg (2.5% yield) of [[2-amino-4-(trifluoromethoxy)phenyl]-[4-[2-[(3S)-tetrahydrofuran-3-yl]oxy-5H-pyrrolo[2,3-b]pyrazin-7-yl]-1-piperidyl]methanone as a white solid. LC/MS (m/z, M+H): calc. 492.2, found 492.2. ¹H NMR (400 MHz, DMSO-d₆) δ ppm 11.49-11.75 (m, 1H) 7.72-7.92 (m, 1H) 7.42-7.61 (m, 1H) 7.03-7.16 (m, 1H) 6.57-6.72 (m, 1H) 6.37-6.57 (m, 1H) 5.49-5.68 (m, 2H) 3.72-4.11 (m, 5H) 3.00-3.20 (m, 3H) 2.14-2.31 (m, 1H) 1.98-2.14 (m, 3H) 1.62-1.98 (m, 3H).

### Example 29: [2-amino-4-(trifluoromethoxy)phenyl]-[4-[2-[(3 S)-tetrahydrofuran-3-yl]oxy-5H-pyrrolo[2,3-b]pyrazin-7-yl]-1-piperidyl]methanone

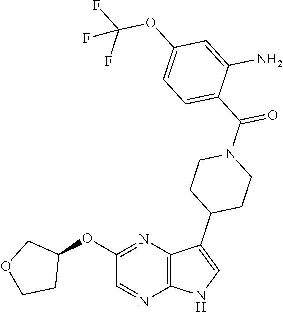 [SMILES: Nc1cc(OC(F)(F)F)ccc1C(=O)N1CCC(c2c[nH]c3ncc(O[C@H]4CCOC4)nc23)CC1]

Example 29 was prepared following the protocols described in steps 1 to 4 of Example 28 using (3S)-tetrahydrofuran-3-ol (259 mg, 0.29 mmol) in step 1, to give 16 mg (7% yield in step 4) of [2-amino-4-(trifluoromethoxy)phenyl]-[4-[2-[(3S)-tetrahydrofuran-3-yl]oxy-5H-pyrrolo[2,3-b]pyrazin-7-yl]-1-piperidyl]methanone as a white solid. LC/MS (m/z, M+H): calc. 492.2, found 492.3. ¹H NMR (400 MHz, DMSO-d₆) δ ppm: 11.61 (d, J=2.4 Hz, 1H) 0.83 (s, 1H) 7.47-7.53 (m, 1H) 7.08-7.14 (m, 1H) 6.63-6.69 (m, 1H) 6.43-6.53 (m, 1H) 5.54-5.64 (m, 2H) 5.44-5.53 (m, 1H) 3.95-4.05 (m, 1H) 3.73-3.92 (m, 4H) 3.00-3.18 (m, 3H) 2.21-2.37 (m, 2H) 1.95-2.11 (m, 3H) 1.69-1.88 (m, 2H).

### Example 30: trans-[2-amino-4-(trifluoromethoxy)phenyl]-[4-[2-(4-hydroxycyclohexyl)-5H-pyrrolo[2,3-b]pyrazin-7-yl]-1-piperidyl]methanone

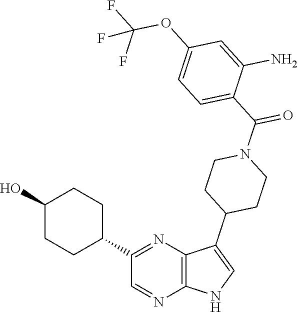 [SMILES: Nc1cc(OC(F)(F)F)ccc1C(=O)N1CCC(c2c[nH]c3ncc([C@H]4CC[C@H](O)CC4)nc23)CC1]

### Step 1: tert-butyl 4-[2-(1,4-dioxaspiro[4.5]dec-7-en-8-yl)-5-(2-trimethylsilylethoxymethyl)pyrrolo[2,3-b]pyrazin-7-yl]-3,6-dihydro-2H-pyridine-1-carboxylate

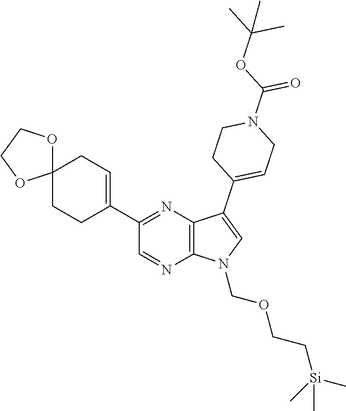 [SMILES: CC(C)(C)OC(=O)N1CC=C(c2cn(COCC[Si](C)(C)C)c3ncc(C4=CCC5(CC4)OCCO5)nc23)CC1]

To a stirred solution of tert-butyl 4-[2-bromo-5-(2-trimethylsilylethoxymethyl)pyrrolo[2,3-b]pyrazin-7-yl]-3,6-dihydro-2H-pyridine-1-carboxylate (1.50 g, 2.94 mmol, prepared in step 3 of Example 1) and 2-(1,4-dioxaspiro[4.5]dec-7-en-8-yl)-4,4,5,5-tetramethyl-1,3,2-dioxaborolane (784 mg, 2.94 mmol) in 1,4-dioxane (30 mL) and water (8 mL), was added K₂CO₃ (1.22 g, 8.83 mmol) and the resulting mixture was bubbled with nitrogen for 15 minutes. Then, PdCl₂(dppf)·DCM (215 mg, 0.26 mmol) was added at room temperature and the reaction mixture was stirred at 90° C. for 4 hours. After 4 hours, the reaction mixture was diluted with water (100 mL), extracted with EtOAc (2×100 mL), and the combined organic layers were dried over sodium sulfate and concentrated under vacuum. The resulting residue was purified by flash chromatography eluting with 40% ethyl acetate in hexane to give 1.5 g (90% yield) tert-butyl 4-[2-(1,4-dioxaspiro[4.5]dec-7-en-8-yl)-5-(2-trimethylsilylethoxymethyl)pyrrolo[2,3-b]pyrazin-7-yl]-3,6-dihydro-2H-pyridine-1-carboxylate as a yellow gummy liquid. LC/MS (m/z, M+H): calc. 569.3, found 569.2.

### Step 2: tert-butyl 4-[2-(1,4-dioxaspiro[4.5]decan-8-yl)-5-(2-trimethylsilylethoxymethyl)pyrrolo[2,3-b]pyrazin-7-yl]piperidine-1-carboxylate

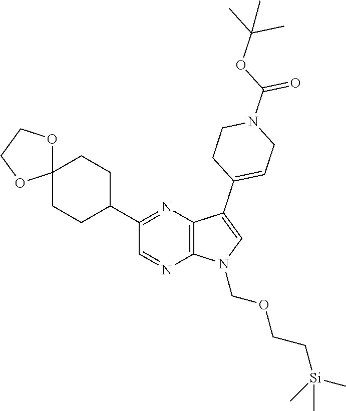 [SMILES: CC(C)(C)OC(=O)N1CC=C(c2cn(COCC[Si](C)(C)C)c3ncc(C4CCC5(CC4)OCCO5)nc23)CC1]

To a stirred solution of tert-butyl 4-[2-(1,4-dioxaspiro[4.5]dec-7-en-8-yl)-5-(2-trimethylsilylethoxymethyl)pyrrolo[2,3-b]pyrazin-7-yl]-3,6-dihydro-2H-pyridine-1-carboxylate (1.50 g, 2.64 mmol) in methanol (20 mL) was added 10% Pd/C (2.81 g, 26.4 mmol) under an inert atmosphere and then the reaction mixture was stirred overnight under 1 atmosphere of H₂ at room temperature for 4 hours. After 4 hours, the reaction mixture was filtered through celite, and the resulting filtrate was dried over sodium sulfate and concentrated under vacuum to give 1.2 g (79% yield) of tert-butyl 4-[2-(1,4-dioxaspiro[4.5]decan-8-yl)-5-(2-trimethylsilylethoxymethyl)pyrrolo[2,3-b]pyrazin-7-yl]piperidine-1-carboxylate as a yellow solid. LC/MS (m/z, M+H): calc. 573.3, found 573.3.

### Step 3: 4-[7-(4-piperidyl)-5H-pyrrolo[2,3-b]pyrazin-2-yl]cyclohexanone, 2,2,2-trifluoroacetic acid

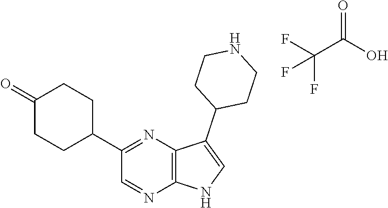

To a stirred solution of tert-butyl 4-[2-(1,4-dioxaspiro[4.5]decan-8-yl)-5-(2-trimethylsilylethoxymethyl)pyrrolo[2,3-b]pyrazin-7-yl]piperidine-1-carboxylate (1.50 g, 2.62 mmol) in dichloromethane (20 mL), was added 2,2,2-trifluoroacetic acid (2.99 g, 26.2 mmol) at 0° C. The reaction mixture was stirred at room temperature for 16 hours. After 16 hours, the reaction mixture was concentrated under vacuum, triturated and washed with n-Hexane (2×25 mL) to give 900 mg (83% yield) of 4-[7-(4-piperidyl)-5H-pyrrolo[2,3-b]pyrazin-2-yl]cyclohexanone; 2,2,2-trifluoroacetic acid as an off white solid. LC/MS (m/z, M−H−TFA): calc. 297.2, found 297.2.

### Step 4: 4-[7-[1-[2-amino-4-(trifluoromethoxy)benzoyl]-4-piperidyl]-5H-pyrrolo[2,3-b]pyrazin-2-yl]cyclohexanone

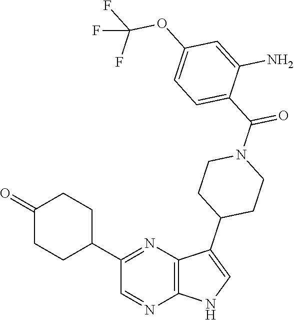 [SMILES: Nc1cc(OC(F)(F)F)ccc1C(=O)N1CCC(c2c[nH]c3ncc(C4CCC(=O)CC4)nc23)CC1]

To a stirred solution of 2-amino-4-(trifluoromethoxy)benzoic acid (500 mg, 2.26 mmol) and 4-[7-(4-piperidyl)-5H-pyrrolo[2,3-b]pyrazin-2-yl]cyclohexanone, 2,2,2-trifluoroacetic acid (675 mg, 1.64 mmol) in N,N-dimethylformamide (10 mL) were added 3-(ethyliminomethyleneamino)-N,N-dimethyl-propan-1-amine; hydrochloride (650 mg, 3.39 mmol), 1-hydroxybenzotriazole; hydrate (519 mg, 3.39 mmol) and N,N-diethylethanamine (686 g, 6.78 mmol) at 0° C. The reaction mixture was stirred at room temperature for 4 hours. After 4 hours, the reaction mixture was concentrated under reduced pressure, diluted with ice water (30 mL), and extracted with ethyl acetate (20 mL×2). The combined organic layers were dried over Na₂SO₄, filtered and concentrated under reduced pressure to give 500 mg (44% yield) of 4-[7-[1-[2-amino-4-(trifluoromethoxy)benzoyl]-4-piperidyl]-5H-pyrrolo[2,3-b]pyrazin-2-yl]cyclohexanone as an off white solid. LC/MS (m/z, M+H): calc. 502.2, found 502.2.

### Step 5: trans-[2-amino-4-(trifluoromethoxy)phenyl]-[4-[2-(4-hydroxycyclohexyl)-5H-pyrrolo[2,3-b]pyrazin-7-yl]-1-piperidyl]methanone

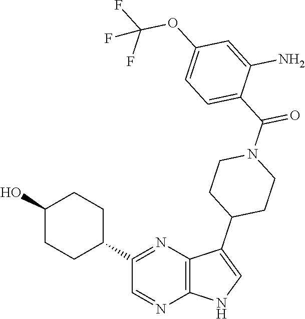 [SMILES: Nc1cc(OC(F)(F)F)ccc1C(=O)N1CCC(c2c[nH]c3ncc([C@H]4CC[C@H](O)CC4)nc23)CC1]

To a stirred solution of 4-[7-[1-[2-amino-4-(trifluoromethoxy)benzoyl]-4-piperidyl]-5H-pyrrolo[2,3-b]pyrazin-2-yl]cyclohexanone (500 g, 0.99 mmol) in methanol (10 mL) was added NaBH₄ (189 mg, 4.99 mmol) at 0° C. The reaction mixture was stirred at room temperature for 1 hour. After one hour, the reaction mixture was concentrated under reduced pressure and the resulting residue was diluted with ice water (20 mL) and extracted with ethyl acetate (20 mL×2). The combined organic layers were dried over Na₂SO₄, filtered and concentrated to give 15 mg (3% yield) of [2-amino-4-(trifluoromethoxy)phenyl]-[4-[2-(4-hydroxycyclohexyl)-5H-pyrrolo[2,3-b]pyrazin-7-yl]-1-piperidyl]methanone as an off white solid. LC/MS (m/z, M+H): calc. 504.2, found 504.2. ¹H NMR (400 MHz, DMSO-d₆) δ ppm 11.50-11.66 (m, 1H) 7.48-7.63 (m, 1H) 7.09-7.18 (m, 1H) 6.63-6.72 (m, 1H) 6.44-6.57 (m, 1H) 5.46-5.64 (m, 2H) 4.40-4.63 (m, 1H) 3.42-3.55 (m, 2H) 3.01-3.20 (m, 3H) 1.83-2.14 (m, 6H) 1.51-1.77 (m, 4H) 1.27-1.42 (m, 3H).

### Example 31: [4-[2-(4-methylpiperazin-1-yl)-5H-pyrrolo[2,3-b]pyrazin-7-yl]-1-piperidyl]-[4-(trifluoromethoxy)phenyl]methanone, formic acid

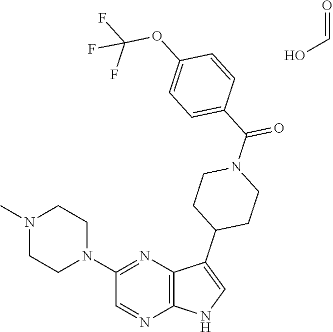 [SMILES: CN1CCN(c2cnc3[nH]cc(C4CCN(C(=O)c5ccc(OC(F)(F)F)cc5)CC4)c3n2)CC1 ; O=CO]

### Step 1: tert-butyl 4-[2-(4-methylpiperazin-1-yl)-5-(2-trimethylsilylethoxymethyl)pyrrolo[2,3-b]pyrazin-7-yl]-3,6-dihydro-2H-pyridine-1-carboxylate

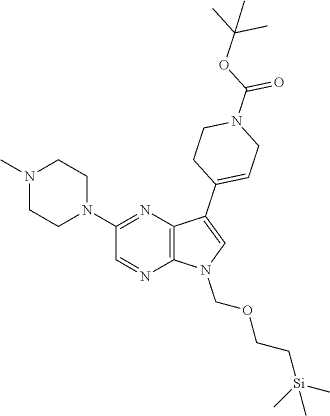

To a stirred solution of tert-butyl 4-[2-bromo-5-(2-trimethylsilylethoxymethyl)pyrrolo[2,3-b]pyrazin-7-yl]-3,6-dihydro-2H-pyridine-1-carboxylate (1 g, 1.96 mmol, prepared in step 3 of Example 1) in 1,4-dioxane (10 mL), were added 1-methylpiperazine (393 mg, 3.93 mmol) and sodium 2-methylpropan-2-olate (566 mg, 5.89 mmol) at room temperature, and the resulting mixture was bubbled with nitrogen for 10 minutes. Then, XPhosPdG4 (169 mg, 0.2 mmol) was added under N₂ atmosphere and the resulting mixture was submitted to microwave for 1 hour at 100° C. After one hour, the reaction mixture was diluted with water (30 mL), extracted with EtOAc (2×100 mL), and the combined organic layers were dried over sodium sulfate and concentrated under vacuum. The resulting residue was purified by flash chromatography eluting with 40% ethyl acetate in hexane to give 500 mg (48% yield) of tert-butyl 4-[2-(4-methylpiperazin-1-yl)-5-(2-trimethylsilylethoxymethyl) pyrrolo[2,3-b]pyrazin-7-yl]-3,6-dihydro-2H-pyridine-1-carboxylate as a yellow solid. LC/MS (m/z, M+H): calc. 529.3, found 529.2.

### Step 2: tert-butyl 4-[2-(4-methylpiperazin-1-yl)-5-(2-trimethylsilylethoxymethyl)pyrrolo[2,3-b]pyrazin-7-yl]piperidine-1-carboxylate

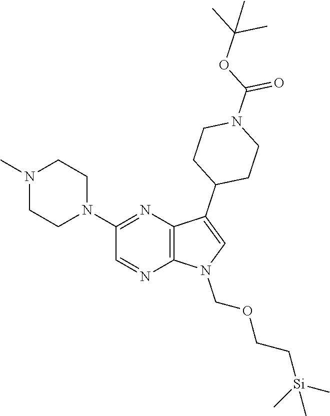

To a stirred solution of tert-butyl 4-[2-(4-methylpiperazin-1-yl)-5-(2-trimethylsilylethoxymethyl) pyrrolo[2,3-b]pyrazin-7-yl]-3,6-dihydro-2H-pyridine-1-carboxylate (500 mg, 0.95 mmol) in ethanol (20 mL), was added 10% Pd/C (302 mg, 2.84 mmol) at room temperature and the resulting reaction mixture was hydrogenated for 12 hours under one atmosphere of H₂. After 12 hours, the reaction mixture was filtered through celite and the resulting filtrate was dried over sodium sulfate and concentrated under vacuum to give 350 mg (70% yield) of tert-butyl 4-[2-(4-methylpiperazin-1-yl)-5-(2-trimethylsilylethoxymethyl)pyrrolo[2,3-b]pyrazin-7-yl]piperidine-1-carboxylate as a yellow liquid. LC/MS (m/z, M+H): calc. 531.3, found 531.2.

### Step 3: [2-(4-methylpiperazin-1-yl)-7-(4-piperidyl)pyrrolo[2,3-b]pyrazin-5-yl]methanol, dihydrochloride

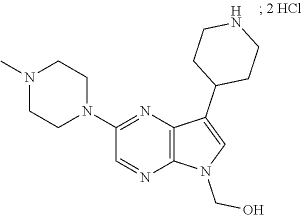 [SMILES: CN1CCN(c2cnc3c(n2)c(C2CCNCC2)cn3CO)CC1]

To a stirred solution of tert-butyl 4-[2-(4-methylpiperazin-1-yl)-5-(2-trimethylsilylethoxymethyl) pyrrolo[2,3-b]pyrazin-7-yl]piperidine-1-carboxylate (320 mg, 0.6 mmol) in dichloromethane (10 mL), was added 4M HCl in dioxane (3 mL, 0.056 mmol) at 0° C. and the resulting reaction mixture was stirred for 4 hours at room temperature. After 4 hours, the reaction mixture was concentrated under vacuum, and the resulting residue was triturated and washed with n-hexane (2×25 mL) to give 350 mg of crude [2-(4-methylpiperazin-1-yl)-7-(4-piperidyl) pyrrolo[2,3-b]pyrazin-5-yl]methanol, dihydrochloride (0.350 g) as a yellow solid which was engaged in the next step without further purification.

### Step 4: 2-(4-methylpiperazin-1-yl)-7-(4-piperidyl)-5H-pyrrolo[2,3-b]pyrazine

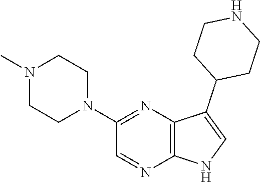 [SMILES: CN1CCN(c2cnc3[nH]cc(C4CCNCC4)c3n2)CC1]

To a stirred solution of [2-(4-methylpiperazin-1-yl)-7-(4-piperidyl) pyrrolo[2,3-b]pyrazin-5-yl]methanol, dihydrochloride (crude 350 mg of step 3) in methanol, was added aqueous NH₃ solution (25% w/v, 3 mL, 0.056 mmol) at 0° C. and the resulting reaction mixture was stirred for 4 hours at room temperature. After 4 hours, the reaction mixture was concentrated under vacuum. The resulting residue was purified by preparative HPLC to give 100 mg (55% yield) of 2-(4-methylpiperazin-1-yl)-7-(4-piperidyl)-5H-pyrrolo[2,3-b]pyrazine as a pale yellow solid. LC/MS (m/z, M+H): calc. 301.2, found 301.1.

### Step 5: [4-[2-(4-methylpiperazin-1-yl)-5H-pyrrolo[2,3-b]pyrazin-7-yl]-1-piperidyl]-[4-(trifluoromethoxy)phenyl]methanone, formic acid

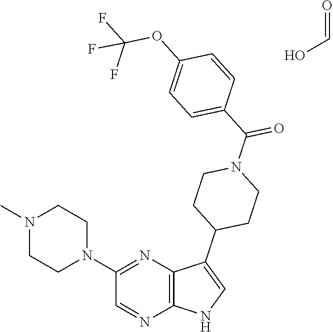 [SMILES: CN1CCN(c2cnc3[nH]cc(C4CCN(C(=O)c5ccc(OC(F)(F)F)cc5)CC4)c3n2)CC1 ; O=CO]

To a stirred solution of 4-(trifluoromethoxy)benzoic acid (70 mg, 0.34 mmol) in N,N-dimethylformamide (10 mL), were added [benzotriazol-1-yloxy(dimethylamino)methylene]-dimethyl-ammonium; tetrafluoroborate (164 mg, 0.51 mmol) and N,N-diethylethanamine (103 mg, 1.02 mmol) and the resulting reaction mixture was stirred for 15 minutes. Then, 2-(4-methylpiperazin-1-yl)-7-(4-piperidyl)-5H-pyrrolo[2,3-b]pyrazine (92 mg, 0.31 mmol) was added at 0° C. and the resulting reaction mixture was stirred for 4 hours at room temperature. After 4 hours, the reaction mixture was diluted with water (20 mL), extracted with ethyl acetate (2×50 mL), and the combined organic layers were dried over sodium sulfate and concentrated under vacuum. The resulting residue was purified by preparative HPLC (Preparative conditions: Mobile Phase: A=0.1% HCOOH in water, B=ACN Column: X SELECT (250 mm×20 mm), 5 μm Flow rate: 15 mL/min), pure fractions were lyophilized to give 12 mg (7% yield) of [4-[2-(4-methylpiperazin-1-yl)-5H-pyrrolo[2,3-b]pyrazin-7-yl]-1-piperidyl]-[4-(trifluoromethoxy)phenyl]methanone, formic acid as an off-white solid. LC/MS (m/z, M+H−HCOOH): calc. 489.2, found 489.1. ¹H NMR (400 MHz, DMSO-d₆) δ ppm 11.18-11.42 (m, 1H) 7.86-8.07 (m, 1H) 7.49-7.64 (m, 2H) 7.26-7.55 (m, 5H) 4.47-4.63 (m, 1H) 3.53-3.67 (m, 1H) 3.42-3.53 (m, 5H) 3.05-3.16 (m, 1H) 2.67 (s, 1H) 2.43-2.47 (m, 4H) 2.30-2.35 (m, 1H) 1.94-2.15 (m, 3H) 1.68-1.83 (m, 2H) 1.37-1.55 (m, 2H).

### Examples 36, 32 & 33: (rac)-[4-[2-[[tetrahydrofuran-3-yl]amino]-5H-pyrrolo[2,3-b]pyrazin-7-yl]-1-piperidyl]-[4-(trifluoromethoxy)phenyl]methanone, and [4-[2-[[tetrahydrofuran-3-yl]amino]-5H-pyrrolo[2,3-b]pyrazin-7-yl]-1-piperidyl]-[4-(trifluoromethoxy)phenyl]methanone, Isomers 1 & 2

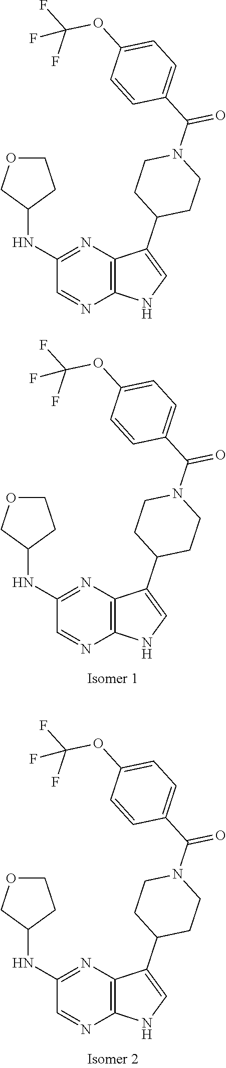 [SMILES: O=C(c1ccc(OC(F)(F)F)cc1)N1CCC(c2c[nH]c3ncc(NC4CCOC4)nc23)CC1 ; O=C(c1ccc(OC(F)(F)F)cc1)N1CCC(c2c[nH]c3ncc(NC4CCOC4)nc23)CC1 ; O=C(c1ccc(OC(F)(F)F)cc1)N1CCC(c2c[nH]c3ncc(NC4CCOC4)nc23)CC1]

### Step 1: (rac)-tert-butyl 4-[2-[[tetrahydrofuran-3-yl]amino]-5-(2-trimethylsilylethoxymethyl)pyrrolo[2,3-b]pyrazin-7-yl]-3,6-dihydro-2H-pyridine-1-carboxylate

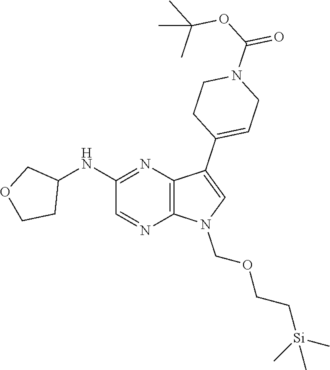 [SMILES: CC(C)(C)OC(=O)N1CC=C(c2cn(COCC[Si](C)(C)C)c3ncc(NC4CCOC4)nc23)CC1]

To a solution of tert-butyl 4-[2-bromo-5-(2-trimethylsilylethoxymethyl)pyrrolo[2,3-b]pyrazin-7-yl]-3,6-dihydro-2H-pyridine-1-carboxylate (950 mg, 1.86 mmol, prepared in step 3 of Example 1) and tetrahydrofuran-3-amine; hydrochloride (461 mg, 3.73 mmol) in dioxane (9 mL) was added t-BuONa (717 mg, 7.46 mmol) and the resulting mixture was bubbled with argon for 5 minutes. Then, Pd₂(dba)₃ (171 mg, 0.19 mmol) and RuPhos (104 mg, 0.22 mmol) were added, and the resulting mixture was submitted to microwave irradiation at 100° C. for 1 hour. Then, the whole mixture was diluted with ethyl acetate, washed with water and brine. The combined aqueous layers were extracted with ethyl acetate and the combined organic layers were dried over sodium sulfate, filtered and concentrated. The resulting residue was purified by flash chromatography on silica gel (SiO₂ 80 g) eluting with DCM/ethyl acetate 7/3, to give 548 mg (57% yield) of (rac)-tert-butyl 4-[2-[[tetrahydrofuran-3-yl]amino]-5-(2-trimethylsilylethoxymethyl)pyrrolo[2,3-b]pyrazin-7-yl]-3,6-dihydro-2H-pyridine-1-carboxylate as an orange solid. LC/MS (m/z, M+H): calc. 516.3, found 516.5.

### Step 2: (rac)-tert-butyl 4-[2-[[tetrahydrofuran-3-yl]amino]-5-(2-trimethylsilylethoxymethyl)pyrrolo[2,3-b]pyrazin-7-yl]piperidine-1-carboxylate

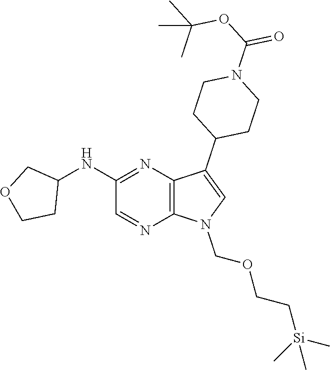 [SMILES: CC(C)(C)OC(=O)N1CCC(c2cn(COCC[Si](C)(C)C)c3ncc(NC4CCOC4)nc23)CC1]

To a solution of (rac)-tert-butyl 4-[2-[[tetrahydrofuran-3-yl]amino]-5-(2-trimethylsilylethoxymethyl)pyrrolo[2,3-b]pyrazin-7-yl]-3,6-dihydro-2H-pyridine-1-carboxylate (577 mg, 1.12 mmol) in EtOH (10 mL) was added ammonium formate (1.41 g, 22.4 mmol) and Pd/C 10% (50% wet) (110 mg) and the whole mixture was stirred at 80° C. for one hour. After one hour, the mixture was cooled down to room temperature, filtered, washed with EtOH, and the resulting filtrated was concentrated under reduced pressure. The resulting residue was diluted with ethyl acetate, washed with a 1 N solution of NaOH, dried over sodium sulfate, filtered and concentrated to give 561 mg (96% yield) of (rac)-tert-butyl 4-[2-[[tetrahydrofuran-3-yl]amino]-5-(2-trimethylsilylethoxymethyl)pyrrolo[2,3-b]pyrazin-7-yl]piperidine-1-carboxylate as a brown solid. LC/MS (m/z, M+H): calc. 518.3, found 518.6.

### Step 3: (rac)-tert-butyl 4-[2-[[tetrahydrofuran-3-yl]amino]-5H-pyrrolo[2,3-b]pyrazin-7-yl]piperidine-1-carboxylate

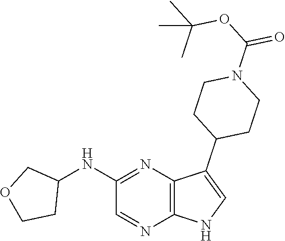 [SMILES: CC(C)(C)OC(=O)N1CCC(c2c[nH]c3ncc(NC4CCOC4)nc23)CC1]

To a solution of (rac)-tert-butyl 4-[2-[[tetrahydrofuran-3-yl]amino]-5-(2-trimethylsilylethoxymethyl)pyrrolo[2,3-b]pyrazin-7-yl]piperidine-1-carboxylate (557 mg, 1.07 mmol) in THE (2 mL) was added ethylenediamine (0.72 mL, 10.76 mmol) and a 1 M solution of tetrabutylammonium fluoride in THE (8.6 mL, 8.6 mmol) and the resulting reaction mixture was stirred under reflux for 12 hours. After 12 hours, the reaction mixture was cooled down to room temperature, concentrated, diluted with ethyl acetate, washed with water, dried over sodium sulfate, filtered and concentrated in vacuo. The resulting residue was purified by flash chromatography on silica gel (SiO₂ 24 g) eluting with ethyl acetate/heptane 8/2, to give 373 mg (89% yield) of (rac)-tert-butyl 4-[2-[[tetrahydrofuran-3-yl]amino]-5H-pyrrolo[2,3-b]pyrazin-7-yl]piperidine-1-carboxylate as a brown solid. LC/MS (m/z, M+H): calc. 388.2, found 388.4.

### Step 4: (rac)-7-(4-piperidyl)-N-tetrahydrofuran-3-yl-5H-pyrrolo[2,3-b]pyrazin-2-amine, trihydrochloride

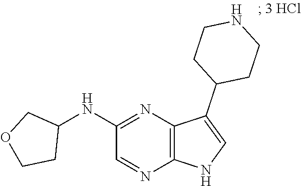 [SMILES: c1nc2[nH]cc(C3CCNCC3)c2nc1NC1CCOC1]

To a solution of (rac)-tert-butyl 4-[2-[[tetrahydrofuran-3-yl]amino]-5H-pyrrolo[2,3-b]pyrazin-7-yl]piperidine-1-carboxylate (370 mg, 0.95 mmol) in MeOH (6 mL), was added a 4 N solution of HCl in dioxane (2.38 mL, 9.55 mmol) and the resulting mixture was stirred at room temperature for 1.5 hours. Then, the resulting mixture was concentrated to dryness to give 367 mg (96% yield) of (rac)-7-(4-piperidyl)-N-tetrahydrofuran-3-yl-5H-pyrrolo[2,3-b]pyrazin-2-amine, trihydrochloride as a yellow solid which was engaged in the next step without purification.

### Step 5 (Example 36): (rac)-[4-[2-[[tetrahydrofuran-3-yl]amino]-5H-pyrrolo[2,3-b]pyrazin-7-yl]-1-piperidyl]-[4-(trifluoromethoxy)phenyl]methanone

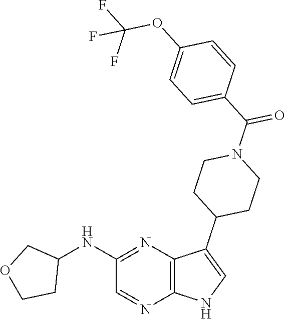 [SMILES: O=C(c1ccc(OC(F)(F)F)cc1)N1CCC(c2c[nH]c3ncc(NC4CCOC4)nc23)CC1]

To a solution of (rac)-7-(4-piperidyl)-N-tetrahydrofuran-3-yl-5H-pyrrolo[2,3-b]pyrazin-2-amine; trihydrochloride (182 mg, 0.46 mmol) in DMF (3 mL) was added DIPEA (237 mg, 1.83 mmol) and the resulting reaction mixture was stirred at room temperature for 5 minutes. Then, 4-(trifluoromethoxy)benzoic acid (94 mg, 0.46 mmol), and TBTU (162 mg, 0.5 mmol) were successively added and the resulting mixture was stirred at room temperature for 2.5 additional hours, then diluted with ethyl acetate, washed with an aqueous saturated solution of NaHCO₃, with water, dried over sodium sulfate, filtered and concentrated in vacuo. The resulting residue was triturated in MeCN, filtered, washed with MeCN and dried in vacuo. The thus obtained pale brown solid was then purified by flash chromatography on silica gel (SiO₂ 12 g) eluting with DCM/MeOH/NH₄OH 95/5/0.5 to give 163 mg (75% yield) of (rac)-[4-[2-[[tetrahydrofuran-3-yl]amino]-5H-pyrrolo[2,3-b]pyrazin-7-yl]-1-piperidyl]-[4-(trifluoromethoxy)phenyl]methanone as a pale brown solid. LC/MS (m/z, M+): calc. 475.2, found 475.0. ¹H NMR (400 MHz, DMSO-d₆, 100° C.) δ ppm 1.82-1.94 (m, 3H) 2.06 (br d, J=12.7 Hz, 2H) 2.20-2.29 (m, 1H) 3.05-3.19 (m, 3H) 3.57 (dd, J=8.8, 4.5 Hz, 1H) 3.72-3.79 (m, 1H) 3.84-3.91 (m, 1H) 4.00 (dd, J=8.8, 6.1 Hz, 1H) 4.03-4.19 (m, 2H) 4.34-4.42 (m, 1H) 6.24 (br d, J=6.0 Hz, 1H) 7.21 (br s, 1H) 7.39 (br d, J=8.4 Hz, 2H) 7.54 (br d, J=8.4 Hz, 2H) 7.63 (s, 1H) 10.78 (br s, 1H)

### Step 6 (Examples 32 & 33): [4-[2-[[tetrahydrofuran-3-yl]amino]-5H-pyrrolo[2,3-b]pyrazin-7-yl]-1-piperidyl]-[4-(trifluoromethoxy)phenyl]methanone, Isomers 1 & 2

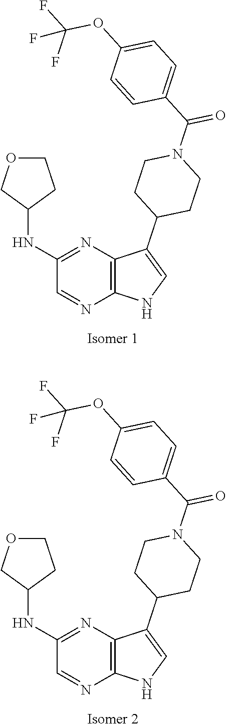 [SMILES: O=C(c1ccc(OC(F)(F)F)cc1)N1CCC(c2c[nH]c3ncc(NC4CCOC4)nc23)CC1 ; O=C(c1ccc(OC(F)(F)F)cc1)N1CCC(c2c[nH]c3ncc(NC4CCOC4)nc23)CC1]

Chiral separation of (rac)-[4-[2-[[tetrahydrofuran-3-yl]amino]-5H-pyrrolo[2,3-b]pyrazin-7-yl]-1-piperidyl]-[4-(trifluoromethoxy)phenyl]methanone (151 mg, 0.32 mmol) was performed using a Chiralcel OZ column (30 μm, 350×80 mm), eluting with (heptane 70/EtOH 30)+0.1% TEA (flow rate 400 mL/min, UV detection at 230 nm) to give 67 mg (44% yield) of [4-[2-[[tetrahydrofuran-3-yl]amino]-5H-pyrrolo[2,3-b]pyrazin-7-yl]-1-piperidyl]-[4-(trifluoromethoxy)phenyl]methanone, Isomer 1. LC/MS (m/z, M+H): calc. 476.2, found 476.4, ¹H NMR (400 MHz, DMSO-d₆, 100° C.) δ ppm 1.82-1.94 (m, 3H) 2.05 (br d, J=12.7 Hz, 2H) 2.20-2.29 (m, 1H) 3.05-3.19 (m, 3H) 3.57 (dd, J=8.8, 4.5 Hz, 1H) 3.72-3.79 (m, 1H) 3.83-3.91 (m, 1H) 4.00 (dd, J=8.8, 6.1 Hz, 1H) 4.03-4.19 (m, 2H) 4.34-4.42 (m, 1H) 6.24 (br d, J=6.0 Hz, 1H) 7.21 (d, J=2.9 Hz, 1H) 7.39 (br d, J=8.4 Hz, 2H) 7.54 (br d, J=8.4 Hz, 2H) 7.63 (s, 1H) 10.78 (br s, 1H); and 63 mg (42% yield) of [4-[2-[[tetrahydrofuran-3-yl]amino]-5H-pyrrolo[2,3-b]pyrazin-7-yl]-1-piperidyl]-[4-(trifluoromethoxy)phenyl]methanone, Isomer 2. LC/MS (m/z, M+H): calc. 476.2, found 476.4, ¹H NMR (400 MHz, DMSO-d₆, 100° C.) δ ppm 1.82-1.94 (m, 3H) 2.05 (br d, J=12.7 Hz, 2H) 2.20-2.29 (m, 1H) 3.05-3.19 (m, 3H) 3.57 (dd, J=8.8, 4.5 Hz, 1H) 3.72-3.79 (m, 1H) 3.84-3.91 (m, 1H) 4.00 (dd, J=8.8, 6.1 Hz, 1H) 4.04-4.19 (m, 2H) 4.34-4.42 (m, 1H) 6.24 (br d, J=6.0 Hz, 1H) 7.21 (d, J=2.9 Hz, 1H) 7.39 (br d, J=8.4 Hz, 2H) 7.54 (br d, J=8.4 Hz, 2H) 7.63 (s, 1H) 10.78 (br s, 1H).

### Examples 37, 34 & 35: (rac)-[2-amino-4-(trifluoromethoxy)phenyl]-[4-[2-[[tetrahydrofuran-3-yl]amino]-5H-pyrrolo[2,3-b]pyrazin-7-yl]-1-piperidyl]methanone, and [2-amino-4-(trifluoromethoxy)phenyl]-[4-[2-[[tetrahydrofuran-3-yl]amino]-5H-pyrrolo[2,3-b]pyrazin-7-yl]-1-piperidyl]methanone, Isomers 1 & 2

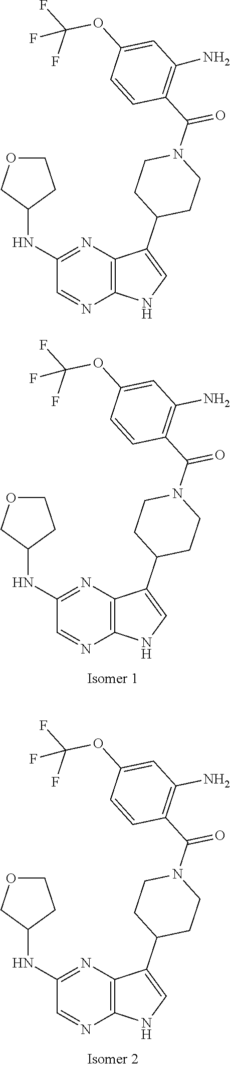 [SMILES: Nc1cc(OC(F)(F)F)ccc1C(=O)N1CCC(c2c[nH]c3ncc(NC4CCOC4)nc23)CC1 ; Nc1cc(OC(F)(F)F)ccc1C(=O)N1CCC(c2c[nH]c3ncc(NC4CCOC4)nc23)CC1 ; Nc1cc(OC(F)(F)F)ccc1C(=O)N1CCC(c2c[nH]c3ncc(NC4CCOC4)nc23)CC1]

### Step 1 (Example 37): (rac)-[2-amino-4-(trifluoromethoxy)phenyl]-[4-[2-[[tetrahydrofuran-3-yl]amino]-5H-pyrrolo[2,3-b]pyrazin-7-yl]-1-piperidyl]methanone

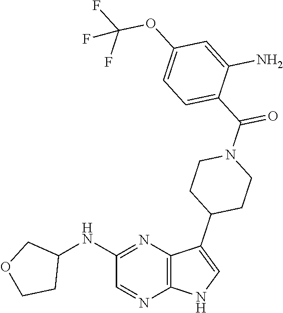 [SMILES: Nc1cc(OC(F)(F)F)ccc1C(=O)N1CCC(c2c[nH]c3ncc(NC4CCOC4)nc23)CC1]

Step 1 was performed following the protocol described in step 5 of Examples 32 & 33, using (rac)-7-(4-piperidyl)-N-tetrahydrofuran-3-yl-5H-pyrrolo[2,3-b]pyrazin-2-amine, trihydrochloride (182 mg, 0.46 mmol) and 2-amino-4-(trifluoromethoxy)benzoic acid (101 mg, 0.46 mmol) to give 189 mg (84% yield) of (rac)-[2-amino-4-(trifluoromethoxy)phenyl]-[4-[2-[[tetrahydrofuran-3-yl]amino]-5H-pyrrolo[2,3-b]pyrazin-7-yl]-1-piperidyl]methanone as a pale brown solid. LC/MS (m/z, M+H): calc. 491.2, found 491.4. ¹H NMR (400 MHz, DMSO-d₆, 100° C.) δ ppm 1.81-1.92 (m, 3H) 2.05 (br d, J=12.7 Hz, 2H) 2.20-2.29 (m, 1H) 3.03-3.17 (m, 3H) 3.57 (dd, J=8.8, 4.5 Hz, 1H) 3.72-3.78 (m, 1H) 3.83-3.90 (m, 1H) 4.00 (dd, J=8.8, 6.1 Hz, 1H) 4.09 (br d, J=12.7 Hz, 2H) 4.34-4.41 (m, 1H) 5.32 (br s, 2H) 6.23 (br d, J=6.0 Hz, 1H) 6.49 (br d, J=8.4 Hz, 1H) 6.69 (br s, 1H) 7.13 (d, J=8.4 Hz, 1H) 7.20 (br s, 1H) 7.63 (s, 1H) 10.77 (br s, 1H).

### Step 2 (Examples 34 & 35): [2-amino-4-(trifluoromethoxy)phenyl]-[4-[2-[[tetrahydrofuran-3-yl]amino]-5H-pyrrolo[2,3-b]pyrazin-7-yl]-1-piperidyl]methanone, Isomers 1 & 2

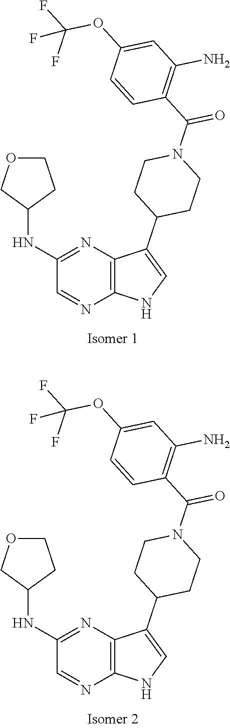 [SMILES: Nc1cc(OC(F)(F)F)ccc1C(=O)N1CCC(c2c[nH]c3ncc(NC4CCOC4)nc23)CC1 ; Nc1cc(OC(F)(F)F)ccc1C(=O)N1CCC(c2c[nH]c3ncc(NC4CCOC4)nc23)CC1]

Chiral separation of (rac)-[2-amino-4-(trifluoromethoxy)phenyl]-[4-[2-[[tetrahydrofuran-3-yl]amino]-5H-pyrrolo[2,3-b]pyrazin-7-yl]-1-piperidyl]methanone (175 mg, 0.36 mmol) was performed using a Chiralcel OZ column (5 μm, 30×250 mm), eluting with (heptane 55/EtOH 45)+0.1% TEA (flow rate 45 mL/min, UV detection at 254 nm) to give 78 mg (44% yield) of [2-amino-4-(trifluoromethoxy)phenyl]-[4-[2-[[tetrahydrofuran-3-yl]amino]-5H-pyrrolo[2,3-b]pyrazin-7-yl]-1-piperidyl]methanone, Isomer 1. LC/MS (m/z, M+H): calc. 491.2, found 491.4, ¹H NMR (400 MHz, DMSO-d₆, 100° C.) δ ppm 1.81-1.92 (m, 3H) 2.05 (br d, J=12.7 Hz, 2H) 2.20-2.30 (m, 1H) 3.03-3.17 (m, 3H) 3.57 (dd, J=8.8, 4.5 Hz, 1H) 3.72-3.79 (m, 1H) 3.83-3.90 (m, 1H) 3.99 (dd, J=8.8, 6.1 Hz, 1H) 4.09 (br d, J=12.7 Hz, 2H) 4.33-4.42 (m, 1H) 5.31 (br s, 2H) 6.22 (br d, J=6.0 Hz, 1H) 6.49 (br d, J=8.4 Hz, 1H) 6.69 (br s, 1H) 7.12 (d, J=8.4 Hz, 1H) 7.20 (br s, 1H) 7.63 (s, 1H) 10.76 (br s, 1H); and 84 mg (48% yield) of [2-amino-4-(trifluoromethoxy)phenyl]-[4-[2-[[tetrahydrofuran-3-yl]amino]-5H-pyrrolo[2,3-b]pyrazin-7-yl]-1-piperidyl]methanone, Isomer 2. LC/MS (m/z, M+H): calc. 491.2, found 491.4, ¹H NMR (400 MHz, DMSO-d₆, 100° C.) δ ppm 1.81-1.92 (m, 3H) 2.05 (br d, J=12.7 Hz, 2H) 2.20-2.30 (m, 1H) 3.03-3.17 (m, 3H) 3.57 (dd, J=8.8, 4.5 Hz, 1H) 3.72-3.78 (m, 1H) 3.83-3.90 (m, 1H) 3.99 (dd, J=8.8, 6.1 Hz, 1H) 4.09 (br d, J=12.7 Hz, 2H) 4.33-4.41 (m, 1H) 5.31 (br s, 2H) 6.22 (br d, J=6.0 Hz, 1H) 6.49 (br d, J=8.4 Hz, 1H) 6.69 (br s, 1H) 7.12 (d, J=8.4 Hz, 1H) 7.20 (br s, 1H) 7.63 (s, 1H) 10.76 (br s, 1H).

### Example 38: [4-[2-(tetrahydropyran-4-ylamino)-5H-pyrrolo[2,3-b]pyrazin-7-yl]-1-piperidyl]-[4-(trifluoromethoxy)phenyl]methanone

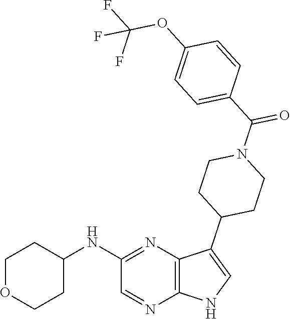 [SMILES: O=C(c1ccc(OC(F)(F)F)cc1)N1CCC(c2c[nH]c3ncc(NC4CCOCC4)nc23)CC1]

Example 38 was prepared using a method analogous to steps 1 to 5 of Examples 36, 32 & 33 to give 105 mg (79% yield in step 5) of [4-[2-(tetrahydropyran-4-ylamino)-5H-pyrrolo[2,3-b]pyrazin-7-yl]-1-piperidyl]-[4-(trifluoromethoxy)phenyl]methanone as a pale brown solid. LC/MS (m/z, M+H): calc. 490.2, found 490.3. ¹H NMR (400 MHz, DMSO-d₆, 100° C.) d ppm 1.47-1.58 (m, 2H), 1.82-1.95 (m, 2H), 1.95-2.08 (m, 4H), 3.04-3.18 (m, 3H), 3.44 (td, J=2.4 and 11.1 Hz, 2H), 3.85-3.97 (m, 3H), 4.01-4.17 (m, 2H), 5.94 (br d, J=7.0 Hz, 1H), 7.20 (s, 1H), 7.40 (d, J=8.0 Hz, 2H), 7.54 (d, =8.0 Hz, 2H), 7.62 (s, 1H), 10.74 (br s, 1H)

### Example 39: [2-amino-4-(trifluoromethoxy)phenyl]-[4-[2-(tetrahydropyran-4-ylamino)-5H-pyrrolo[2,3-b]pyrazin-7-yl]-1-piperidyl]methanone

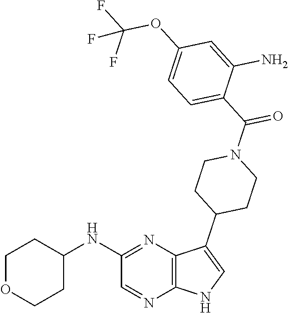 [SMILES: Nc1cc(OC(F)(F)F)ccc1C(=O)N1CCC(c2c[nH]c3ncc(NC4CCOCC4)nc23)CC1]

Example 39 was prepared following the protocol described in step 5 of Examples 36, 32 & 33 using 7-(4-piperidyl)-N-tetrahydropyran-4-yl-5H-pyrrolo[2,3-b]pyrazin-2-amine, trihydrochloride (111 mg, 0.27 mmol) and 2-amino-4-(trifluoromethoxy)benzoic acid (60 mg, 0.27 mmol) to give 106 mg (78% yield) of [2-amino-4-(trifluoromethoxy)phenyl]-[4-[2-(tetrahydropyran-4-ylamino)-5H-pyrrolo[2,3-b]pyrazin-7-yl]-1-piperidyl]methanone as a pale brown solid. LC/MS (m/z, M+H): calc. 505.2, found 505.3. ¹H NMR (400 MHz, DMSO-d₆, 100° C.) d ppm 1.47-1.58 (m, 2H), 1.81-1.93 (m, 2H), 1.96-2.09 (m, 4H), 3.03-3.18 (m, 3H), 3.45 (td, J=2.4 and 11.1 Hz, 2H), 3.85-3.96 (m, 3H), 4.08 (br d, J=12.7 Hz, 2H), 5.33 (br s, 2H), 5.93 (br d, J=7.0 Hz, 1H), 6.49 (br d, J=8.4 Hz, 1H), 6.70 (br s, 1H), 7.13 (d, J=8.4 Hz, 1H), 7.19 (br s, 1H), 7.62 (s, 1H), 10.73 (br s, 1H)

### Example 40: [4-(2-morpholino-5H-pyrrolo[2,3-b]pyrazin-7-yl)-1-piperidyl]-[4-(trifluoromethoxy)phenyl]methanone

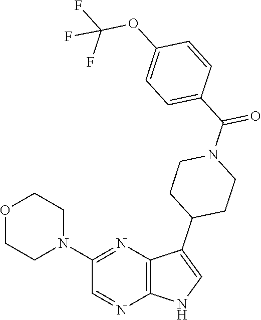 [SMILES: O=C(c1ccc(OC(F)(F)F)cc1)N1CCC(c2c[nH]c3ncc(N4CCOCC4)nc23)CC1]

### Step 1: tert-butyl 4-[2-morpholino-5-(2-trimethylsilylethoxymethyl)pyrrolo[2,3-b]pyrazin-7-yl]-3,6-dihydro-2H-pyridine-1-carboxylate

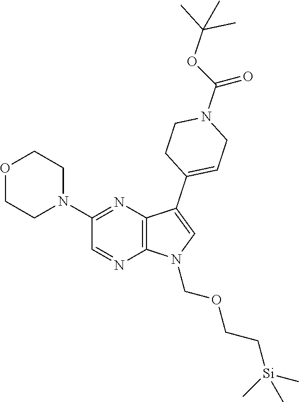 [SMILES: CC(C)(C)OC(=O)N1CC=C(c2cn(COCC[Si](C)(C)C)c3ncc(N4CCOCC4)nc23)CC1]

In a 10-20 mL microwave vial, a solution of morpholine (51 mg, 0.59 mmol), tert-butyl 4-[2-bromo-5-(2-trimethylsilylethoxymethyl)pyrrolo[2,3-b]pyrazin-7-yl]-3,6-dihydro-2H-pyridine-1-carboxylate (250 mg, 0.49 mmol, prepared in step 3 of Example 1), RuPhos (18 mg, 0.04 mmol) in THE (1 mL) was bubbled with argon for 10 minutes, and then, Pd(OAc)₂ (4 mg, 0.02 mmol) was added followed by 1.18 mL of a 1 M solution of LiHMDS (1.18 mmol) in THF. The resulting mixture was then submitted to microwave at 100° C. for one hour. After one hour, the reaction mixture was cooled down to room temperature, diluted with AcOEt and water. The aqueous layer was extracted three times with AcOEt and the combined organic layers were then washed with water, brine, dried over magnesium sulfate, filtered and concentrated in vacuo. The resulting residue was then purified by flash chromatography on silica gel (SiO₂ 24 g) eluting with DCM/AcOEt from 100/0 to 90/10 to give 140 mg (55% yield) of tert-butyl 4-[2-morpholino-5-(2-trimethylsilylethoxymethyl)pyrrolo[2,3-b]pyrazin-7-yl]-3,6-dihydro-2H-pyridine-1-carboxylate as a brown solid. LC/MS (m/z, M+H): calc. 516.3, found 516.4.

### Steps 2-5: [4-(2-morpholino-5H-pyrrolo[2,3-b]pyrazin-7-yl)-1-piperidyl]-[4-(trifluoromethoxy)phenyl]methanone

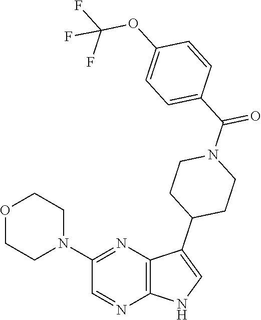 [SMILES: O=C(c1ccc(OC(F)(F)F)cc1)N1CCC(c2c[nH]c3ncc(N4CCOCC4)nc23)CC1]

Steps 2 to 5 of Example 40 were performed following the protocol described in steps 2 to 5 of Examples 36, 32 & 33. Using 4-[7-(4-piperidyl)-5H-pyrrolo[2,3-b]pyrazin-2-yl]morpholine; hydrochloride (50 mg, 0.15 mmol) and 4-(trifluoromethoxy)benzoic acid (32 mg, 0.15 mmol) in step 5 gave 30 mg (41% yield) of [4-(2-morpholino-5H-pyrrolo[2,3-b]pyrazin-7-yl)-1-piperidyl]-[4-(trifluoromethoxy)phenyl]methanone as a solid. LC/MS (m/z, M+H): calc. 476.2, found 476.1. ¹H NMR (400 MHz, DMSO-d₆, 100° C.) d ppm 1.74-1.86 (m, 2H), 2.07 (br d, J=12.7 Hz, 2H), 3.06-3.18 (m, 3H), 3.41-3.48 (m, 4H), 3.72-3.79 (m, 4H), 3.96-4.16 (m, 2H), 7.34 (d, J=2.9 Hz, 1H), 7.37 (br d, J=8.4 Hz, 2H), 7.51 (br d, J=8.4 Hz, 2H), 7.91 (s, 1H), 10.97 (br s, 1H)

### Example 41: [4-[2-(4,4-difluoro-1-piperidyl)-5H-pyrrolo[2,3-b]pyrazin-7-yl]-1-piperidyl]-[4-(trifluoromethoxy)phenyl]methanone

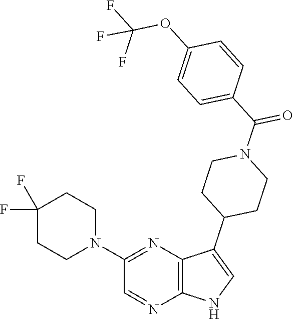 [SMILES: O=C(c1ccc(OC(F)(F)F)cc1)N1CCC(c2c[nH]c3ncc(N4CCC(F)(F)CC4)nc23)CC1]

### Step 1: tert-butyl 4-[2-(4,4-difluoro-1-piperidyl)-5-(2-trimethylsilylethoxymethyl)pyrrolo[2,3-b]pyrazin-7-yl]-3,6-dihydro-2H-pyridine-1-carboxylate

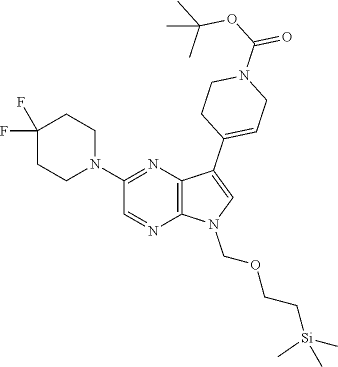 [SMILES: CC(C)(C)OC(=O)N1CC=C(c2cn(COCC[Si](C)(C)C)c3ncc(N4CCC(F)(F)CC4)nc23)CC1]

Step 1 of Example 41 was performed following the protocol described in step 1 of Example 40 using tert-butyl 4-[2-bromo-5-(2-trimethylsilylethoxymethyl)pyrrolo[2,3-b]pyrazin-7-yl]-3,6-dihydro-2H-pyridine-1-carboxylate (300 mg, 0.59 mmol) and 4,4-difluoropiperidine (85 mg, 0.71 mmol) to give 196 mg (60% yield) of tert-butyl 4-[2-(4,4-difluoro-1-piperidyl)-5-(2-trimethylsilylethoxymethyl)pyrrolo[2,3-b]pyrazin-7-yl]-3,6-dihydro-2H-pyridine-1-carboxylate as a brown solid. LC/MS (m/z, M+H): calc. 550.3, found 550.3.

### Steps 2-5: [4-[2-(4,4-difluoro-1-piperidyl)-5H-pyrrolo[2,3-b]pyrazin-7-yl]-1-piperidyl]-[4-(trifluoromethoxy)phenyl]methanone

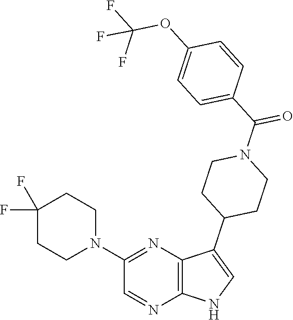 [SMILES: O=C(c1ccc(OC(F)(F)F)cc1)N1CCC(c2c[nH]c3ncc(N4CCC(F)(F)CC4)nc23)CC1]

Steps 2 to 5 of Example 41 were performed following the protocol described in steps 2 to 5 of Examples 36, 32 & 33. Using 2-(4,4-difluoro-1-piperidyl)-7-(4-piperidyl)-5H-pyrrolo[2,3-b]pyrazine, hydrochloride (45 mg, 0.12 mmol) and 4-(trifluoromethoxy)benzoic acid (26 mg, 0.12 mmol) in step 5 gave 35 mg (55% yield) of [4-[2-(4,4-difluoro-1-piperidyl)-5H-pyrrolo[2,3-b]pyrazin-7-yl]-1-piperidyl]-[4-(trifluoromethoxy)phenyl]methanone as a pale brown solid. LC/MS (m/z, M+H): calc. 510.2, found 510.1. ¹H NMR (400 MHz, DMSO-d₆, 100° C.) δ ppm 1.78-1.89 (m, 2H) 2.01-2.13 (m, 6H) 3.09-3.20 (m, 3H) 3.65-3.74 (m, 4H) 4.00-4.19 (m, 2H) 7.37 (d, J=2.5 Hz, 1H) 7.40 (br d, J=8.4 Hz, 2H) 7.54 (br d, J=8.4 Hz, 2H) 8.03 (s, 1H) 11.02 (br s, 1H)

### Example 42: [4-(2-tetrahydropyran-4-yl-5H-pyrrolo[2,3-b]pyrazin-7-yl)-1-piperidyl]-[4-[2,2,2-trifluoro-1-hydroxy-1-(trifluoromethyl)ethyl]phenyl]methanone

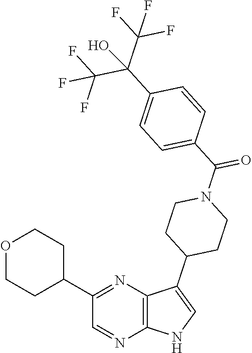 [SMILES: O=C(c1ccc(C(O)(C(F)(F)F)C(F)(F)F)cc1)N1CCC(c2c[nH]c3ncc(C4CCOCC4)nc23)CC1]

### Step 1: tert-butyl 4-(2-tetrahydropyran-4-yl-5H-pyrrolo[2,3-b]pyrazin-7-yl)piperidine-1-carboxylate

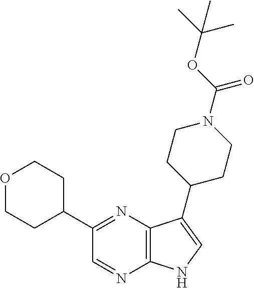 [SMILES: CC(C)(C)OC(=O)N1CCC(c2c[nH]c3ncc(C4CCOCC4)nc23)CC1]

Step 1 of Example 42 was performed following the protocol described in step 3 of Examples 36, 32 & 33 using tert-butyl 4-[2-tetrahydropyran-4-yl-5-(2-trimethylsilylethoxymethyl)pyrrolo[2,3-b]pyrazin-7-yl]piperidine-1-carboxylate (189 mg, 0.36 mmol, prepared in step 2 of Example 2) to give 128 mg (90% yield) of tert-butyl 4-(2-tetrahydropyran-4-yl-5H-pyrrolo[2,3-b]pyrazin-7-yl)piperidine-1-carboxylate as a solid. LC/MS (m/z, M+H): calc. 387.3, found 387.3.

### Step 2: 7-(4-piperidyl)-2-tetrahydropyran-4-yl-5H-pyrrolo[2,3-b]pyrazine; dihydrochloride

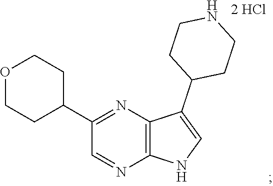 [SMILES: c1nc2[nH]cc(C3CCNCC3)c2nc1C1CCOCC1]

A solution of tert-butyl 4-(2-tetrahydropyran-4-yl-5H-pyrrolo[2,3-b]pyrazin-7-yl)piperidine-1-carboxylate (117 mg, 0.3 mmol) in HCl in dioxane (5N, 4 mL, 20 mmol) was stirred for one hour at room temperature. Then, the reaction mixture was concentrated to dryness to give 109 mg (100% yield) of 7-(4-piperidyl)-2-tetrahydropyran-4-yl-5H-pyrrolo[2,3-b]pyrazine; dihydrochloride as a yellow solid. LC/MS (m/z, M+H−2HCl): calc. 287.2, found 287.1.

### Step 3: [4-(2-tetrahydropyran-4-yl-5H-pyrrolo[2,3-b]pyrazin-7-yl)-1-piperidyl]-[4-[2,2,2-trifluoro-1-hydroxy-1-(trifluoromethyl)ethyl]phenyl]methanone

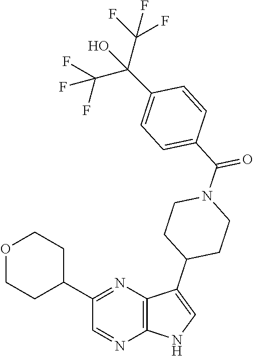 [SMILES: O=C(c1ccc(C(O)(C(F)(F)F)C(F)(F)F)cc1)N1CCC(c2c[nH]c3ncc(C4CCOCC4)nc23)CC1]

Step 3 of Example 42 was performed following the protocol described in step 5 of Examples 36, 32 & 33 using 7-(4-piperidyl)-2-tetrahydropyran-4-yl-5H-pyrrolo[2,3-b]pyrazine; dihydrochloride (50 mg, 0.14 mmol) and 4-(2-hydroxyhexafluoroisopropyl)benzoic acid (45 mg, 0.15 mmol) to give 67 mg (78% yield) of [4-(2-tetrahydropyran-4-yl-5H-pyrrolo[2,3-b]pyrazin-7-yl)-1-piperidyl]-[4-[2,2,2-trifluoro-1-hydroxy-1-(trifluoromethyl)ethyl]phenyl]methanone as a solid. LC/MS (m/z, M+H): calc. 557.2, found 557.1. ¹H NMR (400 MHz, DMSO-d₆, 100° C.) δ ppm 1.79-1.95 (m, 6H) 2.09 (br d, J=12.7 Hz, 2H) 3.03-3.26 (m, 4H) 3.50 (td, J=11.4, 2.9 Hz, 2H) 3.95-4.01 (m, 2H) 4.02-4.25 (m, 2H) 7.52 (s, 1H) 7.54 (br d, J=8.4 Hz, 2H) 7.76 (br d, J=8.4 Hz, 2H) 8.12 (s, 1H) 8.26-8.77 (m, 1H) 11.29 (br s, 1H)

### Example 43: (rac)-[2-amino-4-(pentafluoro-λ⁶-sulfanyl)phenyl]-[3-(2-tetrahydropyran-4-yl-5H-pyrrolo[2,3-b]pyrazin-7-yl)-129-yrrolidine-1-yl]methanone

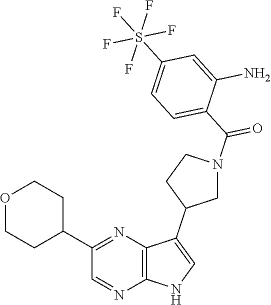 [SMILES: Nc1cc(S(F)(F)(F)(F)F)ccc1C(=O)N1CCC(c2c[nH]c3ncc(C4CCOCC4)nc23)C1]

Example 43 was prepared following the protocol described in steps 3 to 5 of Examples 36, 32 & 33. Using (rac)-7-pyrrolidin-3-yl-2-tetrahydropyran-4-yl-5H-pyrrolo[2,3-b]pyrazine, dihydrochloride (120 mg, 0.35 mmol) and 2-amino-4-(pentafluoro-λ⁶-sulfanyl)benzoic acid (100 mg, 0.38 mmol) in step 5 gave 53 mg (29% yield) of (rac)-[2-amino-4-(pentafluoro-λ⁶-sulfanyl)phenyl]-[3-(2-tetrahydropyran-4-yl-5H-pyrrolo[2,3-b]pyrazin-7-yl)pyrrolidin-1-yl]methanone as a white foam. LC/MS (m/z, M+H): calc. 518.2, found 518.2. ¹H NMR (400 MHz, DMSO-d₆, 100° C.) δ ppm 1.78-1.93 (m, 4H) 2.22-2.31 (m, 1H) 2.34-2.43 (m, 1H) 3.03-3.13 (m, 1H) 3.51 (td, J=11.1, 3.1 Hz, 2H) 3.56-3.76 (m, 4H) 3.93-4.03 (m, 3H) 5.60 (br s, 2H) 6.97 (dd, J=8.4, 2.3 Hz, 1H) 7.26 (d, J=2.3 Hz, 1H) 7.30 (br d, J=8.4 Hz, 1H) 7.60 (d, J=2.5 Hz, 1H) 8.15 (s, 1H) 11.38 (br s, 1H).

### Example 44: [2-amino-4-(trifluoromethoxy)phenyl]-[4-[2-(oxetan-3-yloxy)-5H-pyrrolo[2,3-b]pyrazin-7-yl]-1-piperidyl]methanone

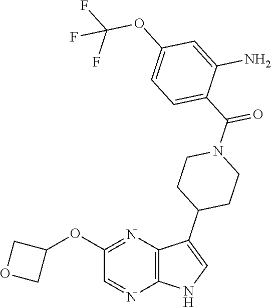 [SMILES: Nc1cc(OC(F)(F)F)ccc1C(=O)N1CCC(c2c[nH]c3ncc(OC4COC4)nc23)CC1]

### Step 1: tert-butyl 4-(2-bromo-5H-pyrrolo[2,3-b]pyrazin-7-yl)-3,6-dihydro-2H-pyridine-1-carboxylate

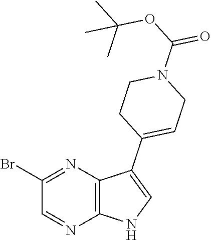 [SMILES: CC(C)(C)OC(=O)N1CC=C(c2c[nH]c3ncc(Br)nc23)CC1]

To a stirred solution of tert-butyl 4-[2-bromo-5-(2-trimethylsilylethoxymethyl)pyrrolo[2,3-b]pyrazin-7-yl]-3,6-dihydro-2H-pyridine-1-carboxylate (1.5 g, 2.94 mmol) in THE (15 mL), was added tetrabutylammonium fluoride; trihydrate (10 mL, 8.83 mmol) at 0° C. and then the reaction mixture was stirred for 6 hours at 70° C. After 6 hours, the reaction mixture was concentrated under reduced pressure. The resulting residue was purified by flash chromatography using 0-70% of ethyl acetate in hexane to give 750 mg (67% yield) of tert-butyl 4-(2-bromo-5H-pyrrolo[2,3-b]pyrazin-7-yl)-3,6-dihydro-2H-pyridine-1-carboxylate as a yellow solid. LC/MS (m/z, M−H): calc. 377.0, found 377.1.

### Step 2: 2-bromo-7-(1,2,3,6-tetrahydropyridin-4-yl)-5H-pyrrolo[2,3-b]pyrazine, hydrochloride

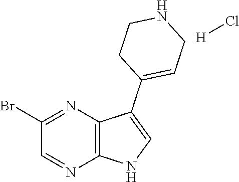

To a stirred solution of tert-butyl 4-(2-bromo-5H-pyrrolo[2,3-b]pyrazin-7-yl)-3,6-dihydro-2H-pyridine-1-carboxylate (0.750 g, 1.98 mmol) in 1,4-dioxane (10 mL), was added a 4M solution of HCl in dioxane (4 mL, 9.89 mmol) at 0° C. and the resulting mixture was then stirred for 3 hours at room temperature. After 3 hours, the reaction mixture was concentrated under reduced pressure and the resulting residue was triturated with n-hexane (2×20 mL) to give 600 mg (97% yield) of 2-bromo-7-(1,2,3,6-tetrahydropyridin-4-yl)-5H-pyrrolo[2,3-b]pyrazine, hydrochloride (0.600 g) as a brown solid. LC/MS (m/z, M+H−HCl): calc. 279.0, found 278.9.

### Step 3: [4-(2-bromo-5H-pyrrolo[2,3-b]pyrazin-7-yl)-3,6-dihydro-2H-pyridin-1-yl]-[2-nitro-4-(trifluoromethoxy)phenyl]methanone

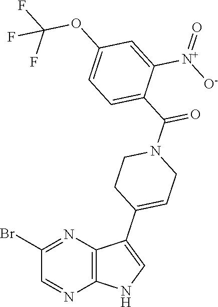 [SMILES: O=C(c1ccc(OC(F)(F)F)cc1[N+](=O)[O-])N1CC=C(c2c[nH]c3ncc(Br)nc23)CC1]

To a stirred solution of 2-nitro-4-(trifluoromethoxy)benzoic acid (400 mg, 1.59 mmol) and 2-bromo-7-(1,2,3,6-tetrahydropyridin-4-yl)-5H-pyrrolo[2,3-b]pyrazine, hydrochloride (489 mg, 1.55 mmol) in DMF (10 mL), was added N,N-diethylethanamine (484 mg, 4.78 mmol) and the resulting mixture was stirred for 15 minutes, then [benzotriazol-1-yloxy(dimethylamino)methylene]-dimethyl-ammonium; tetrafluoroborate (767 mg, 2.39 mmol) was added at 0° C. and the resulting mixture was stirred for 2 hours at room temperature then diluted with water (20 mL) and extracted with EtOAc (2×10 mL). The combined organic layers were dried over Na₂SO₄, concentrated under reduced pressure. The resulting residue was purified by flash chromatography using 0-70% of ethyl acetate in hexane to give 500 mg (63% yield) of [4-(2-bromo-5H-pyrrolo[2,3-b]pyrazin-7-yl)-3,6-dihydro-2H-pyridin-1-yl]-[2-nitro-4-(trifluoromethoxy)phenyl]methanone as a yellow solid. LC/MS (m/z, M+H): calc. 512.0, found 512.0.

### Step 4: [4-[2-bromo-5-(2-trimethylsilylethoxymethyl)pyrrolo[2,3-b]pyrazin-7-yl]-3,6-dihydro-2H-pyridin-1-yl]-[2-nitro-4-(trifluoromethoxy)phenyl]methanone

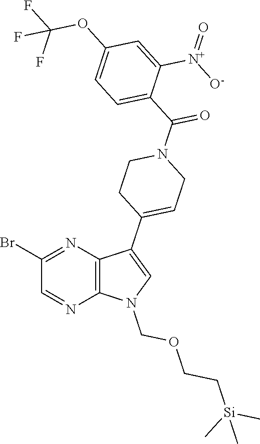 [SMILES: C[Si](C)(C)CCOCn1cc(C2=CCN(C(=O)c3ccc(OC(F)(F)F)cc3[N+](=O)[O-])CC2)c2nc(Br)cnc21]

To a stirred solution of [4-(2-bromo-5H-pyrrolo[2,3-b]pyrazin-7-yl)-3,6-dihydro-2H-pyridin-1-yl]-[2-nitro-4-(trifluoromethoxy)phenyl]methanone (500 mg, 0.98 mmol) in N,N-dimethylformamide (10 mL), was added sodium hydride (60% dispersion in oil, 59 mg, 1.46 mmol) portion wise at 0° C. and the resulting mixture was stirred for 20 min, then 2-(chloromethoxy)ethyl-trimethyl-silane (195 mg, 1.17 mmol) was added at 0° C. and the resulting mixture was stirred for 3 hours at room temperature. After 3 hours, the reaction mixture was diluted with water (20 mL) and extracted with EtOAc (2×100 mL). The combined organic layers were dried over Na₂SO₄, concentrated under reduced pressure. The resulting residue was purified by flash chromatography using 0-70% of ethyl acetate in hexane to give 500 mg (80% yield) of [4-[2-bromo-5-(2-trimethylsilylethoxymethyl)pyrrolo[2,3-b]pyrazin-7-yl]-3,6-dihydro-2H-pyridin-1-yl]-[2-nitro-4-(trifluoromethoxy)phenyl]methanone as a yellow gummy solid. LC/MS (m/z, M+H): calc. 642.1, found 642.1.

### Step 5: [2-nitro-4-(trifluoromethoxy)phenyl]-[4-[2-(oxetan-3-yloxy)-5-(2-trimethylsilylethoxymethyl)pyrrolo[2,3-b]pyrazin-7-yl]-3,6-dihydro-2H-pyridin-1-yl]methanone

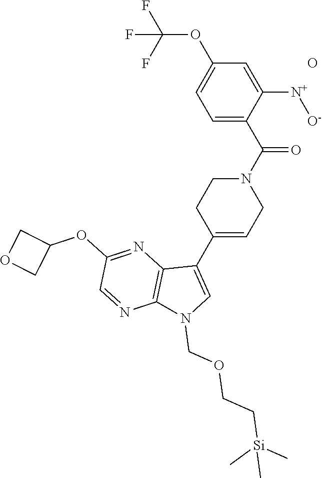

To a stirred solution of [4-[2-bromo-5-(2-trimethylsilylethoxymethyl)pyrrolo[2,3-b]pyrazin-7-yl]-3,6-dihydro-2H-pyridin-1-yl]-[2-nitro-4-(trifluoromethoxy)phenyl]methanone (450 mg, 0.70 mmol) in 1,4-dioxane (20 mL), were added oxetan-3-ol (259 mg, 3.50 mmol) and sodium 2-methylpropan-2-olate (202 mg, 2.1 mmol) at room temperature and the resulting mixture was stirred for 15 minutes, then XPhosPdG4 (60 mg, 0.07 mmol) was added under N₂ atmosphere and the resulting reaction mixture was submitted to microwave for 1 hour at 100° C. After one hour, the reaction mixture was filtered and dried under reduced pressure. The resulting residue was purified by flash chromatography, eluted with 0-60% of EtOAc in hexane, to give 250 mg (56% yield) of [2-nitro-4-(trifluoromethoxy)phenyl]-[4-[2-(oxetan-3-yloxy)-5-(2-trimethylsilylethoxymethyl)pyrrolo[2,3-b]pyrazin-7-yl]-3,6-dihydro-2H-pyridin-1-yl]methanone as a yellow liquid. LC/MS (m/z, M+H): calc. 636.2, found 636.3.

### Step 6: [2-amino-4-(trifluoromethoxy)phenyl]-[4-[2-(oxetan-3-yloxy)-5-(2-trimethylsilylethoxymethyl)pyrrolo[2,3-b]pyrazin-7-yl]-1-piperidyl]methanone

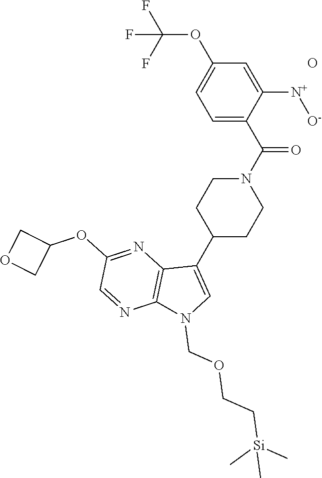

To a stirred solution of [2-nitro-4-(trifluoromethoxy)phenyl]-[4-[2-(oxetan-3-yloxy)-5-(2-trimethylsilylethoxymethyl)pyrrolo[2,3-b]pyrazin-7-yl]-3,6-dihydro-2H-pyridin-1-yl]methanone (250 mg, 0.39 mmol) in ethanol (10 mL), was added 10% palladium on carbon (126 mg, 1.18 mmol) at room temperature and the resulting mixture was stirred for 6 hours at room temperature under one atmosphere of hydrogen. After 6 hours, the reaction mixture was filtered, and the filtrate was concentrated under reduced pressure. The resulting residue was purified by flash chromatography, eluted with 0-80% of EtOAc in hexane, to give 110 mg (46% yield) of [2-amino-4-(trifluoromethoxy)phenyl]-[4-[2-(oxetan-3-yloxy)-5-(2-trimethylsilylethoxymethyl)pyrrolo[2,3-b]pyrazin-7-yl]-1-piperidyl]methanone as a gummy liquid. LC/MS (m/z, M+H): calc. 608.2, found 608.3.

### Step 7: [2-amino-4-(trifluoromethoxy)phenyl]-[4-[2-(oxetan-3-yloxy)-5H-pyrrolo[2,3-b]pyrazin-7-yl]-1-piperidyl]methanone

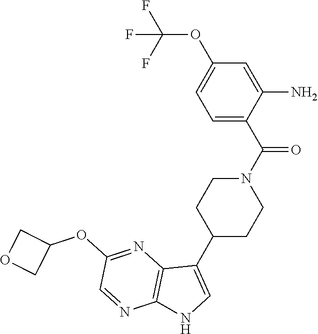 [SMILES: Nc1cc(OC(F)(F)F)ccc1C(=O)N1CCC(c2c[nH]c3ncc(OC4COC4)nc23)CC1]

To a stirred solution of [2-amino-4-(trifluoromethoxy)phenyl]-[4-[2-(oxetan-3-yloxy)-5-(2-trimethylsilylethoxymethyl)pyrrolo[2,3-b]pyrazin-7-yl]-1-piperidyl]methanone (100 mg, 0.165 mmol) in THE (5 mL), was added tetrabutylammonium fluoride; trihydrate (0.5 mL, 0.132 mmol) at 0° C. and the resulting mixture was stirred for 4 hours at 70° C. After 4 hours, the reaction mixture was concentrated under reduced pressure. The resulting residue was purified by preparative HPLC as follows: Mobile phase: A=0.1% HCOOH in water B=ACN; Column: Gemini NX (250 mm×21.2 mm); Flow rate: 18 mL/min) to give 16 mg (20% yield) of [2-amino-4-(trifluoromethoxy)phenyl]-[4-[2-(oxetan-3-yloxy)-5H-pyrrolo[2,3-b]pyrazin-7-yl]-1-piperidyl]methanone as an off white solid. LC/MS (m/z, M+H): calc. 478.2, found 478.1. ¹H NMR (400 MHz, DMSO-d₆) δ ppm 11.63-11.73 (m, 1H) 7.87-7.95 (m, 1H) 7.48-7.56 (m, 1H) 7.08-7.16 (m, 1H) 6.64-6.70 (m, 1H) 6.45-6.52 (m, 1H) 5.53-5.67 (m, 4H) 4.88-4.99 (m, 2H) 4.53-4.65 (m, 2H) 3.00-3.20 (m, 2H) 1.95-2.07 (m, 2H) 1.70-1.85 (m, 2H).

### Example 45—ERK5 Inhibitory Activity of Compounds

Two assays were performed to assess the ERK5 inhibitory activity of compounds of the Examples, a cell-based assay and a cell-free biochemical assay. Results of the assays are shown below in Table 2. Where repeat measurements were taken, the value reported is a mean average.

### Inhibition of Cellular ERK5 Activity

The renal cancer cell line SN12C was transduced by a lentivirus pGreenFire1 MEF2 EF1 Neo (ref TR030VA-N) from SBI using standard infection protocol. pGreenFire1 MEF2 EF1 Neo allows the expression of luciferase gene under the control of minimal promoter with MEF2 transcriptional response elements. Cells harboring the reporter construct were selected by geneticin treatment. The selected cells were then transposed by a piggyback based plasmid pCM4007 allowing the expression of constitutively activated MEK5DD under control of TREG3 promoter, a doxycycline regulated promoter. Transposed cells were selected by puromycin treatment. Upon doxycycline treatment (1 μg/ml) MEK5DD was expressed. MEK5DD activates ERK5 that phosphorylates MEF2C protein. Activated MEF2C protein can bind to its transcriptional response elements. Then the luciferase is expressed. In a 96-well plate (96F nuncleon ref137101 thermofisher), 50,000 cells were inoculated in 142.5 μl of RPMI medium containing 10% fetal calf serum, 1% glutamine, and 1 μg/ml doxycycline. After 24 hours, compounds were added in 7.5 μl culture medium (with 2% DMSO to give a final concentration of 0.1%) in order to obtain the desired concentration (0.3-10000 nM). The luciferase activity was measured using the Kit Bright Glo Luminescent Cell Assay Cat E2610 (Promega) according to the manufacturer's protocol. Luminescence was determined using 0.2 second reading/well using a Tecan SPARK. IC₅₀ values were calculated using XLFIT5 for Microsoft Excel using method 205. The IC₅₀ values represent the concentration of compound which inhibits the measurable luminescence signal by 50% as compared to DMSO-treated control cells.

### Cell-Free Assay of ERK5 Inhibition

An assay was performed to measure the capacity of each compound to inhibit ERK5 enzymatic activity. Compound potency was evaluated by Time-Resolved Forster Resonance Energy Transfer (FRET system). The activated catalytic domain of protein ERK5 (CarnaBiosciences #04-146) was mixed at 4 nM with varying concentrations of compound and incubated for 30 minutes at room temperature. A mixture of 1 mM ATP and 1 μM biotinylated synthetic peptide was added (Biosyntan GmbH). This synthetic peptide represents amino acids 30-52 of Eukaryotic translation initiation factor 4E-binding protein 1 (see, e.g., the sequence under accession No. NP_004086.1) biotinylated at the N-terminus. After 30 minutes at 37° C., the peptide phosphorylation by ERK5 was measured by addition of FRET reagents consisting of 12.5 g/ml Streptavidin-XL665, 1 nM Anti-P-4EBP1 antibody and 300 ng/ml Anti-rabbit-K antibody. Following 90 minutes at room temperature fluorescent signals were read on the Pherastar FSX multimod detector from BMG Labtech (Exc° 340 nm, Em1 620 nm, Em2 665 nm). The IC₅₀ values represent the concentration of compound which inhibits the measurable fluorescence signal by 5000 as compared to the DMSO only control.

TABLE 2
Results of cell-based and cell-free assays

| Compound | Cell-based assay | Cell-free assay |
|---|---|---|
| (Example No.) | (IC₅₀ in nM) | (IC₅₀ in nM) |
| 1 | 20 | 17 |
| 2 | 15 | 37 |
| 3 | 41 | 172 |
| 4 | 60 | 66 |
| 5 | 88 | 212 |
| 6 | 118 | 403 |
| 7 | 56 | 169 |
| 8 | 226 | 25900 |
| 9 | 156 | 2150 |
| 10 | 28 | 23 |
| 11 | 112 | 76 |
| 12 | 21 | 130 |
| 13 | 49 | 93 |
| 14 | 46 | 99 |
| 15 | 96 | 98 |
| 16 | 25 | 38 |
| 17 | 140 | 578 |
| 18 | 34 | 330 |
| 19 | 94 | 176 |
| 20 | 201 | 282 |
| 21 | 352 | 359 |
| 22 | 279 | 1138 |
| 23 | 4797 | 13328 |
| 24 | 261 | — |
| 25 | 230 | — |
| 26 | 149 | 624 |
| 27 | 5428 | 5559 |
| 28 | 190 | 405 |
| 29 | 215 | 399 |
| 30 | 17 | 39 |
| 31 | 86 | 189 |
| 32 | 79 | 361 |
| 33 | 126 | 335 |
| 34 | >10000 | 94 |
| 35 | 246 | 131 |
| 36 | 124 | 291 |
| 37 | 60 | 94 |
| 38 | 166 | 993 |
| 39 | 37 | 307 |
| 40 | 180 | 1009 |
| 41 | 308 | >30000 |
| 42 | 480 | >30000 |
| 43 | 357 | 9725 |
| 44 | 272 | 639 |

“—” denotes that the values were not measured

The data in Table 2 indicate that the compounds synthesised are active in the micromolar or nanomolar concentration range in cell-based and/or cell-free systems. All of the compounds synthesised have an IC₅₀ value below 10 μM in at least one of the cell-based and cell-free assays.

It is to be understood that while the disclosure has been described in conjunction with the above embodiments, that the foregoing description and examples are intended to illustrate and not limit the scope of the disclosure. Other aspects, advantages, and modifications within the scope of the disclosure will be apparent to those skilled in the art to which the disclosure pertains.

In addition, where features or aspects are described in terms of Markush groups, those skilled in the art will recognize that such features or aspects are also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. A compound, being of Formula (I) 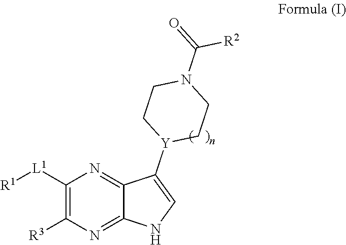
or a pharmaceutically acceptable salt thereof, wherein:
R¹ is selected from —(C₁-C₆)alkyl, —(C₃-C₇)cycloalkyl, and 4- to 10-membered heterocycloalkyl, wherein R¹ is optionally substituted with one or more occurrences of R^{A},
wherein each R^{A} is independently selected from halo, —OH, oxo, —NH₂, ═N—OH, —(C₁-C₃)alkyl, —O(C₁-C₃)alkyl, and —(C₃-C₆)cycloalkyl, wherein each occurrence of —(C₁-C₃)alkyl is optionally substituted by one or more groups independently selected from halo and —OH;
L¹ is selected from a direct bond, —O—, and —NH—;
R² is selected from —(C₃-C₆)cycloalkyl, and —(C₆-C₁₀)aryl, wherein R² is optionally substituted with one, two, or three occurrences of R^{B},
wherein each R^{B} is independently selected from halo, —NH₂, —SF₅, —(C₁-C₃)alkyl, —O(C₁-C₃)alkyl, and —O(C₃-C₆)cycloalkyl, wherein each occurrence of —(C₁-C₃)alkyl and —O(C₁-C₃)alkyl is optionally substituted by one or more groups independently selected from halo and —OH;
R³ is selected from —H, (C₁-C₃)alkyl (e.g., —CH₃), and —OH;
Y is CH, or N; and
n is 0, or 1.

2. The compound of claim 1, wherein R¹ is selected from: 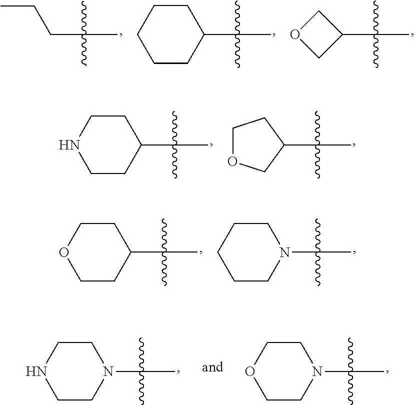
wherein R¹ is optionally substituted by one or two occurrences of R^{A} as defined in claim 1.

3. The compound of claim 2, wherein each R^{A} is independently selected from the group consisting of —F, —OH, methyl, —OCH₃, and cyclopropyl.

4. The compound of any one of claims 1-3, wherein the compound is a compound of Formula (I-G) 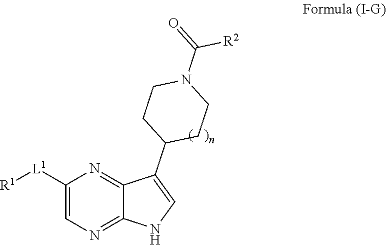
or a pharmaceutically acceptable salt thereof, wherein R¹, R², L¹, and n are as defined in any one of claims 1-3.

5. A compound, being of Formula (II) 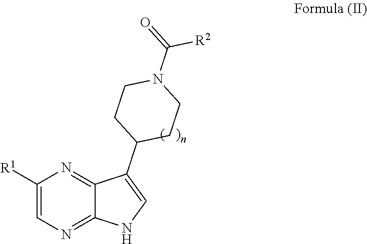
or a pharmaceutically acceptable salt thereof, wherein:
R¹ is selected from —(C₁-C₆)alkyl, —(C₃-C₇)cycloalkyl, and 4- to 10-membered heterocycloalkyl, wherein R¹ is optionally substituted with one or more occurrences of R^{A},
wherein each R^{A} is independently selected from halo (e.g., —F), —OH, —(C₁-C₃)alkyl, —O(C₁-C₃)alkyl, and —(C₃-C₆)cycloalkyl;
R² is selected from —(C₃-C₆)cycloalkyl, and —(C₆-C₁₀)aryl, wherein R² is optionally substituted with one or two occurrences of R^{B},
wherein each R^{B} is independently selected from halo, —NH₂, —SF₅, —(C₁-C₃)alkyl, —O(C₁-C₃)alkyl, and —O(C₃-C₆)cycloalkyl, wherein each occurrence of —(C₁-C₃)alkyl and —O(C₁-C₃)alkyl is substituted by one or more groups independently selected from halo and —OH; and
n is 0, or 1.

6. The compound of claim 5, wherein R² is —(C₆-C₁₀)aryl substituted with one or two occurrences of R^{B}, wherein each R^{B} is independently selected from halo (e.g., —F), —NH₂, —SF₅, —OCF₃, —O-cyclopropyl, —C(OH)(CF₃)₂, and —CF₂CF₃.

7. A compound, being of Formula (III-A) 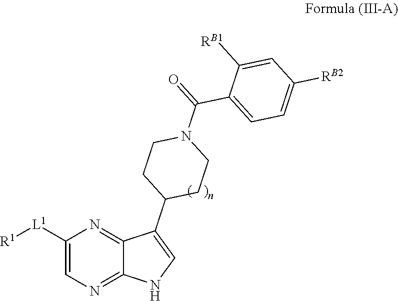
or a pharmaceutically acceptable salt thereof, wherein R¹, L¹, and n are as defined in any one of claims 1-3, and wherein:
R^{B1} is either —H, or is selected from the group consisting of —NH₂, and —F; and
R^{B2} is selected from the group consisting of —OCF₃, —SF₅, —CF₂CF₃, —C(OH)(CF₃)₂, and —O-cyclopropyl.

8. A compound, being of Formula (IV-A) or Formula (V-A) 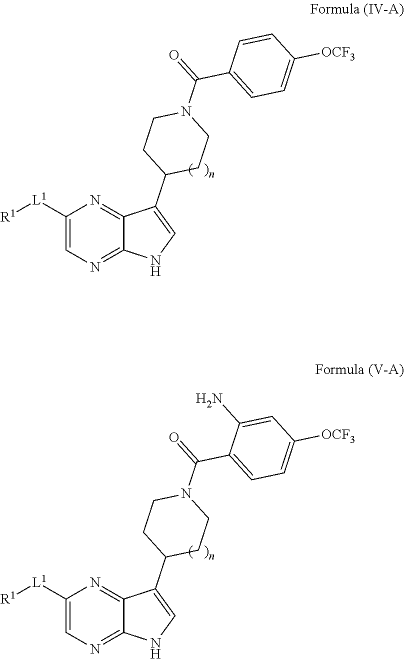
or a pharmaceutically acceptable salt thereof, wherein R¹, L¹, and n are as defined in any one of claims 1-3.

9. A compound, being of Formula (VI), Formula (VII), or Formula (VIII) 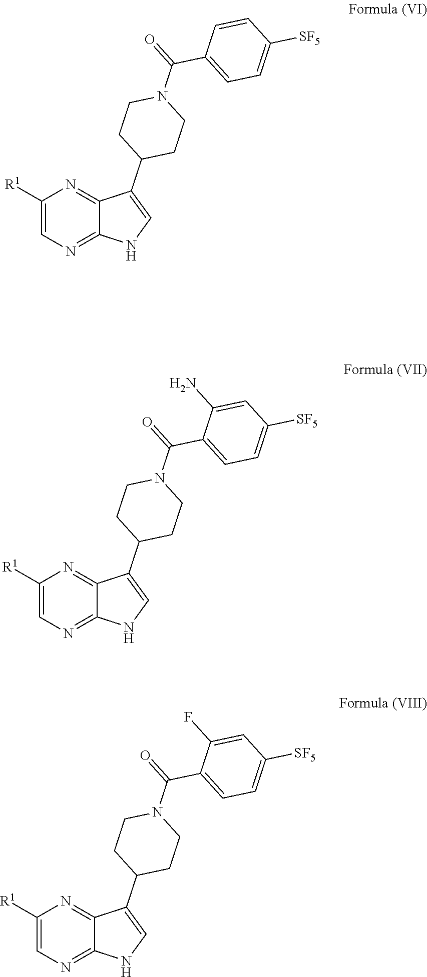
or a pharmaceutically acceptable salt thereof, wherein R¹ is as defined in any one of claims 1-3.

10. A compound selected from the group consisting of:
[2-amino-4-(trifluoromethoxy)phenyl]-[4-[2-(1-methyl-4-piperidyl)-5H-pyrrolo[2,3-b]pyrazin-7-yl]-1-piperidyl]methanone,
[2-amino-4-(trifluoromethoxy)phenyl]-[4-(2-tetrahydropyran-4-yl-5H-pyrrolo[2,3-b]pyrazin-7-yl)-1-piperidyl]methanone,
[4-(2-tetrahydropyran-4-yl-5H-pyrrolo[2,3-b]pyrazin-7-yl)-1-piperidyl]-[4-(trifluoromethoxy)phenyl]methanone,
[4-[2-(1-methyl-4-piperidyl)-5H-pyrrolo[2,3-b]pyrazin-7-yl]-1-piperidyl]-[4-(trifluoromethoxy)phenyl]methanone,
[4-(2-tetrahydrofuran-3-yl-5H-pyrrolo[2,3-b]pyrazin-7-yl)-1-piperidyl]-[4-(trifluoromethoxy)phenyl]methanone,
[4-[2-(3-methoxypropyl)-5H-pyrrolo[2,3-b]pyrazin-7-yl]-1-piperidyl]-[4-(trifluoromethoxy)phenyl]methanone,
[2-amino-4-(trifluoromethoxy)phenyl]-[4-[2-(3-methoxypropyl)-5H-pyrrolo[2,3-b]pyrazin-7-yl]-1-piperidyl]methanone,
[4-(2-cyclohexyl-5H-pyrrolo[2,3-b]pyrazin-7-yl)-1-piperidyl]-[4-(trifluoromethoxy)phenyl]methanone,
[2-amino-4-(trifluoromethoxy)phenyl]-[4-(2-cyclohexyl-5H-pyrrolo[2,3-b]pyrazin-7-yl)-1-piperidyl]methanone,
[2-amino-4-(trifluoromethoxy)phenyl]-[4-[2-(1-cyclopropyl-4-piperidyl)-5H-pyrrolo[2,3-b]pyrazin-7-yl]-1-piperidyl]methanone,
[4-[2-(1-cyclopropyl-4-piperidyl)-5H-pyrrolo[2,3-b]pyrazin-7-yl]-1-piperidyl]-[4-(trifluoromethoxy)phenyl]methanone,
[2-amino-4-(trifluoromethoxy)phenyl]-[4-(2-tetrahydrofuran-3-yl-5H-pyrrolo[2,3-b]pyrazin-7-yl)-1-piperidyl]methanone,
[4-[2-(1-methyl-4-piperidyl)-5H-pyrrolo[2,3-b]pyrazin-7-yl]-1-piperidyl]-[4-(pentafluoro-λ⁶-sulfanyl)phenyl]methanone,
[2-amino-4-(pentafluoro-λ⁶-sulfanyl)phenyl]-[4-[2-(1-methyl-4-piperidyl)-5H-pyrrolo[2,3-b]pyrazin-7-yl]-1-piperidyl]methanone,
[4-(pentafluoro-λ⁶-sulfanyl)phenyl]-[4-(2-tetrahydropyran-4-yl-5H-pyrrolo[2,3-b]pyrazin-7-yl)-1-piperidyl]methanone,
[2-amino-4-(pentafluoro-λ⁶-sulfanyl)phenyl]-[4-(2-tetrahydropyran-4-yl-5H-pyrrolo[2,3-b]pyrazin-7-yl)-1-piperidyl]methanone,
[2-amino-4-(pentafluoro-λ⁶-sulfanyl)phenyl]-[4-[2-(1-cyclopropyl-4-piperidyl)-5H-pyrrolo[2,3-b]pyrazin-7-yl]-1-piperidyl]methanone,
[4-[2-(1-cyclopropyl-4-piperidyl)-5H-pyrrolo[2,3-b]pyrazin-7-yl]-1-piperidyl]-[4-(pentafluoro-λ⁶-sulfanyl)phenyl]methanone,
[4-(cyclopropoxy)phenyl]-[4-(2-tetrahydropyran-4-yl-5H-pyrrolo[2,3-b]pyrazin-7-yl)-1-piperidyl]methanone,
[4-(1,1,2,2,2-pentafluoroethyl)phenyl]-[4-(2-tetrahydropyran-4-yl-5H-pyrrolo[2,3-b]pyrazin-7-yl)-1-piperidyl]methanone,
cyclohexyl-[4-(2-tetrahydropyran-4-yl-5H-pyrrolo[2,3-b]pyrazin-7-yl)-1-piperidyl]methanone,
[2-amino-4-(trifluoromethoxy)phenyl]-[3-(2-tetrahydropyran-4-yl-5H-pyrrolo[2,3-b]pyrazin-7-yl)pyrrolidine-1-yl]methanone,
[3-(2-tetrahydropyran-4-yl-5H-pyrrolo[2,3-b]pyrazin-7-yl)pyrrolidin-1-yl]-[4-(trifluoromethoxy)phenyl]methanone,
[2-fluoro-4-(pentafluoro-λ⁶-sulfanyl)phenyl]-[4-(2-tetrahydropyran-4-yl-5H-pyrrolo[2,3-b]pyrazin-7-yl)-1-piperidyl]methanone,
[2-amino-4-(trifluoromethoxy)phenyl]-[4-[2-(oxetan-3-ylamino)-5H-pyrrolo[2,3-b]pyrazin-7-yl]-1-piperidyl]methanone,
[2-amino-4-(trifluoromethoxy)phenyl]-[4-[2-[tetrahydrofuran-3-yl]oxy-5H-pyrrolo[2,3-b]pyrazin-7-yl]-1-piperidyl]methanone,
trans-[2-amino-4-(trifluoromethoxy)phenyl]-[4-[2-(4-hydroxycyclohexyl)-5H-pyrrolo[2,3-b]pyrazin-7-yl]-1-piperidyl]methanone,
[4-[2-(4-methylpiperazin-1-yl)-5H-pyrrolo[2,3-b]pyrazin-7-yl]-1-piperidyl]-[4-(trifluoromethoxy)phenyl]methanone,
[4-[2-[[tetrahydrofuran-3-yl]amino]-5H-pyrrolo[2,3-b]pyrazin-7-yl]-1-piperidyl]-[4-(trifluoromethoxy)phenyl]methanone,
[2-amino-4-(trifluoromethoxy)phenyl]-[4-[2-[[tetrahydrofuran-3-yl]amino]-5H-pyrrolo[2,3-b]pyrazin-7-yl]-1-piperidyl]methanone,
[4-[2-(tetrahydropyran-4-ylamino)-5H-pyrrolo[2,3-b]pyrazin-7-yl]-1-piperidyl]-[4-(trifluoromethoxy)phenyl]methanone,
[2-amino-4-(trifluoromethoxy)phenyl]-[4-[2-(tetrahydropyran-4-ylamino)-5H-pyrrolo[2,3-b]pyrazin-7-yl]-1-piperidyl]methanone,
[4-(2-morpholino-5H-pyrrolo[2,3-b]pyrazin-7-yl)-1-piperidyl]-[4-(trifluoromethoxy)phenyl]methanone,
[4-[2-(4,4-difluoro-1-piperidyl)-5H-pyrrolo[2,3-b]pyrazin-7-yl]-1-piperidyl]-[4-(trifluoromethoxy)phenyl]methanone,
[4-(2-tetrahydropyran-4-yl-5H-pyrrolo[2,3-b]pyrazin-7-yl)-1-piperidyl]-[4-[2,2,2-trifluoro-1-hydroxy-1-(trifluoromethyl)ethyl]phenyl]methanone,
[2-amino-4-(pentafluoro-λ⁶-sulfanyl)phenyl]-[3-(2-tetrahydropyran-4-yl-5H-pyrrolo[2,3-b]pyrazin-7-yl)pyrrolidin-1-yl]methanone,
[2-amino-4-(trifluoromethoxy)phenyl]-[4-[2-(oxetan-3-yloxy)-5H-pyrrolo[2,3-b]pyrazin-7-yl]-1-piperidyl]methanone,
and the pharmaceutically acceptable salts thereof.

11. A pharmaceutical composition comprising the compound of any one of claims 1-10 and at least one pharmaceutically acceptable excipient or carrier.

12. A compound according to any one of claims 1-10, or a pharmaceutical composition according to claim 11, for use in therapy.

13. A compound according to any one of claims 1-10, or a pharmaceutical composition according to claim 11, for use in the treatment or prevention of cancer.

14. The compound or pharmaceutical composition for use according to claim 13, wherein the cancer is characterized by increased MAPK7 expression and/or increased ERK5 activity.

15. The compound or pharmaceutical composition for use according to claim 13 or claim 14, wherein the cancer is selected from leukaemia, breast cancer, multiple myeloma, colon cancer, colorectal cancer, lung cancer, pancreatic cancer, renal cell carcinoma, mesothelioma, adenocarcinoma, neuroblastoma, melanoma, and hepatocellular carcinoma.
